(19) United States
(12) Patent Application Publication
(10) Pub. No.: US 2023/0348958 A1
(43) Pub. Date: Nov. 2, 2023

(54) GENOME-SCALE IMAGING OF THE 3D ORGANIZATION AND TRANSCRIPTIONAL ACTIVITY OF CHROMATIN

(71) Applicant: President and Fellows of Harvard College, Cambridge, MA (US)

(72) Inventors: Xiaowei Zhuang, Cambridge, MA (US); Bogdan Bintu, Cambridge, MA (US); Seon S. Kinrot, Cambridge, MA (US); Pu Zheng, Cambridge, MA (US); Jun-Han Su, Cambridge, MA (US)

(73) Assignee: President and Fellows of Harvard College, Cambridge, MA (US)

(21) Appl. No.: 17/770,943

(22) PCT Filed: Dec. 18, 2020
(86) PCT No.: PCT/US2020/065797

§ 371 Date: Apr. 21, 2022

Related U.S. Application Data
(60) Provisional application No. 63/060,947, filed on Aug. 4, 2020.
(60) Provisional application No. 62/954,720, filed on Dec. 30, 2019.

(51) Int. Cl. C12Q 1/6841; G16B 40/10; G16B 15/10

(52) U.S. Cl. CPC C12Q 1/6841; G16B 40/10; G16B 15/10; C12Q 2600/16

(57) ABSTRACT

The present invention generally relates to genomics. Some embodiments are directed to imaging the 3D organization of the genome, or part of the genome, with high throughput in the sequence space. Some embodiments are directed to imaging the 3D organization of the genome, or part of the genome, in the context of transcriptional activity and nuclear structures. In addition, certain embodiments are directed to chromatin structures, 3D chromatin organizations, trans-chromosomal interactions and chromatin-nuclear-structure interactions as well as their relationship with transcription, etc. In addition, various embodiments are directed to imaging methods that allow mapping of the 3D organization of the genome, or part of the genome, in the context of nuclear structures and transcriptional activity. Some embodiments are directed to massively multiplexed fluorescence in situ hybridization methods for imaging chromatin loci and/or nascent RNA transcripts at the chromosome or genome scale.

### RELATED APPLICATIONS

This application claims the benefit of U.S. Provisional Patent Application Ser. No. 62/954,720, filed Dec. 30, 2019, entitled “Genome-Scale Imaging of the 3D Organization and Transcriptional Activity of Chromatin,” by Zhuang, et al., and U.S. Provisional Patent Application Ser. No. 63/060,947, filed Aug. 4, 2020, entitled “Genome-Scale Imaging of the 3D Organization and Transcriptional Activity of Chromatin,” by Zhuang, et al. Each of these is incorporated herein by reference in its entirety.

### FIELD

The present invention generally relates to genomics. Some embodiments are directed to imaging the 3D organization of the genome in the context of transcriptional activity and nuclear structures. In addition, certain embodiments are directed to chromatin organization and chromatin-nuclear-structure interactions as well as their relationship with transcription.

### BACKGROUND

The three-dimensional (3D) organization of the genome regulates many essential cellular functions ranging from gene expression to DNA replication. Biochemical and imaging measurements have revealed complex chromatin structures across a wide range of scales. Recently, high-throughput chromosome conformation capture methods, such as Hi-C and other sequencing-based methods, have greatly enriched knowledge of the 3D genome organization, revealing chromatin structures such as loops, domains, and compartments with a genome-wide view. These powerful sequencing-based approaches also have limitations. For instance, these methods provide contact information between pairs of chromatin loci but do not provide direct spatial position information for these loci. Furthermore, most genome-wide insights on chromatin organization are built on population-averaged contact maps across millions of cells. Despite continuous improvement of single-cell Hi-C methods, the capture efficiency of chromatin contacts in single cells and/or the cell throughput of these methods remain relatively low, and hence investigation of 3D genome organization in single cells remains a challenging task. In addition, although methods have emerged to combine Hi-C with other measurement modalities, for example, to provide characterizations of chromatin contacts in the context of interacting proteins, nuclear structures, or DNA modifications, multi-modal measurement by sequencing remains challenging. Notably, a method that allows genome-scale measurements of both chromatin organization and transcriptional activity in the same cells has not emerged, but is in great demand because of the critical importance to understand how chromatin organization regulates transcription and how transcription in turn impacts chromatin organization.

Imaging-based approaches, on the other hand, provide a direct measure of the spatial positions of chromatin loci in individual cells with a high detection efficiency. In particular, fluorescence in situ hybridization (FISH) allows highly specific detection of chromatin loci in fixed cells and, more recently, the clustered regularly interspersed short palindromic repeats (CRISPR) system has substantially enhanced our ability to image specific chromatin loci in live cells. Chromatin imaging can also be combined with RNA and protein imaging to reveal the interplay between chromatin organization and transcriptional activity or interacting protein factors. However, current imaging methods are limited in throughput in the sequence space, traditionally allowing the study of only a few different genomic loci at a time. Genome-scale imaging would require a drastic increase in the number of genomic loci imaged in individual cells. Thus, new improvements are necessary.

### SUMMARY

The present invention generally relates to genomics. Some embodiments are directed to imaging the 3D organization of the genome, or part of the genome, with high throughput in the sequence space. Some embodiments are directed to imaging the 3D organization of the genome, or part of the genome, in the context of transcriptional activity and nuclear structures. In addition, certain embodiments are directed to chromatin structures, 3D chromatin organizations, trans-chromosomal interactions and chromatin-nuclear-structure interactions as well as their relationship with transcription, etc. The subject matter of the present disclosure involves, in some cases, interrelated products, alternative solutions to a particular problem, and/or a plurality of different uses of one or more systems and/or articles.

Certain aspects are generally directed to systems and methods of using multiplexed FISH, and in some cases using multiplexed error-robust FISH (MERFISH), to image chromatin, e.g., in a cell. In addition, certain aspects are generally directed to systems and methods of imaging and/or determining at least 100 or at least 500 distinct genomic loci in a single cell. Some aspects are generally directed to systems and methods of using FISH to image chromatin, e.g., in a cell.

In one set of embodiments, the method comprises associating a plurality of nucleic acid targets of a genome with a plurality of codewords, wherein the codewords comprise a number of positions and values for each position; exposing a sample containing the genome to a plurality of nucleic acid probes; for each nucleic acid probe of the plurality of nucleic acid probes, determining binding of the nucleic acid probe within the sample; creating codewords corresponding to the binding of the plurality of nucleic acid probes within the sample; and determining the identities of the nucleic acid targets based on the codeword assigned.

The method, in another set of embodiments, comprises determining positions of nascent RNA within a nucleus; applying RNAse to the nucleus; and determining positions of DNA within the nucleus.

In one set of embodiments, the method comprises using MERFISH to image chromatin in a cell. In another set of embodiments, the method comprises imaging at least 100 or at least 500 distinct genomic loci in a single cell.

According to one set of embodiments, the method comprises associating a plurality of nucleic acid targets of a genome with a plurality of codewords; exposing a sample containing a cell suspected of containing the genome to a plurality of nucleic acid probes, wherein at least some of the plurality of nucleic acid probes comprises a first portion comprising a target sequence and a second portion comprising one or more readout sequences, wherein each readout sequence represents a value of a position within the plurality of codewords; exposing the sample to a round of one or more adaptors, wherein each adaptor comprises a first portion substantially complementary to one of the readout sequences, and a second portion comprising one identification sequence; exposing the sample to a round of one or more readout probes to determine one or more identification sequences, wherein each readout probe comprises a first portion comprising a sequence substantially complementary to one of the identification sequences, and a second portion comprising a signaling entity; determining the signaling entity in at least some locations in the sample; and inactivating the signaling entity in at least some locations in the sample; repeating the steps of exposing the sample to a round of one or more adaptors and one or more readout probes, determining the signaling entity, and inactivating the signaling entity, wherein one or more distinct signaling entities are used in each of the rounds; determining codewords at the locations based on determining the signaling entity in the sample; and determining nucleic acid targets in the sample based on the codewords.

In yet another set of embodiments, the method comprises associating a plurality of nucleic acid targets of a genome with a plurality of codewords; exposing a sample containing a cell suspected of containing the genome to a plurality of nucleic acid probes, wherein at least some of the plurality of nucleic acid probes comprises a first portion comprising a target sequence and a second portion comprising one or more readout sequences, wherein each readout sequence represents a value of a position within the plurality of codewords; exposing the sample to a round of one or more adaptors, wherein each adaptor comprises a first portion substantially complementary to one of the readout sequences, and a second portion comprising one identification sequence; exposing the sample to a round of one or more readout probes to determine one or more identification sequences, wherein each readout probe comprises a first portion comprising a sequence substantially complementary to one of the identification sequences, and a second portion comprising a signaling entity; determining the signaling entity in at least some locations in the sample; and inactivating the signaling entity in at least some locations in the sample; repeating the steps of exposing the sample to a round of one or more adaptors and one or more readout probes, determining the signaling entity, and inactivating the signaling entity, wherein at least one of the signaling entities is used in more than one of the rounds; determining codewords at the locations based on determining the signaling entity in the sample; and determining nucleic acid targets in the sample based on the codewords.

In still another set of embodiments, the method comprises exposing a sample containing a cell suspected of containing the genome to a plurality of nucleic acid probes, wherein at least some of the plurality of nucleic acid probes comprises a first portion comprising a target sequence and a second portion comprising one or more readout sequences; exposing the sample to a round of one or more adaptors, wherein each adaptor comprises a first portion substantially complementary to one of the readout sequences, and a second portion comprising one identification sequence; exposing the sample to a round of one or more readout probes to determine one or more identification sequences, wherein each readout probe comprises a first portion comprising a sequence substantially complementary to one of the identification sequences, and a second portion comprising a signaling entity; determining the signaling entity in at least some locations in the sample; and inactivating the signaling entity in at least some locations in the sample; repeating the steps of exposing the sample to a round of one or more adaptors and one or more readout probes, determining the signaling entity, and inactivating the signaling entity, wherein one or more distinct signaling entities are used in each of the rounds; determining nucleic acid targets in the sample based on the signaling entities determined in each round.

The method, in another set of embodiments, comprises exposing a sample containing a cell suspected of containing the genome to a plurality of nucleic acid probes, wherein at least some of the plurality of nucleic acid probes comprises a first portion comprising a target sequence and a second portion comprising one or more readout sequences; exposing the sample to a round of one or more readout probes to determine one or more readout sequences, wherein each readout probe comprises a first portion comprising a sequence substantially complementary to one of the readout sequences, and a second portion comprising a signaling entity; determining the signaling entity in at least some locations in the sample; and inactivating the signaling entity in at least some locations in the sample; repeating the steps of exposing the sample to a round of one or more readout probes, determining the signaling entity, and inactivating the signaling entity, wherein one or more distinct signaling entities are used in each of the rounds; determining nucleic acid targets in the sample based on the signaling entities determined in each round.

In yet another set of embodiments, the method comprises exposing a sample containing a cell suspected of containing the genome to a round of a plurality of nucleic acid probes, wherein at least some of the plurality of nucleic acid probes comprises a first portion comprising a target sequence and a second portion comprising a signaling entity; determining the signaling entity in at least some locations in the sample; and inactivating the signaling entity in at least some locations in the sample; repeating the steps of exposing the sample to a round of a plurality of nucleic acid probes, determining the signaling entity, and inactivating the signaling entity, wherein one or more distinct signaling entities are used in each of the rounds; determining nucleic acid targets in the sample based on the signaling entities determined in each round.

In one set of embodiments, the method includes associating a plurality of nucleic acid targets of a genome with a plurality of codewords, wherein the codewords comprise a number of positions and values for each position and the codewords form an error-checking and/or error-correcting code space, and wherein the plurality of nucleic acid targets are separated by at least 100,000 nucleotides within the genome; exposing a nucleus of a cell containing the genome to a plurality of nucleic acid probes, wherein at least some of the plurality of nucleic acid probes comprises a first portion comprising a target sequence and a second portion comprising one or more read sequences, wherein each read sequence represents a value of a position within the codewords; for each nucleic acid probe of the plurality of nucleic acid probes, determining binding of the nucleic acid probe within the nucleus; creating codewords corresponding to the binding of the plurality of nucleic acid probes within the nucleus, wherein the values of the digits of the codewords are based on the read sequences present on the nucleic acid probes; for at least some of the codewords, matching the codeword to a valid codeword wherein, if no match is found, either discarding the codeword or applying error correction to the codeword to form a valid codeword, the valid codewords being a plurality of codewords assigned to the plurality of the nucleic acid targets; and determining a nucleic acid abundance and/or spatial distribution within the nucleus using the valid codewords corresponding to the binding of the plurality of nucleic acid probes within the nucleus.

In another set of embodiments, the method comprises associating a plurality of nucleic acid targets of a genome with a plurality of codewords, wherein the codewords comprise a number of positions and values for each position and the codewords form an error-checking and/or error-correcting code space, and wherein the plurality of nucleic acid targets of the genome are distributed such that each chromosome of the genome contains no more than 200 nucleic acid targets; exposing a nucleus of a cell containing the genome to a plurality of nucleic acid probes, wherein at least some of the plurality of nucleic acid probes comprises a first portion comprising a target sequence and a second portion comprising one or more read sequences, wherein each read sequence represents a value of a position within the codewords; for each nucleic acid probe of the plurality of nucleic acid probes, determining binding of the nucleic acid probe within the nucleus; creating codewords corresponding to the binding of the plurality of nucleic acid probes within the nucleus, wherein the values of the digits of the codewords are based on the read sequences present on the nucleic acid probes; for at least some of the codewords, matching the codeword to a valid codeword wherein, if no match is found, either discarding the codeword or applying error correction to the codeword to form a valid codeword, the valid codewords being a plurality of codewords assigned to the plurality of the nucleic acid targets; and determining a nucleic acid abundance and/or spatial distribution within the nucleus using the valid codewords corresponding to the binding of the plurality of nucleic acid probes within the nucleus.

According to another set of embodiments, the method comprises associating a plurality of between 500 and 1500 nucleic acid targets of a genome with a plurality of codewords, wherein the codewords comprise a number of positions and values for each position and the codewords form an error-checking and/or error-correcting code space; exposing a nucleus of a cell containing the genome to a plurality of nucleic acid probes, wherein at least some of the plurality of nucleic acid probes comprises a first portion comprising a target sequence and a second portion comprising one or more read sequences, wherein each read sequence represents a value of a position within the codewords; for each nucleic acid probe of the plurality of nucleic acid probes, determining binding of the nucleic acid probe within the nucleus; creating codewords corresponding to the binding of the plurality of nucleic acid probes within the nucleus, wherein the values of the digits of the codewords are based on the read sequences present on the nucleic acid probes; for at least some of the codewords, matching the codeword to a valid codeword wherein, if no match is found, either discarding the codeword or applying error correction to the codeword to form a valid codeword, the valid codewords being a plurality of codewords assigned to the plurality of the nucleic acid targets; and determining a nucleic acid abundance and/or spatial distribution within the nucleus using the valid codewords corresponding to the binding of the plurality of nucleic acid probes within the nucleus.

In yet another set of embodiments, the method includes associating a plurality of nucleic acid targets of a genome with a plurality of codewords, wherein the codewords comprise a number of positions and values for each position and the codewords form an error-checking and/or error-correcting code space, and wherein the plurality of nucleic acid targets are separated by at least 100,000 nucleotides within the genome; exposing a nucleus of a cell containing the genome to a plurality of nucleic acid probes; and determining a nucleic acid abundance and/or spatial distribution within the nucleus by determining binding of the plurality of nucleic acid probes within the nucleus using an error-checking and/or error-correcting detection technique.

In still another set of embodiments, the method comprises associating a plurality of nucleic acid targets of a genome with a plurality of codewords; exposing a sample containing a cell suspected of containing the genome to a plurality of nucleic acid probes, wherein at least some of the plurality of nucleic acid probes comprises a first portion comprising a target sequence and a second portion comprising one or more read sequences, wherein each read sequence represents a value of a position within the plurality of codewords; exposing the sample to a plurality of adaptors, wherein at least some of the adaptors comprise a first portion substantially complementary to one or more of the read sequences, and a second portion comprising one or more identification sequences; exposing the sample to a round of one or more readout probes to determine one or more identification sequences, wherein at least some of the readout probes comprise a first portion comprising a sequence substantially complementary to one of the identification sequences, and a second portion comprising a signaling entity; determining the signaling entity in at least some locations in the sample; and inactivating the signaling entity in at least some locations in the sample; repeating the steps of exposing the sample to a round, determining the signaling entity, and inactivating the signaling entity, wherein no more than 10 distinct signaling entities are used in all of the rounds; determining codewords at the locations based on determining the signaling entity in the sample; and determining nucleic acid targets in the sample based on the codewords.

According to still another set of embodiments, the method comprises associating a plurality of nucleic acid targets of a genome with a plurality of codewords; exposing a sample containing a cell suspected of containing the genome to a plurality of nucleic acid probes, wherein at least some of the plurality of nucleic acid probes comprises a first portion comprising a target sequence and a second portion comprising one or more read sequences, wherein each read sequence represents a value of a position within the plurality of codewords; exposing the sample to a plurality of adaptors, wherein at least some of the adaptors comprise a first portion substantially complementary to one or more of the read sequences, and a second portion comprising one or more identification sequences; exposing the sample to a round of one or more readout probes to determine one or more identification sequences, wherein at least some of the readout probes comprise a first portion comprising a sequence substantially complementary to one of the identification sequences, and a second portion comprising a signaling entity; determining the signaling entity in at least some locations in the sample; and inactivating the signaling entity in at least some locations in the sample; repeating the steps of exposing the sample to a round, determining the signaling entity, and inactivating the signaling entity, wherein at least one of the signaling entities is used in more than one of the rounds; determining codewords at the locations based on determining the signaling entity in the sample; and determining nucleic acid targets in the sample based on the codewords.

According to another set of embodiments, the method comprises determining positions of nascent RNA within a nucleus; determining positions of DNA within the nucleus; and determining positions of nuclear speckles within the nucleus.

In yet another set of embodiments, the method comprises determining positions of nascent RNA within a nucleus; determining positions of DNA within the nucleus; and determining positions of a protein within the nucleus. In still another set of embodiments, the method comprises determining positions of nascent RNA within a nucleus; determining positions of DNA within the nucleus; and determining positions of a nucleic acid within the nucleus, wherein the nucleic acid is not the nascent RNA or the DNA.

Some aspects encompass methods of making one or more of the embodiments described herein. Also, some aspects encompasses methods of using one or more of the embodiments described herein.

Other advantages and novel features of the present disclosure will become apparent from the following detailed description of various non-limiting embodiments when considered in conjunction with the accompanying figures.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the disclosure.

FIGS. 1A-1I show genome-scale chromatin imaging, in accordance with certain embodiments;

FIGS. 2A-2E show trans-chromosomal contacts enrichment, in another embodiment;

FIGS. 3A-3H show genome-scale imaging of chromatin and transcription activity in the context of nuclear structures, in still another embodiment;

FIGS. 4A-4F show trans-chromosomal interactions between active chromatin, in another embodiment;

FIGS. 5A-5E illustrates a saturatable amplification system, in one embodiment;

FIGS. 6A-6B show contact frequency matrices, in yet another embodiment;

FIGS. 7A-7C show sub-chromosomal structures derived from genome-scale imaging and comparison with ensemble Hi-C data, in still another embodiment;

FIG. 8 shows reproducibility of the chromatin imaging experiments between replicates, in another embodiment;

FIGS. 9A-9B show distinct spatial distributions in single cells, in certain embodiments;

FIGS. 10A-10B show nascent RNA transcript imaging, in yet other embodiments;

FIG. 11 shows the association of compartment-B loci with nuclear lamina, in certain embodiments;

FIG. 12 shows the association of compartment-A loci with nuclear speckles, in some embodiments;

FIGS. 13A-13C show changes in nuclear lamina and nuclear speckle association upon transcription inhibition, in yet another embodiment:

FIG. 14 shows the local density of trans-chromosomal A loci near each imaged locus, in yet another embodiment;

FIGS. 15A-15B show enrichment of active-active trans-chromosomal interactions among chromatin loci, in still another embodiment;

FIGS. 16A-16B show the enrichment of active-active trans-chromosomal interactions, in yet another embodiment;

FIGS. 17A-17M show high-resolution whole-chromosome tracing by sequential hybridization and characterization of chromatin domains in single cells, in one embodiment;

FIGS. 18A-18I show the compartment structure in single chromosomes and relationship between transcription activity and local chromatin content, in another embodiment;

FIGS. 19A-19H show the dependence of domain-domain interaction on their A/B composition and genomic distance, in yet another embodiment;

FIGS. 20A-20H show genome-scale chromatin imaging by massively multiplexed, combinatorial FISH, in still another embodiment;

FIGS. 21A-21E show enrichment of active-active chromatin interactions in trans-chromosomal interactions, according to one embodiment;

FIGS. 22A-22J show multi-modal genome-scale imaging of chromatin and transcription activity in the context of nuclear structures, in accordance with another embodiment;

FIGS. 23A-23D show the correlation between transcriptional activity and local enrichment of trans-chromosomal active chromatin, in yet another embodiment;

FIGS. 24A-24N show high-resolution whole-chromosome tracing by sequential hybridization, and ensemble statistics of Chr21 structural features in comparison with Hi-C, in still another embodiment;

FIGS. 25A-25G show ensemble A/B compartment analyses for Chr21 and Chr2, in yet another embodiment;

FIGS. 26A-26J show measurements for RNA and DNA FISH probe crosstalk, in still another embodiment;

FIGS. 27A-27J show genome-scale imaging by combinatorial FISH: localization error, reproducibility, and comparison with Hi-C, in one embodiment;

FIGS. 28A-28B show that compartment-A and compartment-B loci display distinct spatial distributions in the nucleus, according to another embodiment;

FIGS. 29A-29F show the effect of transcriptional inhibition on the trans-chromosome chromatin interactions and the nuclear body association rates of chromatin loci, in yet another embodiment; and

FIGS. 30A-30D show enrichment of trans-chromosomal active chromatin interactions in different nuclear environments, in still another embodiment.

### DETAILED DESCRIPTION

The present invention generally relates to genomics. Some embodiments are directed to imaging the 3D organization of the genome, or part of the genome, with high throughput in the sequence space. Some embodiments are directed to imaging the 3D organization of the genome, or part of the genome, in the context of transcriptional activity and nuclear structures. In addition, certain embodiments are directed to chromatin structures, 3D chromatin organizations, trans-chromosomal interactions and chromatin-nuclear-structure interactions as well as their relationship with transcription, etc. In addition, various embodiments are directed to imaging methods that allow mapping of the 3D organization of the genome, or part of the genome, in the context of nuclear structures and transcriptional activity. Some embodiments are directed to massively multiplexed fluorescence in situ hybridization methods for imaging chromatin loci and/or nascent RNA transcripts at the chromosome or genome scale. In some cases, simultaneous imaging of hundreds of genomic loci can be performed. In some cases, simultaneous imaging of ˜1000 genomic loci and/or transcriptional activities of ˜1000 genes within these loci together with various nuclear structures can be performed. In certain cases, chromatin domains and compartments can be observed. In certain cases, extensive trans-chromosomal interactions that were enriched for active chromatin interactions in a transcription-correlated manner can be observed. In some cases, transcription-dependent chromatin interactions with nuclear speckles and nuclear lamina across the genome can be observed.

The three-dimensional (3D) organization of chromatin regulates many genome functions. An understanding of 3D genome organization is hindered by the lack of tools that allow direct visualization of chromatin organization at the chromosome scale and genome scale in its native context. Thus, described in certain embodiments are a multiplexed FISH approach by sequential imaging over multiple hybridization rounds, for example, such that each round targets one or two or three genomic loci using one- or two- or three-color imaging. Described in other embodiments are a combinatorial FISH approach in many chromatin loci are imaged simultaneously in each round and their distinct identities are determined based on the combinations of rounds they appear in. This is generally based on MERFISH and other approaches, e.g., as discussed in Int. Pat. Apl. Pub. No. WO 2016/018960, entitled “Systems and Methods for Determining Nucleic Acids”; and Int. Pat. Apl. Pub. No. WO 2016/018963, entitled “Probe Library Construction,” each incorporated herein by reference in its entirety. Approaches such as those discussed herein may be used to image distinct chromatin loci in single cells and can be used to provide insights into chromatin structures, their relationship with transcription, interaction with nuclear proteins, etc.

Some aspects are generally directed to systems and methods of using multiplexed FISH, or other techniques, in some cases using MERFISH, including those described herein, to image chromosomes or chromatin, e.g., in a cell. In addition, certain embodiments are generally directed to systems and methods of imaging and/or determining at least 100 distinct genomic loci, at least 500 distinct genomic loci, or at least 1,000 distinct genomic loci, etc. in a single cell. In some cases, other parts of the cell, or the nucleus may be determined, for example, RNA present within the nucleus, e.g., nascent RNA, nuclear speckles, nucleoli, nuclear lamina, other nuclear structures or proteins, etc. As a non-limiting example, for a nucleus of a cell, the positions of chromosomes or chromatin, the nascent RNAs, nuclear speckles, nucleoli, and/or nuclear lamina may be determined.

Certain embodiments are directed to determining a sample, which may include a cell culture, a suspension of cells, a biological tissue, a biopsy, an organism, or the like. The sample can also be cell-free but nevertheless contain nucleic acids in some cases. If the sample contains a cell, the cell may be a human cell, or any other suitable cell, e.g., a mammalian cell, a fish cell, an insect cell, a plant cell, or the like. More than one cell may be present in some cases.

Within the sample, the targets to be determined can include nucleic acids, proteins, or the like. For example, these may be present within the nucleus of cells within the sample. In certain embodiments, chromatin within a cell can be determined, for instance, relative to nuclear structures of the cell, including nuclear speckles, nucleoli, nuclear lamina, or nuclear structures or proteins. In some cases, chromatin loci and/or RNA transcripts may be determined within the cell, e.g., at the chromosome or genome scale.

One example of such a method is now discussed. It should be understood, however, that this method is presented by way of explanation and not limitation; other aspects and embodiments are also discussed herein. In one set of embodiments, the nucleic acids within a cell, for example within the nucleus of a cell, are to be determined. These typically include DNA (e.g., genomic DNA, which may be present in the form of chromatin, e.g., packaged together with proteins such as histones) and RNA (e.g., at the start of the transcription phase when the DNA is transcribed into RNA; this RNA within the nucleus is sometimes referred to as nascent RNA). In contrast to techniques that detect RNA that may be present anywhere within a cell, the DNA is highly packed within the nucleus of the cell, making it substantially more difficult to determine its structure. For instance, the DNA may be packed within the cell as chromosomes or chromatin, and such DNA may often be entangled or packed closely together within the nucleus. Thus, in certain embodiments, the DNA targets may be selected to be spatially separated.

In some cases, the sample is subjected to multiple rounds of hybridization with nucleic acid probes, where one or more rounds round targets one or more target nucleic acids with single-color or multi-color imaging. In some cases, the identities of the target nucleic acids are determined based on which round and/or which color channel they are imaged. In some cases, the positions of the target nucleic acids are determined. In some cases, at least 50, at least 100, at least 500, at least 1000, at least 5000, or at least 10,000 target nucleic acids are determined. In some cases, the target nucleic acids are genomic loci. In some cases, the target nucleic acids are genomic loci and/or nascent RNA transcripts. In some cases, the positions of the genomic loci are used to determine the three-dimensional organization of chromatin or the three-dimensional organization of the genome in the cell.

In some cases, primary nucleic acid probes able to target nucleic acids within a cell, for example within the nucleus of a cell, are designed. The probes each contain a target sequence that binds to one of the target nucleic acids. The probes may also contain a portion that comprises one or more “readout sequences” that can be used to determine the identity and the position of the primary nucleic acid probes. In some embodiments, the primary nucleic acid probes may contain a plurality of readout sequences. These can be individually read using one or multiple rounds of secondary nucleic acid probes, called readout probes, that can bind to a readout sequence of the primary nucleic acid probe. The readout probes may also contain a signaling entity, such as a florescent entity, e.g., that can be determined using various microscopy techniques. In some cases, the multiple rounds of readout probes may be applied sequentially, such that one type of readout probe is applied to a sample and fluorescence within the sample determined, then the readout probe, or the signaling entity on the readout probe, is inactivated or removed and the next type of readout probe applied. In some cases, locations within the sample may be associated with a plurality of readout probes, and this information may be digitized for analysis.

In some cases, the multiple rounds of readout probes may be applied sequentially, such that more than one type of readout probes is applied to a sample in each round and/or fluorescence within the sample is determined using multi-color imaging, then the readout probes, and/or the signaling entities on the readout probes, are inactivated or removed and the next set of more than one types of readout probes applied. In some cases, locations within the sample may be associated with a plurality of readout probes, and this information may be digitized for analysis.

In some cases, the locations of the primary nucleic acid probes, and the target nucleic acids, may be determined using one or multiple rounds of readout probes. For example, there may be at least 10, at least 20, at least 30, at least 40, at least 50, at least 60, at least 70, at least 80, at least 90, at least 100, at least 150, at least 200, at least 250, at least 300, at least 400, at least 500, at least 600, at least 700, at least 800, at least 900, at least 1000, etc. rounds of readout probes. Thus, in some cases, a sample may be exposed to multiple rounds of applying readout probes, determining the probes within the sample (e.g., using a signaling entity, such as is described herein), and removing or inactivating the secondary nucleic acid probes.

In addition, it should be understood that the readout probes need not all be different. In some cases, more than one round of identical readout probes may be used, for example, to determine whether any degradation and/or movement has occurred in the sample, e.g., over time, due to the effects of supplying multiple rounds of nucleic acids or other chemicals, as a control, etc.

In some cases, the sample is subjected to multiple rounds of hybridization with nucleic acid probes and each round is subject to single-color or multi-color imaging. In some cases, the identities of the target nucleic acids are determined based on which combination of rounds and/or color channel they are imaged. In some cases, the positions of the target nucleic acids are determined. In some cases, at least 50, at least 100, at least 500, at least 1000, at least 5000, or at least 10,000 target nucleic acids are determined. In some cases, the target nucleic acids are genomic loci. In some cases, the target nucleic acids are genomic loci and/or nascent RNA transcripts. In some cases, the positions of the genomic loci are used to determine the three-dimensional organization of chromatin or the three-dimensional organization of the genome in the cell.

In some cases, primary nucleic acid probes (also called encoding probes) able to target nucleic acids within a cell, for example within the nucleus of a cell, are designed. The probes each comprise a target sequence that binds to one of the target nucleic acids. The probes may also contain a portion that comprises one or more “readout sequences” that can be used to determine the identity and the position of the primary or encoding nucleic acid probes. In some embodiments, the primary or encoding nucleic acid probes may contain a plurality of readout sequences. These can be individually read using one or multiple rounds of readout probes, that can bind to a readout sequence of the primary or encoding nucleic acid probe. The readout probes may also contain a signaling entity, such as a florescent entity, e.g., that can be determined using various microscopy techniques. In some cases, the multiple rounds of readout probes may be applied sequentially, such that one type of readout probe is applied to a sample and fluorescence within the sample determined, then the readout probe, or the signaling entity on the readout probe, is inactivated or removed and the next type of readout probe applied. In some cases, locations within the sample may be associated with a plurality of readout probes, and this information may be digitized for analysis. In some cases, the multiple rounds of readout probes may be applied sequentially, such that more than one type of readout probes are applied to a sample in each round and fluorescence within the sample determined using multi-color imaging, then the readout probes, or the signaling entities on the readout probes, are inactivated or removed and the next set of more than one types of readout probes applied. In some cases, locations within the sample may be associated with a plurality of readout probes, and this information may be digitized for analysis.

In some cases, the locations of the primary or encoding nucleic acid probes, and the target nucleic acids, may be determined using one or multiple rounds of readout probes. For example, there may be at least 2, at least 3, at least 4, at least 5, at least 6, at least 7, at least 8, at least 9, at least 10, at least 12, at least 16, at least 20, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1,000, etc. rounds of readout probes. Thus, in some cases, a sample may be exposed to multiple rounds of applying readout probes, determining the probes within the sample (e.g., using a signaling entity, such as is described herein), and removing or inactivating the secondary nucleic acid probes.

The primary or encoding nucleic acid probes may be designed in some embodiments such that different targets within the sample are determinable using different combination of readout sequences, without necessarily requiring each of the readout sequences to be unique. As a non-limiting example, with 4 possible readout sequences A, B, C, and D, up to 6 different targets may be identified if the set of primary or encoding nucleic acid probes targeting each nucleic acid target only contains 2 readout sequences, e.g., corresponding to AB, AD, CB, CD, AC, and DB.

However, in some embodiments, not all of the possible combinations of readout sequences will be used. Instead, some of the combinations may not be assigned to any target in the nucleus, e.g., no primary or encoding nucleic acid probes having those combinations may be used. In some cases, the valid combinations of readout sequences used in the primary or encoding nucleic acid probes may be arranged so as to form an error-checking and/or an error correcting code space. Using such a method, determinations of readout sequences within the sample that do not correspond with a valid primary nucleic acid probe may be determined using error-checking to be in error, and in some cases, can even be corrected using error correction, e.g., to correspond to a valid primary nucleic acid probe.

While such methods have been described before, e.g., in Int. Pat. Apl. Pub. No. WO 2016/018960, entitled “Systems and Methods for Determining Nucleic Acids”; and Int. Pat. Apl. Pub. No. WO 2016/018963, entitled “Probe Library Construction,” such methods were not applied to imaging DNA in the more constrained environment within the nucleus of a cell. As mentioned, unlike the rest of the cell, the nucleus of the cell contains a very high portion of nucleic acids, including nearly all of its genomic DNA, and typically a high concentration of RNA (e.g., nascent RNA).

Accordingly, to access DNA within the nucleus of a cell, the targets of the primary or encoding nucleic acid probes can be chosen such that binding within the nucleus occurs in a spatially separated manner. For example, the targets can be chosen such that they are separated in genomic space, e.g., separated by at least 10,000 bp at least 30,000 bp at least 100,000 bp, at least 300,000 bp, at least 1,000,000 bp within the genome, or such that the genomic space contains no more than 100, no more than 200, no more than 300, no more than 500, no more than 1000, no more than 5000, no more than 10,000, no more than 50,000, no more than 100,000 nucleic acid targets. In some cases, more than one type of fluorescent probe or “color” can also be used, e.g., to allow more targets to be determined within the nucleus.

In some embodiments, the cell and/or nucleus may also be modified to allow such probes to reach the nucleic acids therein. For instance, the cells may be permeabilized or “fixed” to allow entry of nucleic acid probes. In addition, the DNA may be denatured in some embodiments, e.g., by applying heat, in order to allow more ready access to the DNA by the primary or encoding nucleic acid probes. This is not typically performed for RNA determinations, as RNA is single-stranded while DNA is usually double-stranded. In addition, in certain embodiments, before DNA can be studied, the RNA within the nucleus must be removed and/or inactivated, for example, to prevent probes targeting DNA from binding to the RNA. In some cases, for instance, an enzyme such as an RNase may be applied to the nucleus to prevent RNA from interfering with DNA determination.

In addition, it should be noted that in certain embodiments, the RNA within the nucleus may also be determined. This may be particularly valuable, e.g., when studying the spatial locations of DNA and RNA within a nucleus, and how they relate to each other. Thus, in one set of embodiments, the RNA within a nucleus may be determined, e.g., analogously to that described above for genomic DNA, prior to removal or inactivation of the RNA as described above.

In addition, in certain embodiments, proteins within a cell, for example within the nucleus of a cell, may also be determined. Examples include, but are not limited to nuclear speckle, nucleolis, or histone proteins. A variety of methods for determining proteins can be used. For instance, in one set of embodiments, immunofluorescence assay can be used. In another set of embodiments, a “sandwich assay” may be used, where a primary antibody able to specifically bind to a nuclear protein is applied, then a secondary antibody able to specifically bind to the primary antibody is used, where the secondary antibody contains a signaling entity, such as a florescent entity. Such determinations of proteins can be performed on the same sample or the same nucleus as above, e.g., before or after determination of nucleic acids within the nucleus. Thus, in some cases, proteins and nucleic acids within the nucleus of a cell may be determined, e.g., spatially.

The above discussion is a non-limiting example of one embodiment that can be used to determine nucleic acids, such as genomic DNA and/or nascent RNA, within the nucleus of a cell. However, other embodiments are also possible. Accordingly, more generally, various aspects are directed to various systems and methods for nucleic acids.

As mentioned, in certain embodiments, one, two, or more of DNA, RNA, and protein within a cell, for example, the nucleus of a cell, may be determined. The nucleic acids within a nucleus to be determined may include, for example, DNA (for example, genomic DNA), RNA, or other nucleic acids that are present within a cell (or other sample). The nucleic acids may be endogenous to the cell, or added to the cell. For instance, the nucleic acid may be viral, or artificially created. In some cases, the nucleic acid to be determined may be expressed by the cell. The nucleic acid is RNA in some embodiments. The RNA may be coding and/or non-coding RNA. For example, the RNA may encode a protein. Non-limiting examples of RNA that may be studied within the cell include mRNA, siRNA, rRNA, miRNA, tRNA, lncRNA, snoRNAs, snRNAs, exRNAs, piRNAs, or the like.

In one set of embodiments, all, or at least a significant portion of the genome of a cell may be determined. The determined genomic segments may be continuous or interspersed on the genome. For example, in some cases, at least 4 genomic segments are determined within a cell, and in some cases, at least 3, at least 4, at least 7, at least 8, at least 12, at least 14, at least 15, at least 16, at least 22, at least 30, at least 31, at least 32, at least 50, at least 63, at least 64, at least 72, at least 75, at least 100, at least 127, at least 128, at least 140, at least 255, at least 256, at least 500, at least 1,000, at least 1,500, at least 2,000, at least 2,500, at least 3,000, at least 4,000, at least 5,000, at least 7,500, at least 10,000, at least 12,000, at least 15,000, at least 20,000, at least 25,000, at least 30,000, at least 40,000, at least 50,000, at least 75,000, or at least 100,000 genomic segments may be determined within a cell.

In some cases, the entire genome of a cell may be determined. It should be understood that the genome generally encompasses all DNA molecules produced within a cell, not just chromosome DNA. Thus, for instance, the genome may also include, in some cases, mitochondria DNA, chloroplast DNA, plasmid DNA, etc., e.g., in addition to (or instead of) chromosome DNA. In some embodiments, at least about 0.01%, at least about 0.1%, at least about 1%, at least about 5%, at least about 10%, at least about 15%, at least about 20%, at least about 25%, at least about 30%, at least about 40%, at least about 50%, at least about 60%, at least about 70%, at least about 80%, at least about 90%, or 100% of the genome of a cell may be determined.

In addition, in some embodiments, a significant portion of the nucleic acid within the cell, or within the nucleus of a cell may be studied. For instance, in some cases, the RNA within the nucleus, e.g., nascent RNA, may be determined. In addition, in some cases, enough of the RNA present within a cell may be determined so as to produce a partial or complete transcriptome of the cell. In some cases, at least 4 types of RNAs (e.g., mRNAs, nascent RNA, etc.) are determined within a cell, or within the nucleus of the cell, and in some cases, at least 3, at least 4, at least 7, at least 8, at least 12, at least 14, at least 15, at least 16, at least 20, at least 22, at least 30, at least 31, at least 32, at least 50, at least 63, at least 64, at least 72, at least 75, at least 100, at least 127, at least 128, at least 140, at least 255, at least 256, at least 500, at least 1,000, at least 1,500, at least 2,000, at least 2,500, at least 3,000, at least 4,000, at least 5,000, at least 7,500, at least 10,000, at least 12,000, at least 15,000, at least 20,000, at least 25,000, at least 30,000, at least 40,000, at least 50,000, at least 75,000, or at least 100,000 types of RNAs may be determined within a cell, or within the nucleus of the cell.

In some cases, the transcriptome of a cell may be determined. It should be understood that the transcriptome generally encompasses all RNA molecules produced within a cell, not just mRNA. Thus, for instance, the transcriptome may also include rRNA, tRNA, siRNA, etc. in certain instances. In some embodiments, at least about 0.01%, at least about 0.1%, at least about 1%, at least 5%, at least 10%, at least 15%, at least 20%, at least 25%, at least 30%, at least 40%, at least 50%, at least 60%, at least 70%, at least 80%, at least 90%, or 100% of the transcriptome of a cell may be determined. In addition, in some cases, the transcriptome of the nucleus of a cell may be determined.

Furthermore, in some embodiments, other targets to be determined can include targets that are linked to nucleic acids, proteins, or the like. For instance, in one set of embodiments, a binding entity able to recognize a target may be conjugated to a nucleic acid probe. The binding entity may be any entity that can recognize a target, e.g., specifically or non-specifically. Non-limiting examples include enzymes, antibodies, receptors, complementary nucleic acid strands, aptamers, or the like. For example, an oligonucleotide-linked antibody may be used to determine a target. The target may bind to the oligonucleotide-linked antibody, and the oligonucleotides determined as discussed herein.

The determination of targets, such as nucleic acids within the cell or other sample, may be qualitative and/or quantitative. In addition, the determination may also be spatial, e.g., the position of the nucleic acids, or other targets, within the cell or other sample may be determined in two or three dimensions. In some embodiments, the positions, number, and/or concentrations of nucleic acids, or other targets, within the cell or other sample may be determined.

As mentioned, in one set of embodiments, the DNA within the nucleus of a cell, e.g., the genomic DNA, of the cell may be studied, for example, using nucleic acid probes such as discussed herein, e.g., including using sequential imaging or using combinatorial imaging with error-detecting and/or an error-correcting codes.

In certain embodiments, the DNA targets or the codes associated with the DNA targets within a cell, or within the nucleus of a cell, may be chosen such that the targets are spatially separated in each round of imaging, e.g., in genomic space, or in physical space based on knowledge of chromatin organization, for example, such as the organization of chromosomes into compact territories. This may be useful, for example, to be able to identify different targets within the cell of the nucleus of the cell, e.g., using techniques such as those discussed herein.

The targets within the genomic space may be selected using any suitable technique, e.g., randomly, or having a substantially uniform probabilistic distribution, etc. In certain embodiments, the targets may be selected individually to ensure spatial separation. In addition, in some embodiments, the targets may be selected to be those targets of interest within the genome, e.g., for a particular study.

For instance, in some embodiments, the targets may be chosen within a genomic space such that a nucleus will have no more than a certain number of nucleic acid targets. For instance, the targets may be chosen such that the genomic space contains no more than 100,000, no more than 10,000, no more than 8,000, no more than 6,000, no more than 5,000, no more than 4,000, no more than 3,000, no more than 2,000, no more than 1,500, no more than 1,000, no more than 900, no more than 800, no more than 700, no more than 600, no more than 500, no more than 400, no more than 300, no more than 200, no more than 100 nucleic acid targets, no more than 30 nucleic acid targets, or no more than 10 nucleic acid targets. In addition, in some embodiments, the targets may be chosen such that the genomic space contains at least 10, at least 30, at least 50, at least 100, at least 200, at least 300, at least 500, at least 1,000, at least 1,500, at least 2,000, at least 3,000, at least 5,000, at least 10,000, at least 100,000, etc. nucleic acid targets. Combinations of any of these are also possible in certain embodiments, e.g., there may be between 30 and 100, between 3,000 and 5,000, between 500 and 1,500, etc. nucleic acid targets. Such targets may be chosen, e.g., selectively, randomly, etc., as is discussed herein.

As another example, in some embodiments, the targets may be selected such that the chromosomes within the genome have no more than a certain number of nucleic acid targets (e.g., genomic loci). For instance, the targets may be chosen such that each chromosome has no more than 10,000, no more than 1000, no more than 500, no more than 400, no more than 300, no more than 200, no more than 150, no more than 125, no more than 100, no more than 90, no more than 80, no more than 70, no more than 60, no more than 50, no more than 40, no more than 30, no more than 20, or no more than 10 nucleic acid targets. In some cases, the targets may be chosen to have at least 10, at least 20, at least 30, at least 40, at least 50, at least 60, at least 70, at least 80, at least 90, at least 100, at least 125, at least 150, at least 200, at least 300, at least 400, at least 1,000, at least 10,000 etc. nucleic acid targets. In some cases, combinations of these may be selected, e.g., a chromosome may have between 30 and 50, between 40 and 100, between 50 and 60, between 30 and 80, etc., nucleic acid targets. In addition, different chromosomes may independently have the same or different numbers of nucleic acid targets, e.g., including the ranges described herein.

Such targets may be chosen, e.g., selectively, randomly, etc., for example, as is discussed herein. As non-limiting examples, the nucleic acid targets within the genome may be selected to have specific structural or functional properties, such as promoters, enhancers, and loci bound by specific nuclear architecture proteins. In some cases, some or all of the nucleic acid targets may be nucleic acid targets that are unique to their respective chromosomes.

In yet another embodiment, the targets may be selected to be separated by a minimum of a certain number of nucleotides, e.g., to facilitate a distribution of targets that are spatially separated. For instance, targets may be selected within the genome such that every target is separated by at least 1,000, at least 3,000, at least 5,000, at least 10,000, at least 30,000, at least 50,000, at least 100,000, at least 300,000, at least 500,000, at least 1,000,000, at least 3,000,000, at least 5,000,000, at least 10,000,000, etc. nucleotides. In addition, in certain embodiments, the targets may be selected within the genome such that every target is separated by no more than 10,000,000, no more than 5,000,000, no more than 3,000,000, no more than 1,000,000, no more than 500,000, no more than 300,000, no more than 100,000, no more than 50,000, no more than 30,000, no more than 10,000 nucleotides. Combinations of any of these are also possible in certain embodiments, e.g., the targets may be separated by between 30,000 and 100,000 between 3,000,000 and 5,000,000, between 500,000 and 1,000,000 etc. nucleotides. Such targets may be chosen, e.g., selectively, randomly, etc., as is discussed herein.

In addition, in one set of embodiments, the RNA within the nucleus of a cell, e.g., the nascent RNA, of the cell may be studied, for example, instead of or in addition to the DNA within the nucleus as described above. In some cases, for instance, the RNA within a nucleus may be determined, then the DNA within the nucleus may be determined.

In some cases, after determining the RNA, the RNA may be removed or inactivated before determining the DNA. This may facilitate separation of the DNA and RNA determinations, e.g., since no RNA signal will be present that could complicate the DNA determination. Examples of methods of removing or inactivating RNA include the use of RNases such as endoribonucleases or exoribonucleases. Specific non-limiting examples include RNase A, RNase H, RNase III, RNase L, RNase P, RNase PhyM, RNase T1, RNase T2, RNase U2, RNase V, PNPase, RNase PH, RNase R, RNase D, RNase T, oligoribonuclease, exoribonuclease I, exoribonuclease II, or the like.

However, it should be understood that in other embodiments, the DNA may be determined before the RNA, and/or both may be determined simultaneously. For instance, DNA may be removed or inactivated after determination using DNases such as an exodeoxyribonuclease or an endodeoxyribonuclease. Examples include, but are not limited to, deoxyribonuclease I (DNase I), deoxyribonuclease II (DNase II), DNase IV, UvrABC endonuclease, or the like. As another example, the DNA may be degraded via exposure to a restriction endonuclease. Many such nucleases are available commercially.

The RNA within the nucleus may be determined using any suitable technique, and may be determined using the same or different techniques than used to determine the DNA within the nucleus. In one embodiment, the RNA can be determined using MERFISH. See, e.g., Int. Pat. Apl. Pub. No. WO 2016/018960, entitled “Systems and Methods for Determining Nucleic Acids”; and Int. Pat. Apl. Pub. No. WO 2016/018963, entitled “Probe Library Construction,” each incorporated herein by reference in its entirety. In another embodiment, the RNA may be determined using a plurality of nucleic acid probes, e.g., as discussed herein. For example, in some embodiments, RNA may be determined using nucleic acids such as encoding nucleic acid probes, primary amplifier nucleic acids, secondary amplifier nucleic acids, etc., as described below. In some cases, the nucleic acid probes may define an error-detecting and/or an error-correcting code, e.g., as discussed herein.

In some embodiments, DNA such as genomic DNA may be determined using nucleic acids such as encoding nucleic acid probes, primary amplifier nucleic acids, secondary amplifier nucleic acids, etc., as described herein. In some cases, the nucleic acid probes may define an error-detecting and/or an error-correcting code, e.g., as discussed herein.

In addition, in one set of embodiments, proteins within the nucleus of a cell may be studied, e.g., in addition to nucleic acids present within the nucleus, using techniques such as those described above. Examples of proteins that can be studied include, but are not limited to nuclear speckles, nucleoli, the nuclear lamina, or histone proteins, etc. Speckles are structures that are enriched in pre-messenger RNA splicing factors and may be located in the interchromatin regions of the nucleoplasm of mammalian cells. Nucleoli are structures formed around the highly transcribed genomic loci encoding ribosomal RNA (rRNA), and may be enriched for rRNA and the transcriptional machinery associated with it. The nuclear lamina is a protein structure associated with the inner nuclear membrane, and may be enriched for intermediate filaments (lamins), as well as chromatin that is transcriptionally inactive. Histones are proteins used to wrap or fold the DNA into more compact complexes within the nucleus, forming chromatin.

A variety of methods for determining proteins can be used. For instance, in one set of embodiments, an immunofluorescence assay may be used. In another set of embodiments, a “sandwich assay” may be used, where a primary antibody able to specifically bind to a nuclear protein is applied, then a secondary antibody able to specifically bind to the primary antibody is used, where the secondary antibody contains a signaling entity, such as a florescent entity or an oligonucleotide that can be detected, e.g., using a complementary oligonucleotide linked to a fluorescent entity. Such determinations of proteins can be performed on the same sample or the same nucleus as above, e.g., before or after determination of nucleic acids within the nucleus. Thus, in some cases, proteins and nucleic acids within the nucleus of a cell may be determined, e.g., spatially.

As mentioned, in various embodiments such as those described herein, a variety of nucleic acid probes may be used to determine one or more targets within a cell or other sample, e.g., within the nucleus of the cell. The probes may comprise nucleic acids (or entities that can hybridize to a nucleic acid, e.g., specifically) such as DNA, RNA, LNA (locked nucleic acids), PNA (peptide nucleic acids), and/or combinations thereof. Examples of nucleic acid probes include, but are not limited to, those described in Int. Pat. Apl. Pub. No. WO 2016/018960, entitled “Systems and Methods for Determining Nucleic Acids”; and Int. Pat. Apl. Pub. No. WO 2016/018963, entitled “Probe Library Construction,” each incorporated herein by reference in its entirety. In some cases, additional components may also be present within the nucleic acid probes, e.g., as discussed below. In addition, any suitable method may be used to introduce nucleic acid probes into a cell, e.g., to target its nucleus.

For example, in some embodiments, the cell is fixed prior to introducing the nucleic acid probes, e.g., to preserve the positions of the nucleic acids or other targets within the cell, e.g., within its nucleus. Techniques for fixing cells are known to those of ordinary skill in the art. As non-limiting examples, a cell may be fixed using chemicals such as formaldehyde, paraformaldehyde, glutaraldehyde, ethanol, methanol, acetone, acetic acid, or the like. In one embodiment, a cell may be fixed using HEPES-glutamic acid buffer-mediated organic solvent (HOPE).

In addition, in some cases, the cell (or other sample) may be fixed more than once, e.g., during relatively long experiments. As an example, the sample may be re-fixed after the start of an experiment, e.g., after exposing the nucleus of the cell to the plurality of nucleic acid probes. For instance, the cell or other sample may be fixed at least once every 7 days, at least once every 4 days, at least once every 2 days, at least once every day, at least once every 12 hours, at least once every 6 hours, at least once every 3 hours, etc. In some cases, this may be done between various rounds, e.g., of exposure to nucleic acid probes (e.g., primary or secondary nucleic acid probes), etc. In some cases, the sample may be fixed a certain number of times, e.g., 2, 3, 4, 5, 6, 7, 8, 9, 10, or any other suitable number of times. If multiple fixes occur, these may independently use the same or different fixation techniques.

The nucleic acid probes may be introduced into the cell (or other sample) using any suitable method. In some cases, the cell may be sufficiently permeabilized such that the nucleic acid probes may be introduced into the cell by flowing a fluid containing the nucleic acid probes around the cells. In some cases, the cells may be sufficiently permeabilized as part of a fixation process; in other embodiments, cells may be permeabilized by exposure to certain chemicals such as ethanol, methanol, Triton, or the like. In addition, in some embodiments, techniques such as electroporation or microinjection may be used to introduce nucleic acid probes into a cell or other sample.

Certain aspects are thus generally directed to nucleic acid probes that are introduced into a cell (or other sample). The probes may comprise any of a variety of entities that can hybridize to a nucleic acid, typically by Watson-Crick base pairing, such as DNA, RNA, LNA, PNA, etc., depending on the application. The nucleic acid probe typically contains a target sequence that is able to bind to at least a portion of a target, e.g., a target nucleic acid. In some cases, the binding may be specific binding (e.g., via complementary binding). When introduced into a cell or other system, the target sequence may be able to bind to a specific target (e.g., nascent RNA, genomic DNA, an mRNA, or other nucleic acids as discussed herein). The nucleic acid probe may also contain one or more readout sequences, as discussed below.

In some cases, more than one type of nucleic acid probe may be applied to a sample, e.g., sequentially or simultaneously. For example, there may be at least 2, at least 5, at least 10, at least 25, at least 50, at least 75, at least 100, at least 300, at least 1,000, at least 3,000, at least 10,000, at least 30,000, at least 100,000, at least 300,000, at least 1,000,000 distinguishable nucleic acid probes that are applied to a sample, e.g., to a cell to target its nucleus. In some cases, the nucleic acid probes may be added sequentially. However, in some cases, more than one nucleic acid probe may be added simultaneously.

The nucleic acid probe may include one or more target sequences, which may be positioned anywhere within the nucleic acid probe. The target sequence may contain a region that is substantially complementary to a portion of a target, e.g., a target nucleic acid, which may be within the nucleus. For instance, in some cases, the portions may be at least 50%, at least 60%, at least 70%, at least 75%, at least 80%, at least 85%, at least 90%, at least 92%, at least 94%, at least 95%, at least 96%, at least 97%, at least 98%, at least 99%, or 100% complementary, e.g., to produce specific binding. Typically, complementarity is determined on the basis of Watson-Crick nucleotide base pairing.

In some cases, the target sequence may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the target sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the target sequence may have a length of between 10 and 30 nucleotides, between 20 and 40 nucleotides, between 5 and 50 nucleotides, between 10 and 200 nucleotides, or between 25 and 35 nucleotides, between 10 and 300 nucleotides, etc.

In some embodiments, the nucleic acid targets or the codes associated with the nucleic acid targets within a cell or the nucleus of a cell may be chosen such that the targets are spatially separated in each round of imaging, e.g., in genomic space, or in physical space based on previous knowledge of chromatin organization such as the organization of chromosomes into compact territories.

In addition, in some cases, the target sequence of a nucleic acid probe may be determined with reference to a target suspected of being present within a cell or other sample, e.g., within the nucleus of the cell. For example, a target nucleic acid to a protein (e.g., nuclear speckles, nuclear lamina, etc.) may be determined using the protein's sequence, e.g., by determining the nucleic acids that are expressed to form the protein. In some cases, only a portion of the nucleic acids encoding the protein are used, e.g., having the lengths as discussed above.

More than one target sequence that can be used to identify a particular target may be used, in accordance with certain embodiments. For instance, multiple probes can be used, sequentially and/or simultaneously, that can bind to or hybridize to the same or different regions of the same target. Hybridization typically refers to an annealing process by which complementary single-stranded nucleic acids associate through Watson-Crick nucleotide base pairing (e.g., hydrogen bonding, guanine-cytosine and adenine-thymine) to form double-stranded nucleic acid.

In some embodiments, a nucleic acid probe may also comprise one or more “readout” sequences. The readout sequences may be used, to identify the nucleic acid probe, e.g., through association with signaling entities, as discussed below. In some embodiments, the nucleic acid probe may comprise 1, 2, 3, 4, 5, 6, 7, 8, 9, 10, 11, 12, 13, 14, 15, 16 or more, 20 or more, 24 or more, 32 or more, 40 or more, 48 or more, 50 or more, 64 or more, 75 or more, 100 or more, 128 or more readout sequences. The readout sequences may be positioned anywhere within the nucleic acid probe. If more than one readout sequence is present, the readout sequences may be positioned next to each other, and/or interspersed with other sequences.

The readout sequences may be of any length. If more than one readout sequence is used, the readout sequences may independently have the same or different lengths. For instance, the readout sequence may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the readout sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the readout sequence may have a length of between 10 and 30 nucleotides, between 20 and 40 nucleotides, between 5 and 50 nucleotides, between 10 and 200 nucleotides, or between 25 and 35 nucleotides, between 10 and 300 nucleotides, etc.

The readout sequence may be arbitrary or random in some embodiments. In certain cases, the readout sequences are chosen so as to reduce or minimize homology with other components of the cell or other sample, e.g., such that the readout sequences do not themselves bind to or hybridize with other nucleic acids suspected of being within the cell or other sample. In some cases, the homology may be less than 10%, less than 8%, less than 7%, less than 6%, less than 5%, less than 4%, less than 3%, less than 2%, or less than 1%. In some cases, there may be a homology of less than 20 basepairs, less than 18 basepairs, less than 15 basepairs, less than 14 basepairs, less than 13 basepairs, less than 12 basepairs, less than 11 basepairs, or less than 10 basepairs. In some cases, such basepairs are sequential.

In addition, in some embodiments, some or all of the readout sequences may be selected such that they do not exhibit specific binding towards each other and/or towards the genome or other nucleic acids suspected of being present in the sample. For instance, a population of readout sequences may be “blasted” or tested for specific binding or complementarity. In some case, the readout sequences may exhibit no specific binding towards each other, and/or such that none of the readout sequences in the population of readout sequences has a complementary of more than 5, 6, 7, 8, 9, 10, etc. nucleotides to another readout sequence within the population of readout sequences.

In one set of embodiments, a population of nucleic acid probes may contain a certain number of readout sequences, which may be the same as the number of nucleic acid targets to be determined in the sample, for example, with each unique readout sequence corresponding to a unique target. In another set of embodiments, a population of nucleic acid probes may contain a certain number of readout sequences, which may be less than the number of nucleic acid targets to be determined in the sample. Those of ordinary skill in the art will be aware that if there is one signaling entity and n readout sequences, then in general 2ⁿ−1 different nucleic acid targets may be uniquely identified. However, not all possible combinations need be used. For instance, a population of nucleic acid probes may target 12 different nucleic acid targets, yet contain no more than 8 readout sequences. As another example, a population of nucleic acid probes may target 140 different nucleic acid targets, yet contain no more than 16 readout sequences. Different nucleic acid targets may be separately identified by using different combinations of readout sequences within each probe. For instance, the population of nucleic acid probes may contain 1, 2, 3, 4, 5, 6, 7, 8, 9, 10, 11, 12, 13, 14, 15, 16, etc. or more readout sequences. In some cases, a population of nucleic acid probes may each contain the same number of readout sequences, although in other cases, there may be different numbers of readout sequences present on the various probes.

As a non-limiting example, a first nucleic acid probe may contain a first target sequence, a first readout sequence, and a second readout sequence, while a second, different nucleic acid probe may contain a second target sequence, the same first readout sequence, but a third readout sequence instead of the second readout sequence. Such probes may thereby be distinguished by determining the various readout sequences present or associated with a given probe or location, as discussed herein. For example, the probes can be sequentially identified and encoded using “codewords,” as discussed below. Optionally, the codewords may also be subjected to error detection and/or correction.

As another non-limiting example, a first population of nucleic acid probes may contain a first target sequence, a first readout sequence, and a second readout sequence, while a second, different population of nucleic acid probes may contain a second target sequence, the same first readout sequence, but a third readout sequence instead of the second readout sequence. Such probes may thereby be distinguished by determining the various readout sequences present or associated with a given probe or location, as discussed herein. For example, the populations of probes can be sequentially identified and encoded using “codewords,” as discussed below. Optionally, the codewords may also be subjected to error detection and/or correction.

In addition, the population of nucleic acid probes, in certain embodiments, may be made using only 2 or only 3 of the 4 naturally occurring nucleotide bases, such as leaving out all the “G”s or leaving out all of the “C”s within the population of probes. Sequences lacking either “G”s or “C”s may form very little secondary structure in certain embodiments, and can contribute to more uniform, faster hybridization. Thus, in some cases, the nucleic acid probes may contain only A, T, and G; only A, T, and C; only A, C, and G; or only T, C, and G.

In one aspect, the readout sequences on the nucleic acid probes may be able to bind (e.g., specifically) to corresponding recognition sequences on the primary amplifier nucleic acids. Thus, when a nucleic acid probe recognizes a target within a biological sample, e.g., a DNA or RNA target, the primary amplifier nucleic acid are also able to associate with the target via the nucleic acid probe, with interactions between the readout sequences of the nucleic acid probes and corresponding recognition sequences on the primary amplifier nucleic acids, e.g., complementary binding. For instance, the recognition sequence may be able to recognize a target readout sequence, but not substantially recognize or bind to other, non-target readout sequence. The primary amplifier nucleic acids may also comprise any of a variety of entities able to hybridize a nucleic acid, e.g., DNA, RNA, LNA, and/or PNA, etc., depending on the application. For instance, such entities may form some or all of the recognition sequence.

In some cases, the recognition sequence may be substantially complementary to the target readout sequence. In some cases, the sequences may be at least 50%, at least 60%, at least 70%, at least 75%, at least 80%, at least 85%, at least 90%, at least 92%, at least 94%, at least 95%, at least 96%, at least 97%, at least 98%, at least 99%, or 100% complementary. Typically, complementarity is determined on the basis of Watson-Crick nucleotide base pairing. The structures of the target readout sequence may include those previously described.

In some cases, the recognition sequence may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the recognition sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the recognition sequence may have a length of between 10 and 30 nucleotides, between 20 and 40 nucleotides, between 5 and 50 nucleotides, between 10 and 200 nucleotides, or between 25 and 35 nucleotides, between 10 and 300 nucleotides, etc.

In some embodiments, a primary amplifier nucleic acid may also comprise one or more readout sequences able to bind to secondary amplifier nucleic acids, as discussed below. For example, a primary amplifier nucleic acid may comprise 1, 2, 3, 4, 5, 6, 7, 8, 9, 10, 11, 12, 13, 14, 15, 16 or more, 20 or more, 32 or more, 40 or more, 50 or more, 64 or more, 75 or more, 100 or more, 128 or more readout sequences. The readout sequences may be positioned anywhere within the primary amplifier nucleic acid. If more than one readout sequence is present, the readout sequence may be positioned next to each other, and/or interspersed with other sequences. In one embodiment, the primary amplifier nucleic acid comprises a recognition sequence at a first end and a plurality of readout sequences at a second end.

In some cases, a readout sequence within the primary amplifier nucleic acid may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the readout sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the readout sequence may have a length of between 10 and 20 nucleotides, between 10 and 30 nucleotides, between 20 and 40 nucleotides, between 5 and 50 nucleotides, between 10 and 200 nucleotides, or between 25 and 35 nucleotides, between 10 and 300 nucleotides, etc.

There may be any number of readout sequences within a primary amplifier nucleic acid. For example, there may be 1, 2, 3, 4, 5, 6, 7, 8, 9, 10, 11, 12, 13, 14, 15, 16, 17, 18, 19, 20, or more readout sequences present within a primary amplifier nucleic acid. If more than one read sequence is present within a primary amplifier nucleic acid, the readout sequences may be the same or different. In some cases, for example, the readout sequences may all be identical.

In some embodiments, the population of primary amplifier nucleic acids may be made using only 2 or only 3 of the 4 naturally occurring nucleotide bases, such as leaving out all the “G”s or leaving out all of the “C”s within the population of nucleic acids. Sequences lacking either “G”s or “C”s may form very little secondary structure in certain embodiments, and can contribute to more uniform, faster hybridization. Thus, in some cases, the primary amplifier nucleic acids may contain only A, T, and G; only A, T, and C; only A, C, and G; or only T, C, and G.

In some cases, more than one type of primary amplifier nucleic acid may be applied to a sample, e.g., sequentially or simultaneously. For example, there may be at least 2, at least 5, at least 10, at least 25, at least 50, at least 75, at least 100, at least 300, at least 1,000, at least 3,000, at least 10,000, or at least 30,000 distinguishable primary amplifier nucleic acids that are applied to a sample. In some cases, the primary amplifier nucleic acids may be added sequentially. However, in some cases, more than one primary amplifier nucleic acid may be added simultaneously.

In one set of embodiments, the readout sequences on the primary amplifier nucleic acids may be able to bind (e.g., specifically) to corresponding recognition sequences on the secondary amplifier nucleic acids. Thus, when a nucleic acid probe recognizes a target within a biological sample, e.g., a DNA or RNA target, the secondary amplifier nucleic acids are also able to associate with the target, via the primary amplifier nucleic acids, with interactions between the readout sequences of the primary amplifier nucleic acids and corresponding recognition sequences on the secondary amplifier nucleic acids, e.g., complementary binding. For instance, the recognition sequence on a secondary amplifier nucleic acid may be able to recognize a readout sequence on a primary amplifier nucleic acid, but not substantially recognize or bind to other, non-target readout sequence. The secondary amplifier nucleic acids may also comprise any of a variety of entities able to hybridize a nucleic acid, e.g., DNA, RNA, LNA, and/or PNA, etc., depending on the application. For instance, such entities may form some or all of the recognition sequence.

In some cases, the recognition sequence on the secondary amplifier nucleic acid may be substantially complementary to a readout sequence on a primary amplifier nucleic acid. In some cases, the sequences may be at least 50%, at least 60%, at least 70%, at least 75%, at least 80%, at least 85%, at least 90%, at least 92%, at least 94%, at least 95%, at least 96%, at least 97%, at least 98%, at least 99%, or 100% complementary.

In some cases, the recognition sequence on the secondary amplifier nucleic acid may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the recognition sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the recognition sequence may have a length of between 10 and 30 nucleotides, between 20 and 40 nucleotides, between 5 and 50 nucleotides, between 10 and 200 nucleotides, or between 25 and 35 nucleotides, between 10 and 300 nucleotides, etc.

In some embodiments, a secondary amplifier nucleic acid may comprise a signaling entity, and/or may comprise one or more readout sequences able to bind to a signaling entity, as discussed herein. For example, a secondary amplifier nucleic acid may comprise 1, 2, 3, 4, 5, 6, 7, 8, 9, 10, 11, 12, 13, 14, 15, 16 or more, 20 or more, 32 or more, 40 or more, 50 or more, 64 or more, 75 or more, 100 or more, 128 or more readout sequences able to bind to a signaling entity. The read sequences may be positioned anywhere within the secondary amplifier nucleic acid. If more than one readout sequences is present, the readout sequences may be positioned next to each other, and/or interspersed with other sequences. In one embodiment, the secondary amplifier nucleic acid comprises a recognition sequence at a first end and a plurality of readout sequences at a second end. This structure may also be the same or different than the structure of the primary amplifier nucleic acid.

In some cases, the readout sequence within the secondary amplifier nucleic acid may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the readout sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the readout sequence within the secondary amplifier nucleic acid may have a length of between 10 and 20 nucleotides, between 10 and 30 nucleotides, between 20 and 40 nucleotides, between 5 and 50 nucleotides, between 10 and 200 nucleotides, or between 25 and 35 nucleotides, between 10 and 300 nucleotides, etc.

There may be any number of readout sequences within a secondary amplifier nucleic acid. For example, there may be 1, 2, 3, 4, 5, 6, 7, 8, 9, 10, 11, 12, 13, 14, 15, 16, 17, 18, 19, 20, or more readout sequences present within a secondary amplifier nucleic acid. If more than one readout sequence is present within a secondary amplifier nucleic acid, the readout sequences may be the same or different. In some cases, for example, the readout sequences may all be identical. In addition, there may independently be the same or different numbers of readout sequences in the primary and in the secondary amplifier nucleic acids.

The population of secondary amplifier nucleic acids may be made using only 2 or only 3 of the 4 naturally occurring nucleotide bases, in certain embodiments such as leaving out all the “G”s or leaving out all of the “C”s within the population of nucleic acids. Sequences lacking either “G”s or “C”s may form very little secondary structure in certain embodiments, and can contribute to more uniform, faster hybridization. Thus, in some cases, the secondary amplifier nucleic acids may contain only A, T, and G; only A, T, and C; only A, C, and G; or only T, C, and G.

In some cases, more than one type of secondary amplifier nucleic acid may be applied to a sample, e.g., sequentially or simultaneously. For example, there may be at least 2, at least 5, at least 10, at least 25, at least 50, at least 75, at least 100, at least 300, at least 1,000, at least 3,000, at least 10,000, or at least 30,000 distinguishable secondary amplifier nucleic acids that are applied to a sample. In some cases, the secondary amplifier nucleic acids may be added sequentially. However, in some cases, more than one secondary amplifier nucleic acid may be added simultaneously.

In addition, in certain embodiments, this pattern can instead be repeated prior to the signaling entity, e.g., with tertiary amplifier nucleic acids, quaternary nucleic acids, etc., similar to the above discussion. The signaling entities may thus be bound to the ending amplifier nucleic acid. Thus, as non-limiting examples, to a target may be bound an encoding nucleic acid probe, to which a primary amplifier nucleic acid is bound, to which a secondary amplifier nucleic acid is bound, to which a tertiary amplifier nucleic acid is bound, to which a signaling entity is bound, or to a target may be bound an encoding nucleic acid probe, to which a primary amplifier nucleic acid is bound, to which a secondary amplifier nucleic acid is bound, to which a tertiary amplifier nucleic acid is bound, to which a quaternary amplifier nucleic acid is bound, to which a signaling entity is bound, etc. Accordingly, the ending amplifier nucleic acid need not necessarily be the secondary amplifier nucleic acid in all embodiments.

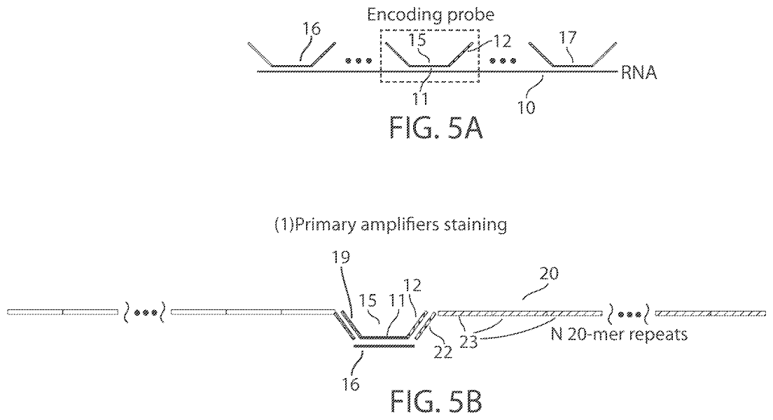

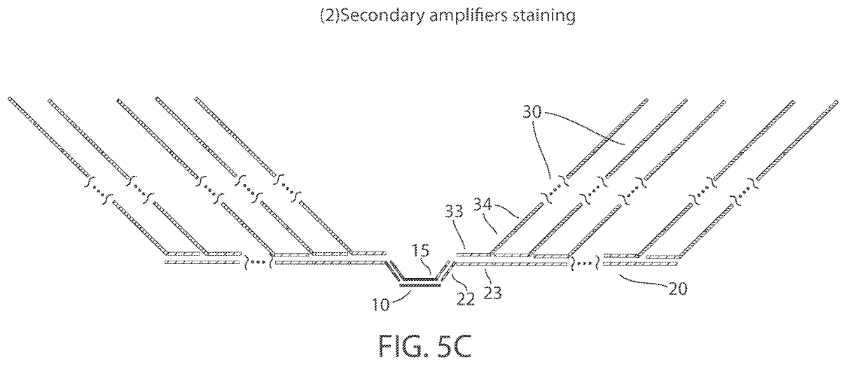

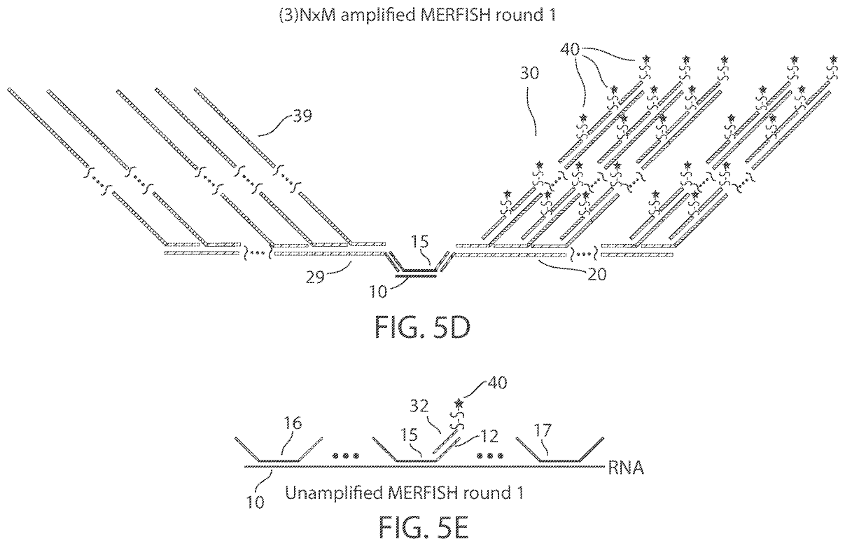

A non-limiting example of such a system is illustrated in FIG. 5. FIGS. 5A-5E show the creation of a saturatable system. FIG. 5A shows an example of an encoding nucleic acid probe, where an encoding nucleic acid probe 15 has bound to a target RNA. FIG. 5B shows a primary amplifier nucleic acid being used, in accordance with certain embodiments. FIG. 5C shows a secondary amplifier nucleic acid that may be bound to the primary amplifier nucleic acid. FIG. 5D shows that a plurality of signaling entities has been bound to the readout sequences of the secondary amplifier nucleic acids. FIG. 5E shows that if no amplification is applied, the nucleic acid probe may be exposed to a suitable secondary nucleic acid probe containing a signaling entity.

Other components may also be present within a nucleic acid probe or an amplifier nucleic acid as well, in certain cases. For example, in one set of embodiments, one or more primer sequences may be present, e.g., to facilitate enzymatic amplification. Those of ordinary skill in the art will be aware of primer sequences suitable for applications such as amplification (e.g., using PCR or other suitable techniques). Many such primer sequences are available commercially. Other examples of sequences that may be present within a primary or encoding nucleic acid probe include, but are not limited to promoter sequences, operons, identification sequences, nonsense sequences, or the like.

Typically, a primer is a single-stranded or partially double-stranded nucleic acid (e.g., DNA) that serves as a starting point for nucleic acid synthesis, allowing polymerase enzymes such as nucleic acid polymerase to extend the primer and replicate the complementary strand. A primer is (e.g., is designed to be) complementary to and to hybridize to a target nucleic acid. In some embodiments, a primer is a synthetic primer. In some embodiments, a primer is a non-naturally-occurring primer. A primer typically has a length of 10 to 50 nucleotides. For example, a primer may have a length of 10 to 40, 10 to 30, 10 to 20, 25 to 50, 15 to 40, 15 to 30, 20 to 50, 20 to 40, or 20 to 30 nucleotides. In some embodiments, a primer has a length of 18 to 24 nucleotides.

In some aspects, as previously discussed, certain embodiments use code spaces that encode various binding events, and optionally can use error detection and/or correction to determine the binding of nucleic acid probes to their targets. In some cases, a population of nucleic acid probes may contain certain “readout sequences” which can bind certain amplifier nucleic acids, as discussed above, and the locations of the nucleic acid probes or targets can be determined within the sample using signaling entities associated with the amplifier nucleic acids, for example, within a certain code space, e.g., as discussed herein. See also Int. Pat. Apl. Pub. Nos. WO 2016/018960 and WO 2016/018963, each incorporated herein by reference in its entirety. In some cases, a population of readout sequences within the nucleic acid probes may be combined in various combinations, e.g., such that a relatively small number of readout sequences may be used to determine a relatively large number of different nucleic acid probes, as discussed herein.

Thus, in some cases, a population of nucleic acid probes may each contain a certain number of readout sequences, some of which are shared between different nucleic acid probes such that the total population of nucleic acid probes may contain a certain number of readout sequences. A population of nucleic acid probes may have any suitable number of readout sequences. For example, a population of nucleic acid probes may have 1, 2, 3, 4, 5, 6, 7, 8, 9, 10, 11, 12, 13, 14, 15, 16, 17, 18, 19, 20, etc. read sequences. More than 20 are also possible in some embodiments. In addition, in some cases, a population of nucleic acid probes may, in total, have 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 20 or more, 24 or more, 32 or more, 40 or more, 50 or more, 60 or more, 64 or more, 100 or more, 128 or more, etc. of possible readout sequences present, although some or all of the probes may each contain more than one readout sequence, as discussed herein. In addition, in some embodiments, the population of nucleic acid probes may have no more than 100, no more than 80, no more than 64, no more than 60, no more than 50, no more than 40, no more than 32, no more than 24, no more than 20, no more than 16, no more than 15, no more than 14, no more than 13, no more than 12, no more than 11, no more than 10, no more than 9, no more than 8, no more than 7, no more than 6, no more than 5, no more than 4, no more than 3, or no more than two readout sequences present. Combinations of any of these are also possible, e.g., a population of nucleic acid probes may comprise between 10 and 15 readout sequences in total.

As a non-limiting example of an approach to combinatorially identifying a relatively large number of nucleic acid probes from a relatively small number of readout sequences contained within the nucleic acid probes, in a population of 6 different types of nucleic acid probes or 6 different group of nucleic acid probes (for example, each group of probes bind to a nucleic acid target), each type or group of nucleic acid probes comprising one or more readout sequences, the total number of readout sequences within the population may be no greater than 4. It should be understood that although 4 readout sequences are used in this example for ease of explanation, in other embodiments, larger numbers of nucleic acid probes may be realized, for example, using 5, 8, 10, 16, 32, etc. or more readout sequences, or any other suitable number of readout sequences described herein, depending on the application. For example, if each of the nucleic acid probes or each group of the nucleic acid probes contain two different readout sequences, then by using 4 such read sequences (A, B, C, and D), up to 6 probes or 6 groups of probes may be separately identified. It should be noted that in this example, the ordering of readout sequences on a nucleic acid probe or a group of nucleic acid probes is not essential, i.e., “AB” and “BA” may be treated as being synonymous (although in other embodiments, the ordering of read sequences may be essential and “AB” and “BA” may not necessarily be synonymous). Similarly, if 5 readout sequences are used (A, B, C, D, and E) in the population of nucleic acid probes, up to 10 probes or 10 groups of probes may be separately identified (e.g., AB, AC, AD, AE, BC, BD, BE, CD, CE, DE). For example, one of ordinary skill in the art would understand that, for k readout sequences in a population with n readout sequences on each probe or each group of groups, up to

$$( \begin{matrix}n \\ k\end{matrix} )$$

different probes may be produced, assuming that the ordering of readout sequences is not essential; because not all of the probes or all of the groups of probes need to have the same number of readout sequences and not all combinations of readout sequences need to be used in every embodiment, either more or less than this number of different probes may also be used in certain embodiments. In addition, it should also be understood that the number of readout sequences on each probe or each group of probes need not be identical in some embodiments. For instance example, some probes or some group of probes may contain 2 read sequences while other probes or other groups of probes may contain 3 read sequences. In some embodiments of each group of probes bind to a nucleic acid target.

In some aspects, the readout sequences and/or the pattern of binding of nucleic acid probes within a sample may be used to define an error-detecting and/or an error-correcting code, for example, to reduce or prevent misidentification or errors of the nucleic acids. Thus, for example, if binding is indicated (e.g., as determined using a signaling entity), then the location may be identified with a “1”; conversely, if no binding is indicated, then the location may be identified with a “0” (or vice versa, in some cases). Multiple rounds of binding determinations, e.g., using different readout probes complementary to readout sequences, can then be used to create a “codeword,” e.g., for that spatial location. In some embodiments, the codeword may be subjected to error detection and/or correction. For instance, the codewords may be organized such that, if no match is found for a given set of readout sequences or binding pattern of nucleic acid probes, then the match may be identified as an error, and optionally, error correction may be applied sequences to determine the correct target for the nucleic acid probes. In some cases, the codewords may have fewer “letters” or positions than the total number of nucleic acids encoded by the codewords, e.g. where each codeword encodes a different nucleic acid.

Such error-detecting and/or the error-correction code may take a variety of forms. A variety of such codes have previously been developed in other contexts such as the telecommunications industry, such as Golay codes or Hamming codes. In one set of embodiments, the readout sequences or binding patterns of the nucleic acid probes are assigned such that not every possible combination is assigned.

For example, if 4 readout sequences are possible and a nucleic acid probe or a group of nucleic acid probes contains 2 readout sequences, then up to 6 nucleic acid probes or 6 groups of nucleic acid probes (e.g., such that each group of nucleic acid probes binds to a nucleic acid target) could be identified; but the number of nucleic acid probes or the number of groups of nucleic acid probes used may be less than 6. Similarly, for k readout sequences in a population with n readout sequences on each nucleic acid probe or each group of nucleic acid probes,

$$( \begin{matrix}n \\ k\end{matrix} )$$

different probes or different groups of probes may be produced, but the number of nucleic acid probes or the number of groups of nucleic acid probes that are used may be any number more or less than

$$( \begin{matrix}n \\ k\end{matrix} ) .$$

In addition, these may be randomly assigned, or assigned in specific ways to increase the ability to detect and/or correct errors.

As another example, if multiple rounds of nucleic acid probes (e.g., such that multiple rounds of readout probes that can bind to readout sequences on primary or encoding probes) are used, the number of rounds may be arbitrarily chosen. If in each round, each target can give two possible outcomes, such as being detected or not being detected, up to 2ⁿ different targets may be possible for n rounds of probes, but the number of targets that are actually used may be any number less than 2ⁿ. In another example, if in each round, each target can give more than two possible outcomes, such as being detected in different color channels, more than 2ⁿ (e.g. 3ⁿ, 4ⁿ, . . . ) different targets may be possible for n rounds of probes. In some cases, the number of targets that are actually used may be any number less than this number. In addition, these may be randomly assigned, or assigned in specific ways to increase the ability to detect and/or correct errors.

The codewords may be used to define various code spaces. Each nucleic acid target is associated with a codeword. For example, in one set of embodiments, the codewords may be assigned within a code space such that the assignments are separated by a Hamming distance, which measures the number of incorrect “reads” in a given pattern that cause the codeword or the associated nucleic acid target to be misinterpreted as a different valid codeword or nucleic acid target. In certain cases, the Hamming distance may be at least 2, at least 3, at least 4, at least 5, at least 6, or the like. In addition, in one set of embodiments, the assignments may be formed as a Hamming code, for instance, a Hamming(7, 4) code, a Hamming(15, 11) code, a Hamming(31, 26) code, a Hamming(63, 57) code, a Hamming(127, 120) code, etc. In another set of embodiments, the assignments may form a SECDED code, e.g., a SECDED(8,4) code, a SECDED(16,4) code, a SCEDED(16, 11) code, a SCEDED(22, 16) code, a SCEDED(39, 32) code, a SCEDED(72, 64) code, etc. In yet another set of embodiments, the assignments may form an extended binary Golay code, a perfect binary Golay code, or a ternary Golay code. In another set of embodiments, the assignments may represent a subset of the possible values taken from any of the codes described above.

For example, an error-correcting code may be formed by using only binary words that contain a fixed or constant number of “1” bits (or “0” bits) to encode the targets. For example, the code space may only include 1, 2, 3, 4, 5, 6, 7, 8, 9, 10, 11, 12, 13, 14, 15, 16, etc. “1” bits (or “0” bits), e.g., all of the codes have the same number of “1” bits or “0” bits, etc. In another set of embodiments, the assignments may represent a subset of the possible values taken from codes described above for the purpose of addressing asymmetric readout errors. For example, in some cases, a code in which the number of “1” bits may be fixed for all used binary words may eliminate the biased measurement of words with different numbers of “F”s when the rate at which “0” bits are measured as “F”s or “1” bits are measured as “0”s are different.

Accordingly, in some embodiments, once the codeword is determined (e.g., as discussed herein), the codeword may be compared to the valid nucleic acid codewords. If a match is found, then the nucleic acid target can be identified or determined. If no match is found, then an error in the reading of the codeword may be identified. In some cases, error correction can also be applied to determine the correct codeword, and thus resulting in the correct identity of the nucleic acid target. In some cases, the codewords may be selected such that, assuming that there is only one error present, only one possible correct codeword is available, and thus, only one correct identity of the nucleic acid target is possible. In some cases, this may also be generalized to larger codeword spacings or Hamming distances; for instance, the codewords may be selected such that if two, three, or four errors are present (or more in some cases), only one possible correct codeword is available, and thus, only one correct identity of the nucleic acid targets is possible.

The error-correcting code may be a binary error-correcting code, or it may be based on other numbering systems, e.g., ternary or quaternary error-correcting codes. For instance, in one set of embodiments, more than one type of signaling entity may be used and assigned to different numbers within the error-correcting code. Thus, as a non-limiting example, a first signaling entity (or more than one signaling entity, in some cases) may be assigned as “1” and a second signaling entity (or more than one signaling entity, in some cases) may be assigned as “2” (with “0” indicating no signaling entity present), and the codewords distributed to define a ternary error-correcting code. Similarly, a third signaling entity may additionally be assigned as “3” to make a quaternary error-correcting code, etc.

In one set of embodiments, nucleic acid targets in a sample are each assigned with a codeword. For example, these codewords could be chosen from one of the codespaces as describe herein. In some cases, the codewords form an error-detecting and/or error correcting code. The sample may be subjected to hybridization to a population of primary or encoding nucleic acid probes in some cases. Some or all of the primary or encoding probe may comprise a target sequence that can bind to one of the nucleic acid targets and/or may also comprises one or more readout sequences. The readout sequences on the collection of primary or encoding probes that bind to each nucleic acid target may form a unique codeword that corresponds to the codeword assigned to the nucleic acid target. The sample are then subject to one or more rounds of hybridization with readout probes. The readout probes may be able to bind to a readout sequence and/or may be associated with a signaling entity. The collection of readout sequences may be associated with a nucleic acid target, and hence the codeword assigned to the nucleic acid target can then be identified, e.g., through the binding of readout probes.

In some cases, multi-color imaging can be used in each round to allow simultaneous imaging and determination of multiple readout probes associated with different signaling entities. In some cases, the positions of the nucleic acid targets are determined. In some cases, at least 50, at least 100, at least 500, at least 1000, at least 5000, or at least 10,000 nucleic acid targets are determined this way. In some cases, the target nucleic acids are genomic loci. In some cases, the target nucleic acids are genomic loci and/or nascent RNA transcripts. In some cases, the positions of the genomic loci are used to determine the three-dimensional organization of chromatin or the three-dimensional organization of the genome in the cell. In some cases, primary amplifier nucleic acids and/or secondary amplifier nucleic acids and/or tertiary amplifier nucleic acids and/or quaternary amplifier nucleic acids are used to amplify the signal from each readout sequence. In some cases, adaptors are used as described below.

In one aspect, a plurality of adapters may be used to facilitate detection of targets within a sample. Such adapters may be useful, for example, in allowing a relatively small number of distinguishable signaling entities to be used, while still allowing for relatively large numbers of targets to be determined in a sample. For instance, at least 3, at least 4, at least 7, at least 8, at least 12, at least 14, at least 15, at least 16, at least 20, at least 22, at least 30, at least 31, at least 32, at least 50, at least 63, at least 64, at least 72, at least 75, at least 100, at least 127, at least 128, at least 140, at least 255, at least 256, at least 500, at least 1,000, at least 1,500, at least 2,000, at least 2,500, at least 3,000, at least 4,000, at least 5,000, at least 7,500, at least 10,000, at least 12,000, at least 15,000, at least 20,000, at least 25,000, at least 30,000, at least 40,000, at least 50,000, at least 75,000, or at least 100,000, etc. targets in a sample may be determined, while using a smaller number of signaling entities, for example, no more than 20, no more than 15, no more than 10, no more than 5, no more than 4, no more than 3, or no more than 2 signaling entities.

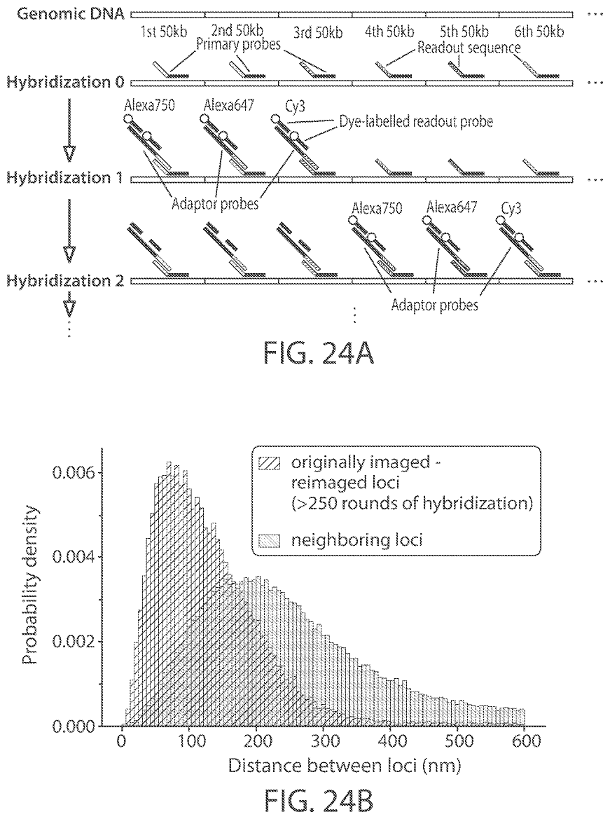

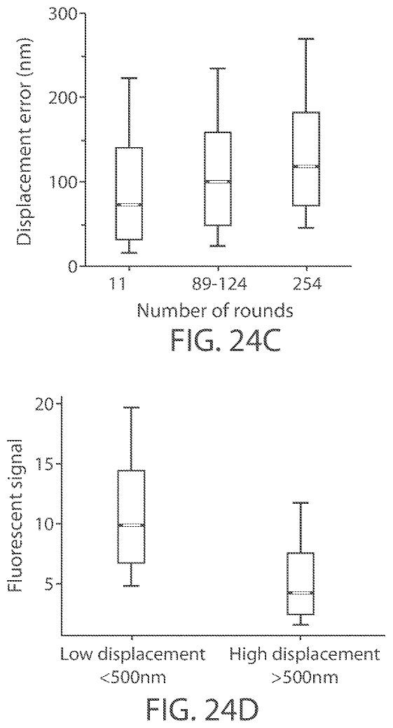

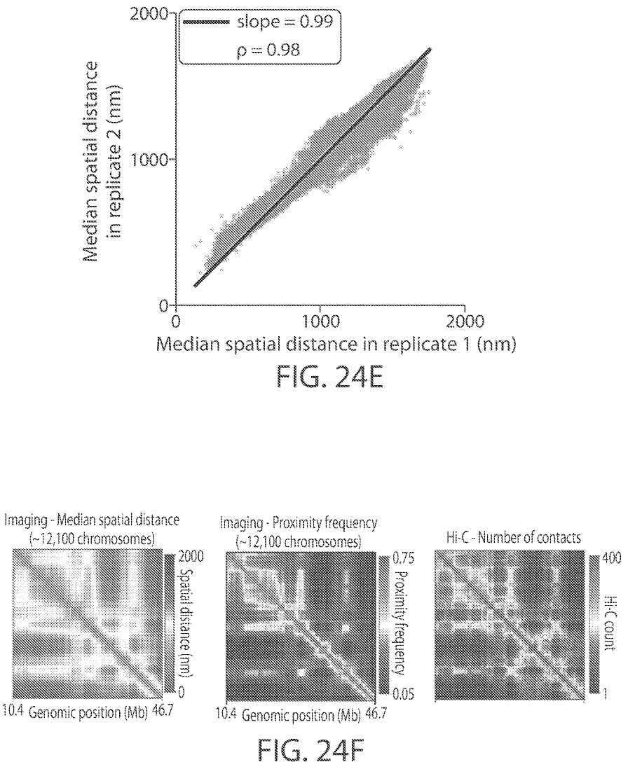

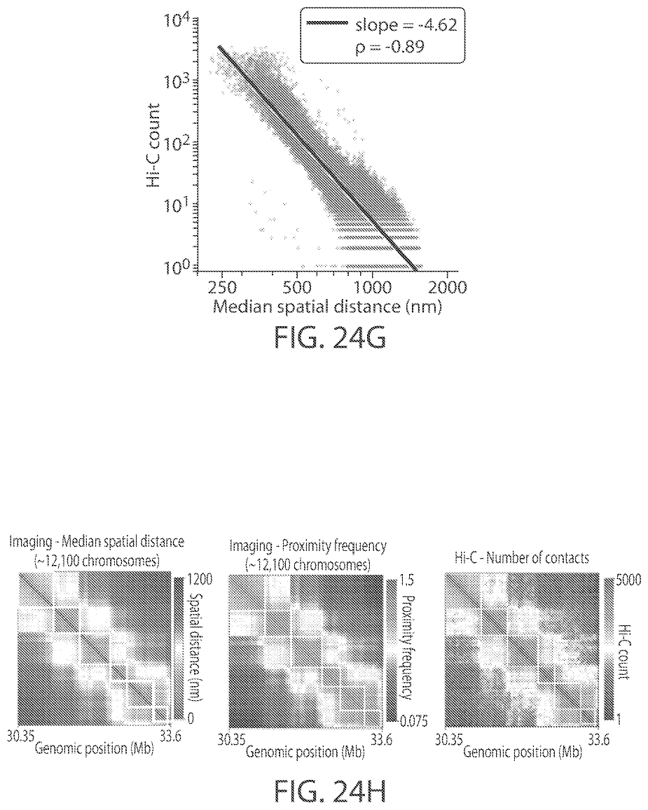

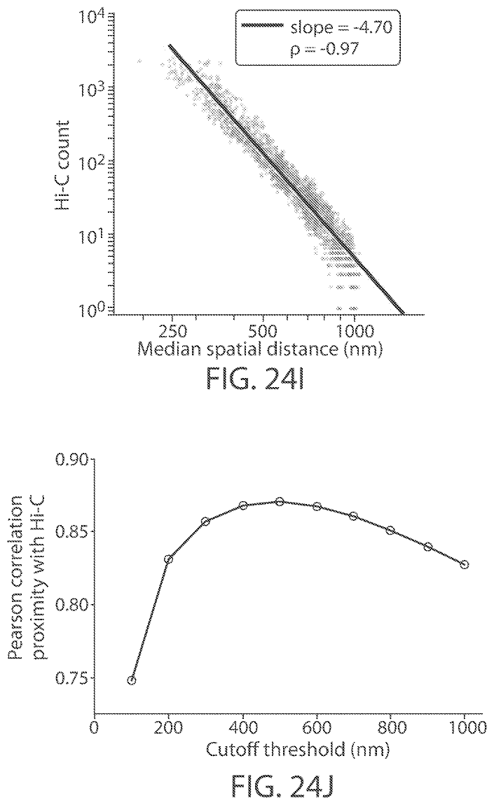

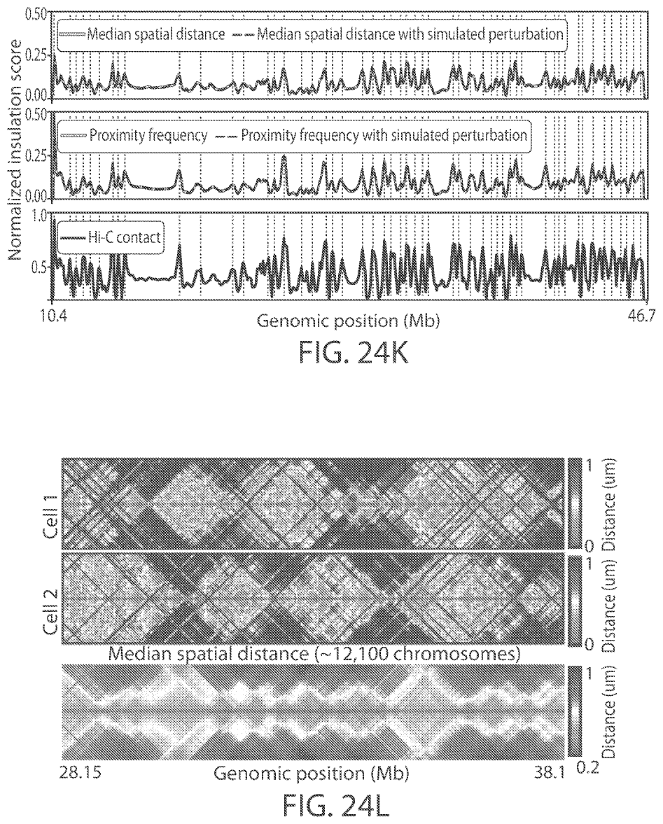

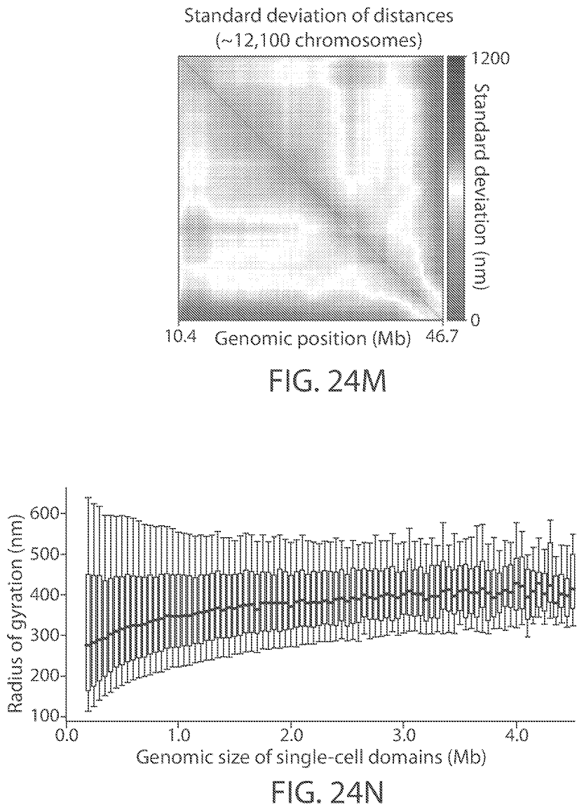

In one set of embodiments, a plurality of adaptors may be used. The adaptors may comprise a first portion substantially complementary to one or more readout sequences on a nucleic acid probe (e.g., a primary nucleic acid probe), and a second portion comprising one or more identification sequences. The adaptor sequences are thus able to bind to specific nucleic acid probes that may be bound to a target in the sample. The identification sequences are then available for binding, e.g., via readout probes or secondary nucleic acid probes, such as those discussed herein. Thus, in some cases, the adaptor may be present between the primary nucleic acid probes and the secondary nucleic acid probes. One non-limiting example of this is shown in FIG. 24A.

In some cases, the adaptors may be chosen to allow a relatively small number of signaling entities to be used, as noted above. For instance, the identification sequences may act as readout sequences that the secondary nucleic acid probes are able to bind to. In a round of detection, a relatively small number of secondary nucleic acid probes, e.g., containing a signaling entity and a sequence substantially complementary to one of the identification sequences, may be used, and the signaling entities determined, e.g., as discussed herein. The secondary nucleic acid probes may then be removed and/or deactivated, e.g., as described herein, before the next round of detection. The next and subsequent rounds may use the same or different signaling entities, e.g., on secondary nucleic acid probes containing sequences substantially complementary to different identification sequences.

In addition, in some embodiments, to reduce contamination or “cross-talk,” the adapters used in the previous round may be deactivated in some fashion. For instance, blocking nucleic acid probes may be added that contain sequences substantially complementary to the previous identification sequences, such that they are able to bind to the previous adaptors, but since they are not generally detectable without the presence of a signaling entity. Accordingly, in subsequent rounds of detection, signals due to prior rounds may be minimized.

Accordingly, in some cases, relatively large numbers of identification sequences may be determined using no more than a relatively small number of signaling entities. For instance, at least 3, at least 4, at least 7, at least 8, at least 12, at least 14, at least 15, at least 16, at least 20, at least 22, at least 30, at least 31, at least 32, at least 50, at least 63, at least 64, at least 72, at least 75, at least 100, at least 127, at least 128, at least 140, at least 255, at least 256, at least 500, at least 1,000, at least 1,500, at least 2,000, at least 2,500, at least 3,000, at least 4,000, at least 5,000, at least 7,500, at least 10,000, at least 12,000, at least 15,000, at least 20,000, at least 25,000, at least 30,000, at least 40,000, at least 50,000, at least 75,000, or at least 100,000, etc. identification sequences may be determined using no more than 20, no more than 15, no more than 10, no more than 5, no more than 4, no more than 3, or no more than 2 signaling entities.

The identification sequences may be of any length. If more than one identification sequence is used, the identification sequences may independently have the same or different lengths. For instance, the identification sequence may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the identification sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the identification sequence may have a length of between 10 and 30 nucleotides, between 20 and 40 nucleotides, between 5 and 50 nucleotides, between 10 and 200 nucleotides, or between 25 and 35 nucleotides, between 10 and 300 nucleotides, etc.

The identification sequence may be arbitrary or random in some embodiments. In certain cases, the identification sequences are chosen so as to reduce or minimize homology with other components of the cell or other sample, e.g., such that the identification sequences do not themselves bind to or hybridize with other nucleic acids suspected of being within the cell or other sample. In some cases, the homology may be less than 10%, less than 8%, less than 7%, less than 6%, less than 5%, less than 4%, less than 3%, less than 2%, or less than 1%. In some cases, there may be a homology of less than 20 basepairs, less than 18 basepairs, less than 15 basepairs, less than 14 basepairs, less than 13 basepairs, less than 12 basepairs, less than 11 basepairs, or less than 10 basepairs. In some cases, such basepairs are sequential.

In addition, in some embodiments, some or all of the identification sequences may be selected such that they do not exhibit specific binding towards each other and/or towards the genome or other nucleic acids suspected of being present in the sample, such as readout sequences. For instance, a population of identification sequences may be “blasted” or tested for specific binding or complementarity. In some case, the identification sequences may exhibit no specific binding towards each other, and/or such that none of the identification sequences in the population of identification sequences has a complementary of more than 5, 6, 7, 8, 9, 10, etc. nucleotides to another sequence within the population of identification sequences, and/or within a population of readout sequences.

In some embodiments, a sample is first subject to hybridization to a population of primary or encoding nucleic acid probes. One or more of the primary or encoding probe comprises a target sequence that can bind to one of the nucleic acid targets and may also comprises one or more readout sequences. The sample are then subject to multiple rounds of hybridization with adaptor probes and readout probes. The adaptor probes may comprise a sequence that can bind to a readout sequence and also comprises one or more identification sequence. The readout probe may be able to bind to an identification sequence and is also associated with a signaling entity. In some cases, multi-color imaging can be used in each round to allow simultaneous imaging and determination of multiple readout probes associated with different signaling entities.

As discussed herein, in certain aspects, signaling entities are determined, e.g., by imaging, to determine nucleic acid probes and/or to create codewords. Examples of signaling entities include those discussed herein. In some cases, signaling entities within a sample may be determined, e.g., spatially, using a variety of techniques. In some embodiments, the signaling entities may be fluorescent, and techniques for determining fluorescence within a sample, such as fluorescence microscopy or confocal microscopy, may be used to spatially identify the positions of signaling entities within a cell. In some cases, the positions of entities within the sample may be determined in two or even three dimensions. In addition, in some embodiments, more than one signaling entity may be determined at a time (e.g., signaling entities with different colors or emissions), and/or sequentially.

In addition, in some embodiments, a confidence level for an identified target, e.g., a nucleic acid target, may be determined. For example, the confidence level may be determined using a ratio of the number of exact matches to the number of matches having one or more one-bit errors. In some cases, only matches having a confidence ratio greater than a certain value may be used. For instance, in certain embodiments, matches may be accepted only if the confidence ratio for the match is greater than about 0.01, greater than about 0.03, greater than about 0.05, greater than about 0.1, greater than about 0.3, greater than about 0.5, greater than about 1, greater than about 3, greater than about 5, greater than about 10, greater than about 30, greater than about 50, greater than about 100, greater than about 300, greater than about 500, greater than about 1000, or any other suitable value. In addition, in some embodiments, matches may be accepted only if the confidence ratio for the identified target is greater than an internal standard or false positive control by about 0.01, about 0.03, about 0.05, about 0.1, about 0.3, about 0.5, about 1, about 3, about 5, about 10, about 30, about 50, about 100, about 300, about 500, about 1000, or any other suitable value

In some embodiments, the spatial positions of the entities (and thus, nucleic acid probes that the entities may be associated with) may be determined at relatively high resolutions. For instance, the positions may be determined at spatial resolutions of better than about 100 micrometers, better than about 30 micrometers, better than about 10 micrometers, better than about 3 micrometers, better than about 1 micrometer, better than about 800 nm, better than about 600 nm, better than about 500 nm, better than about 400 nm, better than about 300 nm, better than about 200 nm, better than about 100 nm, better than about 90 nm, better than about 80 nm, better than about 70 nm, better than about 60 nm, better than about 50 nm, better than about 40 nm, better than about 30 nm, better than about 20 nm, or better than about 10 nm, etc.

There are a variety of techniques able to determine or image the spatial positions of entities optically, e.g., using fluorescence microscopy. More than one color can be used in some embodiments. In some cases, the spatial positions may be determined at super resolutions, or at resolutions better than the wavelength of light or the diffraction limit. Non-limiting examples include STORM (stochastic optical reconstruction microscopy), STED (stimulated emission depletion microscopy), NSOM (Near-field Scanning Optical Microscopy), 4Pi microscopy, SIM (Structured Illumination Microscopy), SMI (Spatially Modulated Illumination) microscopy, RESOLFT (Reversible Saturable Optically Linear Fluorescence Transition Microscopy), GSD (Ground State Depletion Microscopy), SSIM (Saturated Structured-Illumination Microscopy), SPDM (Spectral Precision Distance Microscopy), Photo-Activated Localization Microscopy (PALM), Fluorescence Photoactivation Localization Microscopy (FPALM), LIMON (3D Light Microscopical Nanosizing Microscopy), Super-resolution optical fluctuation imaging (SOFI), Expansion Microscopy, or the like. See, e.g., U.S. Pat. No. 7,838,302, issued Nov. 23, 2010, entitled “Sub-Diffraction Limit Image Resolution and Other Imaging Techniques,” by Zhuang, et al.; U.S. Pat. No. 8,564,792, issued Oct. 22, 2013, entitled “Sub-diffraction Limit Image Resolution in Three Dimensions,” by Zhuang, et al.; or Int. Pat. Apl. Pub. No. WO 2013/090360, published Jun. 20, 2013, entitled “High Resolution Dual-Objective Microscopy,” by Zhuang, et al., each incorporated herein by reference in their entireties.

As an illustrative non-limiting example, in one set of embodiments, the sample may be imaged with a high numerical aperture, oil immersion objective with 100× magnification and light collected on an electron-multiplying CCD camera. In another example, the sample could be imaged with a high numerical aperture, oil immersion lens with 40× magnification and light collected with a wide-field scientific CMOS camera. With different combinations of objectives and cameras, a single field of view may correspond to no less than 1×1 microns, 10×10 microns, 40×40 microns, 80×80 microns, 120×120 microns, 240×240 microns, 340×340 microns, or 500×500 microns, etc. in various non-limiting embodiments. Similarly, a single camera pixel may correspond, in some embodiments, to regions of the sample of no less than 10×10 nm, 20×20 nm, 40×40 nm, 80×80 nm, 120×120 nm, 160×160 nm, 240×240 nm, or 300×300 nm, etc. In another example, the sample may be imaged with a low numerical aperture, air lens with 10× magnification and light collected with a sCMOS camera. In additional embodiments, the sample may be optically sectioned by illuminating it via a single or multiple scanned diffraction limited foci generated either by scanning mirrors or a spinning disk and the collected passed through a single or multiple pinholes. In another embodiment, the sample may also be illuminated via a thin sheet of light generated via any one of multiple methods known to those versed in the art.

In one embodiment, the sample may be illuminated by single Gaussian mode laser lines. In some embodiments, the illumination profiled may be flattened by passing these laser lines through a multimode fiber that is vibrated via piezo-electric or other mechanical means. In some embodiments, the illumination profile may be flattened by passing single-mode, Gaussian beams through a variety of refractive beam shapers, such as the piShaper or a series of stacked Powell lenses. In yet another set of embodiments, the Gaussian beams may be passed through a variety of different diffusing elements, such as ground glass or engineered diffusers, which may be spun in some cases at high speeds to remove residual laser speckle. In yet another embodiment, laser illumination may be passed through a series of lenslet arrays to produce overlapping images of the illumination that approximate a flat illumination field.

In some embodiments, the centroids of the spatial positions of the entities may be determined. For example, a centroid of a signaling entity may be determined within an image or series of images using image analysis algorithms known to those of ordinary skill in the art. In some cases, the algorithms may be selected to determine non-overlapping single emitters and/or partially overlapping single emitters in a sample. Non-limiting examples of suitable techniques include a maximum likelihood algorithm, a least squares algorithm, a Bayesian algorithm, a compressed sensing algorithm, or the like. Combinations of these techniques may also be used in some cases.

In some embodiments, one or more signaling entities may be determined. For instance, a signaling entity may be bound to the readout probe or the recognition entities on the secondary amplifier nucleic acids (or other ending amplifier nucleic acid). Non-limiting examples of signaling entities include fluorescent entities (fluorophores) or phosphorescent entities, e.g., as discussed herein. The signaling entities may then be determined, e.g., to determine the nucleic acid probes or the targets. In some cases, the determination may be spatial, e.g., in two or three dimensions. In addition, in some cases, the determination may be quantitative, e.g., the amount or concentration of signaling entity and/or of a target may be determined.

In one set of embodiments, the signaling entities may be attached to the secondary amplifier nucleic acid (or other ending amplifier nucleic acid). The signaling entities may be attached to the secondary amplifier nucleic acid (or other ending amplifier nucleic acid) before or after association of the secondary amplifier nucleic acid to targets within the sample. For example, the signaling entities may be attached to the secondary amplifier nucleic acid initially, or after the secondary amplifier nucleic acids have been applied to a sample. In some cases, the signaling entities are added, then reacted to attach them to the amplifier nucleic acids.

In one set of embodiments, the signaling entities may be attached to a nucleotide sequence via a bond that can be cleaved to release the signaling entity. For example, after determining the distribution of nucleic acid probes within a sample, the signaling entities may be released or inactivated, prior to another round of nucleic acid probes and/or amplifier nucleic acids. Thus, in some embodiments, the bond may be a cleavable bond, such as a disulfide bond or a photocleavable bond. Examples of photocleavable bonds are discussed in detail herein. In some cases, such bonds may be cleaved, for example, upon exposure to reducing agents or light (e.g., ultraviolet light). See below for additional details. In some cases, the signaling entity is deactivated by photobleaching. Other examples of systems and methods for inactivating and/or removing the signaling entity are discussed in more detail herein.

In certain embodiments, the use of primary and secondary amplifier nucleic acids may be used to create a maximum number of signaling entities that can be bound to a given nucleic acid probe. For instance, there may be a maximum number of signaling entities that are able to bind to a nucleic acid probe, e.g., due to a maximum number of readout probes with signaling entities that are able to bind to a finite number of secondary amplifier nucleic acids, due to a maximum number of secondary amplifier nucleic acids that are able to bind to a finite number of primary amplifier nucleic acids, and/or due to a maximum number of primary amplifier nucleic acids that are able to bind to the finite number of read sequences on the nucleic acid probes. While each potential location need not actually be filled with a signaling entity, this structure suggests that there is a saturation limit of signaling entities, beyond which any additional signaling entities that may happen to be present are unable to associate with a nucleic acid probe or its target.

Accordingly, certain embodiments are generally directed to systems and methods of amplifying a signal indicating a nucleic acid probe or its target that are saturatable, i.e., such that there is an upper, saturation limit of how many signaling entities can associate with the nucleic acid probe or its target. Typically, that number is greater than 1. For instance, the upper limit of signaling entities may be at least 2, at least 3, at least 5, at least 10, at least 15, at least 20, at least 25, at least 30, at least 40, at least 50, at least 75, at least 100, at least 125, at least 150, at least 175, at least 200, at least 250, at least 300, at least 400, at least 500, etc. In some cases, the upper limit may be less than 500, less than 400, less than 300, less than 250, less than 200, less than 175, less than 150, less than 125, less than 100, less than 75, less than 50, less than 40, less than 30, less than 25, less than 20, less than 15, less than 10, less than 5, etc. In some cases, the upper limit may be determined as the maximum number of signaling entities that can bind to a secondary amplifier nucleic acid, multiplied by the maximum number of secondary amplifier nucleic acids that can bind to a primary amplifier nucleic acid, multiplied by the maximum number of primary amplifier nucleic acids that can bind to a nucleic acid probe that binds to a target. In contrast, techniques such as rolling circle amplification or hairpin unfolding allow the amplification of a signal in an uncontrolled manner, i.e., when sufficient reagents are present, amplification can continue without a predetermined endpoint or saturation limit. Thus, such techniques have no theoretical upper limit as to the number of signaling entities that can associate with the nucleic acid probe or its target.

It should be understood, however, that the average number of signaling entities actually bound to a nucleic acid probe or its target need not actually be the same as its upper limit, i.e., the signaling entities may not actually be at full saturation (although they can be). For instance, the amount of saturation (or the number of signaling entities bound, relative to the maximum number that can bind) may be less than 97%, less than 95%, less than 90%, less than 85%, less than 80%, less than 75%, etc., and/or at least 50%, at least 75%, at least 80%, at least 85%, at least 90%, at least 95%, at least 97%, etc. In some cases, allowing more time for binding to occur and/or increasing the concentration of reagents may increase the amount of saturation.

Because of the potential upper limit on the number of signaling entities actually bound to a nucleic acid probe or its target, the binding events distributed within a sample, e.g., spatially, may present substantially uniform sizes and/or brightnesses, in contrast to uncontrolled amplifications, such as those discussed above. For instance, due to the specific number of secondary amplifier nucleic acids that can bind to a primary amplifier nucleic acids, the secondary amplifier nucleic acids cannot be found greater than a fixed distance from the nucleic acid probe or its target, which may limit the “spot size” or diameter of fluorescence from the signaling entities, indicating binding.

In certain embodiments, at least 50%, at least 60%, at least 70%, at least 75%, at least 80%, at least 85%, at least 90%, or at least 95% of the binding events may exhibit substantially the same brightnesses, sizes (e.g., apparent diameters), colors, or the like, which may make it easier to distinguish binding events from other events, such as nonspecific binding, noise, or the like.

In addition, the signaling entity may be inactivated in some cases. For example, in some embodiments, a first secondary nucleic acid probe or readout probe that can associate with a signaling entity (e.g., using amplifier nucleic acids) may be applied to a sample that can recognize a first readout sequence (e.g., on the primary or encoding nucleic acid probe), then the signaling entity can be inactivated before a second secondary nucleic acid probe or readout probe is applied to the sample, e.g., that can associate with a signaling entity (e.g., using amplifier nucleic acids). If multiple signaling entities are used, the same or different techniques may be used to inactivate the signaling entities, and some or all of the multiple signaling entities may be inactivated, e.g., sequentially or simultaneously.

Inactivation may be caused by removal of the signaling entity (e.g., from the sample, or from the nucleic acid probe, etc.), and/or by chemically altering the signaling entity in some fashion (e.g., by photobleaching the signaling entity, bleaching or chemically altering the structure of the signaling entity, for example, by reduction, etc.). For instance, in one set of embodiments, a fluorescent signaling entity may be inactivated by chemical or optical techniques such as oxidation, photobleaching, chemically bleaching, stringent washing or enzymatic digestion or reaction by exposure to an enzyme, dissociating the signaling entity from other components (e.g., a probe), chemical reaction of the signaling entity (e.g., to a reactant able to alter the structure of the signaling entity) or the like. For instance, bleaching may occur by exposure to oxygen, reducing agents, or the signaling entity could be chemically cleaved from the nucleic acid probe and washed away via fluid flow.

In some embodiments, various nucleic acid probes may be associated with one or more signaling entities, e.g., using amplifier nucleic acids as discussed herein. If more than one nucleic acid probe (or secondary nucleic acid probes or readout probes) is used, the signaling entities may each be the same or different. In certain embodiments, a signaling entity is any entity able to emit light. For instance, in one embodiment, the signaling entity is fluorescent. In other embodiments, the signaling entity may be phosphorescent, radioactive, absorptive, etc. In some cases, the signaling entity is any entity that can be determined within a sample at relatively high resolutions, e.g., at resolutions better than the wavelength of visible light or the diffraction limit. The signaling entity may be, for example, a dye, a small molecule, a peptide or protein, or the like. The signaling entity may be a single molecule in some cases. If multiple secondary nucleic acid probes or readout probes are used, the nucleic acid probes may associate with the same or different signaling entities.

Non-limiting examples of signaling entities include fluorescent entities (fluorophores) or phosphorescent entities, for example, cyanine dyes (e.g., Cy2, Cy3, Cy3B, Cy5, Cy5.5, Cy7, etc.), Alexa Fluor dyes, Atto dyes, photoswitchable dyes, photoactivatable dyes, fluorescent dyes, metal nanoparticles, semiconductor nanoparticles or “quantum dots,”

In one set of embodiments, the signaling entity may be attached to an oligonucleotide sequence via a bond that can be cleaved to release the signaling entity. In one set of embodiments, a fluorophore may be conjugated to an oligonucleotide via a cleavable bond, such as a photocleavable bond. Non-limiting examples of photocleavable bonds include, but are not limited to, 1-(2-nitrophenyl)ethyl, 2-nitrobenzyl, biotin phosphoramidite, acrylic phosphoramidite, diethylaminocoumarin, 1-(4,5-dimethoxy-2-nitrophenyl)ethyl, cyclo-dodecyl (dimethoxy-2-nitrophenyl)ethyl, 4-aminomethyl-3-nitrobenzyl, (4-nitro-3-(1-chlorocarbonyloxyethyl)phenyl)methyl-S-acetylthioic acid ester, (4-nitro-3-(1-thlorocarbonyloxyethyl)phenyl)methyl-3-(2-pyridyldithiopropionic acid) ester, 3-(4,4′-dimethoxytrityl)-1-(2-nitrophenyl)-propane-1,3-diol-[2-cyanoethyl-(N,N-diisopropyl)]-phosphoramidite, 1-[2-nitro-5-(6-trifluoroacetylcaproamidomethyl)phenyl]-ethyl-[2-cyano-ethyl-(N,N-diisopropyl)]-phosphoramidite, 1-[2-nitro-5-(6-(4,4′-dimethoxytrityloxy)butyramidomethyl)phenyl]-ethyl-[2-cyanoethyl-(N,N-diisopropyl)]-phosphoramidite, 1-[2-nitro-5-(6-(N-(4,4′-dimethoxytrityl))-biotinamidocaproamido-methyl)phenyl]-ethyl-[2-cyanoethyl-(N,N-diisopropyl)]-phosphoramidite, or similar linkers. The oligonucleotide sequence may be, for example, a primary or secondary (or other) amplifier nucleic acid, such as those discussed herein.

In another set of embodiments, the fluorophore may be conjugated to an oligonucleotide via a disulfide bond. The disulfide bond may be cleaved by a variety of reducing agents such as, but not limited to, dithiothreitol, dithioerythritol, beta-mercaptoethanol, sodium borohydride, thioredoxin, glutaredoxin, trypsinogen, hydrazine, diisobutylaluminum hydride, oxalic acid, formic acid, ascorbic acid, phosphorous acid, tin chloride, glutathione, thioglycolate, 2,3-dimercaptopropanol, 2-mercaptoethylamine, 2-aminoethanol, tris(2-carboxyethyl)phosphine, bis(2-mercaptoethyl) sulfone, N,N′-dimethyl-N,N′-bis(mercaptoacetyl)hydrazine, 3-mercaptoproptionate, dimethylformamide, thiopropyl-agarose, tri-n-butylphosphine, cysteine, iron sulfate, sodium sulfite, phosphite, hypophosphite, phosphorothioate, or the like, and/or combinations of any of these. The oligonucleotide may be, for example, a primary nucleic acid probe, an encoding nucleic acid probe, a readout probe, a primary or secondary (or other) amplifier nucleic acid, such as those discussed herein.

In another embodiment, the fluorophore may be conjugated to an oligonucleotide via one or more phosphorothioate modified nucleotides in which the sulfur modification replaces the bridging and/or non-bridging oxygen. The fluorophore may be cleaved from the oligonucleotide, in certain embodiments, via addition of compounds such as but not limited to iodoethanol, iodine mixed in ethanol, silver nitrate, or mercury chloride. In yet another set of embodiments, the signaling entity may be chemically inactivated through reduction or oxidation. For example, in one embodiment, a chromophore such as Cy5 or Cy7 may be reduced using sodium borohydride to a stable, non-fluorescence state. In still another set of embodiments, a fluorophore may be conjugated to an oligonucleotide via an azo bond, and the azo bond may be cleaved with 2-[(2-N-arylamino)phenylazo]pyridine. In yet another set of embodiments, a fluorophore may be conjugated to an oligonucleotide via a suitable nucleic acid segment that can be cleaved upon suitable exposure to DNAse, e.g., an exodeoxyribonuclease or an endodeoxyribonuclease. Examples include, but are not limited to, deoxyribonuclease I or deoxyribonuclease II. In one set of embodiments, the cleavage may occur via a restriction endonuclease. Non-limiting examples of potentially suitable restriction endonucleases include BamHI, BsrI, NotI, XmaI, PspAI, DpnI, MboI, MnlI, Eco57I, Ksp632I, DraIII, AhaII, SmaI, MluI, HpaI, ApaI, MI, BstEII, TaqI, EcoRI, SacI, HindII, HaeII, DraII, Tsp509I, Sau3AI, PacI, etc. Over 3000 restriction enzymes have been studied in detail, and more than 600 of these are available commercially. In yet another set of embodiments, a fluorophore may be conjugated to biotin, and the oligonucleotide conjugated to avidin or streptavidin. An interaction between biotin and avidin or streptavidin allows the fluorophore to be bound to the oligonucleotide, while sufficient exposure to an excess of addition, free biotin could “outcompete” the linkage and thereby cause the fluorophore to unbind from the oligonucleotide. In addition, in another set of embodiments, the probes may be removed using corresponding “toe-hold-probes,” which comprise the same sequence as the secondary or readout probe, as well as an extra number of bases of homology to the primary or encoding probes (e.g., 1-20 extra bases, for example, 5 extra bases). These probes may remove the labeled secondary or readout probe through a strand-displacement interaction. The oligonucleotide may be, for example, a primary nucleic acid probe, an encoding nucleic acid probe, a readout probe, a primary or secondary (or other) amplifier nucleic acid, such as those discussed herein.

As used herein, the term “light” generally refers to electromagnetic radiation, having any suitable wavelength (or equivalently, frequency). For instance, in some embodiments, the light may include wavelengths in the optical or visual range (for example, having a wavelength of between about 400 nm and about 700 nm, i.e., “visible light”), infrared wavelengths (for example, having a wavelength of between about 300 micrometers and 700 nm), ultraviolet wavelengths (for example, having a wavelength of between about 400 nm and about 10 nm), or the like. In certain cases, as discussed herein, more than one entity may be used, i.e., entities that are chemically different or distinct, for example, structurally. However, in other cases, the entities may be chemically identical or at least substantially chemically identical.

In one set of embodiments, the signaling entity is “switchable,” i.e., the entity can be switched between two or more states, at least one of which emits light having a desired wavelength. In the other state(s), the entity may emit no light, or emit light at a different wavelength. For instance, an entity may be “activated” to a first state able to produce light having a desired wavelength, and “deactivated” to a second state not able to emit light of the same wavelength. An entity is “photoactivatable” if it can be activated by incident light of a suitable wavelength. As a non-limiting example, Cy5 or Alexa 647, can be switched between a fluorescent and a dark state in a controlled and reversible manner by light of different wavelengths, i.e., 633 nm (or 642 nm, 647 nm, 656 nm) red light can switch or deactivate Cy5 or Alexa 647 to a stable dark state, while 405 nm green light can switch or activate the Cy5 or Alexa 647 back to the fluorescent state. In some cases, the entity can be reversibly switched between the two or more states, e.g., upon exposure to the proper stimuli. For example, a first stimuli (e.g., a first wavelength of light) may be used to activate the switchable entity, while a second stimuli (e.g., a second wavelength of light) may be used to deactivate the switchable entity, for instance, to a non-emitting state. Any suitable method may be used to activate the entity. For example, in one embodiment, incident light of a suitable wavelength may be used to activate the entity to emit light, i.e., the entity is “photoswitchable.” Thus, the photoswitchable entity can be switched between different light-emitting or non-emitting states by incident light, e.g., of different wavelengths. The light may be monochromatic (e.g., produced using a laser) or polychromatic. In another embodiment, the entity may be activated upon stimulation by electric field and/or magnetic field. In other embodiments, the entity may be activated upon exposure to a suitable chemical environment, e.g., by adjusting the pH, or inducing a reversible chemical reaction involving the entity, etc. Similarly, any suitable method may be used to deactivate the entity, and the methods of activating and deactivating the entity need not be the same. For instance, the entity may be deactivated upon exposure to incident light of a suitable wavelength, or the entity may be deactivated by waiting a sufficient time.

Typically, a “switchable” entity can be identified by one of ordinary skill in the art by determining conditions under which an entity in a first state can emit light when exposed to an excitation wavelength, switching the entity from the first state to the second state, e.g., upon exposure to light of a switching wavelength, then showing that the entity, while in the second state can no longer emit light (or emits light at a much reduced intensity) when exposed to the excitation wavelength.

In one set of embodiments, as discussed, a switchable entity may be switched upon exposure to light. In some cases, the light used to activate the switchable entity may come from an external source, e.g., a light source such as a laser light source, another light-emitting entity proximate the switchable entity, etc. The second, light emitting entity, in some cases, may be a fluorescent entity, and in certain embodiments, the second, light-emitting entity may itself also be a switchable entity.

In some embodiments, the switchable entity includes a first, light-emitting portion (e.g., a fluorophore), and a second portion that activates or “switches” the first portion. For example, upon exposure to light, the second portion of the switchable entity may activate the first portion, causing the first portion to emit light. Examples of activator portions include, but are not limited to, Alexa Fluor 405 (Invitrogen), Alexa Fluor 488 (Invitrogen), Cy2 (GE Healthcare), Cy3 (GE Healthcare), Cy3B (GE Healthcare), Cy3.5 (GE Healthcare), or other suitable dyes. Examples of light-emitting portions include, but are not limited to, Cy3B (GE Healthcare), Cy5, Cy5.5 (GE Healthcare), Cy7 (GE Healthcare), Alexa Fluor 647 (Invitrogen), Alexa Fluor 680 (Invitrogen), Alexa Fluor 700 (Invitrogen), Alexa Fluor 750 (Invitrogen), Alexa Fluor 790 (Invitrogen), DiD, DiR, YOYO-3 (Invitrogen), YO-PRO-3 (Invitrogen), TOT-3 (Invitrogen), TO-PRO-3 (Invitrogen) or other suitable dyes. See, e.g., U.S. Pat. No. 7,838,302, incorporated herein by reference in its entirety. In some cases, the first, light-emitting portion can subsequently be deactivated by any suitable technique (e.g., by directing 647 nm red light to the Cy5 portion of the molecule).

In some embodiments, a plurality of nucleic acid probes are used that have different sequences, and the distribution of each of the nucleic acid probes is sequentially analyzed and used to create “codewords” for each location, based on the binding patterns of each of the nucleic acid probes. By selecting nucleic acid probes that define a suitable code space, apparent errors in the observed binding patterns can be identified, and/or discarded and/or corrected to identify the correct codeword, and thus the correct target of the nucleic acid probes within the sample. This error-robustness and error-correction system was first introduced for multiplexed error-robust fluorescence in situ hybridization (MERFISH), and has also been subsequently used in various related techniques. See, e.g., Int. Pat. Apl. Pub. Nos. WO 2016/018960 and WO 2016/018963, each incorporated herein by reference in its entirety.

As mentioned, in certain embodiments, such techniques may be combined with error correction, e.g., as is used in MERFISH or other similar techniques. For example, codewords may be based on the binding (or non-binding) of the plurality of readout probes that can bind to readout sequences on primary or encoding nucleic acid probes, and in some cases, the codewords may define an error-correcting code to help reduce or prevent misidentification of the nucleic acid probes. In some cases, a relatively large number of different targets may be identified using a relatively small number of readout probes, e.g., by using various combinatorial approaches. Fluorescence microscopy, wide-field fluorescence microscopy, epi-fluorescence microscopy, confocal microscopy, or light-sheet microscopy can be used for image acquisition. Image acquisition techniques such as STORM or other super-resolution imaging methods can also be used to image such samples and facilitate determination of the nucleic acid probes. See, e.g., U.S. Pat. Nos. 9,712,805 or 10,073,035, or Int. Pat. Apl. Pub. Nos. WO 2008/091296 or WO 2009/085218, each incorporated herein by reference in its entirety, for additional details regarding techniques such as MERFISH. In some cases, expansion microscopy can also be used in which the sample is expanded before imaging. See, e.g., Int. Pat. Apl. Pub. No. WO 2018/089445, entitled “Matrix Imprinting and Clearing,” or Int. Pat. Apl. Pub. No. WO 2018/089438, entitled “Multiplexed Imaging Using MERFISH and Expansion Microscopy,” each incorporated herein by reference in its entirety.

Another aspect is directed to a computer-implemented method. For instance, a computer and/or an automated system may be provided that is able to automatically and/or repetitively perform any of the methods described herein. As used herein, “automated” devices refer to devices that are able to operate without human direction, i.e., an automated device can perform a function during a period of time after any human has finished taking any action to promote the function, e.g. by entering instructions into a computer to start the process. Typically, automated equipment can perform repetitive functions after this point in time. The processing steps may also be recorded onto a machine-readable medium in some cases.

For example, in some cases, a computer may be used to control imaging of the sample, e.g., using fluorescence microscopy, wide-field fluorescence microscopy, epi-fluorescence microscopy, confocal microscopy, light-sheet microscopy, diffraction-limited light microscopy, STORM or other super-resolution techniques such as those described herein. In some cases, the computer may also control operations such as drift correction, physical registration, hybridization and cluster alignment in image analysis, cluster decoding (e.g., fluorescent cluster decoding), error detection or correction (e.g., as discussed herein), noise reduction, identification of foreground features from background features (such as noise or debris in images), or the like. As an example, the computer may be used to control activation and/or excitation and/or deactivation of signaling entities within the sample, and/or the acquisition of images of the signaling entities. In one set of embodiments, a sample may be excited using light having various wavelengths and/or intensities, and the sequence of the wavelengths of light used to excite the sample may be correlated, using a computer, to the images acquired of the sample containing the signaling entities. For instance, the computer may apply light having various wavelengths and/or intensities to a sample to yield different average numbers of signaling entities in each region of interest (e.g., one activated entity per location, two activated entities per location, etc.). In some cases, this information may be used to construct an image and/or determine the locations of the signaling entities, in some cases at high resolutions, as noted above.

In some aspects, the sample is positioned on a microscope. In some cases, the microscope may contain one or more channels, such as fluidic or microfluidic channels, to direct or control fluid to or from the sample. For instance, in one embodiment, nucleic acid probes such as those discussed herein may be introduced and/or removed from the sample by flowing fluid through one or more channels to or from the sample. In some cases, there may also be one or more chambers or reservoirs for holding fluid, e.g., in fluidic communication with the channel, and/or with the sample. Those of ordinary skill in the art will be familiar with channels, including fluidic or microfluidic channels, for moving fluid to or from a sample.

The following documents are incorporated herein by reference: Int. Pat. Apl. Pub. Nos. WO 2018/218150, entitled “Systems and Methods for High-Throughput Image-Based Screening”; WO 2016/018960, entitled “Systems and Methods for Determining Nucleic Acids”; WO 2016/018963, entitled “Probe Library Construction”; WO 2018/089445, entitled “Matrix Imprinting and Clearing”; WO 2018/089438, entitled “Multiplexed Imaging Using MERFISH and Expansion Microscopy”; and U.S. Pat. Apl. Ser. Nos. 62/836,578, entitled “Imaging-Based Pooled CRISPR Screening” and 62/779,333, entitled “Amplification Methods and Systems for MERFISH and Other Applications.” The following documents are also incorporated herein by reference in their entireties: U.S. Pat. Nos. 2017/0220733 and 2017/0212986.

In addition, U.S. Pat. Apl. Pub. No. 62/954,720, entitled “Genome-Scale Imaging of the 3D Organization and Transcriptional Activity of Chromatin,” and U.S. Pat. Apl. Pub. No. 63/060,947, entitled “Genome-Scale Imaging of the 3D Organization and Transcriptional Activity of Chromatin,” are each incorporated herein by reference in its entirety.

The following examples are intended to illustrate certain embodiments of the present invention, but do not exemplify the full scope of the invention.

### Example 1

The following examples show a massively multiplexed FISH method for imaging the 3D organization of chromatin at the genome scale in single cells and further demonstrate the ability to place 3D genome organization in its native structural and functional context by combining chromatin and nascent transcript imaging, both at the genome scale, with nuclear-structure identification.

This first example reports a massively multiplexed FISH approach to allow genome-scale imaging of chromatin organization in single cells. Using this approach, imaging and identification of >1,000 distinct genomic loci (2,000 chromatin loci counting homologous pairs of chromosomes) across the human genome in single cells was demonstrated. Moreover, simultaneous imaging of these genomic loci was demonstrated with nascent RNA transcripts of >1,000 genes residing in these loci in the context of various nuclear structures, including nuclear speckles, nucleoli and nuclear lamina. This approach was used to explore the relationship between chromatin organization, transcriptional activity, and nuclear context in single cells.

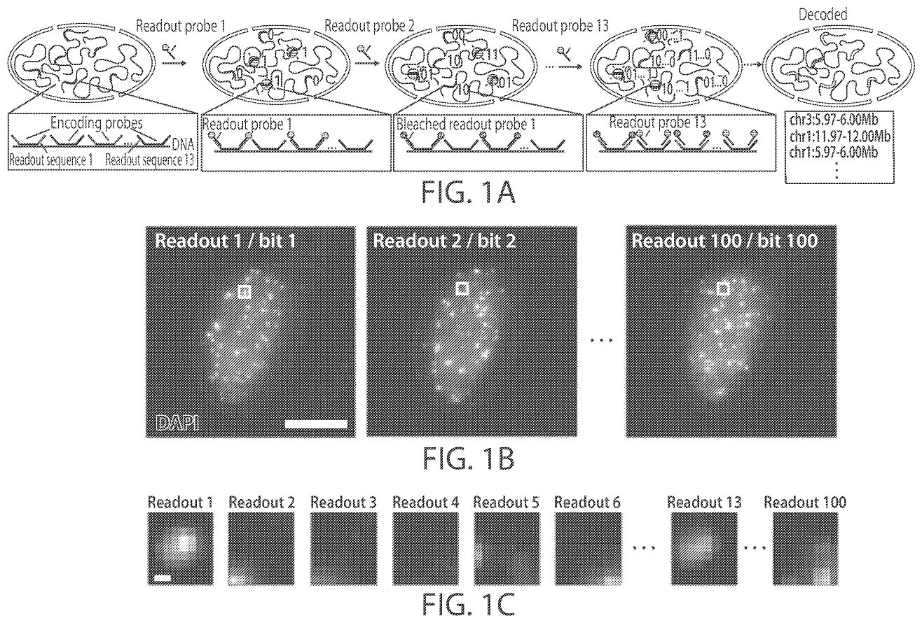

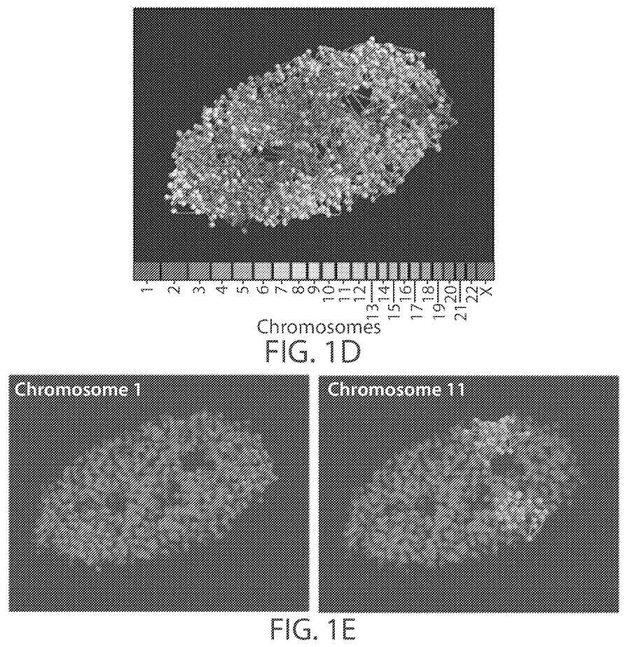

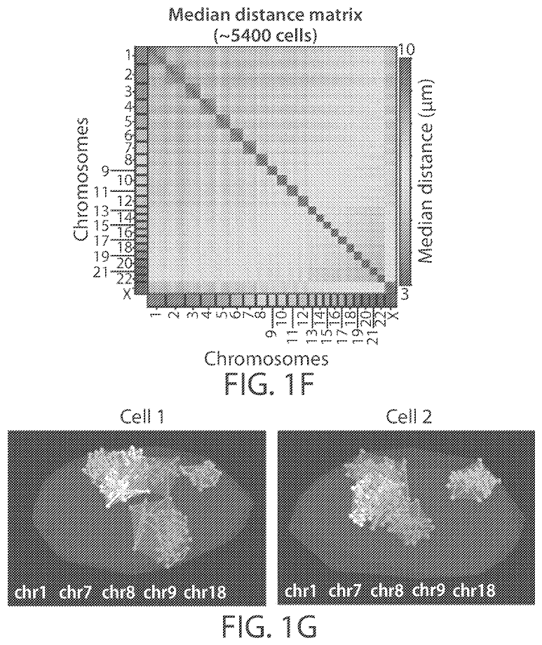

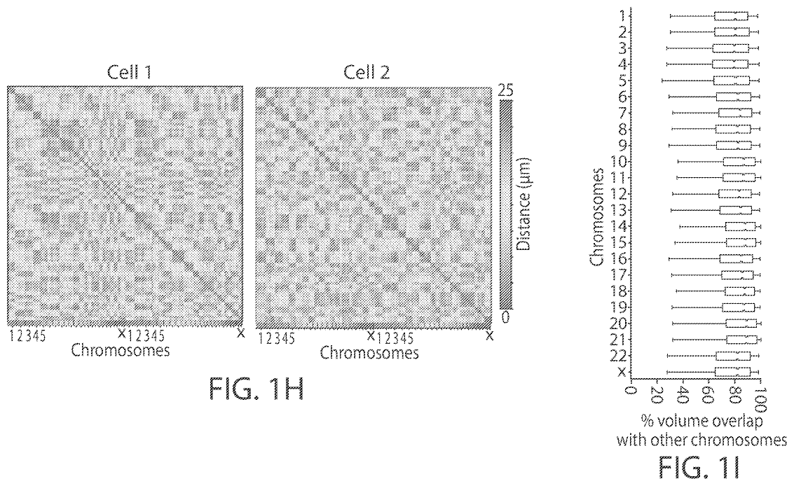

To achieve genome-scale chromatin imaging, a combinatorial FISH approach was devised, inspired by the multiplexed error-robust FISH method that was previously developed for transcriptome imaging, but with significant modifications specifically designed for chromatin imaging by considering both the polymeric nature of chromatin (i.e. adjacent loci in the genomic sequence are spatially close) and the territorial organization of chromosomes (i.e. distinct chromosomes tend to occupy separate spatial territories). See, e.g., WO 2016/018960, entitled “Systems and Methods for Determining Nucleic Acids”; WO 2016/018963, entitled “Probe Library Construction,” each incorporated herein by reference in its entirety. To allow combinatorial imaging, each genomic locus was assigned a unique 100-bit binary code with a Hamming weight of 2, i.e. each barcode containing two “1” bits and 98 “0” bits (FIG. 1A). The bit values in these barcodes determined the presence (1) or absence (0) of signal for each locus across sequential rounds of imaging. In order to avoid imaging spatially close chromatin regions simultaneously in the same bit, from these 100-bit Hamming weight 2 barcodes, a subset was further selected to encode the targeted genomic loci and optimized assignment of these barcodes, such that loci with a “1” bit in the same barcode position were maximally separated in genomic space. This strategy allowed for minimizing detection errors caused by overlapping signals from nearby chromatin loci. Moreover, because the vast majority of the possible 100-bit binary codes were invalid (i.e. not assigned to any targeted locus), this design allowed for identifying and discarding detection errors and further improve measurement accuracy.

The barcodes were physically imprinted onto the targeted genomic loci using a high-diversity library of encoding probes, each containing a 40-nt target region for binding to one of the targeted loci and a 20-nt readout sequence chosen from 100 pre-designed readout sequences (FIG. 1A). Each readout sequence corresponded to one of the 100 bits, and the encoding probe set for each genomic locus (˜400 probes per locus) contained only two distinct readout sequences, corresponding to the two bits that read “1” in the barcode assigned to that locus. After encoding probe binding, the barcodes imprinted on the chromatin loci were detected by sequential hybridization of fluorescently labeled readout probes, each complementary to one of the 100 readout sequences (FIG. 1A). Two distinct readout probes were introduced per hybridization round and imaged in two color channels, such that all ˜1000 genomic loci were imaged and identified after 50 rounds of hybridization (FIGS. 1A-1C). In contrast, a straight sequential approach to image 1000 genomic loci would instead have required 500 rounds of hybridization with two-color imaging. Since each chromosome in diploid cells had two homologs, the homolog identities of the imaged loci were further assigned using a clustering algorithm, exploiting the tendency of chromosomes to occupy distinct territories in each nucleus.

In this example, 1,041 genomic loci were selected for imaging, each ˜30-kb in size, uniformly covering the 22 autosomes and the X chromosome in human lung fibroblast (IMR90) cells. It was also required that each chromosome contain at least 30 targeted loci, hence the number of loci imaged per chromosome homolog ranged between 30 and 80 depending on the length of the chromosome. These 1,041 genomic loci in ˜5,400 individual cells across 5 biological replicates were imaged with a detection efficiency of ˜80% for each locus, yielding ˜1700 chromatin loci detected in each cell considering two homologs per chromosome (FIGS. 1D-1E).

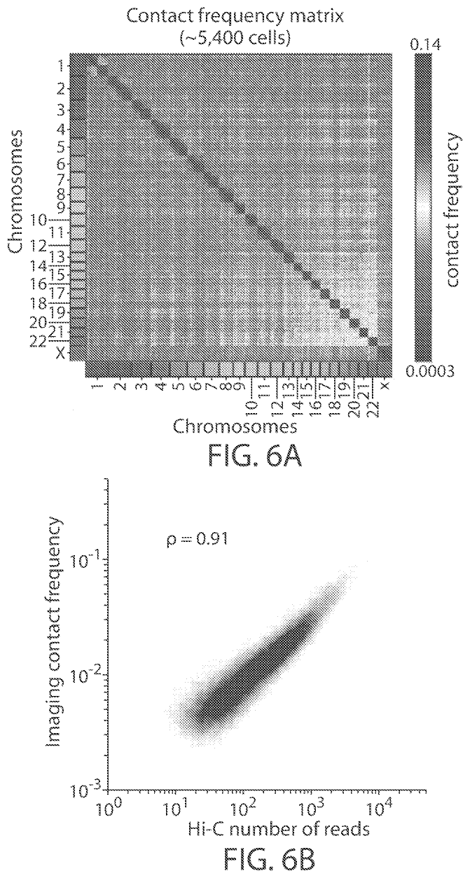

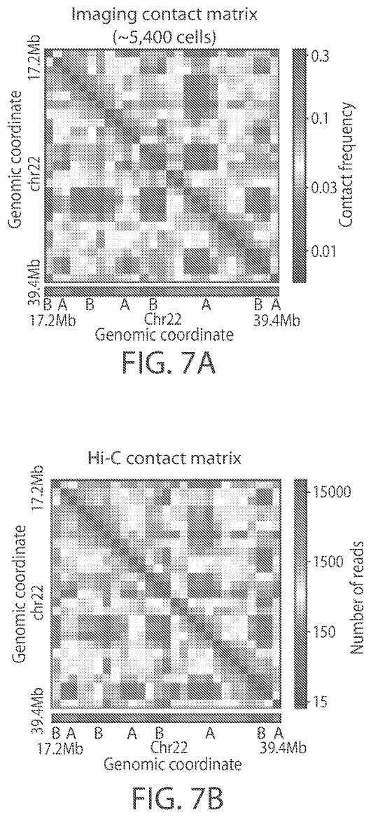

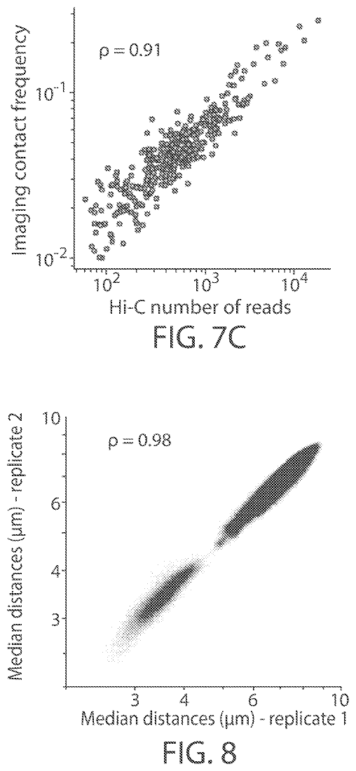

To obtain a population-averaged view of chromatin organization, in each cell, the spatial distance between every pair of imaged chromatin loci was calculated and then both the median distance and the contact frequency between every pair of loci were determined across all imaged cells (FIG. 1F and FIG. 6A). The contact frequencies between pairs of chromatin loci within the same chromosome determined from the imaging data showed high correlation with the contact frequencies detected by ensemble Hi-C, with a Pearson correlation coefficient of 0.91 (FIG. 6B). The imaging data captured chromatin structures at multiple scales, from the organization of chromosomes into territories (FIG. 1F and FIG. 6A) to the formation of A and B compartments within chromosome arms (FIG. 7A), which also agreed well with compartments identified by ensemble Hi-C measurements (FIGS. 7B-7C). Moreover, the imaging results showed high reproducibility between independent biological replicates (FIG. 8).

By exploring chromatin organization in individual cells, chromosomes, while occupying distinct territories within each cell (FIGS. 1F-1G), also displayed substantial overlap with each other (FIGS. 1G-1H). On average, ˜80% of the convex-hull volume occupied by any given chromosome was shared with other chromosomes in the same cell (FIG. 1I), suggesting a high degree of trans-chromosomal interactions. Since these interactions have been underexplored, the analyses below focused on these trans-chromosomal interactions.

FIGS. 1A-1I show genome-scale chromatin imaging. FIG. 1A shows the imaging scheme. The targeted genomic loci were assigned error-robust barcodes, e.g. 100-bit binary barcodes with a Hamming weight of 2 (i.e. two of the 100 bits reading “1”). The barcodes were imprinted onto the genomic loci with encoding oligonucleotide probes, which recognized the loci and associated two distinct readout sequences with each locus, corresponding to the two bits that read “1” in the barcode assigned to the locus. Each locus was labeled by a total of 400 encoding probes, but only 4 are shown. Fluorescent readout probes complementary to the readout sequences were sequentially added and imaged, allowing the bits that read “1” at each locus and hence the barcode identity of that locus to be determined. ˜1000 genomic loci were imaged. FIG. 1B shows representative images from multiple imaging rounds in the nucleus of a single cell. Fluorescent signal of the chromatin loci from readout probes is shown in a lighter shade, while the signal of 4′,6-diamidino-2-phenylindole (DAPI), used as a nuclear marker, is shown in a darker shade. Scale bar: 5 micrometers. FIG. 1C is zoomed-in images of a small spatial region (box in FIG. 1B) centered around one chromatin locus across all imaging rounds. The locus identity was determined based on the two readout probes (1 and 13) that give signals. Scale bar: 300 nm. FIG. 1D is a 3D rendering of all detected chromatin loci in a single cell, grayscaled according to the chromosomes they belong to. Adjacent loci in genomic sequence are connected by a thin line. FIG. 1E shows chromatin loci of the same cell as in FIG. 1D, but with two homologs of the indicated chromosomes shown in a different greyscale than all other loci. FIG. 1F is a median distance matrix computed from ˜5,400 single cells. For each pair of loci, the median of all observed 3D spatial distances between the loci is presented. FIG. 1G show example images showing the positions of multiple chromosomes territories in single cells. Shaded areas represent the convex hull surrounding each chromosome, which was used as an operational definition of the chromosome territory. FIG. 1H shows distance matrices for the same cells shown in FIG. 1G. The spatial distance between each pair of chromatin loci is shown. Chromosome order is as noted beneath the heatmaps, with the two homologs of each chromosome separately shown. FIG. 1I is a quantification of the fraction of the volume of each chromosome territory that is shared by at least one other chromosome in the same cell. The median (center lines), 25th to 75th percentiles (boxes) and 5th to 95th percentiles (whiskers) are shown. n=10,910 copies of chromosomes (5,455 cells and two homologous copies per cell for each chromosome).

FIGS. 6A-6B show contact frequency matrices derived from genome-scale imaging and comparison with ensemble Hi-C data. FIG. 6A shows a contact frequency matrix for all 1041 genomic loci imaged in this example. The contact frequency between a pair of loci was calculated as the number of incidences in which the measured distance between the loci is shorter than 500 nm divided by the total number of measured distances between the two loci. FIG. 6B shows a correlation plot for the contact frequencies between pairs of loci within chromosomes derived from the imaging data and those derived from ensemble Hi-C experiments, binned at 500 kb and centered around the target loci. The Pearson correlation coefficient was 0.91.

FIGS. 7A-7C show sub-chromosomal structures derived from genome-scale imaging and comparison with ensemble Hi-C data. FIG. 7A shows a contact frequency matrix generated from the imaging data for one arm of chromosome 22. Assignment of each locus to the A or B compartment based on this matrix is shown in the bar beneath the matrix. FIG. 7B shows a contact frequency matrix of the same arm of chromosome 22, computed from Hi-C data, binned at 500 kb and centered around the target loci. The bar beneath the matrix shows the A- and B-compartment assignment of each locus based on this matrix, assigned using the same procedure as FIG. 7A. The A/B compartment assignment derived from the imaging data and Hi-C data are identical. FIG. 7C shows a correlation plot for the contact frequencies between locus pairs in chromosome 22 derived from the imaging data and those derived from ensemble Hi-C experiments. The Pearson correlation coefficient was 0.91.

FIG. 8 shows reproducibility of the chromatin imaging experiments between replicates. Shown in the plot is the correlation of pairwise distances between chromatin loci observed in two independent biological replicates of 1041 genomic loci imaging experiments. The Pearson correlation coefficient between replicates was 0.98. The upper right cloud represents the trans-chromosomal pairwise distances and the lower-left cloud represents the intra-chromosomal pairwise distances.

### Example 2

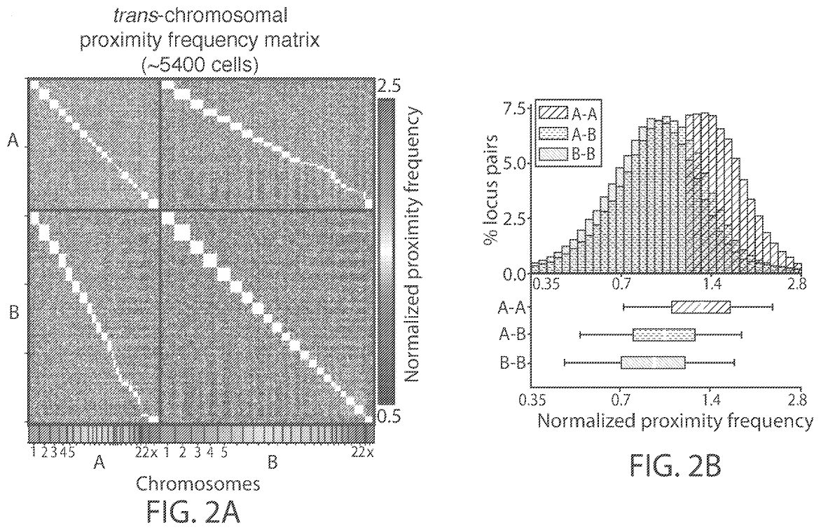

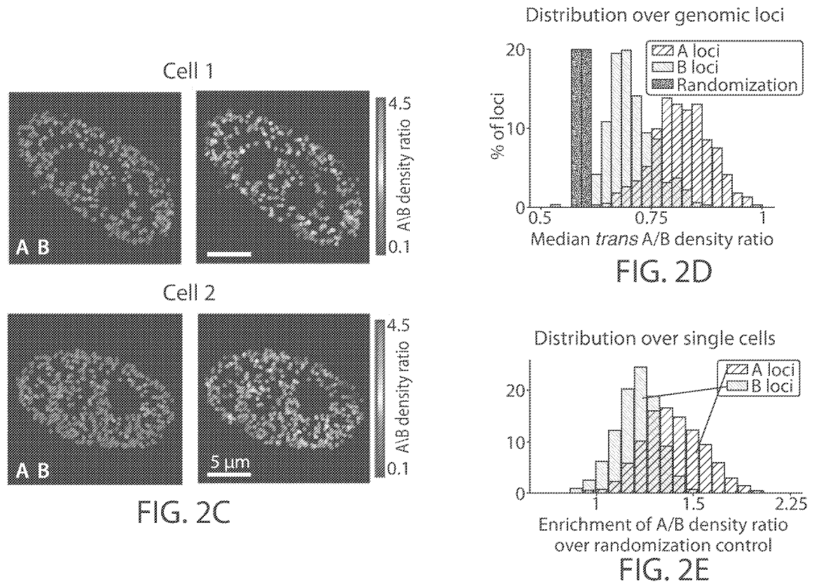

How trans-chromosomal interactions depend on the epigenetic properties of chromatin was studied in this example. It was previously shown by Hi-C and imaging analyses that chromatin is segregated into A and B compartments, respectively enriched with active and inactive chromatin. Distinct mechanisms could mediate active-active and inactive-inactive chromatin interactions, such as HP1-mediated heterochromatin condensation and transcription factor- and cofactor-mediated active chromatin condensation. In this example, each of the imaged genomic loci were classified into compartments A and B using an established calling method based on published ensemble Hi-C data. 38% of the imaged loci belonged to compartment A and tended to be relatively gene rich and enriched with active chromatin markers, such as H3K27Ac, while 62% belonged to compartment B and tended to be enriched with inactive chromatin markers, such as H3K9me3. To examine whether the extent of trans-chromosomal interactions differ for active and inactive chromatin, the genomic loci were ordered in the trans-chromosomal contact frequency matrix, placing all A loci next to each other and likewise all B loci together. This matrix showed that compartment-A loci had a substantially stronger tendency to interact trans-chromosomally with a compartment-A locus than with a compartment-B locus (FIGS. 2A-2B). In contrast, compartment-B loci did not show similar trans-chromosomal affinity towards each other, but instead showed a slightly higher probability to interact with compartment-A chromatin trans-chromosomally (FIGS. 2A-2B). In other words, trans-chromosomal A-A interactions appeared with a substantially stronger tendency than A-B interactions, which in turn appeared with a slightly stronger tendency than B-B interactions. This was in striking contrast with cis-interactions within the same chromosomes, in which A and B compartments tended to segregate, leading to enrichment of both A-A and B-B interactions over A-B interactions.

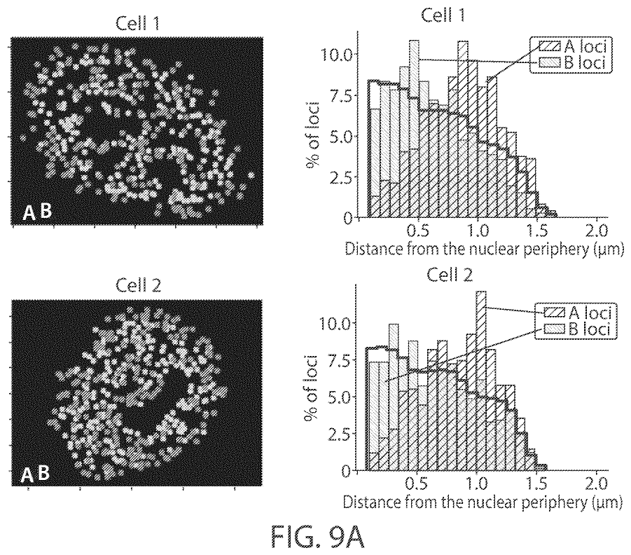

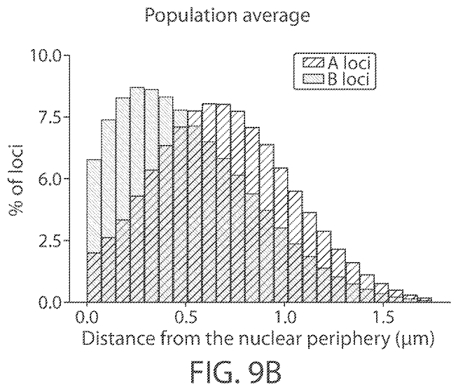

Next, the epigenetic dependence of trans-chromosomal interactions were examined at the single-cell level. In individual cells, compartment-A and compartment-B loci adopted different spatial distributions, with A loci exhibiting a tendency to be more centrally localized than B loci in the nucleus (FIG. 2C and FIGS. 9A-9B). There was also a substantial degree of intermixing between A and B loci (FIG. 2C and FIGS. 9A-9B). For each imaged locus in each chromosome, its local densities of A loci and B loci from all other chromosomes were calculated, and the ratio of these two densities was determined (referred to hereafter as the trans-chromosomal A/B density ratio) (FIG. 2C). This quantity provided a measure of the local enrichment of trans-chromosomal active chromatin near the locus. The majority (62%) of the imaged loci belonged to compartment-B, creating an overall bias for the A/B ratio to be smaller than 1. To control for this bias, distributions of the trans-chromosomal A/B density ratios observed for A loci and for B loci were compared with the distribution obtained in a randomization control where the A and B identities of imaged loci were randomly shuffled among the imaged loci, while keeping the numbers of A and B loci unchanged. Notably, the trans-chromosomal A/B density ratios observed for A loci were substantially higher than the values observed for B loci, which were in turn higher than the values derived from the randomization control (FIG. 2D), and this trend was observed in most single cells (FIG. 2E). These single-cell analyses again supported the notion that trans-chromosomal interactions were preferentially enriched for interactions between active chromatin.

FIGS. 2A-2E show that trans-chromosomal contacts are preferentially enriched for interactions between active chromatin. FIG. 2A shows normalized trans-chromosomal contact frequency matrix. The contact frequency between each trans-chromosomal locus pair (pair of loci on different chromosomes) is shown. The loci are reordered such that compartment-A loci appear first, followed by compartment-B loci, hence the top left block represents interactions between pairs of A loci and the bottom right represents interactions between pairs of B loci. Each entry in the matrix is normalized by the median contact frequency of all locus pairs originating from the same pair of chromosomes to account for varying basal levels of interaction between pairs of chromosomes. FIG. 2B shows distributions of trans-chromosomal contact frequencies for pairs of A loci (AA, right; n=72,771 locus pairs), for pairs of B loci (BB, left; n=193,753 locus pairs), and pairs comprised of one A and one B locus (AB, n=237,986 locus pairs), derived from the matrix shown in FIG. 2A. Distributions are represented in the top panel as histograms and in the bottom panel as box plots, showing the median (center lines), 25th to 75th percentiles (boxes) and 5th to 95th percentiles (whiskers). FIG. 2C shows distributions of compartment-A and compartment-B loci in single cells. The left panels represent the locations of all detected loci within a single z-plane in a single nucleus. Compartment-A loci are shown at the top of the scale, while compartment-B loci are shown at the bottom. In the right panels, the shade of each locus represents the ratio of the local densities of trans-chromosomal A and B loci, in accordance with the shade scale bar shown on the right. FIG. 2D shows distributions of the local trans-chromosomal A/B density ratio for imaged genomic loci. For each locus, the median A/B density ratio across all cells was determined, and the distributions for different loci are shown with A compartment loci (n=382 loci) and B compartment loci (n=623 loci). 36 of the 1041 imaged loci were not assigned A/B identity due to different versions of genome assemblies used in this study and the Hi-C dataset used for compartment calling. The dark grey histogram represents a randomization control where the A and B compartment identity is randomly shuffled, while keeping the total number of A loci and the total number of B loci unchanged. FIG. 2E shows a distribution of the enrichment of trans-chromosomal A/B density ratio over randomization control. For each imaged cell, the median A/B density ratio across all A loci was divided by the median A/B density ratio of the randomization control, as described in FIG. 2D, and the distribution of this value across all imaged cells is presented (n=5,455 cells). The distribution of the same enrichment for B loci is shown (n=5,455 cells). The line marks the value of 1, i.e. no enrichment.

FIGS. 9A-9B show that compartment-A and compartment-B loci display distinct spatial distributions in single cells. FIG. 9A, left panels show example images displaying compartment-A loci and compartment-B loci in a single z-plane of single cells. The right panel shows the distribution of distances to the nuclear periphery for compartment-A loci and compartment-B loci in these single cells. The nuclear periphery is identified as a convex hull surrounding all detected chromatin loci. The histogram shows the distribution of distances from the nuclear periphery for points sampled uniformly within the convex hull surrounding the detected chromatin loci. FIG. 9B shows the population average distributions of the distance to nuclear periphery for compartment-A loci and compartment-B loci. n=382 A loci; n=623 B loci.

### Example 3

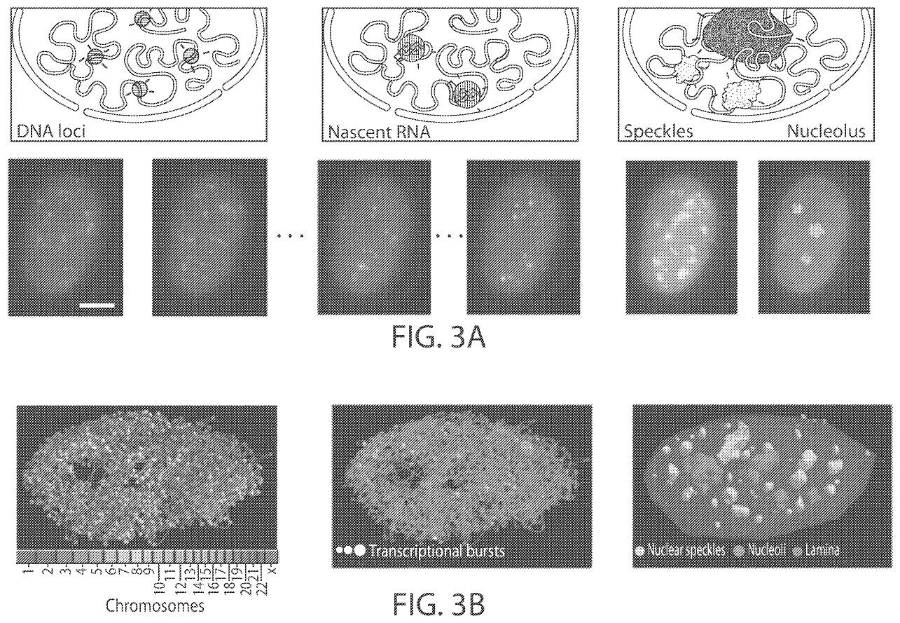

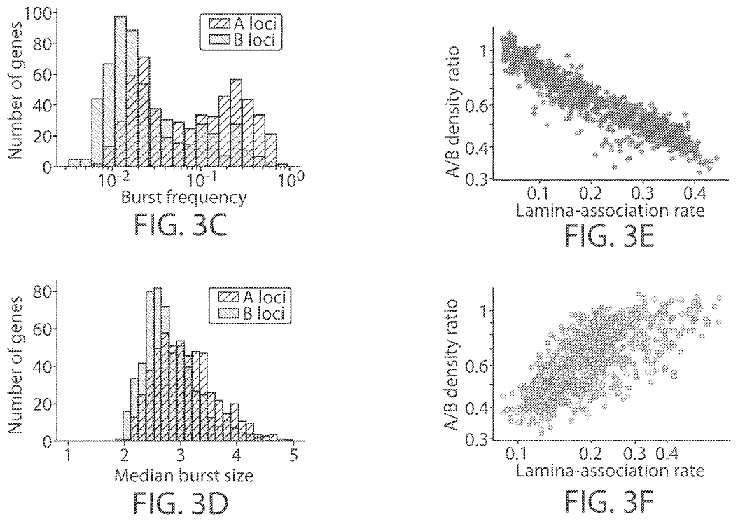

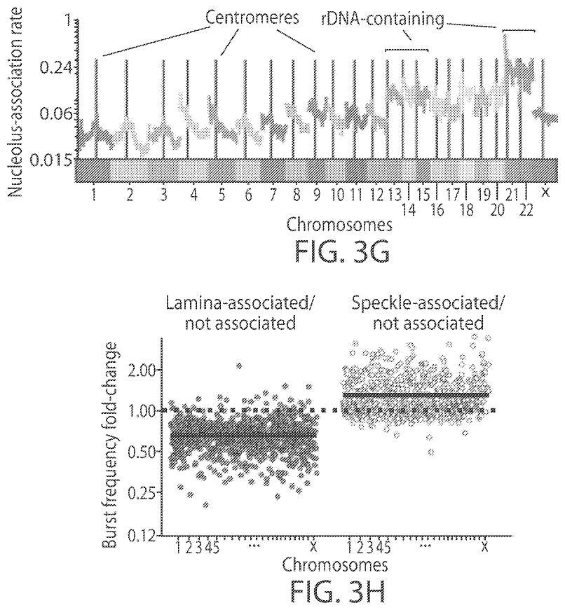

To place the 3D organization of chromatin in the context of its functional activity and other nuclear structures, the imaging method was extended in this example to allow simultaneous measurements of the chromatin organization together with transcriptional activities of numerous genomic loci as well as landmark structures within the nucleus. Specifically, the 1,041 genomic loci were imaged together with the nascent RNA transcribed from each of the 1,137 genes located at these loci and simultaneously with important nuclear structures, including nuclear speckles and nucleoli (FIG. 3A).

To allow DNA, RNA and nuclear-structure imaging within the same cells, multiplexed imaging of the intronic RNAs of the 1,137 genes was performed by adopting a similar combinatorial imaging strategy to the one described above for chromatin (FIG. 3A). Considering that not all genes would be transcribed in each individual cell, and hence the density of transcription foci should not be as high as that of the chromatin loci, the RNAs were encoded with a 54-bit, Hamming weight 2 code, and selected 1,137 of the possible barcodes to encode the genes, in a way similar to how the barcodes for chromatin imaging were selected to minimize the chance of imaging spatially proximal genes in the same bit. After RNA imaging was completed, the RNA transcripts were enzymatically digested (a step also carried out in our single-modal chromatin imaging experiments) and multiplexed DNA FISH was performed as described above to image the 1,041 genomic loci (FIG. 3A). Decoding of genomic loci and nascent RNA transcripts was performed largely independently, with the additional constraint for the transcripts to colocalize with their harboring genomic loci. This procedure further improved detection accuracy for transcribing RNAs and allowed for estimation of the detection efficiency (˜90%) for the transcription bursts at each genomic locus. Finally, nuclear speckles and nucleoli were imaged using immunofluorescence against known molecular components of these structures (FIG. 3A). The positions of nuclear lamina were estimated by computing a convex hull encompassing all imaged genomic loci and determining the boundary of the convex hull. Together, these multi-modal measurements allowed an integrated single-cell view of 3D genome structure, transcriptional activity and nuclear organization (FIG. 3B). These multi-modal imaging experiments were performed on ˜3700 individual cells, in two biological replicates. Chromatin imaging data from these multi-modal experiments were also included in the 5 replicates (5,400 cells) described above for 3D genome organization analyses.

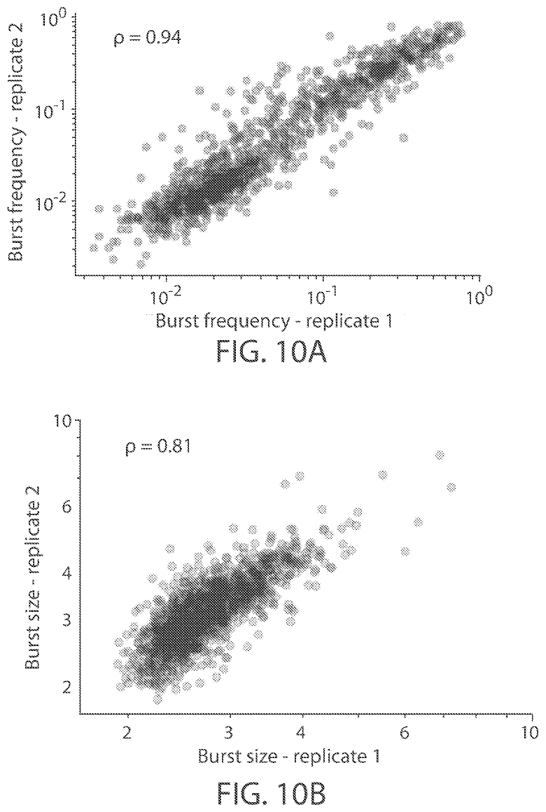

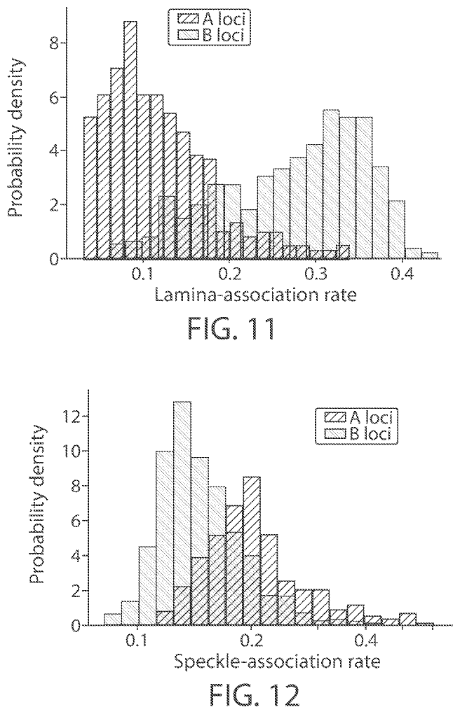

From the nascent RNA transcript measurements of these multi-modal experiments, both the transcriptional burst frequency as the fraction of cells actively transcribing the gene (FIG. 3C) and the median burst size (FIG. 3D) from the brightness of the RNA intron signals were quantified for each gene. These measures showed high correlation across replicate experiments (FIGS. 10A-10B). The burst frequency displayed a bimodal behavior, with high burst frequency genes primarily harbored in compartment A and low burst frequency genes present in both compartments (FIG. 3C). Furthermore, it was estimated whether specific chromatin loci were associated with nuclear bodies using a cut-off spatial distance of 250 nm, and found a higher association frequency of compartment-B loci with nuclear lamina (FIG. 11) and higher association frequency of compartment-A loci with nuclear speckles (FIG. 12). These results were consistent with previous observations of preferential association of inactive and active chromatin with lamina and nuclear speckles, respectively. For individual loci, their local trans-chromosomal A/B density ratio exhibited a negative correlation with the lamina association frequency (FIG. 3E) and a positive correlation with nuclear speckle association frequency (FIG. 3F). Finally, nucleoli showed preferential association with centromeres, with telomeres of certain chromosomes, and with chromosomes containing ribosome-encoding genes (FIG. 3G). These biological results provided further validation to the multi-modal measurements.

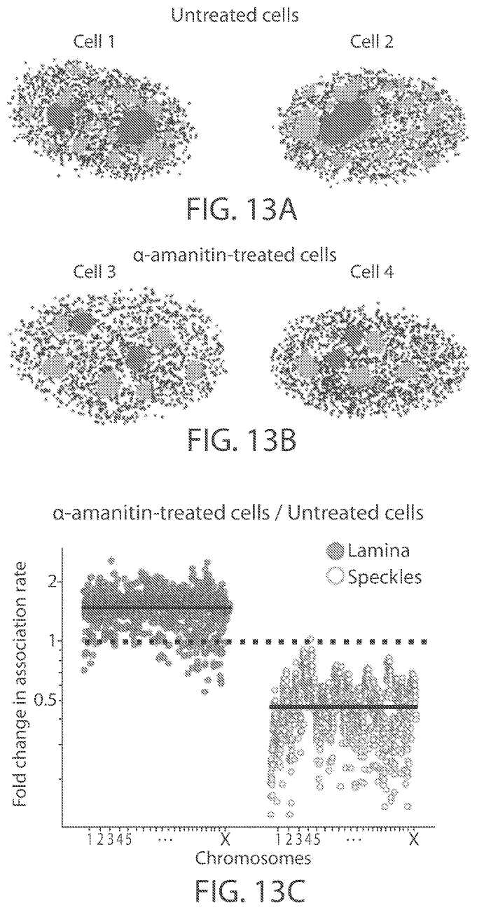

Notably, for essentially all imaged loci, lamina association reduced the observed transcriptional activity (FIG. 3H), while nuclear speckle association correlated with higher transcription activity for most imaged loci (FIG. 3H). In addition, upon treatment with alpha-amanitin to inhibit transcription, the rate of association with lamina globally increased for nearly all loci whereas the rate of association with nuclear speckles globally decreased (FIGS. 13A-13C). These results expanded upon previous imaging studies on the nuclear repositioning of single or a few genomic regions upon transcriptional activation or inhibition and provided a genome-scale view of the relationship between transcriptional activity and interactions with nuclear structures.

FIGS. 3A-3H show genome-scale imaging of chromatin and transcription activity in the context of nuclear structures. FIG. 3A is an illustration of the multi-modal imaging scheme that combines chromatin (left panel), nascent RNA transcripts (middle panel) and nuclear bodies (right panel) imaging to generate an integrated view of chromatin organization in the context of nuclear structures and functional activity. ˜1000 genomic loci, nascent RNA transcripts of ˜1100 genes in the targeted loci, and two types of nuclear bodies (nuclear speckles and nucleoli) are imaged. Below are representative raw images for each imaging modality: chromatin loci across multiple imaging rounds (left), nascent RNA transcripts across multiple imaging rounds (middle) and nuclear bodies (right: nuclear speckles; left: nucleoli). Scale bar: 5 micrometers. FIG. 3B is 3D renderings of chromatin loci, transcriptional bursts and nuclear bodies in a single cell. Left: All detected chromatin loci, grayscaled by chromosome (based on the chromosome index shown below). Middle: All detected intronic RNAs shown as spheres, with shading indicating the identities of the imaged genes and sphere size representing transcription burst size. Chromatin loci are shown in the background. Right: Volume-filling representations of detected nuclear bodies. Nucleoli and nuclear speckles are shown in different shadings. The nuclear lamina is identified as the surface of the convex hull surrounding all detected chromatin loci. FIGS. 3C-3D are distributions of transcription burst frequencies (FIG. 3C) and burst sizes (FIG. 3D) for genes residing in compartment-A loci (n=494 genes) and compartment-B loci (n=625 genes). FIGS. 3E-3F are scatter plots of the local trans-chromosomal A/B density ratio for each imaged genomic locus as a function of the frequency with which the locus is found associated with the nuclear lamina (FIG. 3E) and nuclear speckles (FIG. 3F). A locus is considered associated with a nuclear structure if its measured distance to the structure is smaller than 250 nm. The values of trans-chromosomal A/B density ratio shown in the plots are the median values across all imaged cells. FIG. 3G shows association frequency with nucleoli for all imaged genomic loci, ordered by genomic position. Vertical lines are the locations of centromeres and brackets highlight chromosomes containing ribosome-encoding genes (rDNAs). FIG. 3H shows the effect of nuclear-structure association on transcription. Circles are the fold-change in the transcriptional burst frequency for each locus when comparing the populations of cells in which the locus is lamina-associated versus non-lamina-associated (left) and speckle-associated versus non-speckle associated (right). The dotted line highlights no change and the solid lines represent the median fold-change in each case.

FIGS. 10A-10B show reproducibility of the nascent RNA transcript imaging experiments between replicates. FIGS. 10A-10B show the correlation between replicates of RNA imaging for each gene's burst frequency (FIG. 10A) and burst size (FIG. 10B). Pearson correlation coefficients were 0.94 and 0.81, respectively.

FIG. 11 shows the preferential association of compartment-B loci with nuclear lamina. The distributions of association rates for compartment-A loci (n=382 loci) and compartment-B loci (n=623 loci) with nuclear lamina are shown. A locus is operationally defined as being lamina-associated if its distance to the nuclear periphery is smaller than 250 nm.

FIG. 12 shows the preferential association of compartment-A loci with nuclear speckles. Distributions of association rates for compartment-A loci (n=382 loci) and compartment-B loci (n=623 loci) with nuclear speckles are shown. A locus is operationally defined as being speckle-associated if its distance to the nearest speckle is smaller than 250 nm.

FIGS. 13A-13C show changes in nuclear lamina and nuclear speckle association upon transcription inhibition. FIGS. 13A-13B show representative images of individual nuclei with imaged chromatin loci, nucleoli, and nuclear speckles shown for untreated cells (FIG. 13A) and cells treated with the transcriptional inhibitor alpha-amanitin (FIG. 13B). FIG. 13C shows the fold change in the rate of associate of each locus with lamina (left) and nuclear speckles (right) upon transcription inhibition by alpha-amanitin. The data point for each genomic locus is shown in circles, the solid lines are the median fold changes of all loci in each case, and the dotted line represents no change.

### Example 4

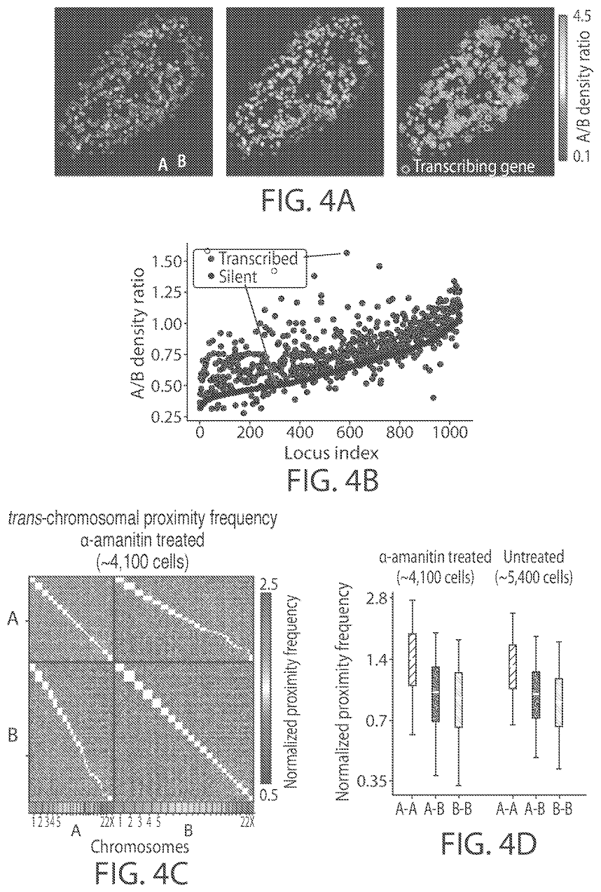

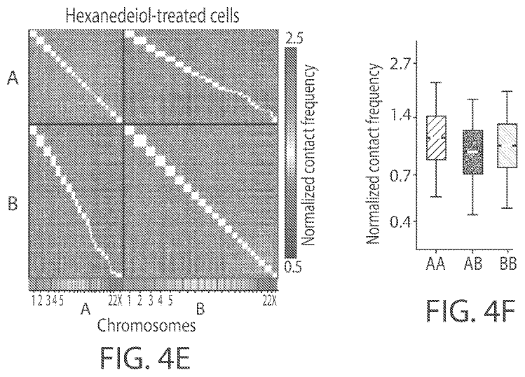

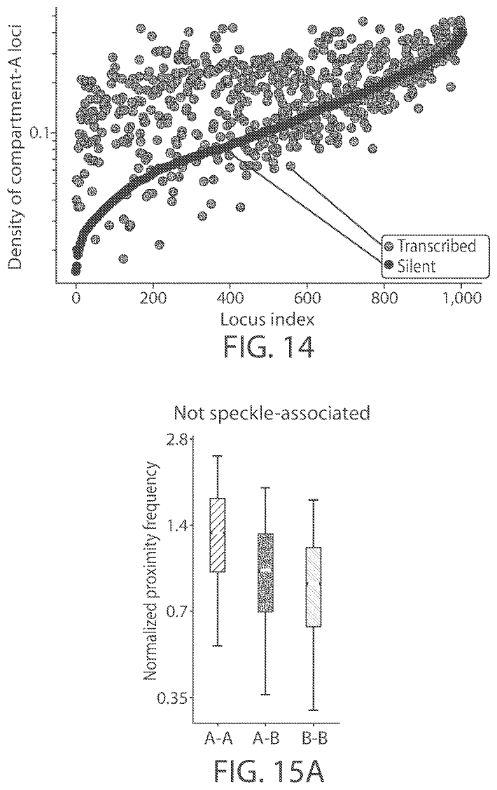

In this example, these multi-modal single-cell measurements were used to further characterize trans-chromosomal interactions in the context of transcriptional activity and nuclear structures. Given the observation that trans-chromosomal interactions were preferentially enriched for interactions between compartment-A loci, it was tested whether these interactions correlate with the transcriptional activity of chromatin. To this end, the local densities of A and B chromatin from other chromosomes and the trans-chromosomal A/B density ratio were calculated for each locus in each cell, and the median values of these quantities for two populations of cells were determined: (i) the cells where the locus under consideration exhibited transcriptional activity (i.e. RNA burst signal), and (ii) the cells where the locus appeared transcriptionally silent at least momentarily (FIG. 4A). Notably, in addition to the observation that compartment-A loci showed higher local trans-chromosomal A/B density ratios than compartment-B loci (FIG. 2D-2E), even for the same locus, a consistent trend was observed for higher trans-chromosomal A density and A/B density ratio when the locus was actively transcribed (FIG. 4B and FIG. 14). This correlation observed between transcriptional activity and trans-chromosomal interactions was consistent with both of following interpretations: higher epigenetic or transcriptional activity of a chromatin locus increases its rate of trans-chromosomal interactions, or positioning of a locus in an environment enriched with active chromatin enhances its transcriptional activity.

FIGS. 4A-4F show preferential trans-chromosomal interactions between active chromatin are correlated with transcription and are disrupted upon treatment that perturbs condensate formation. FIG. 4A is single-cell images of chromatin loci and transcriptional activities. Left: Locations of all imaged compartment-A (upper portion of scale) and -B (lower) loci in a single z-plane from a single nucleus. Middle: Local trans-chromosomal A/B density ratios for the same loci, based on the greyscale scale bar. Right: Same as the middle panel, with detected transcriptional bursts overlaid and displayed as circles. FIG. 4B shows a comparison of local trans-chromosomal A/B density ratio for each locus in the transcribed versus silent state. For each genomic locus containing at least one imaged gene, the trans-chromosomal A/B density ratio was calculated for the cells in which it was actively transcribed (designated as transcribed) and for the cells in which it was not transcribed (designated as silent). Median values across cells are shown for each state. Loci were ordered by their A/B density ratio in the silent state and the A/B density ratios were plotted for both the silent and transcribed states. FIG. 4C shows normalized trans-chromosomal contact frequency matrix for cells treated with alpha-amanitin to inhibit transcription. The matrix is ordered and normalized as described in FIG. 2A. FIG. 4D shows a distribution of AA, BB and AB contact frequencies shown as box plots, as described in FIG. 2B. n=72,771 locus pairs for AA, n=193,753 locus pairs for BB, and n=237,986 locus pairs for AB. FIGS. 4E-4F are the same as FIGS. 4C-4D but for cells treated with 1,6-hexanediol.

FIG. 14 shows the local density of trans-chromosomal A loci near each imaged locus when the locus is in the active transcribed state or the silent state. For each locus, cells were divided into two groups, depending on whether the locus is actively transcribed or silent. The median local density of A loci is shown for these two groups of cells (transcribed and silent). The loci are ordered based on their local trans-chromosomal A-locus density in the silent state.

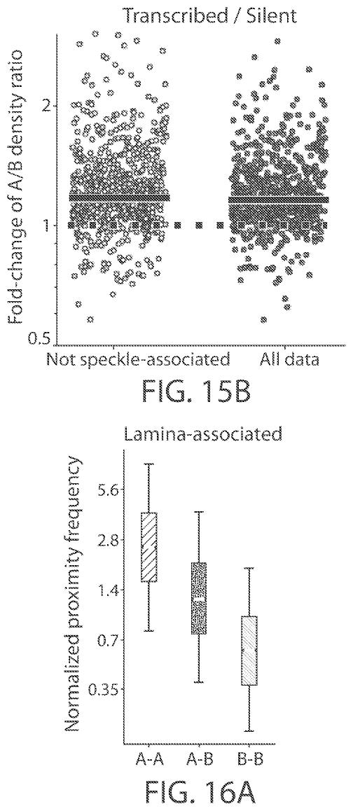

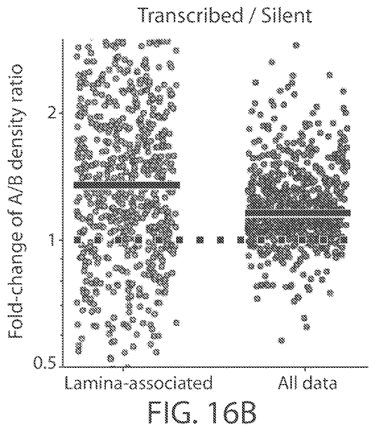

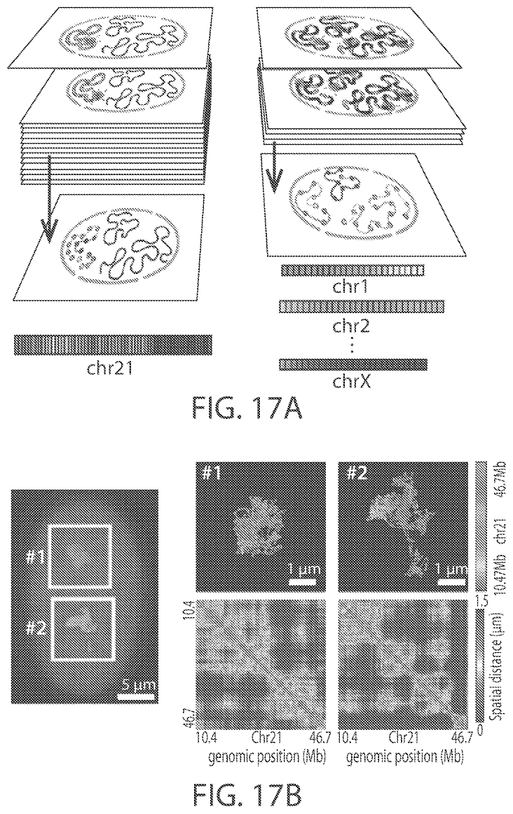

Because nuclear speckles are one of the most prominent nuclear bodies that concentrate actively transcribed loci, it was unknown whether association with nuclear speckle could provide a simple explanation for the observed preferential occurrence of active-active chromatin interactions trans-chromosomally. Interestingly, when analysis was restricted to the loci that were not associated with nuclear speckles, the same trend for enrichment of A-A over A-B and B-B interactions among trans-chromosomal contacts (FIG. 15A) and the same trend for actively transcribing loci to exhibit higher local A/B density ratios over silent loci was observed (FIG. 15B). Remarkably, these trends were observed even when only loci that were lamina-associated and thus in an environment enriched with compartment-B chromatin were considered (FIGS. 16A-16B). This latter result also indicated that the observed enrichment for active-active trans-chromosomal interactions could not be simply accounted for by the fact that active chromatin is more concentrated towards the center of the nucleus.

FIGS. 15A-15B show enrichment of active-active trans-chromosomal interactions among chromatin loci not associated with nuclear speckles. FIG. 15A shows trans-chromosomal contact frequencies between A locus pairs (AA), B locus pairs (BB), and pairs comprised of one A and one B locus (AB), considering only the cells in which both loci are not associated with nuclear speckles. The contact frequencies were normalized as described for FIG. 2A. Distributions are represented as box plots, as described in FIG. 2B. n=72,771 locus pairs for AA (left), n=193,753 locus pairs for BB (right), and n=237,986 locus pairs for AB (center). For comparison, the median values for all data, regardless of the speckle association status, are show as triangles. FIG. 15B shows the fold-change of local trans-chromosomal A/B density ratios between transcribed and silent states for loci not associated with nuclear speckles. For each genomic locus, the fold change in the local trans-chromosomal A/B density ratio between transcribed and silent states of the locus was computed, considering only the cells in which the locus was not associated with a nuclear speckle. The median A/B density ratio in each state (transcribed or silent) was determined for each locus and the fold change between the two states is shown on the left (each circle corresponding to a genomic locus). The corresponding fold changes derived from all data regardless of nuclear-speckle association status of the loci are shown on the right for comparison. The dotted line represents no change and the solid lines represent the median fold change across all loci in each case.

FIGS. 16A-16B show the enrichment of active-active trans-chromosomal interactions among chromatin loci associated with nuclear lamina. FIG. 16A shows trans-chromosomal contact frequencies between A locus pairs (AA), B locus pairs (BB), and pairs comprised of one A and one B locus (AB), considering only pairs of lamina-associated loci (within 250 nm). The contact frequencies were normalized as described for FIG. 2A. Distributions are represented as box plots, as described in FIG. 2B. n=72,771 locus pairs for AA (left), n=193,753 locus pairs for BB (right), and n=237,986 locus pairs for AB (center). For comparison, the median values for all data, regardless of the lamina association status, are shown as triangles. The variance is relatively large in these cases because only a relatively small fraction of loci is associated with lamina, nonetheless the differences between the different types of pairs AA, BB and AB are statistically significant (P-values <10-10). FIG. 16B shows the fold-change of local trans-chromosomal A/B density ratios between transcribed and silent states for loci associated with nuclear lamina. For each genomic locus, the fold change in the local trans-chromosomal A/B density ratio between transcribed and silent states of the locus was computed, considering only cells in which the locus was associated with the nuclear lamina. The median A/B density ratio in each state (transcribed or silent) was determined for each locus and the fold change between the two states is shown on the left (each circle corresponding to a locus). Outliers (33 loci above and 18 loci below the presented scale) were omitted to allow a clearer visualization of the median fold change. The fold changes derived from all data regardless of lamina-association status are shown on the right for comparison. The dotted line represents no fold change and the solid lines represent the median fold change across all loci in each case.

The results showed that trans-chromosomal interactions occurred preferentially between active chromatin loci, and that this behavior was consistently observed across multiple distinct nuclear environments. Explored next, was what could potentially cause this preferential, widespread active-active chromatin interaction. Since RNA polymerase II (Pol II) contains low-complexity domains (LCD) and can form condensates, whether transcription by Pol II could be responsible for these preferential trans-chromosomal interactions was tested by using a transcription inhibition drug, alpha-amanitin, which causes Pol II dissociation and degradation. Despite abolishing transcription and altering nuclear structures and their association with chromatin (FIGS. 13A-13C), treatment with alpha-amanitin did not substantially reduce the preferential trans-chromosomal interactions between active chromatin (FIGS. 4C-4D), suggesting that additional or other active chromatin binding factors were involved in these trans-chromosomal interactions. It was shown that multiple other proteins associated with active chromatin contain LCDs that could potentially mediate condensate formation. Therefore, the aim was to perturb condensate formation more generally by using 1,6-hexanediol, a drug that is known to disrupt hydrophobic interactions between LCDs. Notably, the preferential enrichment for active chromatin interactions in trans-chromosomal contacts was largely abolished upon treatment of cells with 2% 1,6-hexanediol for 45 minutes (FIGS. 4E-4F), suggesting a potential role for condensate formation in the establishment or maintenance of these interactions.

In summary, these examples developed a massively multiplexed FISH method for imaging the 3D organization of chromatin at the genome scale in single cells and further demonstrated the ability to place 3D genome organization in its native structural and functional context by combining chromatin and nascent transcript imaging, both at the genome scale, with nuclear-structure identification. This provides an integrated view of nuclear organization in single cells. While target loci were chosen uniformly across all chromosomes here to provide an unbiased view of the overall 3D genome organization, this method could also be used to target genomic loci with specific structural and functional properties, such as promotors, enhancers, and loci bound by specific nuclear architecture proteins, to study the interactions among these loci and their relationship with transcription and other chromatin functions. The broad applications of this approach to a wide range of questions related to genome organization, could illuminate both the mechanisms governing chromatin organization and the role of chromatin structures in regulating genome functions.

### Example 5

This example illustrates various materials and methods usable in the above examples.

Target genomic regions. For chromatin imaging, genomic loci were chosen for imaging in the following way. For each human autosome and X chromosome, a 30-kb segment every ˜3 Mb of spacing was selected. If this spacing resulted in less than 30 selected loci on a given chromosome, the spacing was reduced for that chromosome, until all chromosomes had at least 30 loci selected. This resulted in a total of 1,041 target genomic loci for imaging, and the number of loci in individual chromosomes ranged from 30-80. Encoding probes were then designed for each 30-kb segment (˜400 oligonucleotide probes) for the combinatorial FISH imaging.

For imaging of nascent RNA transcripts, all intron-containing genes that completely or partially overlap with the targeted genomic loci were selected. Encoding probes were then designed for the introns of all of these RNAs such that each RNA had ˜20 encoding probes and that the targeting sequences of the encoding probes were kept as close as possible to the transcription start site (TSS). A total of 1,137 genes were targeted.

Barcode design for combinatorial FISH imaging. Binary barcodes for imaging the 1,041 genomic loci were chosen in the following fashion. First, all possible 100-bit binary barcodes with a Hamming weight of 2 (i.e. each barcode containing two “1” bits and 98 “0” bits) were generated and 1,041 barcodes from this list were randomly selected. The selected barcodes were then arbitrarily assigned to the 1041 genomic loci first. Next, barcodes were exchanged randomly between the used and unused code pool, as well as between loci from different chromosomes, in order to minimize, for each chromosome, the variance in the number of loci appearing (i.e. reading “1”) across different bits. This resulted in an approximately equal number of loci imaged per bit for each chromosome. To optimize association of barcodes to loci within each chromosome, loci within the same chromosome were allowed to exchange barcodes and optimized for the largest minimal genomic distance between loci with barcodes reading “1” at the same code position. When comparing code assignments with identical minimal genomic distances, the one that minimized the coefficient of variation of genomic distances was selected (so that genomic distances have both larger means and smaller standard deviations).

Barcodes for imaging the nascent RNA transcripts of the 1,137 genes were chosen similarly, but using a 54-bit, Hamming distance 2 code instead of a 100-bit, Hamming distance 2 code.

Encoding probe design. Encoding probes for chromatin imaging were synthesized from a pool of oligonucleotides purchased from Twist Biosciences. Each oligo in this pool used the following sub-sequences (from 5′ to 3′):
1. A 20-nucleotide (nt) forward priming region for PCR amplification and reverse transcription (RT)
2. A 20-nt readout sequence corresponding to one of the bits in which the genomic loci targeted by the probe will be imaged
3. A 40-nt target sequence, designed to bind uniquely to a single targeted genomic locus
4. An additional copy of the 20-nt readout sequence described above
5. A 20-nt reverse priming sequence for PCR amplification

The forward and reverse priming sequences were chosen from a previously generated list of random 20-nt sequences optimized for PCR.

The readout sequences were chosen via the following process. First, a list of 30-nt sequences with minimal homology to the human genome was created. Then, a subset of these sequences were ranked by observed signal to noise ratio (SNR) and the top 100 were chosen as DNA readout probes. Lastly, the readout sequences were chosen by reverse-complementing the last 20 nt of each of these sequences.

The 40-nt target sequence was chosen similarly. Briefly, the following procedure was repeated for each genomic region of interest (see the “Target genomic regions” discussion above). First, a list of all 40-nt sequences complementary to the genomic region of interest was created (starting at each possible base in the targeted region). Then, sequences were filtered by requiring them to be within a defined range of melting temperatures and GC content. The remaining sequences were then further filtered by limiting the allowed degree of homology to the human genome, the human transcriptome and a database containing repetitive sequences. Homology was determined by creating a table of all possible 17-nt sequences and the number of times they appear in the target database (e.g. the human genome, the human transcriptome), and calculating the total number of exact 17-nt matches a given candidate sequence has with it. Finally, target sequences were selected from the remaining sequences after the final filtering step such that no genomic overlap exists between any pair of target sequences.

To generate the full-length probes, each of the chosen 40-nt target sequences for each target genomic locus was alternatingly assigned to 2 groups spanning the entire target locus. Each of these groups were associated with a single readout sequence, corresponding to one of the two bits in which the locus would be imaged. Then, each target sequence was concatenated to two identical copies of the readout sequence assigned to its group, and then concatenated to the forward and reverse PCR primers.

Probes for RNA imaging were designed similarly, with the exception that they contained 3 copies of an identical readout sequence on every probe, one at the 5′ end and two at the 3′ end of the target region. Readout sequences for RNA imaging were orthogonal to those used for DNA imaging and were selected from the same ranked list of tested readout sequences.

Encoding probe synthesis. Encoding probes were amplified from the template library described above (see “Encoding probe design” above). This was done using an amplification protocol involving the following steps:
1. The initial oligo pool was expanded using limited-cycle PCR for approximately 20 cycles. The reverse primer used in this step also introduced a T7 promoter sequence via primer extension.
2. The resulting product was purified via column purification and underwent further amplification and conversion to RNA by a high-yield in-vitro transcription reaction.
3. The RNA product was converted back to single-stranded DNA by a reverse transcription reaction.
4. The product of the previous step was subjected to alkaline hydrolysis (to remove residual RNA and primer DNA) and column purified.
5. If necessary, the product of the previous step was dried in vacuum and resuspended in water to achieve the desired concentration of primary probe.

All primers were purchased from Integrated DNA Technologies (IDT).

Cell culture and encoding probe hybridization. Cell preparation were done as follows. IMR-90 cells were purchased from American Type Culture Collection (ATCC, CCL-186) and grown according to the recommended protocol. To avoid potential alterations to chromatin structure, all cells in this study were plated within 6 weeks of culture initiation at the density specified below.

To prepare for DNA imaging, cells were plated on 40-mm, round #1.5 coverslips (Bioptechs, 0420-0323-2), at a density of ˜500,000 cells per coverslip. Cells were allowed to grow for ˜2 days until confluency at 37° and 5% CO₂. In the transcription-inhibition experiments, cell media was replaced with fresh media containing 100 micrograms/mL alpha-amanitin (Sigma-Aldrich, A2263) 6 hours prior to cell fixation. For experiments with 1,6-hexanediol (Sigma-Aldrich, 240117), coverslips were coated with 10 micrograms/mL fibronectin (Sigma-Aldrich, F1141) prior to cell plating and replaced media with fresh media containing 2% w/v 1,6-hexanediol for 45 minutes. The culture was then fixed using 4% paraformaldehyde (PFA) in PBS for 10 minutes at room temperature and washed in PBS 2-3 times. Cells were then permeabilized in two steps: first, they were treated with 0.5% v/v Triton-X (Sigma-Aldrich, T8787) in PBS for 10 minutes at room temperature. Then, cells were treated with 0.1 M hydrochloric acid (HCl) for 5 minutes at room temperature and washed in PBS 2-3 times. Following HCl treatment, cells were treated with a solution of 0.1 mg/mL RNase A (ThermoFisher, EN0531) dissolved in PBS for 30-45 minutes at 37° C., to remove potential sources of off-target binding to RNA. Following this treatment, cells were incubated in pre-hybridization buffer, consisting of 2× saline-sodium citrate buffer (SSC; Ambion, AM9763) and 50% formamide (Ambion, AM9342) for approximately 10 minutes. Next, the cell coverslip was inverted and placed on a drop of 50 microliters of hybridization buffer (2×SSC, 50% formamide, 10% dextran sulfate (Sigma-Aldrich, D8906) containing a mixture of encoding probes at −25 micmolar total concentration with or without 10 micrograms Human Cot-1 DNA (ThermoFisher, 15279011)) in a 60-mm petri dish. The dish was partially submerged in a water bath at −90° C. for 3 minutes and incubated at 47° C. in a humidified chamber for 16-36 hours. After incubation with encoding probes, the sample was washed in 2×SSC and 40% formamide for 30 minutes and post-fixed with 4% PFA in 2×SSC for 10 minutes at room temperature. The sample was then incubated for 2-3 minutes with fiducial beads (either ThermoFisher F8805 or ThermoFisher F8792) in 2×SSC and stained with 1 micromolar 4′,6-diamidino-2-phenylindole (DAPI; ThermoFisher D1306) in 2×SSC for 5-10 minutes, and then stored in 2×SSC until imaging.

For experiments including RNA imaging, all buffers used from the point at which cells were fixed contained a 1:10-1:1,000 dilution of RNAse inhibitor (either NEB M0314 or Fisher Scientific N2615). Treatment for RNA staining was identical to the above-described protocol up to treatment with HCl. After this step, cells were incubated in pre-hybridization buffer for 10 minutes, and the cell coverslip was then inverted and placed on a drop of hybridization buffer containing encoding probes targeting the RNA introns at −1 micromolar total concentration, as described for DNA staining. In this case, however, no 90° C. heat denaturation was performed, and cells were immediately incubated at 47° C. in a humidified chamber for 16-36 hours. After incubation with encoding probes, the sample was washed in a formamide solution and post-fixed with PFA as described for DNA above. It was then incubated with fiducial beads and stained with 1 micromolar DAPI, before being stored in 2×SSC until imaging. After RNA imaging, the sample was removed from the microscope, the cells were treated with RNase A and then the DNA hybridization proceeded in the same manner as described above for DNA imaging without RNA imaging.

Sequential hybridization of readout probes for FISH imaging. All fluid exchanges in this part of the protocol were achieved via the use of a custom-built fluidics system, with the coverslip mounted in a FCS2 flow chamber (Bioptechs, 060319-2). The fluidics system used 3-4 computer-controlled eight-way valves (Hamilton, MVP and HVXM 8-5) and a computer-controlled peristaltic pump (Gilson, MINIPLUS 3). Put together, these components allowed control of both the rate of fluid flow and of the type of fluid flowing at any given time.

Each round of hybridization used the following steps:
1. Flow in the hybridization buffer with a set of oligonucleotide probes specific to each round, as described below
2. Incubate for 10 minutes at room temperature
3. Flow wash buffer
4. Incubate for ˜200 seconds
5. Flow imaging buffer

The imaging buffer was prepared and included 60 mM Tris pH 8.0, 10% w/v glucose, 1% Glucose Oxidase Oxygen Scavenger Solution (containing −100 mg/mL Glucose Oxidase (Sigma-Aldrich, G2133) and a 1:3 dilution of catalase (Sigma-Aldrich, C3155)), 0.5 mg/mL 6-hydroxy-2,5,7,8-tetramethylchroman-2-carboxylic acid (Trolox; Sigma-Aldrich, 238813) and 50 micromolar Trolox Quinone (generated by UV irradiation of a Trolox solution). Trolox was dissolved in methanol before being added to the solution. After preparation, the imaging buffer was covered by a ˜0.5 cm thick layer of mineral oil to prevent exposure to oxygen.

The hybridization buffer and wash buffer were made up of 35% and 30% formamide in 2×SSC, respectively, with the hybridization buffer also containing 0.01% v/v Triton-X. The hybridization buffer was kept separately for each hybridization round and contained two or three (for DNA and RNA imaging, respectively) sets of readout probes. Fluorescent signal was introduced in one of two ways:
1. For DNA imaging, each round's hybridization buffer included two fluorescent readout probes, one labeled with Cy5 or Alexa647 and the other labeled with Alexa750. Fluorescent readout probes used either: 1) a fluorescently labeled oligo complementary to a readout sequence common to all encoding probes imaged in a given bit, added at 100 nM concentration, or 2) a combination of an adaptor oligo having the sequence complementary to a readout sequence, concatenated to an additional readout sequence (referred to as the secondary readout sequence) common to all adaptors (more accurately, common to all adaptors in each color channel) and orthogonal to all other used readout sequences, and a fluorescently labeled oligo probe complementary to this secondary readout sequence. The adaptor and secondary readout probes were pre-mixed in a 1:1.5 ratio and added to a final concentration of ˜100 nM. For some experiments the adaptor and readout probes were hybridized sequentially to the sample. This allowed for using lower concentration of the more expensive secondary readout probe.
2. For RNA imaging, each round's hybridization buffer contained three adaptor oligos (to be detected in three different color channels), each binding to a different readout sequence and each containing an additional, secondary readout sequence. All adaptors corresponding to the same color channel shared the same secondary readout sequence. Each round included two discrete hybridization steps: first the adaptors were flowed in, hybridized, and excess material was washed. Then three fluorescent readout probes, complementary to the secondary readout sequences on the adaptors, respectively labeled with Cy3, Cy5, and Alexa750, were flowed in sequentially. Fluorescent readout probes used for RNA imaging contained a disulfide bond linking the fluorophore to the secondary oligo, to allow efficient removal of signal between rounds. After the fluorescent readouts were hybridized, imaging buffer was flowed in and signal was collected.

Before the next round of readout probe or adaptor probe hybridization, fluorescent signals from the readout probes or secondary readout probes in the current round were removed as described in “Signal removal between rounds of hybridization,” below.

Before the first round of hybridization, a round of imaging was performed to acquire the DAPI signal and identify nuclear boundaries. Then, the entire set of 1,041 genomic loci were imaged in 50 rounds of hybridization and 2 color channels per round. In each round, the genomic loci were imaged in 3D by stepping the stage in the z-dimension. Nascent RNA transcripts for 1,137 genes were imaged likewise in 3D in 18 rounds in 3 colors. Additional rounds were used to relabel sets of genomic loci and assess chromatic aberration and bleedthrough between color channels. Imaging of ˜60 fields of view containing a total of ˜1,000-2,000 cells took ˜3 days.

The 3-4 valve system allowed loading up to 20-30 different hybridization solutions. As a result, after exhausting all the fluidic system's channels, the sample chamber was bypassed and all the channels used for hybridization were washed with 30% formamide in water. Next, the chamber was reconnected and the next set of hybridization and imaging rounds were performed.

Antibody labeling and imaging. Antibody imaging was performed immediately after RNA or DNA imaging. Following completion of imaging via the protocols described above, samples underwent the following steps:
1. Samples were incubated with blocking solution (PBS with 0.1% v/v Tween-20 (Sigma-Aldrich P9416) and 1% w/v bovine serum albumin (BSA; Jackson Immunoresearch 001-000-162)) for 30 minutes.
2. Samples were incubated with primary antibody diluted in blocking solution for 1 hour
3. Samples were washed 3 times in PBS with 0.1% Tween-20 for 5 minutes each
4. Steps 2 and 3 were repeated for a fluorescently-tagged secondary antibody

All buffer exchanges were done on the microscope, using the microfluidic system described above. The Cy5 color channel was used for imaging and extinguished the signal between sequential antibody labelling using photobleaching.

The following sets of primary and secondary antibodies were used:
1. For imaging nuclear speckles, a primary antibody was used against SC35 (Abcam, ab11826)—a splicing factor commonly used as a marker of nuclear speckles—at 1:200 dilution from stock and a donkey anti-mouse secondary antibody labeled by a Cy5 dye (Jackson Immunoresearch, 715-175-150) diluted 1:1,000 from stock concentration.
2. For imaging nucleoli, anti-fibrillarin antibody was used (Abcam, ab5821), at 1:200 dilution from stock, and a donkey anti-rabbit secondary antibody labeled by an Alexa 657 dye (Jackson Immunoresearch, 711-605-152), diluted 1:1,000 from stock concentration. Signal removal between rounds of hybridization. Before each round of imaging, the signal from the previous round (or endogenous background, in the case of the first round) was extinguished. This was achieved via photobleaching of the signal. Photobleaching was performed by changing the buffer to 2×SSC and illuminating each field of view with the maximum available power of the 647 and 750 lasers (as well as the 560 laser when imaging RNA) for 10 seconds. In RNA imaging experiments, the buffer used for bleaching also contained 50 mM tris(2-carboxyethyl)phosphine (TCEP; Sigma-Aldrich, C4706) to cleave the disulfide bond connecting fluorophores to readout probes. The DAPI signal was extinguished as a result of the high formamide concentration in the hybridization and wash buffers.

Image Acquisition.

Image acquisition was performed using a custom-built microscope system. The system was built around a Nikon Ti-U microscope body with a Nikon CFI Plan Apo Lambda 60× oil immersion objective with 1.4 NA. Illumination was based on one of two alternatives:
1. Solid-state, single-mode lasers with the following wavelengths: 405 nm (Coherent, Obis 405 nm LX 200 mW), 560 nm (MPB Communications, 2RU-VFL-P-2000-560-B1R), 647 nm (MPB Communication, 2RU-VFL-P-1500-647-B1R) and 750 nm (MPB Communication, 2RU-VFL-P-500-750-B1R). In this case, the output of the 560-nm, 647-nm and 750-nm lasers were controlled by an acousto-optic tunable filter (AOTF) while the 405-nm laser was controlled directly via its laser control box. A custom dichroic (Chroma, zy405/488/561/647/752RP-UF1) and emission filter (Chroma, ZET405/488/461/647-656/752m) were used to separate excitation and emission illuminations.
2. A Lumencor CELESTA light engine (a fiber-coupled solid-state laser-based illumination system) with the following wavelengths: 405 nm, 446 nm, 477 nm, 520 nm, 546 nm, 638 nm and 749 nm. This system was used with a penta-bandpass dichroic (IDEX, FF421/491/567/659/776-Di01-25×36) and a penta-bandpass filter (IDEX, FF01-441/511/593/684/817-25).

A scientific CMOS camera (Hamamatsu FLASH4.0 or Hamamatsu C13440 with factory calibration for single-molecule imaging) was used for image acquisition. Sample position in three dimensions was controlled using a XYZ stage (Ludl). A custom-built auto-focus system was used to maintain a constant focal plane over prolonged periods of time. This was achieved by comparing the relative position of two IR laser (Thorlabs, LP980-SF15) beams reflected from the glass-fluid interface and imaged on a separate CMOS camera (Thorlabs, uc480).

For each experiment, approximately ˜60 fields of view (FOVs) were selected for imaging, avoiding regions where cells are sparse (we typically identified 10-50 cells per FOV). Each camera FOV had 1,000×1,000 pixels, with a camera pixel corresponding to 153 nm in each dimension in the imaging plane, or 2048×2048 pixels, with a camera pixel corresponding to 108 nm in each dimension in the imaging plane.

After each round of hybridization (see “Sequential hybridization of readout probes for FISH imaging” above), z-stack images of each FOV were acquired in 3 or 4 colors: 647 nm and 750 nm illumination (or 560 nm, 647 nm, and 750 nm illumination for RNA imaging in the case of combined DNA and RNA imaging) were used to acquire FISH images, 560 nm illumination (or 405 nm illumination in the case of combined RNA and DNA imaging) was used to image fiducial beads. For the first round of imaging, 405 nm illumination was used to image the DAPI signal, while for antibody imaging, the 647 nm excitation channel was used after RNA or DNA imaging. Consecutive z-sections were separated by 85, 100 or 150 nm, covering the entirety of the nuclear volume for all imaged cells. At each z position, images were acquired in all channels before the stage was moved and images were acquired at a rate of ˜10 Hz.

Image analysis and spot fitting for DNA and RNA imaging. The following analysis pipeline was applied to each imaged FOV in order to obtain the three-dimensional (3D) positions of all loci of interest:
1. Fiducials were fitted in all rounds of imaging and used for image alignment
2. In the first imaging round (preceding the first round of hybridization), DAPI signal was used to identify the borders of individual nuclei, as well as for image registration between RNA and DNA imaging
3. Diffraction-limited spots within each identified nucleus were fitted to a 3D Gaussian function to identify their center of mass and brightness above local background
4. Fitted spots were compared with other localizations within the same nucleus across all rounds of hybridization to identify the loci from which they originated, using a custom algorithm and software (described in detail in “Decoding algorithm for fitted DNA spots” and “Decoding algorithm for fitted RNA spots” sections).

Spot fitting for DNA and RNA imaging. Signal from individual FISH imaging rounds was fitted using 3D Gaussians. To make analysis more manageable, the number of fitted spots per image that would be retained for decoding to 125 were fixed (˜3-fold greater than the number of distinct loci expected without noise).

Drift correction. Fiducial bead spot fitting was performed in the same way as described above. The set of fiducial bead positions was then compared between rounds of hybridization and a rigid transformation was applied to minimize the sum of square difference of the relative position of beads.

Correction of chromatic effects. Bleedthrough and chromatic aberration for multi-color imaging were performed by labeling the same set of genomic loci in each imaging channel independently and comparing the signals of the same loci in the different channels, respectively.

Nuclei segmentation. DAPI images from the first round of imaging were used to identify the volume of individual nuclei and allowed for cell segmentation. This was achieved via a convolutional neural network, built and trained, which took the maximum projection of the DAPI image onto the xy plane as input.

Additional image analysis: Image registration between DNA and RNA imaging. In experiments including both DNA and RNA imaging, DAPI signal was first used for rough image-registration (to camera pixel precision) across the two sets of images via 2D image correlation (all images within each set were aligned to the DAPI image using fiducial beads). After an initial round of RNA decoding was performed (see “Decoding algorithm for fitted RNA spots” below), a finer alignment was calculated by assuming that the displacement between nascent RNA localization and their harboring DNA loci should average to zero when considered across all imaged genes and cells in a field of view. In accordance, an additional rigid transformation was calculated to minimize the mean displacement between imaged nascent RNA and their corresponding DNA loci and used this as the final alignment.

Identification of nuclear bodies from immunofluorescence imaging. The location of nuclear bodies (nuclear speckles and nucleoli) was extracted from immunofluorescence signals by applying a threshold to the intensity of the immunofluorescence signals, resulting in a pixelized mask identifying high immunofluorescence signals. This was then treated as a pixelized set of locations “containing” nuclear bodies.

Decoding algorithm for fitted DNA spots. Identification and 3D localization of each genomic locus were achieved through the following steps:
1. A list was generated for the drift- and aberration-corrected locations of all identified spots in each round of imaging.
2. For each detected spot in every imaging round, it was found that all spots from other rounds that were within a set cutoff distance (˜150 nm in x, y and z) from its location. All such pairs of spots were retained for further analysis, whether or not the barcode produced by a spot pair (based on which round and color channel they appeared in) corresponded to a valid barcode (a barcode that was assigned to a genomic locus).
3. For each pair of spots, three quality metrics were calculated:
   A. The displacement between the 3D localizations of the two spots
   B. The difference in brightness between the two spots
   C. The mean brightness of the two spots
   The brightness of each spot was normalized by the median brightness of all spots in the corresponding bit.
4. Spot pairs were then separated into two groups, based upon whether they correspond to a valid barcode (and hence potentially to a genomic locus) or not. Within each group, the distributions of the quality metrics were calculated. For convenience, the distribution of spot-pair quality metrics from the invalid barcodes were referred to as the “invalid distribution” and from all valid barcodes as the “valid distribution.”
5. For each spot pair the three quality metrics in step 3 were combined into a single measure by calculating the combined Fisher p-value for every candidate spot pair against the “valid distributions” in step 4. This was thought of as the overall quality score of each spot-pair, and was calculated per pair in the following way: for each of the three metrics the fraction of other spot-pairs in the “valid distribution” was calculated with lower quality metric and multiplied these fractions together. An expectation-maximization procedure was then used to sequentially select the two spot-pairs with the highest quality score corresponding to each targeted chromatin locus and reupdated the “valid distribution,” and this optimization procedure was repeated until convergence. After convergence, the final sets of spot pairs, each corresponding to a chromatin locus, were used to determine the 3D spatial positions of the loci.
6. After step 5, a modified K-means algorithm was used to separate the chromatin loci belonging to the same chromosome into two homologs. As opposed to the standard K-means clustering algorithm which splits points into two groups and minimizes the radius of gyration within each group, points were switched progressively between the groups to first maximize the fraction of assigned points in each homolog and then the radius of gyration of each homolog was minimized.
7. After separating the two homologs their center of mass and the distance of each spot-pair from step 2 were calculated to their parent chromosome's center of mass. The distance to the chromosome center was added as another quality metric in addition to the 3 metrics considered in step 3 and steps 3-6 were repeated.
8. Finally, the spot pairs from step 7 were filtered to remove the pairs whose quality scores remained similar to the “invalid distribution.”

The remaining spot pairs after step 8 were used to determine the final positions of the chromatin loci and trace the chromatin structure.

Decoding algorithm for fitted RNA spots. Signals from the RNA imaging rounds was decoded using the following procedure:
1. A list was generated for the drift- and aberration-corrected locations of all identified spots in each round of imaging.
2. For each detected spot in every imaging round, it was found that all spots from other rounds that were within a set cutoff distance from its location and these spot pairs were retained as candidate RNA bursts if they formed a valid barcode.
3. The location of each of these candidate RNA bursts was then compared to the location of the DNA locus harboring the relevant gene, after initial image registration (based on DAPI images) and drift and aberration correction, and kept if they were within a set threshold distance.
4. The registration between DNA and RNA imaging was refined based on the displacement between the initial decoded RNA localizations (from step 3) and the location of the DNA locus harboring them as described in the “Image registration between DNA and RNA imaging” section above.
5. Locations of all candidate RNA bursts were compared again to the location of the DNA locus harboring the gene to which they decode, this time with the refined image registration. If the nascent RNA localization was within a cutoff distance from its harboring DNA locus at this stage, it was considered as a detected transcriptional burst.

Additional analysis: Identification of the nuclear lamina. The position of the nuclear lamina was estimated by generating the minimal 3D convex hull (using Python's scipy package) surrounding the locations of all decoded chromatin loci in a given cell.

Spatial distance. The spatial distance between any pair of loci was simply calculated as the Euclidean distance between their fitted 3D Gaussian centers, multiplied by the appropriate ratios relating camera pixels and z steps to physical distance. In the case of distance to nuclear bodies, the minimal Euclidean distance to all identified nuclear body “locations” or the minimal distance to the surface of the convex hull defining the nuclear lamina was calculated.

Contact frequency matrices from imaging. To calculate the contact frequency between any given pair of loci, the number of measured distances between that locus-pair that was smaller than a set threshold was counted. This number was then divided by the total number of distances measured for that pair of loci.

Local density analysis. To calculate the trans-chromosomal local density of compartment-A and compartment-B loci at each decoded location, the spatial distances between each pair of chromatin loci for each cell were calculated. For each locus, the local A/B density ratio was calculated in the following way:
1. The density contribution of each other locus was calculated form a different chromosome by evaluating a Gaussian function value with a standard deviation of 500 nm (adjusted to account for variability in cell size) at the distance between the two loci.
2. The total A density at the locus was then computed as the sum of this Gaussian function value for all trans-chromosomal A loci, and the total B density was computed in an analogous way.
3. The total density of trans-chromosomal compartment-A loci was divided by the density of trans-chromosomal compartment-B loci to find the A/B density ratio at the locus.

Estimation of detection efficiency in multiplexed RNA imaging. Estimation of the detection efficiency of transcriptional burst events was performed in the following way:
1. All targeted genomic loci that harbored a gene whose RNA introns were imaged were considered. For any of these genomic loci, their corresponding RNA signal should have appeared in two pre-defined bits if the gene is transcribed. Knowing the rate with which each of these two bits is not detected (p) allowed derivation of the detection efficiency of the RNA. The set of genomic loci that colocalized (within ˜150 nm) with RNA signal in at least one of the two expected bits of their corresponding genes were identified.
2. From the total set of chromatin loci identified in step 1, the fraction (f) of loci that colocalized with RNA signal from exactly one of its gene's corresponding bits (and not with both bits) were determined. From the measured f (8.4%), which should be equal to

$$\frac{2 p ( 1 - p )}{1 - {p}^{2}} ,$$

p (4.4%) was estimated.
3. The overall detection efficiency for detecting a colocalized signal in both bits was calculated using the equation: η=(1−p)², and was found to be ˜92%.

Hi-C data analysis. Hi-C data for IMR-90 cells was procured and loaded using a straw. For identification of A/B compartments in individual chromosomes, established published protocols were followed. For comparison of contact frequencies derived from the imaging data to Hi-C, bins centered around the targeted regions were created and Hi-C data for these bins was procured by summing the number of reads in higher resolution Hi-C data.

### Example 6

The three-dimensional (3D) organization of chromatin regulates many genome functions. The understanding of 3D genome organization is, however, hindered by a lack of tools to directly visualize chromatin conformation in its native context. Reported herein is an imaging platform for visualizing chromatin organization across multiple scales in single cells with high genomic throughput. First, multiplexed imaging of hundreds of genomic loci by sequential hybridization was demonstrated, which allowed high-resolution conformation tracing of whole chromosomes. Next, a combinatorial imaging method for genome-scale chromatin tracing was developed and demonstrated simultaneous imaging of >1000 genomic loci and nascent transcripts of >1000 genes together with landmark nuclear structures. Using this platform, chromatin domains, compartments, and trans-chromosomal interactions, and their relationship to transcription in single cells were characterized. There is a broad range of applications of this high-throughput, multi-scale and multi-modal imaging technology, which provides an integrated view of chromatin organization in its native structural and functional context.

The 3D organization of the genome regulates many essential cellular functions ranging from gene expression to DNA replication. Biochemical and imaging measurements have unveiled complex chromatin structures across a wide range of scales. In particular, high-throughput chromosome conformation capture methods, such as Hi-C and other sequencing-based methods, have revealed chromatin structures, such as domains and compartments, with a genome-wide view. Notably, chromatin is partitioned into genomic regions with enhanced self-interaction, termed topologically associated domains (TADs), which appear as block-like structures on Hi-C contact maps. These TADs, ranging from hundreds of kilobases (kb) to several megabases (Mb) in size, often harbor co-regulated genes and have boundaries coinciding with regulatory epigenetic elements. At a larger scale, chromatin is partitioned into two major compartments, called A and B compartments, which are respectively enriched for active and inactive chromatin, as revealed by an alternating “plaid” pattern in Hi-C maps, consistent with previous imaging-based observations that gene-rich and gene-poor segments of chromatin tend to spatially segregate. Recent imaging experiments show that compartment A and compartment B chromatin indeed tend to spatially segregate in single cells. The physiological significance of A/B compartmentalization is implicated by its changes during development and between cell types.

Overall, high-throughput sequencing-based approaches have greatly enriched knowledge of 3D genome organization. Nonetheless, these powerful approaches also have limitations. For instance, these methods provide contact information for pairs of chromatin loci but do not provide direct spatial position information for these loci. Furthermore, most insights on chromatin organization are built on population-averaged contact maps across millions of cells. Despite continuous improvement of single-cell Hi-C methods, the capture efficiency of chromatin contacts in single cells and/or the cell throughput of these methods remain relatively low, and hence investigation of 3D genome organization in single cells remains a challenging task. In addition, although methods have emerged to combine Hi-C with other measurement modalities, for example, to provide characterizations of chromatin contacts in the context of interacting proteins, nuclear structures, or DNA modifications, multi-modal measurement by sequencing remains challenging. Notably, a method that allows genome-scale measurements of both chromatin organization and transcriptional activity in the same cells has not emerged, despite the demand for such a method to further understanding on how chromatin organization regulates transcription and how transcription, in turn, impacts chromatin organization.

Imaging-based approaches, on the other hand, provide a direct measure of the spatial positions of chromatin loci in individual cells with a high detection efficiency. In particular, fluorescence in-situ hybridization (FISH) allows highly specific detection of chromatin loci in fixed cells and, more recently, the clustered regularly interspersed short palindromic repeats (CRISPR) system substantially enhanced the ability to image specific chromatin loci in live cells. Chromatin imaging can also be combined with RNA and protein imaging to investigate the interplay between chromatin organization and transcriptional activity or interacting protein factors. However, current imaging methods have limited throughput in genomic (sequence) space, traditionally allowing the study of only a few different genomic loci at a time. Recently developed was a chromatin tracing approach by sequential rounds of FISH imaging, each round targeting one or two genomic loci using one- or two-color imaging. This approach has allowed imaging of tens of distinct chromatin loci in single cells and has been used to provide insights into chromatin structures and their relationship with transcription. However, because the number of genomic loci that can be simultaneously imaged in individual cells remains limited, a high-resolution view of whole chromosomes in single cells is still missing, let alone a genome-scale view of chromatin organization in individual cells.

Reported herein is a multi-scale, multiplexed FISH imaging platform that allows simultaneous imaging of hundreds to >1,000 distinct genomic loci at various resolutions and genomic coverages in single cells. First, the sequential imaging approach was substantially advanced to allow imaging of hundreds of genomic loci and this method was applied to provide a high-resolution view of entire chromosomes, elucidating chromatin domain and compartment structures, their relationship with each other, as well as the relationship between chromatin organization and transcription in single cells. Next, a massively multiplexed FISH approach was developed based on combinatorial labeling and imaging, which allowed more genomic loci to be imaged with much fewer hybridization rounds. Using this approach, simultaneous imaging of >1,000 genomic loci in individual cells was demonstrated, as well as simultaneous imaging of these genomic loci together with nascent RNA transcripts of >1,000 genes residing in these loci and landmark nuclear structures, including nuclear speckles and nucleoli, which allowed chromatin organization to be placed in its native structural and functional context. This approach was used to explore the relationship between trans-chromosomal interactions, transcriptional activity and nuclear structures in single cells.

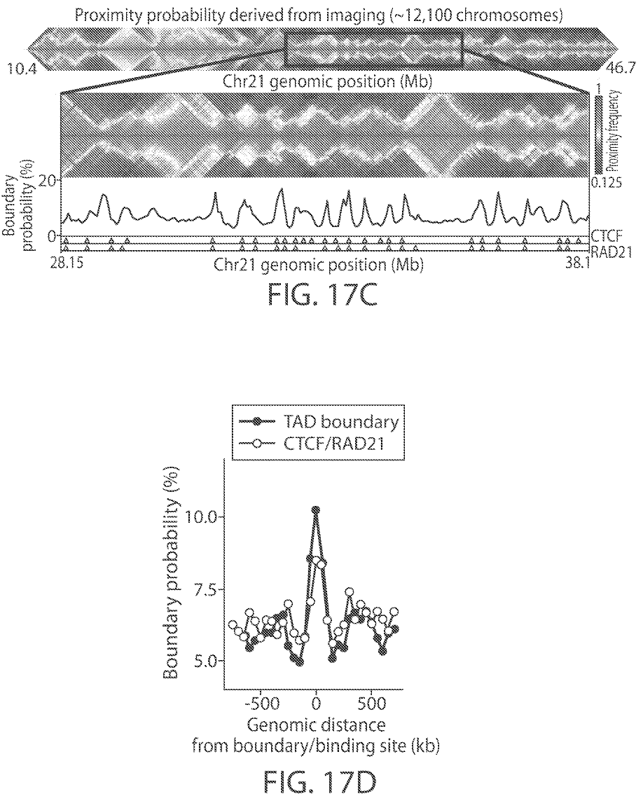

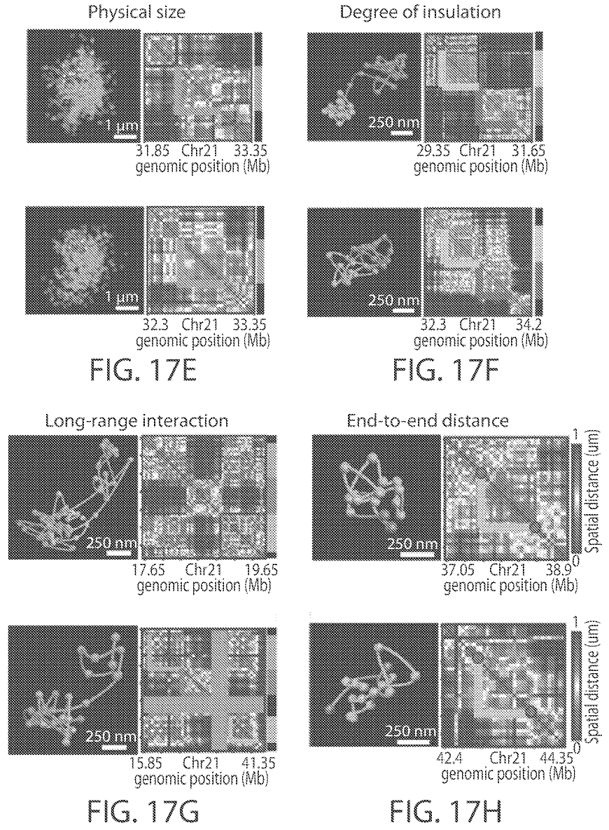

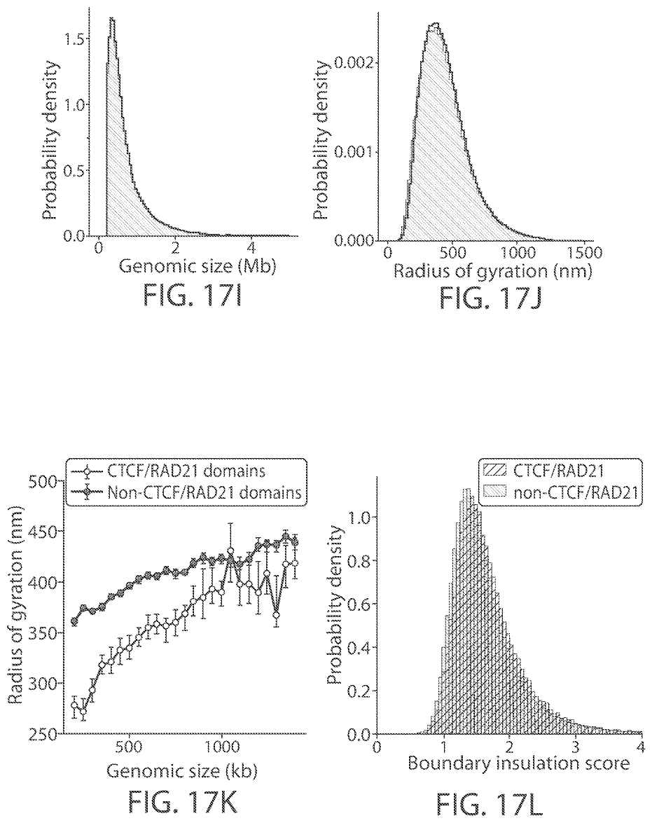

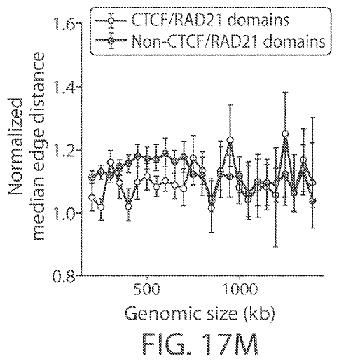

To allow a systematic view of chromatin structures across multiple scales, an imaging platform, using a custom microscope and fluidics setup (see Example 19), was developed for direct visualization of chromatin with exceptionally high throughput in sequence space, up to the genome scale. This platform included two complementary approaches (FIG. 17A). First, for imaging of chromatin structures that were relatively small, such that different loci contained therein would be difficult to resolve in any single image, the previously reported sequential imaging strategy was expanded to allow tracing of hundreds of chromatin loci in single cells. In this approach, chromatin was imaged one locus at a time (or 2-3 loci at a time with 2-3 color imaging) across many imaging rounds (FIG. 17A, left). This approach was demonstrated by using it to trace the conformation of whole chromosomes in single cells at high resolution. Second, to image chromatin structures that were dispersed over an area substantially larger than the diffraction-limited resolution, such as structures spread across the entire nucleus, a more efficient, combinatorial strategy was developed, in which many chromatin loci were imaged simultaneously in each round and their distinct identities were determined based on the different combinations of rounds they appeared in (FIG. 17A, right). This latter approach allowed a large number of genomic loci to be imaged in much fewer imaging rounds. This approach was used to provide a genome-scale view of chromatin organization in the context of transcriptional activity and important nuclear structures, in single cells.

FIGS. 17A-17M show high-resolution whole-chromosome tracing by sequential hybridization and characterization of chromatin domains in single cells. FIG. 17A shows schematics of the multi-scale chromatin tracing platform. Left: Schematic of chromatin tracing of whole chromosomes by sequential hybridization and imaging. When the target chromatin structure is comparable to or smaller than the diffraction limited resolution, a single chromatin locus is imaged in each color channel per imaging round. After all rounds of imaging, a chromatin trace can be generated in 3D for each copy of the targeted chromosome. Right: Schematic of genome-scale imaging by combinatorial FISH. When target loci are expected to be spread out in a space that is substantially larger than the diffraction limited resolution, such as when loci are dispersed in the entire nucleus, multiple loci can be imaged and resolved in each round, and the identity of each locus can be derived from a barcode based on the combination of imaging rounds in which the locus is detected. This approach significantly reduces the number of rounds required to image the same number of loci compared to the sequential imaging approach.

### Example 7

High-resolution chromatin tracing of whole chromosomes. In this section, a high-resolution whole-chromosome tracing by the sequential imaging approach is described (FIG. 17A, left; FIG. 24A). Human chromosome 21 (Chr21) was focused on first and the non-repetitive portion of the chromosome (Chr21: 10.4-46.7 Mb) was partitioned into >600 contiguous segments (i.e. >600 genomic loci), each 50-kb in length. A library of primary oligonucleotide probes was designed, each containing a variable target sequence for hybridizing to the chromosome and a readout sequence that was unique to each of the 50-kb loci (FIG. 24A). All primary probes bound to a specific 50-kb locus shared the same readout sequence, and hence the readout sequences could be used to identify each locus through hybridization of complementary readout probes labeled with fluorescent dyes (FIG. 24A). However, identifying these genomic loci with >600 distinct, fluorescently readout probes would be prohibitively expensive due to the high cost of dye-labeled oligonucleotides. To overcome this challenge, a two-step labeling strategy was devised to detect the distinct readout sequences with a common set of three dye-labeled oligonucleotide probes (called readout probes, one readout probe for each color channel), mediated by unlabeled adaptor probes that convert each locus-specific readout sequence into one of the three common readout sequences (FIG. 24A). Using this strategy, >600 chromatin loci in Chr21 in human lung fibroblast (IMR-90) cells were sequentially imaged, using >200 rounds of hybridization of adaptor and readout probes with three-color imaging in each round. To allow stable imaging over such a large number of hybridization rounds, the imaging protocol was further optimized in the following ways: (i) to maintain sample integrity and primary probe binding stability, the sample was re-fixed with formaldehyde periodically during the course of imaging; (ii) to ensure complete removal of fluorescence signal after each imaging round and minimize the accumulation of residual signal across hundreds of labeling rounds, a combined chemical cleavage and photobleaching approach was used to remove the fluorescence signal of the readout probes, and unlabeled readout probes were added to block any unoccupied binding sites on adaptor probes after each round of imaging; (iii) to minimize perturbations and the experiment time, the duration of each hybridization round and the flow rates of the fluidics system were optimized.

After imaging, the centroid position of each chromatin locus was determined in 3D and the conformation of each homologous copy of Chr21 in each cell was reconstructed (FIG. 17B). To estimate how stable the sample and imaging instruments were over many hybridization rounds, the same chromatin loci were reimaged after different numbers of hybridization/imaging rounds and the displacement between the original loci's locations and those of the corresponding re-imaged loci were used as a measure of the measurement accuracy. The median displacement between the original and re-imaged loci increased from ˜70 nm when 11 hybridization rounds separated the two imaging instances to −120 nm when the initial and re-imaging instances were separated by −250 hybridization rounds (FIG. 24B-24C), with the loci that displayed greater displacement upon re-imaging also having lower fluorescent signal intensity (FIG. 24D). It was noted that the median displacement error, even after >250 rounds of hybridization, was substantially smaller than the median distance between neighboring chromatin loci (˜250 nm) (FIG. 24B). In addition, median pairwise distances between imaged loci were highly reproducible between biological replicates (FIG. 24E). The detection efficiency of the chromatin loci in these experiments was >90% (i.e. >90% of the targeted chromatin loci were detected in each chromosome).

To obtain a population-averaged view of the chromatin conformation of Chr21, the pairwise interaction between imaged loci was quantified by calculating their median spatial distance and the probability that the loci come into proximity across the −3,500 imaged cells (FIG. 17C and FIGS. 24F-24I). A high correlation between the median pairwise distances from the imaging data and previously published Hi-C data across all length-scales present in Chr21 (Pearson correlation of 0.89; FIGS. 24F-24G) was observed, with agreement being particularly high for shorter genomic distances (Pearson correlation of 0.97; FIGS. 24H-24I). To choose the cutoff distance below which two loci were considered to be in proximity, the Pearson correlation coefficient between the Hi-C data and the proximity frequency (i.e. the fraction of instances in which the two loci were in proximity) derived from the imaging data across a range of cutoff distances was calculated. The Pearson correlation coefficient remained high for a wide range of cut-off distances but peaked at 0.88 when the cutoff distance was −400-500 nm (FIG. 24J). Thus, 500 nm was chosen as the cutoff distance to generate proximity frequency maps throughout this work (See Example 19 for a more detailed rationale for the selection of the cut-off distance).

Both the median distance and the proximity frequency maps showed block-like TAD structures (FIG. 17C and FIGS. 24F and 24H). TAD boundaries identified from both the distance and proximity frequency maps from the imaging data were highly similar to those determined from ensemble Hi-C data (FIG. 24K). In addition, it was confirmed that the locus localization error in the chromatin traces (˜100 nm) had little effect on domain boundary identification and its accuracy (FIG. 24K).

FIG. 17B shows 3D structural rendering and spatial distance matrices of the two copies of Chr21 in a single IMR90 cell imaged by the sequential hybridization approach. Left: The two copies of Chr21 in a single cell are overlaid on a DAPI image of the nucleus. Scale bar: 5 micrometers. Right, top: 3D rendering of all detected chromatin loci (spheres) in the two Chr21 copies according to their genomic coordinates along the chromosome (genomic position shown to the right). Flexible lines connect adjacent chromatin loci. Scale bar: 1 micrometer. Right, bottom: Pairwise spatial distance matrices corresponding to the chromosome copies displayed above (genomic regions without proper reference genomes or containing highly repetitive sequences are not imaged).

FIG. 17C shows ensemble proximity frequency matrix of Chr21 and preferential positioning of single-cell domain boundaries at CTCF/RAD21-binding sites. Top: Proximity frequency matrix for Chr21 derived from imaging data. Each matrix element is defined as the frequency with which the measured distance between a pair of loci is shorter than a cutoff distance of 500 nm. Middle: zoomed-in version of the proximity frequency matrix for a 10-Mb portion of Chr21. Bottom: the probability of single-cell domain boundary formation at each of the imaged 50-kb genomic segment. Triangles show CTCF and RAD21 ChIP-seq peaks.

FIGS. 24A-24N show high-resolution whole-chromosome tracing by sequential hybridization, and ensemble statistics of Chr21 structural features in comparison with Hi-C. FIG. 24A shows labeling and imaging scheme for sequential hybridization with adaptor probes. First, the sample is hybridized with primary probes, each containing a target sequence that allows specific binding to a targeted genomic locus and a readout sequence. Each locus is labeled by a total of 350-500 primary probes, but only one is shown. Each targeted genomic locus is assigned a unique readout sequence (shown in various colors), common to all primary probes that bind to the locus. The readout sequences are then detected using sequential rounds of hybridization. During each round of hybridization, the readout sequences corresponding to the targeted loci (one for each of the three color channels, Alexa750, Alexa647, and Cy3) are labeled with oligonucleotide adaptor probes that each consist of two portions: a segment complementary to the locus-specific readout sequence and a segment containing a color-channel-specific common readout sequence. Each color channel contains a unique common readout sequence that is shared by all the adaptors visualized in the same color channel. The common readout sequences are then hybridized to dye-conjugated, complementary readout probes in corresponding color channels. This procedure allows three genomic loci to be imaged in three color channels during each round of hybridization. Following each round of imaging, fluorescent dyes, which are connected to the readout probes by a di-sulfide bond, are cleaved from the common readout probes by TCEP, and any unoccupied readout sequences on the adaptors are blocked with unlabeled common readout probes to prevent crosstalk between rounds of hybridization. The process is iterated over hundreds of rounds until the detection of all readout sequences, and hence all targeted genomic loci, is completed.

FIG. 24B shows the displacement of loci over the course of a single experiment. Consecutive 50-kb segments within a 900-kb region in Chr21 (chr21:32.45-33.35 Mb) were imaged both at the beginning and at the end of the experiment, separated by >250 rounds of hybridization. The distribution of the displacement between the re-imaged spot and its originally imaged counterpart are shown. For comparison, the distribution of the distances between adjacent 50-kb segments in the same 900-kb region measured in the original imaging rounds are shown.

FIG. 24C shows boxplots of the displacement of chromatin loci between the original and re-imaging rounds separated by different number of hybridization rounds. The medians (center lines), 25th-75th percentiles (boxes) and 10th-90th percentiles (whiskers) are shown.

FIG. 24D shows boxplots of the fluorescent signal of chromatin loci with low (<500 nm) and high (>500 nm) displacements errors between original and re-imaging experiments separated by >250 rounds of hybridization. The medians (center lines), 25th-75th percentiles (boxes) and 10th-90th percentiles (whiskers) are shown.

FIG. 24E shows a comparison of median inter-loci spatial distances between two replicate experiments. Median spatial distances between pairs of imaged chromatin loci were calculated separately for two biological replicate experiments of Chr21 and plotted against each other. The Pearson correlation coefficient between data measured in the two replicates is ρ=0.98.

FIG. 24F shows a comparison of median spatial distance matrix derived from imaging (left), proximity frequency matrix derived from imaging (middle), and ensemble Hi-C contact matrix (right) for Chr21. For the imaging data, two chromatin loci are considered to be in proximity when the spatial distance between the two loci is smaller than a cutoff distance of 500 nm. The Hi-C contact matrix is binned at 50 kb and centered around the target regions.

FIG. 24G shows a log-log scatter plot of number of contacts derived from ensemble Hi-C and median pairwise distances derived from imaging for individual pairs of chromatin loci. Line represents linear regression of the data (slope=−4.43). The Pearson correlation coefficient between imaging and Hi-C data is ρ=0.89.

FIG. 24H is the same as FIG. 24F but for a 3-Mb region in Chr21 (chr21: 30.30-33.38 Mb). TAD boundaries are marked with lines.

FIG. 24I is the same as FIG. 24H, but for the 3-Mb region shown in (H). slope=−4.51. ρ=0.97.

FIG. 24J shows a Pearson correlation between Hi-C contact map and imaging-derived proximity frequency maps generated with varying cutoff distances. To generate a proximity frequency map, a cutoff distance is chosen and two loci with a distance smaller this cutoff value are considered to be in proximity. Then, proximity frequency between a pair of loci was calculated as the number of incidences in which the measured distance between the loci is shorter than the cutoff distance divided by the total number of measured distances between the two loci.

FIG. 24K shows normalized insulation scores as a function of the genomic position on Chr21 calculated from: 1) the median pairwise distances from imaging (top), 2) proximity frequencies from imaging (middle), and 3) Hi-C contact reads (bottom). To calculate the insulation score, a fixed-length (250 kb) genomic segment upstream and a same-length segment downstream of the position of interest were first selected. The normalized insulation score is then defined as the difference between median inter-segment pairwise distance and median intra-segment pairwise distance, normalized by the sum of these two median distances. TAD boundaries are defined as local maxima of the normalized insulation score along the chromosome, identified by a standard peak-calling algorithm (see Example 19). The vertical dotted lines are the ensemble TAD boundaries called from the Hi-C data. Also shown in the top and middle panels are the median distances (black line, top panel) and proximity frequencies (black line, middle panel) after perturbing the loci positions with a 3D Gaussian noise term with a standard deviation of 100 nm, comparable to the estimated localization measurement error.

FIG. 24L shows chromatin domains in a 10-Mb region of Chr21 (chr21: 28.2-38.1 Mb) in two example single cells. Pairwise distances from two individual copies of Chr21 in single cells are shown (Top, middle) together with population median pairwise distances derived from all image cells (bottom).

### Example 8

Chromatin domains in single chromosomes. At the single-cell level, it was observed that chromosomes were partitioned into domains that manifest as block-like features in single-cell spatial distance matrices (FIG. 24L). These domains and the inter-loci distances showed high variability from cell to cell (FIGS. 24L-24M), consistent with the substantial cell-to-cell variability in chromatin contacts observed in single-cell Hi-C data. Similar domain structures in single cells were previously observed when imaging small (˜2 Mb) regions of the chromosome with similar resolution. However, within these previously measured small regions, a sizable fraction of cells did not display clear single-cell domain boundaries and it remained uncertain whether domains did not form within those cells or whether the entire imaged regions were within a single domain. Furthermore, due to the small size of these previously imaged regions, many domains were artificially truncated at the ends of the genomic regions imaged, thus prohibiting accurate characterization of certain basic domain properties such as their physical and genomic sizes. The high genomic throughput in this study provided a whole-chromosome view of these single-cell domain structures, revealing their prominent presence across the entire chromosome in essentially all imaged cells, thus allowing the characterization of their properties in a more systematic manner.

The genomic locations of the boundaries of these single-cell domains were first identified and quantified the probability of boundary formation at each 50-kb genomic locus. While a non-zero probability of boundary formation was observed at all imaged genomic loci, the domain boundaries were preferentially positioned near the binding sites of CTCF and cohesin (FIGS. 17C-17D).

In addition to the cell-to-cell variations in the location of domain boundaries, substantial heterogeneity in other features of these single-cell domains was observed, ranging from the physical sizes of the domains to the degree of insulation or interaction between domains (FIGS. 17E-17H). Specifically, it was observed that single-cell domains were variable in both their genomic sizes (FIG. 17I) and their physical sizes as measured by the radius of gyration (FIGS. 17E and 17J). Neither the distribution of genomic sizes nor the distribution of physical sizes of these domains were sensitive to the estimated locus localization error of ˜100 nm (FIGS. 17I-17J). Notably, domains bounded by the same genomic regions, or having the same genomic size, fluctuated considerably in their physical sizes from cell to cell (FIGS. 17E and 24N). Interestingly, domains bounded by interacting CTCF/cohesin binding sites tended to be smaller in physical size than domains not bounded by such genomic loci (FIG. 17K). In addition, the degree of physical segregation between neighboring domains also varied substantially (FIGS. 17F and 17L), with some neighboring domains completely segregated and only connected by a linker region while others displayed partial overlap and less sharp boundaries (FIG. 17F). Moreover, even domains that were completely segregated from their neighboring domains could partially overlap in space with non-neighboring domains separated by small or large genomic distances (FIG. 17G). Lastly, it was observed that the two chromatin loci at the ends of these single-cell domains also exhibited a variable distance from each other and did not exhibit a tendency to be closer to each other compared to chromatin loci separated by a similar genomic distance in the interior of a domain, regardless of whether domains were bounded by CTCF/cohesin-binding sites (FIGS. 17H and 17M).

FIG. 17D average probability of domain boundary formation in single cells at genomic locations centered around CTCF/Rad21-binding sites or around ensemble TAD boundaries (grey).

FIG. 17E is an example of two single-cell chromatin domains with identical genomic coordinates, occupying large (top) or small (bottom) volumes in the physical space. Left: 3D rendering of the chromatin domains, with green balls representing imaged genomic loci within the domain and flexible linkers connecting adjacent loci in genomic sequence. Grey spots represent imaged loci in the rest of the chromosome. Scale bar: 1 micrometer. Right: pairwise distance matrix for the chromatin domain shown on the left (marked with lines), with flanking regions.

FIG. 17F shows an example of two pairs of chromatin domains with high (top) and low (bottom) insulation scores. Left: 3D rendering of the chromatin domains, as in FIG. 17E. Scale bar: 250 nm. Right: pairwise distance matrix for the chromatin domains shown on the left, with rendered domains marked in corresponding colors.

FIG. 17G shows two examples of long-range contact between chromatin domains with partially overlapping volumes. Left: 3D rendering of the chromatin domains, as in (E). Shadings represent different domains. Scale bar: 250 nm. Right: pairwise distance matrix for the chromatin domains, with rendered domains marked in corresponding colors. The grey space indicates a gap in genomic distance of 22.85 Mb.

FIG. 17H shows an example of chromatin domains flanked by CTCF binding sites, showing small (top) and large (bottom) distances between the CTCF sites. Left: 3D rendering of the chromatin domain, as in (E), but with the loci of CTCF sites at the domain ends. Scale bar: 250 nm. Right: pairwise distance matrix for the chromatin domain with domain and border CTCF marked correspondingly.

FIG. 17I shows a distribution of measured genomic sizes of chromatin domains in Chr21 in single cells. Shown in black line is the distribution of genomic sizes of chromatin domains in Chr21 in single cells derived from simulated data that considers a localization error of 100 nm. In this simulation, the positions of the imaged loci are perturbed with a 3D Gaussian noise with standard deviation of 100 nm, similar to our measurement error.

FIG. 17J shows a distribution of measured physical sizes, as defined by the radii of gyration, of chromatin domains in Chr21 in single cells. Shown in black line is the distribution of physical sizes of chromatin domains in Chr21 in single cells derived from simulated data that considers a localization error of 100 nm, as in FIG. 17I.

FIG. 17K shows a median radius of gyration as a function of genomic size for chromatin domains with boundary loci containing interacting CTCF/Rad21 sites and with neither boundary locus containing CTCF/Rad21 sites. Error bars indicate 95% confidence intervals derived by resampling.

FIG. 17L shows a distribution of insulation scores between neighboring domains with domain boundaries occurring at CTCF/Rad21-binding sites and non-CTCF/Rad21-binding sites.

FIG. 17M shows a median of normalized end-to-end distance of domains as a function of genomic size for chromatin domains with boundary loci containing interacting CTCF/Rad21 sites and with neither boundary locus containing CTCF/Rad21 sites. The normalized end-to-end distance is defined as the domain's end-to-end distance divided by the median distances between likewise locus pairs separated by the same genomic distance but lying in the interior of a single domain. Error bars indicate 95% confidence intervals derived by resampling.

FIG. 24M shows a standard deviation matrix of the inter-loci spatial distances for Chr21. For each pair of regions, the standard deviation of the distances between the corresponding pair of loci in all single chromosome copies is shown.

FIG. 24N shows boxplots of the physical sizes of chromatin domains of different genomic sizes in Chr21, measured by the radii of gyration. For each genomic size, the median (center lines), 25th-75th percentiles (boxes) and 10th-90th percentiles (whiskers) are shown.

### Example 9

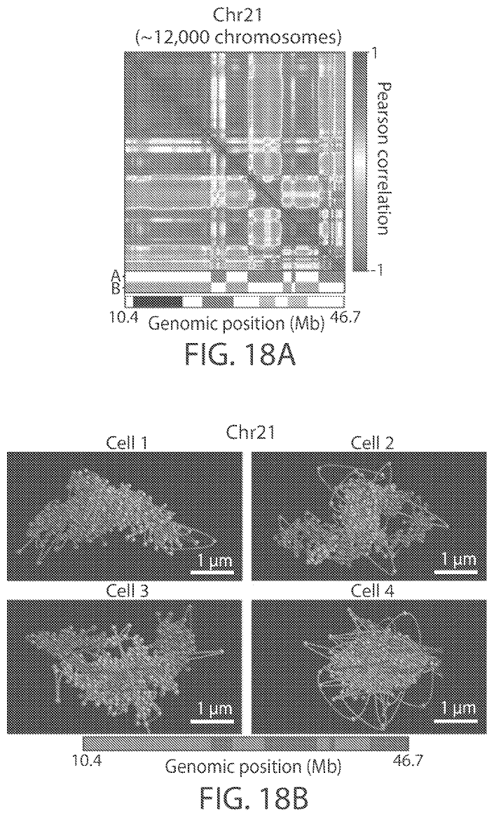

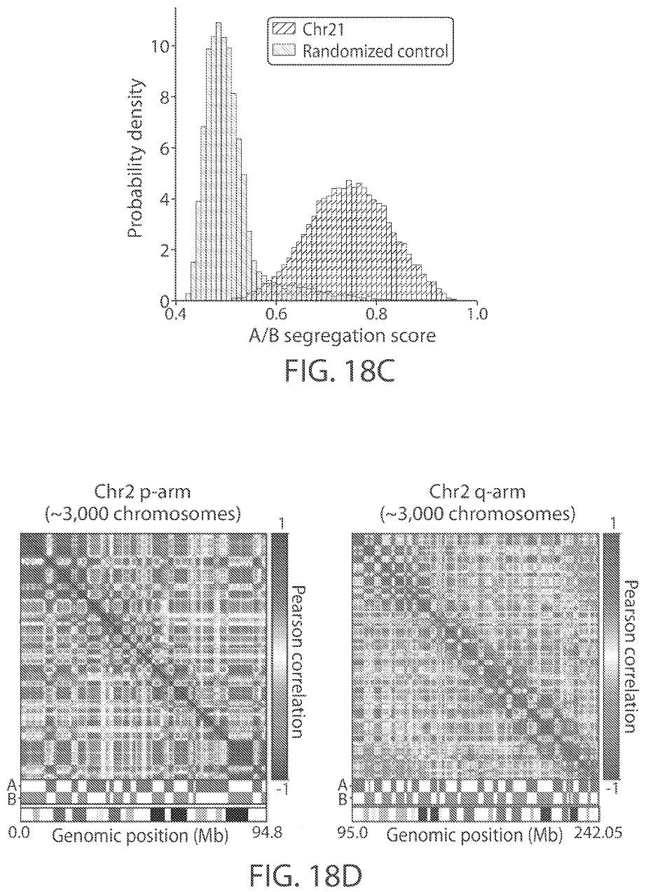

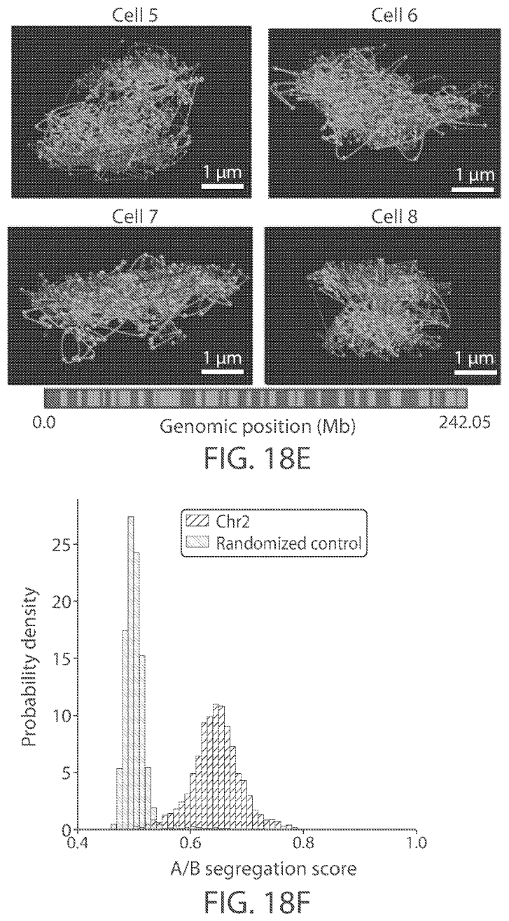

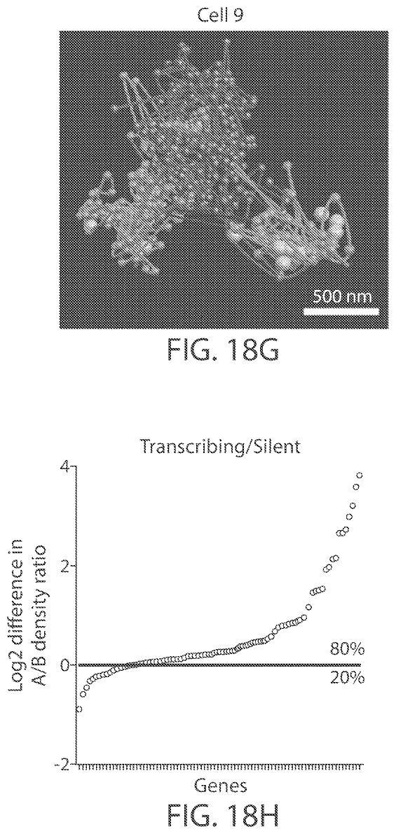

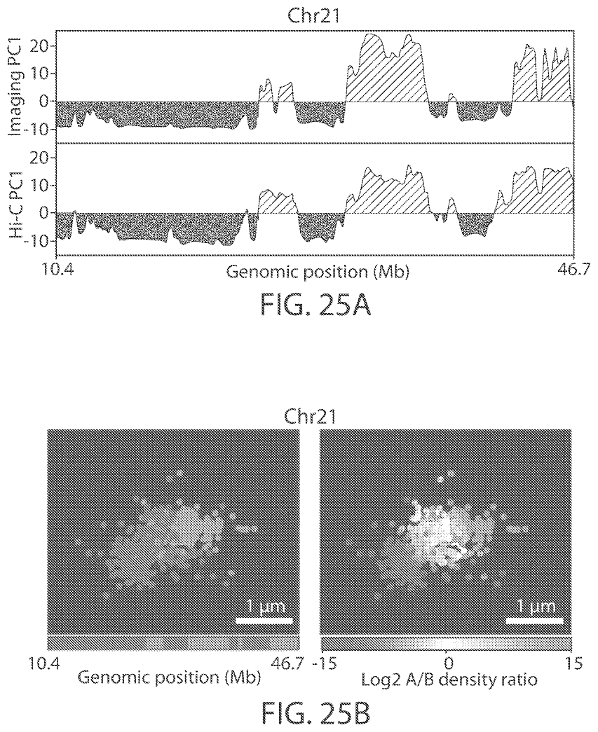

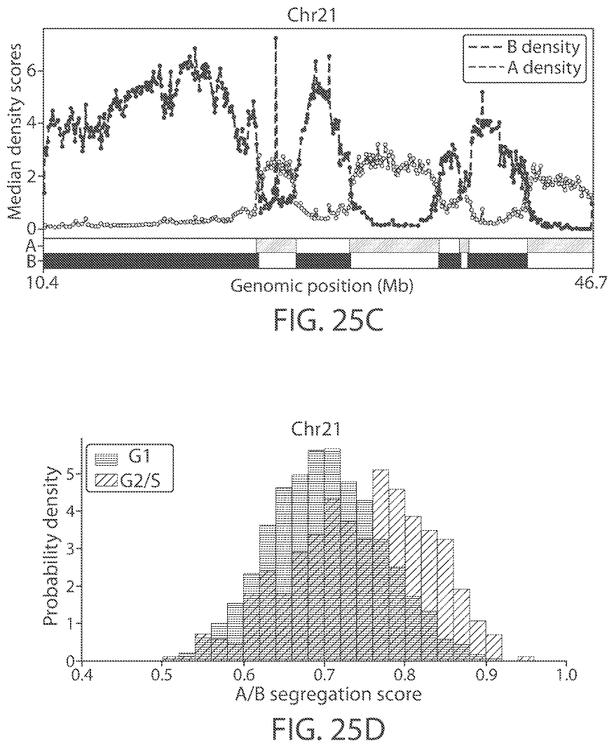

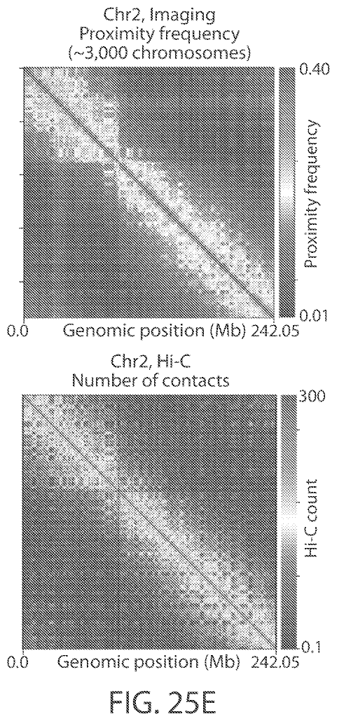

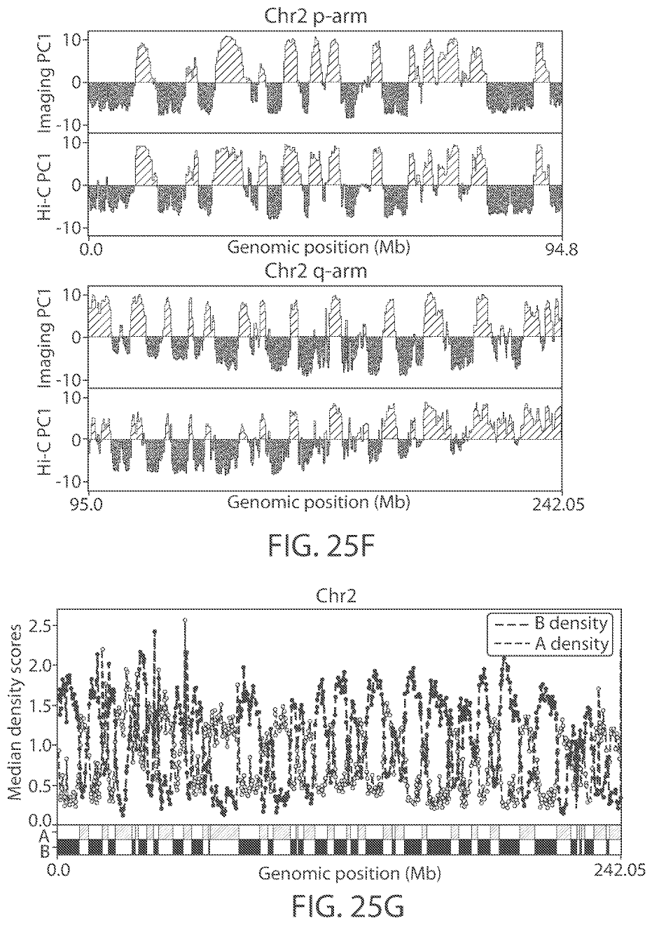

Chromatin compartments in single chromosomes. Next, the high-resolution view of whole chromosomes was used to examine how chromatin loci in A and B compartments are arranged in single cells. First, the ensemble A/B compartment boundaries were determined using principal component analysis (PCA) of the Pearson correlation matrix of the proximity frequency map for Chr21, derived from the imaging data, using a previously described algorithm (FIG. 18A; FIG. 25A). The compartment boundaries obtained from the imaging data were highly similar to those determined from previously published ensemble Hi-C data (FIG. 25A). Below, the compartment boundaries obtained from the ensemble proximity frequency map were used to assign A/B identity for individual loci in individual cells.

The >10-fold increase in resolution compared to the previous study allowed a detailed view of compartment-A loci and compartment-B loci organization in single chromosomes. A high degree of variation in the arrangement of A and B loci was observed between individual chromosome copies from cell to cell (FIG. 18B). While in some chromosomes, A and B loci were segregated into essentially non-overlapping spatial territories, other chromosomes exhibited substantial spatial overlap between A and B loci. Intriguingly, compartment A loci in the same chromosome were sometimes separated into multiple “micro-compartments” (FIG. 18B).

To quantify the degree of spatial segregation of A and B loci in individual chromosomes, a local-density-based approach was devised and for each imaged locus, the local density of other A and B loci was computed (FIG. 25B). As expected, compartment-A loci, on average, tended to be surrounded by A loci, and the same was true for B loci (FIG. 25C). An A/B segregation score was further defined for each individual chromosome based on the purity of loci observed in the spatial volumes harboring the majority of A or B loci (FIG. 18C). Complete physical segregation of A and B loci was expected to result in a segregation score of 1 and thorough mixing of A and B loci to result in a segregation score of 0.5 (see Example 19). It was observed that, for the vast majority of Chr21 copies in cells, the segregation scores were considerably higher than the scores of the randomization controls centered around 0.5 (obtained by randomly shifting compartment boundaries along the genomic axis while keeping compartment sizes unchanged) (FIG. 18C), demonstrating a tendency for A and B loci to spatially segregate in single cells. It is also worth noting that the spatial segregation of A and B loci was often incomplete (FIG. 18C). This potentially reflected an incomplete spatial segregation of active and inactive chromatin, but could have also been caused, in part, by a cell-to-cell variability in epigenetic modifications, which could make the ensemble A/B compartment identity an imperfect proxy for active/inactive chromatin delineation in single cells. Notably, the degree of A/B segregation was found to be cell-cycle dependent: A/B segregation was stronger for cells in the G2/S phase compared to cells in the G1 phase (FIG. 25D), which was consistent with previous findings of the gradual establishment of A/B compartments during the cell cycle.

It was noted that Chr21 is one of the smallest chromosomes, with a size of only −48 Mb, and is partitioned into only a small number of contiguous A and B regions. To extend the findings to larger chromosomes and investigate how general they are, one of the largest chromosomes, Chromosome 2 (Chr2), which exhibits a high number of transitions (˜50 transitions) between A and B compartments along its genomic sequence, was imaged. Specifically, Chr2 was traced by labeling and imaging 50-kb segments at intervals of 250-kb along its genomic sequence. The same approach described above was used to call A and B compartments in the p and q arms of the chromosome based on the imaging data (FIG. 18D; FIG. 25E), and quantitative agreement with the A and B compartments determined from previously published ensemble Hi-C data was observed (FIGS. 25F-25G). At the single-chromosome level, a variety of spatial arrangements of A and B loci was again observed, ranging from nearly complete spatial segregation to substantial spatial overlap between A and B loci (FIG. 18E). Interestingly, some chromosomes showed a “sandwich” configuration in which A loci were placed between two layers of B loci, possibly due to the preferential association of the B loci with nuclear lamina near the top and bottom of the cell nucleus. Quantitatively, the A/B segregation score distribution across individual copies of Chr2 again showed a global tendency for A and B loci to segregate in individual chromosomes compared to the randomization control (FIG. 18F). The degree of spatial segregation appeared to be smaller in Chr2 than in Chr21 (FIGS. 18C and 18F).

FIGS. 18A-18I show the compartment structure in single chromosomes and relationship between transcription activity and local chromatin content. FIG. 18A shows a Pearson correlation matrix for genomic-distance-normalized proximity frequencies of Chr21 derived from our imaging data. Two loci are considered in proximity if their distance is smaller than a cutoff distance of 500 nm. Two bars at the bottom shows the A/B calling derived from proximity frequency matrix (shown for A compartments and B compartments) and G-banding of each genomic locus in the chromosome.

FIG. 18B shows 3D renderings of individual copies of Chr21 in single cells, with A and B loci shown as balls. Flexible lines connect adjacent loci in genomic sequence. The bar at the bottom shows the A/B calling of each genomic locus in the chromosome derived from proximity frequency matrix. Scale bar: 1 micrometer.

FIG. 18C shows a distribution of the A/B segregation score for individual copies of Chr21. To calculate the A/B segregation score, an A (or B) dense volume is defined for each chromosome by thresholding the local A (or B) density such that ⅔ of A (or B) loci were contained within the volume (note that the A and B dense volumes can overlap for chromosomes that show spatial overlap between A and B loci). The purity of loci in the A (or B) dense volume of the chromosome was defined as the fraction of all loci within the volume being A (or B) loci, and the A/B segregation score of a chromosome copy was defined as the mean purity of the A and B volumes. The histogram represents the distribution of A/B segregation scores for a randomization control, where the boundaries between contiguous A and B regions are randomly shifted along the genomic sequence, while keeping the number and sizes of A and B regions unchanged. n=−7,500 chromosomes.

FIG. 18D shows a Pearson correlation matrix for genomic-distance-normalized proximity frequencies for the p and q arms of Chr2 derived from our imaging data, and corresponding A/B calling and G-banding, as in FIG. 18A.

FIG. 18E shows 3D renderings of individual copies of Chr2, as in FIG. 18B. Scale bar: 1 micrometer.

FIG. 18F shows a distribution of A/B segregation scores for Chr2 in single cells, as in FIG. 18C. n=˜3,100 chromosomes.

FIGS. 25A-25G show ensemble A/B compartment analyses for Chr21 and Chr2. FIG. 25A shows a compartment calling based on principal component analysis for Chr21. First principal component (PC1) calculated for the Pearson correlation matrices from genomic-distance-normalized proximity frequency derived from imaging (top) and ensemble Hi-C (bottom) experiments are shown, with PC1 value >0 corresponding to compartment A and PC1 value <0 corresponding to compartment B.

FIG. 25B shows a 3D rendering of compartment-A loci (A loci) and compartment-B loci (B loci), and A/B density ratios in a single copy of Chr21. Left: A and B loci of a representative copy of Chr21. A and B compartment calling from the ensemble proximity frequency map derived from imaging is shown at the bottom bar. Right: the same chromosome but with each locus colored by its local A/B density ratios.

FIG. 25C shows mean A and B density scores for each imaged locus in Chr21 averaged across all imaged cells. The bottom panel represents the A or B compartment calling of each locus from the proximity frequency map derived from imaging.

FIG. 25D shows histograms of the distributions of A/B segregation scores for individual copies of Chr21 in cells in the G1 and G2/S phases of the cell cycle.

FIG. 25E shows a proximity frequency matrix derived from imaging (left) and ensemble Hi-C contact matrix (right) for Chr2. The Hi-C contact matrix is binned at 50 kb, but only the contacts from the imaged segments (selected at 250-kb intervals) are shown.

FIG. 25F shows the same PC analyses as in FIG. 25A, but for the p-arm (top) and q-arm (bottom) of Chr2.

FIG. 25G shows the same mean A and B density analysis as in FIG. 25C, but for Chr2.

### Example 10

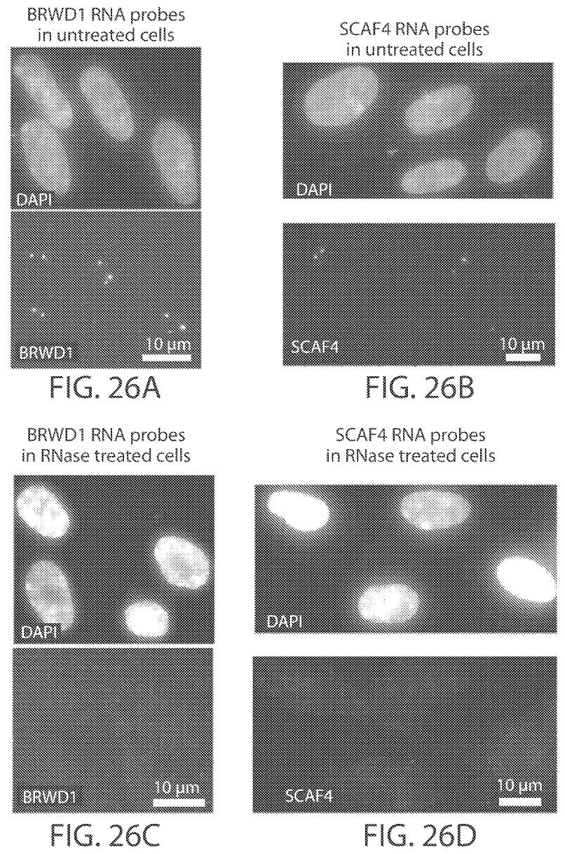

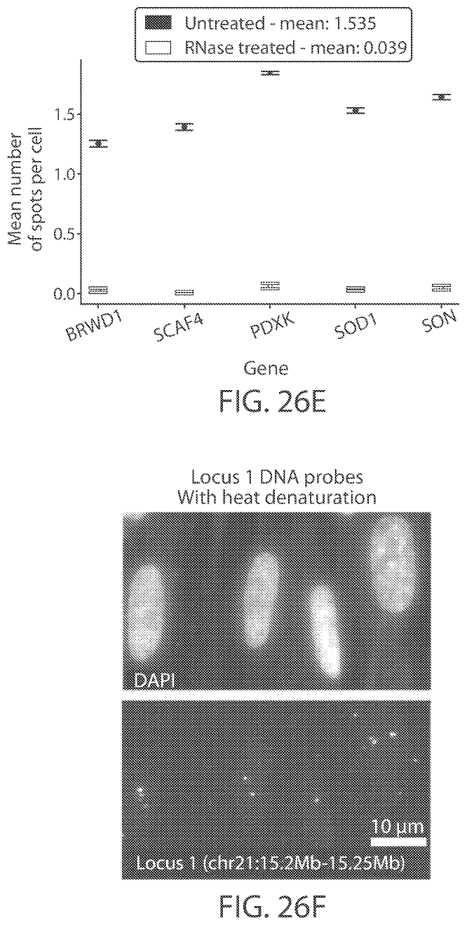

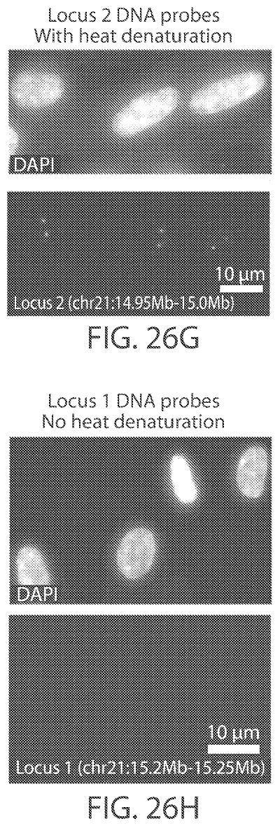

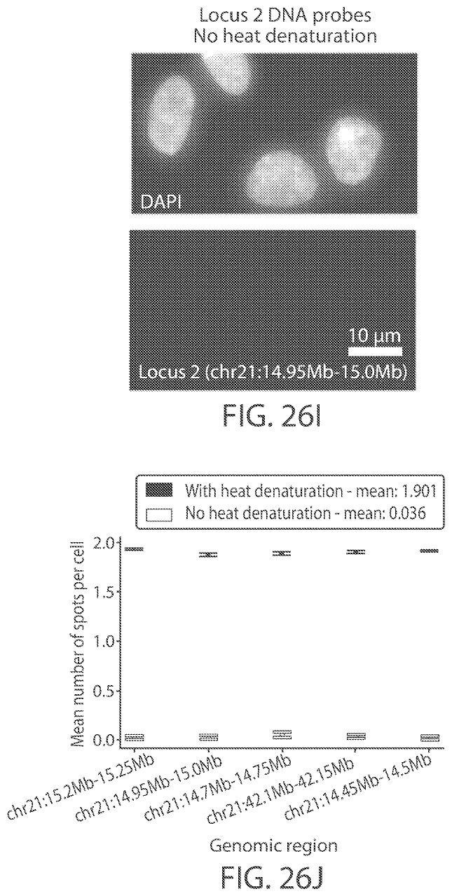

Relationship of transcription to local A/B chromatin content. To study whether chromatin compartmentalization is correlated with active transcription in single chromosomes, oligonucleotide probes targeting the first intron of 86 of the genes harbored in Chr21 were designed and sequential rounds of hybridization were performed to image nascent RNA transcripts of these genes followed by chromatin tracing. Furthermore, to more accurately detect the spatial position of the genes, the 5-kb genomic loci centered around the transcription start site (TSS) of each target gene was imaged. To prevent RNA probes from binding genomic DNA and vice versa, RNA probe hybridization was performed without heat denaturing the double-stranded genome, and then the RNA molecules were digested using an RNase treatment (a step that was also included when imaging chromatin alone) before performing chromatin tracing. Crosstalk between the RNA and DNA signals was confirmed to be negligible using this strategy (FIGS. 26A-26J).

Typically, a subset of the imaged genes showed transcription activity in any individual cell (FIG. 18G). It was examined how transcriptional activity was correlated with the local chromatin environment. To characterize the local A/B chromatin content, the local density of nearby A and B loci were calculated for each gene and their ratio (referred to hereafter as the A/B density ratio) was used as a metric for the local enrichment of active chromatin. It was found that for ˜80% of the genes studied, when a gene was actively transcribing, the local A/B density ratio at its TSS was higher than when the gene was not firing (FIG. 18H). As a corollary, the firing rate of a gene also tended to be higher in cells where the TSS of the gene had a higher local A/B density ratio (FIG. 18I). These results indicated that the same gene tended to have a higher transcriptional activity in cells with higher enrichment of A loci and/or de-enrichment of B loci in the neighborhood of the gene. This increase in transcriptional activity could potentially be due to a local enrichment of transcription machinery and/or de-enrichment of silencing factors. Alternatively, it was also possible that actively transcribing chromatin, with transcriptional machinery associated, has a stronger tendency to interact with other active chromatin, given that transcription machinery and co-factors can form condensates. It was noted that these two possible mechanisms are not mutually exclusive but could work cooperatively to reinforce each other.

FIG. 18G shows a 3D rendering of a single copy of Chr21 shown together with the transcriptional bursts of the measured genes. Balls represent all detected nascent RNA bursts in this chromosome. Scale bar: 500 nm.

FIG. 18H shows the change (measured in log difference) in the A/B density ratios at the transcription start sites (TSS) of the imaged genes between actively firing and non-firing states. For each gene, the median A/B density ratio is computed at its TSS in chromosomes where the gene is firing and in chromosomes where it is not firing. The log-difference of these values for the 84 genes imaged on Chr21 is rank-ordered according to the magnitude of change in their median A/B density ratio. 79% of the imaged genes exhibited an increase in the A/B density ratio when they are actively firing, compared to not firing.

FIG. 18I shows the change (measured in log difference) in the firing rates of the imaged genes as the local environment of the TSS of the gene is changed from low (bottom quartile) to high (top quartile) A/B density ratios. The log-difference in firing rate for the 84 genes imaged on Chr21 is rank-ordered according to the magnitude of firing rate. 79% of the imaged genes showed a higher firing rate when their TSS is in the top quartile, compared to the bottom quartile, of A/B density ratios.

FIGS. 26A-26J show measurements for RNA and DNA FISH probe crosstalk. FIG. 26A shows example cells with their nucleus marked by DAPI (top) and the fluorescent signal of the FISH probes targeting the nascent RNA of a gene (BRWD1) (bottom).

FIG. 26B is the same as FIG. 26A but for a different gene (SCAF4). The staining of FIGS. 26A and 26B follows the protocol described in the RNA FISH protocol described in the “Cell culture preparation and primary/encoding probe hybridization” section in Example 19.

FIGS. 26C and 26D are the same as FIGS. 26A and 26B, respectively, except that the RNA FISH protocol was modified to include an additional RNase treatment step to remove the cellular RNAs prior to addition of the FISH probes. The cells in FIG. 26C and FIG. 26D were imaged under similar illumination conditions to FIGS. 26A and 26B and their fluorescent signal is displayed with the same contrast as in FIGS. 26A and 26B.

FIG. 26E shows the number of spots per cell with a signal-to-noise ratio >3 for untreated and RNase-treated cells across 5 measured genes.

FIG. 26F shows example cells with their nucleus marked by DAPI (top) and the fluorescent signal of the probes targeting a genomic locus (chr21:15.2 Mb-15.25 Mb) (bottom).

FIG. 26G is the same as FIG. 26F, but for a different locus (chr21:14.95 Mb-15 Mb). The staining of FIG. 26F and FIG. 26G follows the protocol described in the DNA FISH protocol described in the “Cell culture preparation and primary/encoding probe hybridization” section in Example 19.

FIGS. 26H and 261 are the same as FIGS. 26F and 26G, respectively, except that the DNA FISH protocol was modified to omit the heat denaturation step and hence to remove the accessible genomic DNA sites. The cells in FIGS. 26H and 26I were imaged under similar illumination conditions to FIGS. 26F and 26G and their fluorescent signal is displayed with the same contrast as in FIGS. 26H and 261.

FIG. 26J shows the number of spots per cell with a signal-to-noise ratio >3 for the cells treated with the heat denaturation step and the cells for which this step was omitted.

### Example 11

Relationship between chromatin domains and compartments in single chromosomes. Next, the interactions between single-cell chromatin domains and how these interactions correlate with compartment identities were investigated. Because of the large size of Chr2 and the large number of compartment partitions therein, analyses on Chr2 was expected to provide more insights and thus this chromosome was focused on.

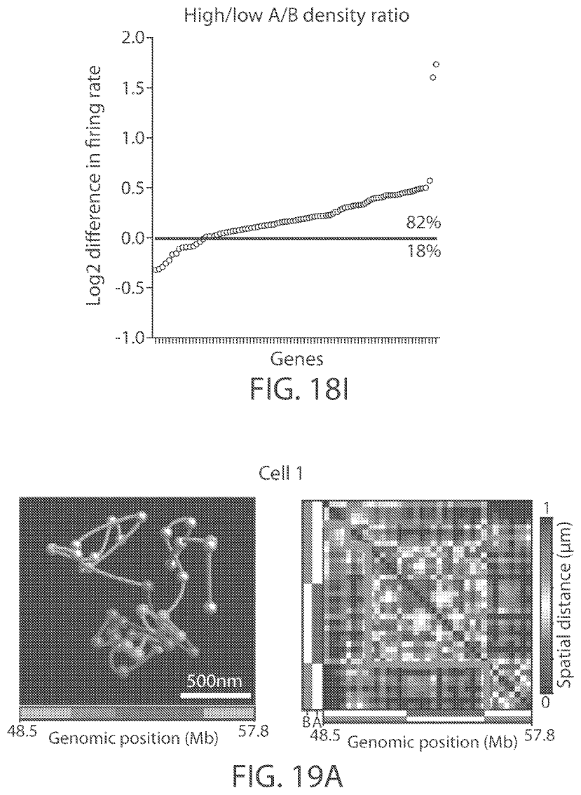

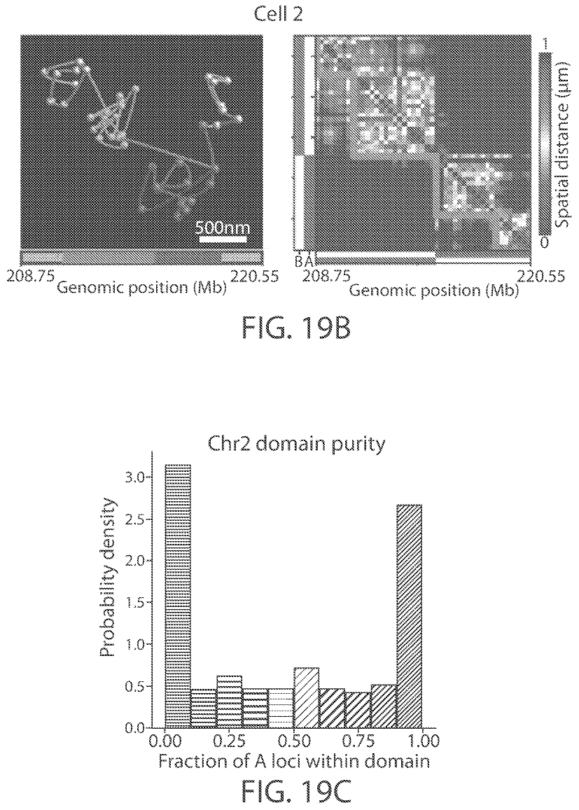

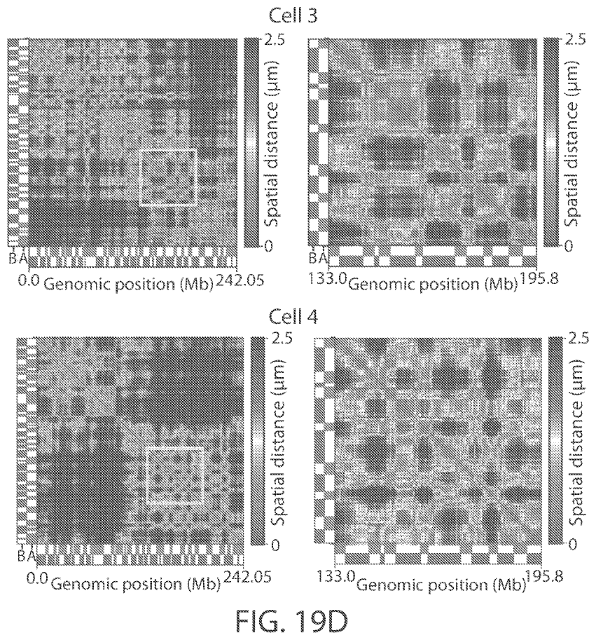

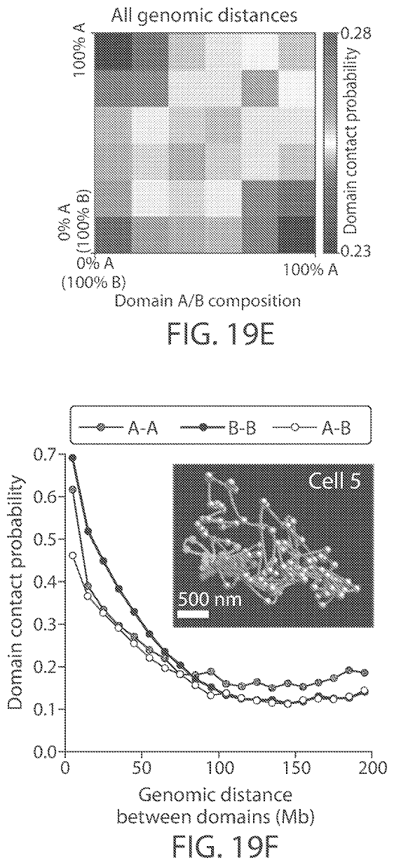

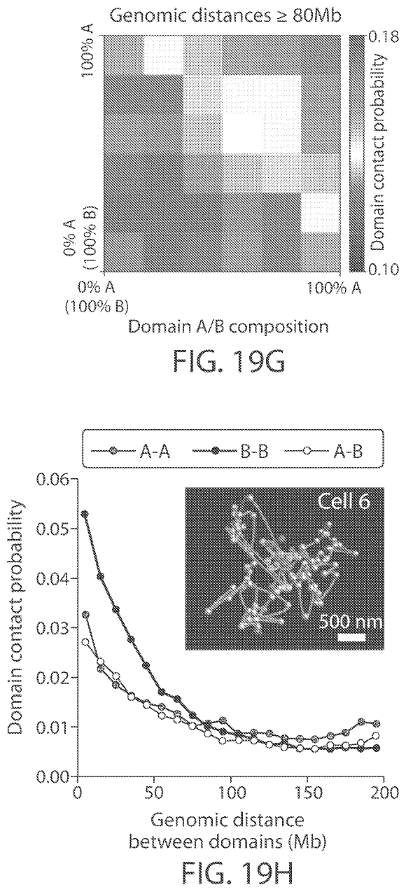

Although the majority of the single-cell domains in Ch2 were “pure” domains comprised entirely of either A or B loci, a substantial fraction of single-cell domains crossed ensemble A/B boundaries, and contained both A and B loci (FIGS. 19A-19C). The presence of these “mixed” domains suggested that domain formation in single cells may not be strongly coupled to the chromatin properties determining compartment identity, but it is also possible that cell-to-cell variability in epigenetic modifications caused a shift of active/inactive chromatin boundaries in some single cells.

It was next examined how domains interacted with each other, focusing on how inter-domain interactions depend on the A and B composition of the domains, as well as the genomic distances between the domains. Domains came into contact both at short and long genomic separations, and such contacts were manifested as off-diagonal box features in the spatial distance maps of individual chromosomes. Such contact patterns vary substantially from cell to cell (FIG. 19D). Despite this heterogeneity, domain-domain interactions appeared to be modulated by the A/B composition of their underlying chromatin (FIG. 19E): the contact frequencies between domains containing mainly B loci were, on average, higher than those between domains containing mainly A loci, which were in turn higher than the contact frequencies between domains dominated by chromatin of different A/B identities. This average picture was consistent with the hierarchy of A and B chromatin interaction strengths recently proposed based on chromatin structure modeling of the A/B compartmentalization measured by Hi-C and the global arrangement of A and B loci measured by imaging.

Further examination of domain contact frequencies as a function of genomic distance revealed a more complex picture. For simplicity, “pure” A and “pure” B domains which contained loci of a single compartment identity were focused on. As expected, contact frequency decreased with genomic distance for domains pairs of all compositions (FIG. 19F). However, the contact frequencies between pairs of B domains (B-B) were higher than those between pairs of A domains (A-A) at shorter genomic distances (up to −75 Mb for Chr2), whereas A-A domain contacts dominated over B-B domain contacts at larger genomic separation (FIG. 19F). The results were consistent with the genomic-distance dependence between A-A and B-B chromatin interactions reported in a recent ensemble Hi-C study, and provided further insights into how preferential interactions between single-cell domains can give rise to these ensemble trends. Notably, at relatively large genomic distances, B-B domain contact probability decayed to a level similar to the contact probability between A and B domains (A-B), whereas A-A domain contact probability remained higher than A-B domain contact probability even at large genomic separations (FIG. 19F). This led to a clear domination of A-A domain interactions at large genomic distances (FIG. 19G). In addition, contacting domain pairs also showed different degrees of spatial overlap, with some pairs of domains showing relatively superficial contact (FIG. 19F inset) whereas other pairs showed strong intermixing (FIG. 19H inset). Interestingly, compared to A-A domain pairs, B-B domain pairs showed a substantially stronger tendency to form such intermingled globules (FIG. 19H).

Overall, these results suggested that preferential A-A and B-B domain interactions give rise to spatial segregation of chromatin compartments and that the nature of A-A and B-B domain interactions are distinct. The differences in the nature of these interactions might arise from the different molecular factors involved in A-A and B-B association. For example, heterochromatin factors such as HP1 are thought to be involved in B-B interactions, whereas transcription activating factors or co-activators such as BRD4 and Mediator may be involved in active chromatin interactions. Whether these different molecular factors are responsible for the observed differences in the genomic-distance dependence between A-A and B-B domain interactions and their tendency to intermix awaits further investigations.

FIGS. 19A-19H show the dependence of domain-domain interaction on their A/B composition and genomic distance. FIG. 19A, Left is a 3D rendering of a “mixed” chromatin domain containing both A and B loci, flanked by “pure” domains comprised of only B loci in a copy of Chr2 in a single cell. Scale bar: 500 nm. Right: pairwise distance matrix for the same region displayed on the left. The bars on the bottom and left of the matrix display the A and B calling of loci and the outline highlights the boundaries of chromatin domains. A/B calling is determined from the ensemble proximity frequency map of Chr2.

FIG. 19B is the same as in FIG. 19A but for two pure domains, one comprised entirely of A loci and one entirely of B loci, instead of a mixed domain.

FIG. 19C is a distribution of the fraction of loci being A loci in single-cell chromatin domains in Chr2.

FIG. 19D is a single-cell spatial distance matrices of two example copies of Chr2. The first and third panels show the matrix for two whole chromosomes, while the second and last panels show a zoomed-in matrix for the region highlighted in yellow in the first and third panels, respectively. The side bars show the A/B compartment calling from the ensemble proximity frequency map.

FIG. 19E shows domain contact probabilities for domains of different A/B compositions in Chr2. X and Y axes represent the fraction of loci within a domain being A loci (0% corresponds to pure B domain and 100% corresponds to pure A domain). Two domains are defined to be in contact if their insulation score is <2. See Example 19 for the calculation of the insulation score.

FIG. 19F shows domain contact probability between two pure A domains (A-A), between two pure B domains (B-B), and between one pure A and one pure B domain (A-B) in Chr2, plotted as a function of the genomic distance between the two interacting domains. The inset contains the 3D rendering of an example pair of domains displaying long-range interaction with an insulation score=2. Scale bar: 500 nm.

FIG. 19G is the same as FIG. 19E, but for domain pairs with genomic distances larger than 80 Mb.

FIG. 19H is the same as FIG. 19F, but restricted to domain pairs with a high degree of intermixing (as defined by a low insulation score <1). The inset contains the 3D rendering of an example pair of domains displaying long-range interaction with a high degree of intermixing (insulation score=1). Scale bar: 500 nm.

### Example 12

Genome-scale chromatin imaging. The sequential imaging approach described above allowed a high-resolution view of chromatin in individual chromosomes to be obtained. This straight sequential imaging approach is well suited for imaging chromatin structures that are comparable to or smaller than the diffraction-limited resolution. However, the number of genomic loci imaged increases only linearly with the number of imaging rounds in this approach. For genome-scale chromatin imaging, because many genomic loci could be simultaneously resolved and localized in the nucleus, it was reasoned that a much more efficient, non-linear scaling of the number of imaged loci with the number of imaging rounds would be possible.

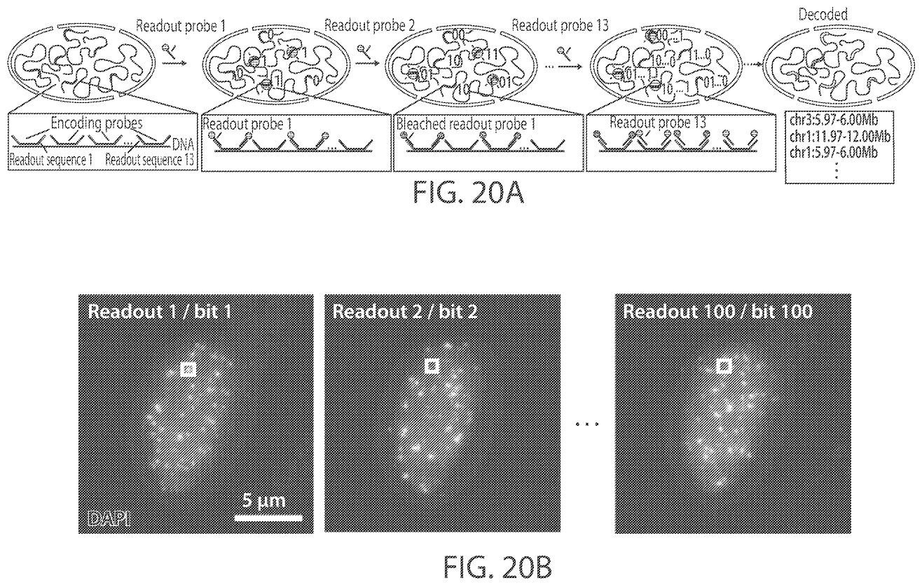

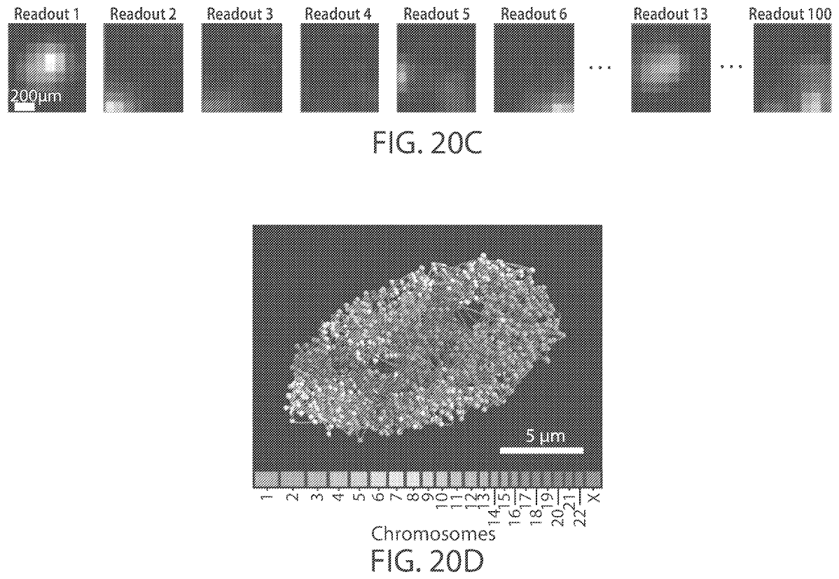

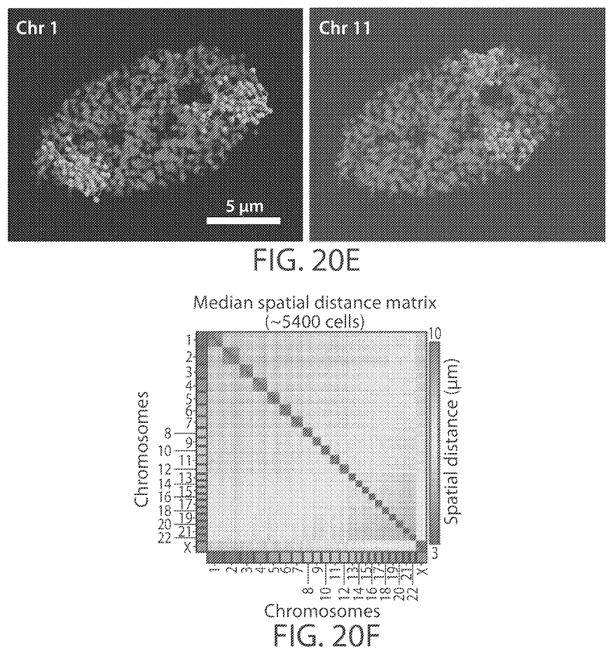

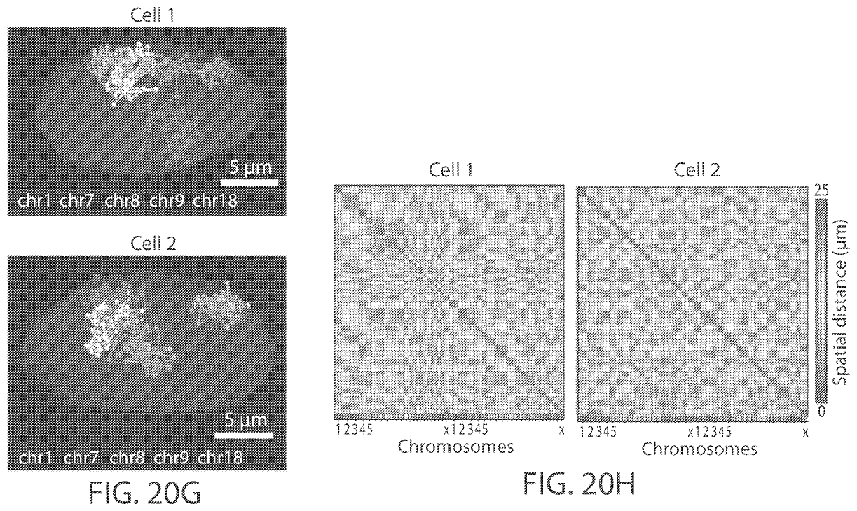

To achieve this goal, a combinatorial FISH approach was devised, inspired by the multiplexed error-robust FISH method that was previously developed for transcriptome imaging, but with important modifications specifically designed for chromatin imaging by considering both the polymeric nature of chromatin (i.e. adjacent loci in the genomic sequence are spatially close) and the territorial organization of chromosomes (i.e. distinct chromosomes tend to occupy separate spatial territories). To allow combinatorial imaging, each genomic locus was assigned a unique 100-bit binary barcode with a Hamming weight of 2, i.e. each barcode containing two “1” bits and 98 “0” bits (FIG. 20A). The bit values (“1” or “0”) in these barcodes determined the presence or absence of signal for each locus across sequential rounds of imaging. In order to avoid imaging spatially close chromatin regions simultaneously in the same bit, from these 100-bit Hamming weight 2 barcodes a subset was further selected to encode the targeted genomic loci and optimized assignment of barcodes, such that loci with a “1” bit in the same barcode position were maximally separated in genomic space (see Example 19). This strategy allowed detection errors caused by overlapping signals from nearby chromatin loci to be minimized. Moreover, because the vast majority of the possible 100-bit binary codes were invalid (i.e. not assigned to any targeted locus), this design allowed detection errors to be identified and discarded and measurement accuracy to be further improved.

The barcodes were physically imprinted onto the targeted genomic loci using a high-diversity library of encoding probes, each containing a target region for binding to one of the targeted loci and a readout sequence chosen from 100 pre-designed readout sequences (FIG. 20A). Each readout sequence corresponds to one of the 100 bits, and the encoding probe set for each genomic locus (˜400 probes per locus) contains only two distinct readout sequences, corresponding to the two bits that read “1” in the barcode assigned to that locus. After encoding probe binding, the barcodes imprinted on the chromatin loci were detected by sequential hybridization of fluorescently labeled readout probes, each complementary to one of the 100 readout sequences (FIG. 20A). In some cases, the adaptor probe strategy described for high-resolution whole-chromosome tracing was also used here. Two distinct adaptor/readout probes were introduced per hybridization round and imaged in two color channels, such that 2 bits were read out in each hybridization round. This allowed for ˜1000 genomic loci to be imaged and identified with only 50 rounds of hybridization (FIGS. 20A-20C). This represented approximately 10-fold fewer rounds of hybridization and thus a 10-fold shorter experiment time compared to sequential imaging of the same number of loci with the same number of color channels. Since each chromosome in diploid cells has two homologs, the homolog identities of the imaged loci were further assigned using a clustering algorithm, exploiting the tendency of chromosomes to occupy distinct territories in each nucleus.

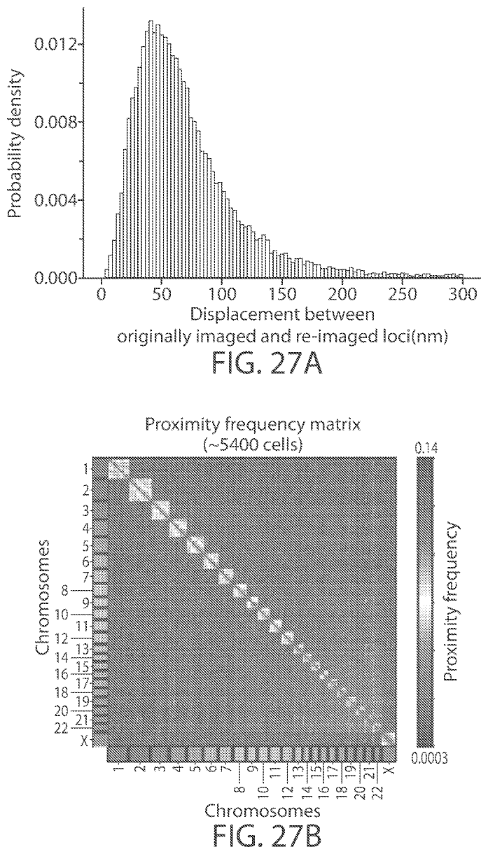

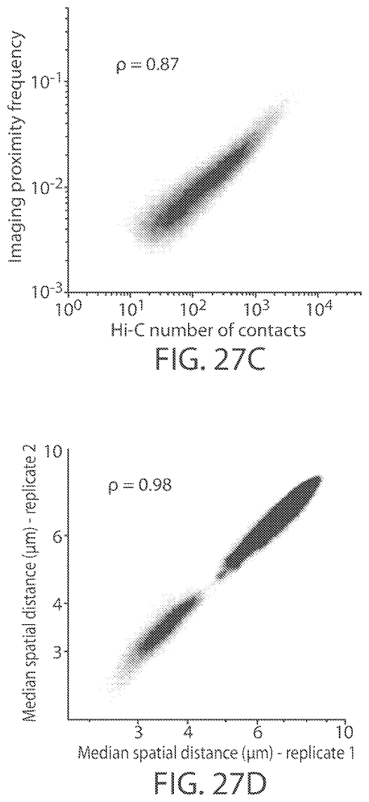

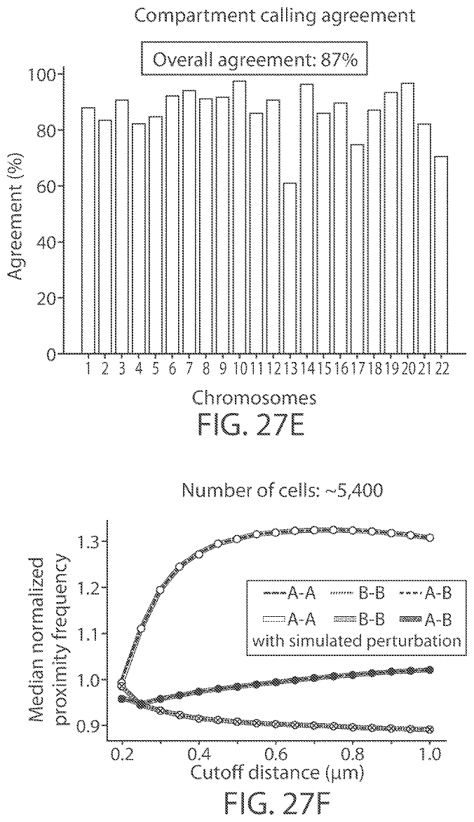

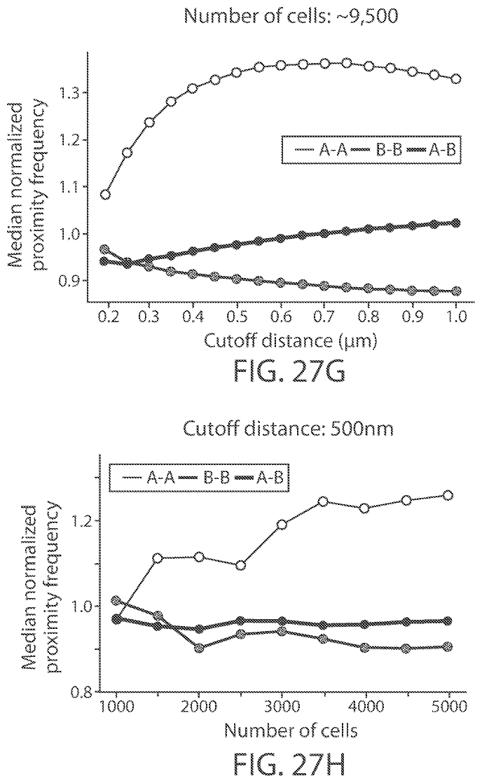

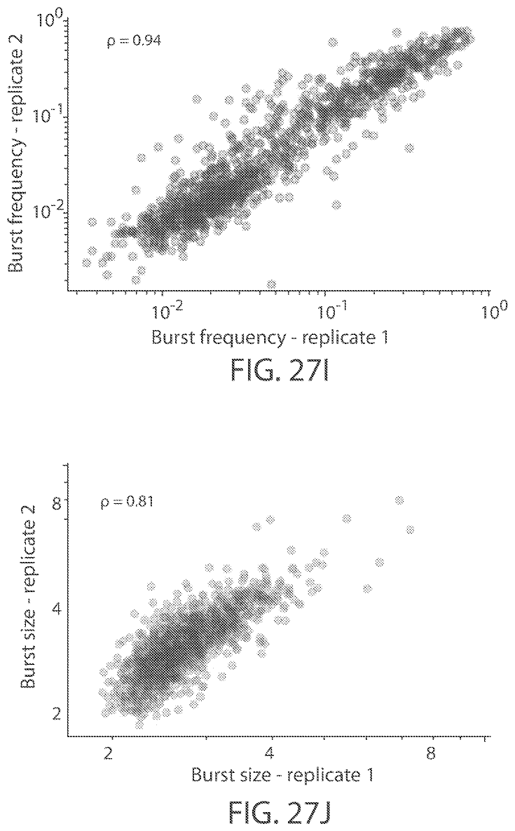

In this work, 1,041 genomic loci were selected for imaging, each ˜30-kb in size, uniformly covering the 22 autosomes and the X chromosome in IMR-90 cells. Another requirement was that each chromosome contained at least 30 targeted loci, hence the number of loci imaged per chromosome homolog ranged between 30 and 80 depending on the length of the chromosome. These 1,041 genomic loci in ˜5,400 individual cells across 5 biological replicates were imaged with a detection efficiency of ˜80% for each locus, yielding ˜1700 chromatin loci detected in each cell (FIGS. 20D-20E). At the end of the combinatorial imaging process, a small subset of the genomic loci were re-imaged with sequential imaging, one locus at a time. The displacement between the locus positions determined by combinatorial imaging and the re-imaged positions determined by sequential imaging were only ˜50 nm (FIG. 27A), indicating both a high decoding accuracy of the combinatorial imaging approach and minimal sample degradation/deformation during the course of imaging.

To obtain a population-averaged view of chromatin organization, the spatial distance between every pair of imaged chromatin loci was calculated in each cell and then both the median distance and the proximity frequency between every pair of loci across all imaged cells were determined (FIG. 20F; FIG. 27B). The proximity frequencies between pairs of chromatin loci within the same chromosome determined from the imaging data showed high correlation with the contact frequencies detected by ensemble Hi-C, with a Pearson correlation coefficient of 0.89 (FIG. 27C). Moreover, the imaging results showed high reproducibility between independent biological replicates (FIG. 27D).

By exploring chromatin organization in individual cells, it was noticed that chromosomes, while having a tendency to occupy distinct territories within each cell (FIGS. 20F-20G), also displayed substantial overlap with each other (FIGS. 20G-20H). These results were consistent with and expanded upon observations from earlier imaging studies. Since this observation suggested a high degree of trans-chromosomal interaction, further analyses were focused on exploring them.

FIGS. 20A-20H show genome-scale chromatin imaging by massively multiplexed, combinatorial FISH. FIG. 20A shows an imaging scheme. The targeted genomic loci are assigned error-robust barcodes, e.g. a subset of 100-bit binary barcodes with a Hamming weight of 2 (i.e. two of the 100 bits reading “1”). The barcodes are imprinted onto the genomic loci with encoding oligonucleotide probes, which recognize the loci and associate two distinct readout sequences with each locus, corresponding to the two bits that read “1” in the barcode assigned to the locus. Each bit is uniquely assigned a readout sequence. Each locus is labeled by a total of 400 encoding probes, but only 4 are shown. Fluorescent readout probes complementary to the readout sequences are sequentially added and imaged, allowing the bits that read “1” at each locus and hence the barcode identity of that locus to be determined.

FIG. 20B shows representative images from multiple imaging rounds in the nucleus of a single cell. Fluorescent signal of the chromatin loci from readout probes and signals of 4′,6-diamidino-2-phenylindole (DAPI), used as a nuclear marker, are shown. Scale bar: 5 micrometer.

FIG. 20C shows zoom-in images of a small region (white box in B) centered around one chromatin locus across all imaging rounds. The locus identity is determined based on the two readout probes (1 and 13) that give signals. Scale bar: 300 nm.

FIG. 20D is a 3D rendering of all detected chromatin loci (spheres) in a single IMR-90 cell, color-coded according to the chromosomes that they belong to (index for chromosomes shown below the image). Adjacent loci in genomic sequence are connected by a flexible line. ˜1000 genomic loci are imaged.

FIG. 20E shows chromatin loci of the same cell as in FIG. 20D but with two homologs of the indicated chromosomes highlighted.

FIG. 20F shows a median distance matrix computed from ˜5,400 single cells. For each pair of loci, the median of observed 3D spatial distances between the loci across all cells is presented.

FIG. 20G shows example images showing the positions of multiple chromosomes territories in single cells. Chromosomes are coded as indicated and shaded areas represent the convex hull surrounding all imaged loci. Only one homolog is shown per chromosome for clarity.

FIG. 20H shows spatial distance matrices for the same cells shown in FIG. 20G. The spatial distance between each pair of chromatin loci is shown. Chromosome order is as noted beneath the matrices, with the two homologs of each chromosome separately shown.

FIGS. 27A-27J show genome-scale imaging by combinatorial FISH: localization error, reproducibility, and comparison with Hi-C. FIG. 27A shows a distribution of the displacement between the localizations of genomic loci measured in the combinatorial imaging run and those of the same loci re-imaged individually using sequential hybridization after completing the combinatorial imaging. 10 genomic regions in Chr6 were re-imaged across ˜2000 cells. The median displacement is ˜50 nm.

FIG. 27B shows a proximity frequency matrix for all 1,041 genomic loci imaged by combinatorial FISH. The proximity frequency between a pair of loci was calculated as the number of incidences in which the measured distance between the loci is smaller than a cutoff distance of 500 nm divided by the total number of measured distances between the two loci.

FIG. 27C shows a correlation plot for the proximity frequencies between pairs of loci within chromosomes derived from our imaging data and the number of contacts derived from ensemble Hi-C experiments, binned at 500 kb and centered around the target loci. The Pearson correlation coefficient is 0.91. The available Hi-C data for IMR90 cells is sparse for trans-chromosomal contacts, precluding a reliable comparison of trans-chromosomal interactions between our imaging data and the Hi-C data.

FIG. 27D shows the correlation of pairwise distances between chromatin loci observed in two independent biological replicates of the genome-scale imaging experiments. The Pearson correlation coefficient between replicates is 0.98. The upper right cloud represents the trans-chromosomal pairwise distances and the lower-left cloud represents the intra-chromosomal pairwise distances.

### Example 13

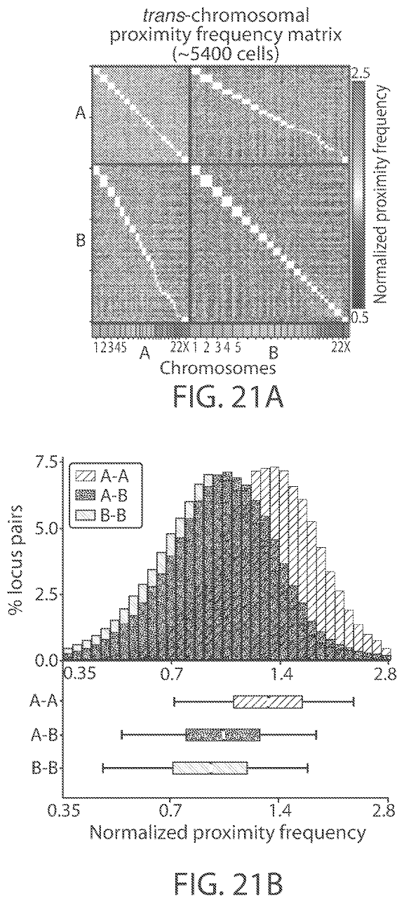

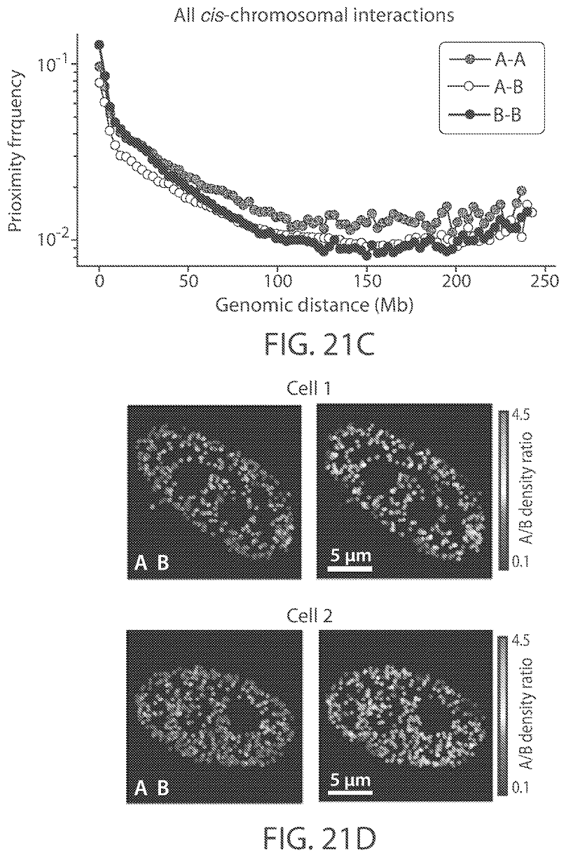

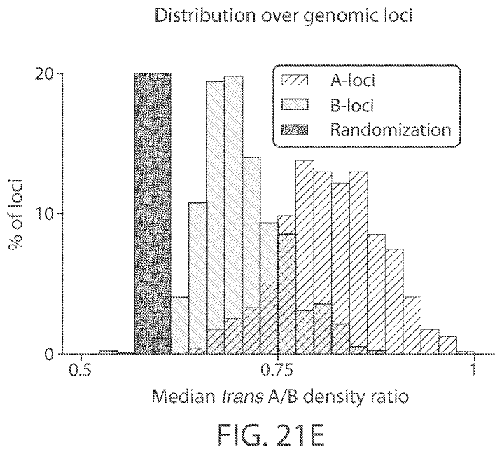

Enrichment of trans-chromosomal A-A interactions. Next, how trans-chromosomal interactions depended on the A/B compartment identity of chromatin were studied. Each of the imaged genomic loci were classified into compartments A and B based on published ensemble Hi-C data. Although the genome-scale imaging data also allowed fairly accurate A/B compartment calling, giving ˜80% agreement with calling based on Hi-C data (FIG. 27E), Hi-C calling was used to classify the A/B compartment identity of the imaged loci because of the higher genomic resolution of the ensemble Hi-C data. 38% of the imaged loci belonged to compartment A, while 62% belonged to compartment B. To examine whether the extent of trans-chromosomal interactions differs for active and inactive chromatin, the genomic loci in the trans-chromosomal proximity frequency matrix were rearranged, placing all A loci next to each other followed by all B loci. This matrix showed that a compartment-A locus had on average a stronger tendency to interact trans-chromosomally with another compartment-A locus than with individual compartment-B loci (FIGS. 21A-21B), consistent with previous observations of trans-chromosomal interactions between active chromatin. In contrast, compartment-B loci showed comparable or lower trans-chromosomal affinity towards each other than towards compartment-A loci (FIGS. 21A-21B). In other words, trans-chromosomal A-A interactions appeared with a substantially stronger tendency than A-B interactions, which in turn appeared with a slightly stronger tendency than B-B interactions. A similar trend was observed for a wide range of cutoff distances used to construct the proximity frequency map, provided that a sufficiently large number of cells were included in the analysis (FIGS. 27F-27H). Notably, this was in striking contrast with the overall hierarchy for cis interactions within the same chromosomes, in which B-B interactions had a stronger tendency to form than A-A interactions, which in turn had a stronger tendency to form than A-B interactions (FIG. 19E). Intriguingly, however, this observed trend for trans-chromosomal interactions (A-A>A-B≈B-B) was similar to that observed for cis-chromosomal interactions at large genomic distances in the high-resolution Chr2 data (FIGS. 19F-19G). This trend was also observed for long-range cis-chromosomal interactions in the genome-scale data, aggregated across all imaged chromosomes (FIG. 21C).

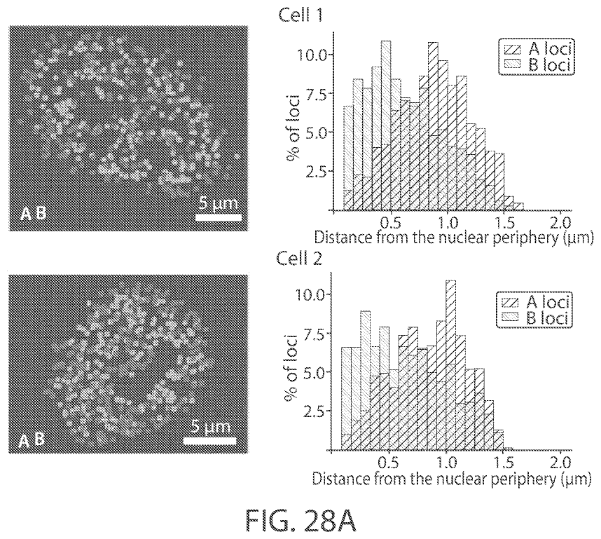

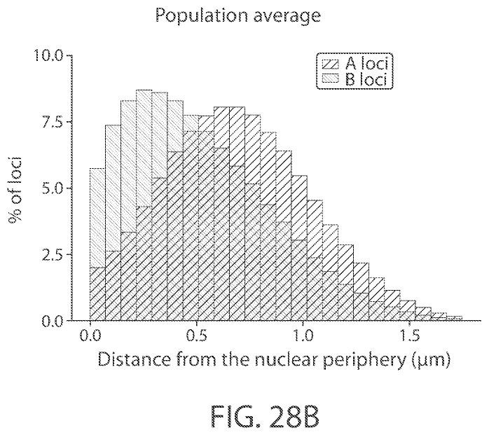

Next, the relationship of trans-chromosomal interactions and chromatin compartment identity at the single-cell level were examined. In individual cells, A and B loci adopted different spatial distributions, with A loci exhibiting a tendency to be more centrally localized than B loci in the nucleus (FIG. 21D; FIG. 28), as expected. To further characterize chromatin interactions in trans, a density-based approach similar to that presented earlier was adopted for high-resolution whole-chromosome tracing, except that trans-chromosomal interactions only were considered here. Briefly, for each imaged locus in each chromosome, the local densities of A loci and B loci from all other chromosomes in the same cell were calculated, and the ratio of these two densities was determined (referred to hereafter as the trans A/B density ratio) (FIGS. 21D-21E). This quantity provided a measure of the local enrichment of trans-chromosomal active chromatin near the locus. It was noted that the majority (62%) of the imaged loci belonged to the B compartment, creating an overall bias for the A/B ratio to be smaller than 1. To control for this bias, distributions of the trans A/B density ratios observed for A loci and for B loci with the distribution obtained in a randomization control where the A and B identities of imaged loci were randomly shuffled among the imaged loci, while keeping the numbers of A and B loci unchanged, were compared. Notably, the trans A/B density ratios observed for A loci were substantially higher than the values observed for B loci, which were in turn higher than the values derived from the randomization control (FIG. 21E). These single-cell analyses further supported the notion that trans-chromosomal interactions are preferentially enriched for interactions between active chromatin.

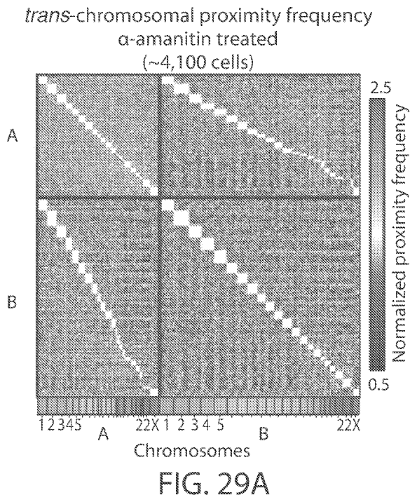

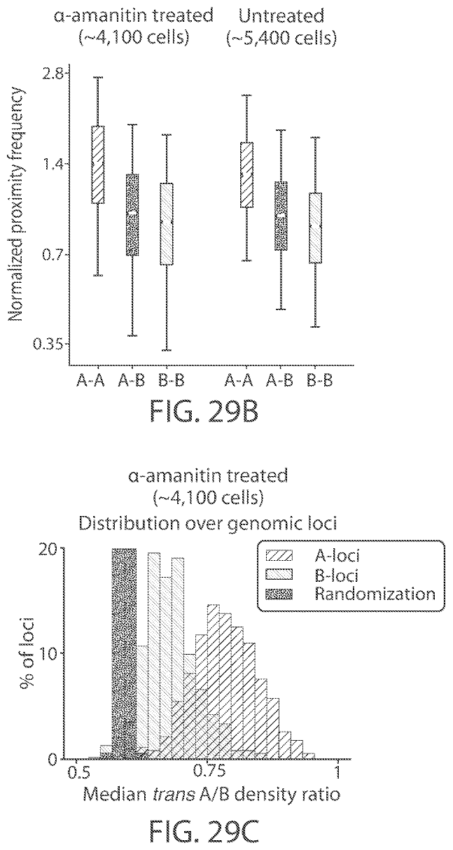

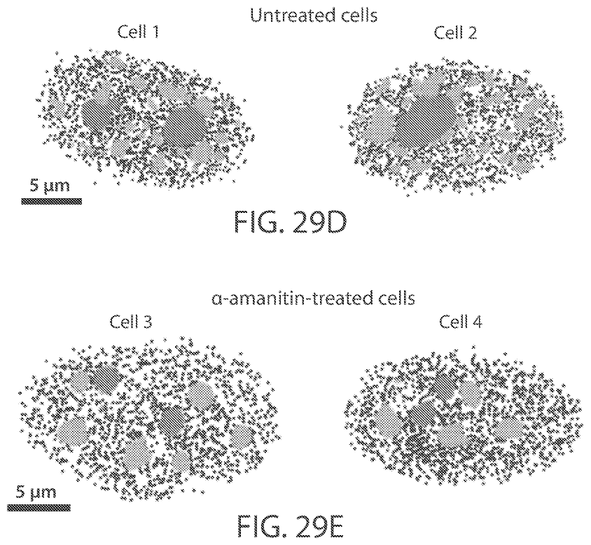

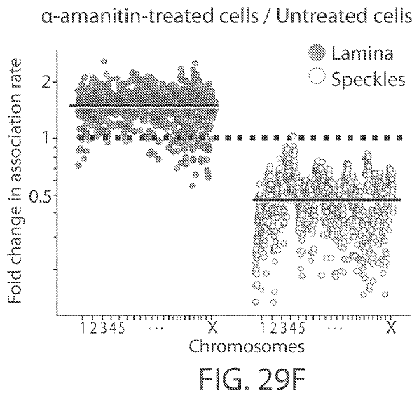

It was further asked whether the enrichment of trans-chromosomal A-A interactions required transcription. To address this, transcription was inhibited by alpha-amanitin treatment and it was found that this treatment did not lead to a substantial reduction in the enrichment for trans-chromosomal A-A interactions (FIGS. 29A-29C). This observation was consistent with and expanded upon a previous study, which showed that the long-range and trans-chromosomal interactions of the activated beta-globin locus with other active chromatin loci were not inhibited by transcription inhibition.

FIGS. 21A-21E show enrichment of active-active chromatin interactions in trans-chromosomal interactions. FIG. 21A shows normalized trans-chromosomal proximity frequency matrix. The proximity frequency between each trans-chromosomal locus pair (pair of loci on different chromosomes) is shown, with pairs of loci considered to be in proximity if their distances are smaller than a cutoff distance of 500 nm. The loci are reordered such that compartment-A loci appear first, followed by compartment-B loci, hence the top left block represents interactions between pairs of A loci and the bottom right represents interactions between pairs of B loci. Each entry in the matrix is normalized by the median proximity frequency of all locus pairs originating from the same pair of chromosomes to account for varying basal levels of interaction between pairs of chromosomes.

FIG. 21B shows distributions of trans-chromosomal proximity frequencies for pairs of A loci (A-A; n=72,771 locus pairs), pairs of B loci (B-B; n=193,753 locus pairs), and pairs comprised of one A and one B locus (A-B; n=237,986 locus pairs), derived from the matrix shown in FIG. 21A. Distributions are represented in the top panel as histograms and in the bottom panel as box plots, showing the median (center lines), 25th-75th percentiles (boxes) and 5th-95th percentiles (whiskers).

FIG. 21C shows the median proximity frequency between pairs of chromatin loci within the same chromosomes as a function of their genomic distance, averaged for pairs of loci separated by the same genomic distance across all chromosomes. Median contact frequencies are shown for pairs of A loci (A-A), pairs of B loci (B-B) and for mixed pairs of A and B loci (A-B).

FIG. 21D shows distributions of compartment-A and compartment-B loci in two single cells. The left panels represent the locations of all detected loci within a single z-plane in a single nucleus, with compartment-A loci and compartment-B loci. In the right panels, the shading of each locus represents the ratio of the local densities of trans-chromosomal A and B loci, i.e. the trans A/B density ratio, in accordance with the scale bar shown on the right.

FIG. 21E shows distributions of the local trans A/B density ratio for imaged genomic loci. For each locus, the median trans A/B density ratio across all cells was determined, and the trans A/B density ratio distributions for A loci are shown (n=382 loci) and that for B loci are shown (n=623 loci). 36 of the 1041 imaged loci were not assigned A/B identity due to different versions of genome assemblies used in this study and the Hi-C dataset used for compartment calling. The dark grey histogram represents a randomization control where the A and B compartment identity is randomly shuffled, while keeping the total number of A loci and the total number of B loci unchanged.

FIG. 27E are bar plots for the percentage of loci whose A/B compartment assignment agree between the genome-scale imaging data and Hi-C data for each human autosome. On average, ˜81% of the loci in each chromosome showed agreement in A/B assignment between our imaging data and the Hi-C data.

FIG. 27F shows the median normalized trans-chromosomal A-A, A-B, and B-B proximity frequencies (defined as in FIGS. 21A and 21B) as a function of the cutoff distance used to evaluate proximity. The normalized proximities are calculated from ˜5,400 IMR-90 cells. Also shown are the median normalized trans-chromosomal A-A, A-B, and B-B proximity frequencies after perturbing the loci positions with a 3D Gaussian noise term with a standard deviation of 50 nm, comparable to the estimated localization measurement error, as shown in FIG. 27A.

FIG. 27G is the same as FIG. 27F, but when additional data from alpha-amanitin treated cells is pooled with the untreated cells (for a total of ˜9,500 cells). alpha-amanitin treated cells showed similar enrichment of trans-chromosomal A-A over A-B and B-B proximity frequencies, as untreated cells (FIGS. 29A, 29B). This pooling result suggests that the lower enrichment for A-A interactions observed at lower cutoffs distance in FIG. 27F is likely a result of poorer statistics with a lower number of cells.

FIG. 27H shows the median normalized trans-chromosomal A-A, A-B, and B-B proximity frequencies, as a function of the number of cells included in the analysis. Cells were subsampled randomly from the −5,400 untreated IMR-90 cells imaged and the proximity cutoff distance was fixed to 500 nm.

FIGS. 29A-29F show the effect of transcriptional inhibition on the trans-chromosome chromatin interactions and the nuclear body association rates of chromatin loci. FIG. 29A shows normalized trans-chromosomal proximity frequency matrix, as in FIG. 21A, but for cells treated with alpha-amanitin to inhibit transcription.

FIG. 29B shows a distribution of normalized trans-chromosomal A-A, B-B and A-B proximity frequencies shown as box plots, as in FIG. 21B, but for cells treated with alpha-amanitin. For comparison, the normalized trans-chromosomal A-A, B-B and A-B proximity frequencies for untreated cells from FIG. 21B is reproduced here.

FIG. 29C shows distributions of the local trans A/B density ratio across imaged A and B loci, as in FIG. 21E, but for cells treated with alpha-amanitin. The histogram represents a randomization control where the A and B compartment identity is randomly shuffled, while keeping the total number of A loci and the total number of B loci unchanged.

### Example 14

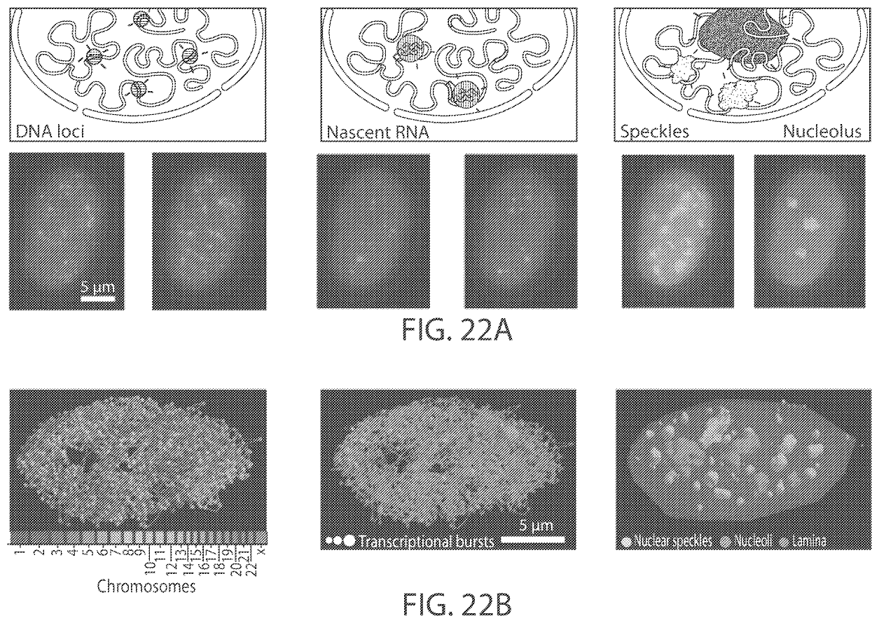

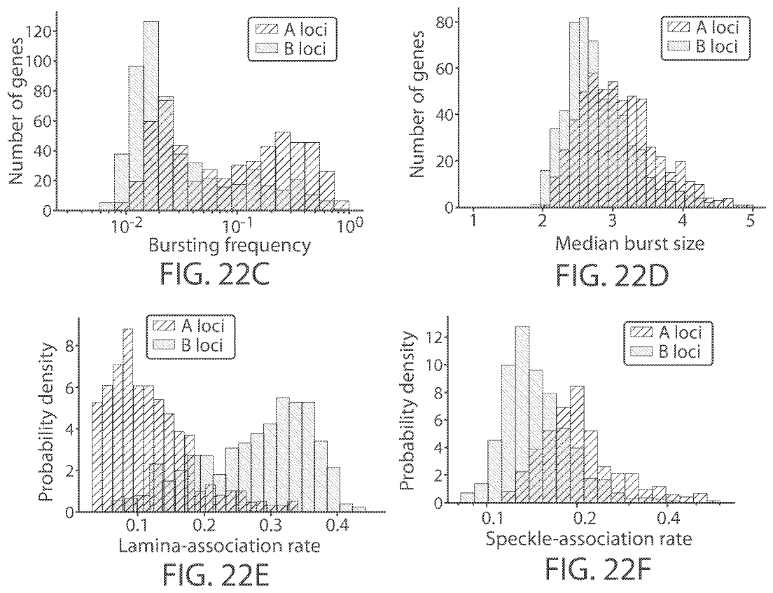

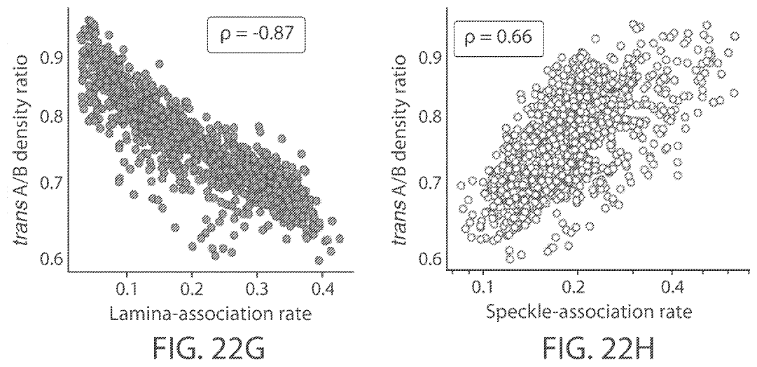

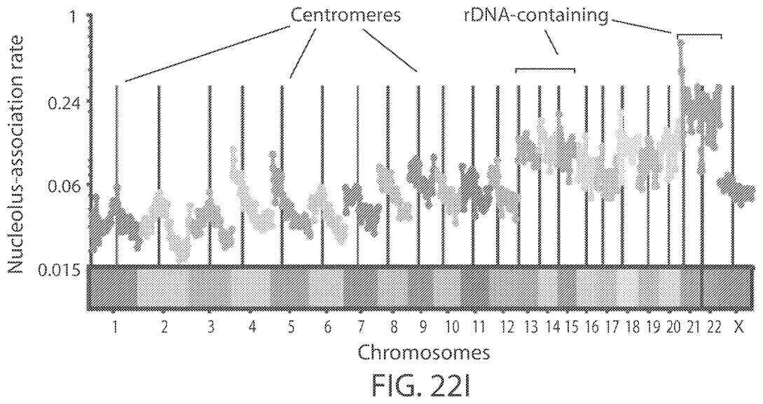

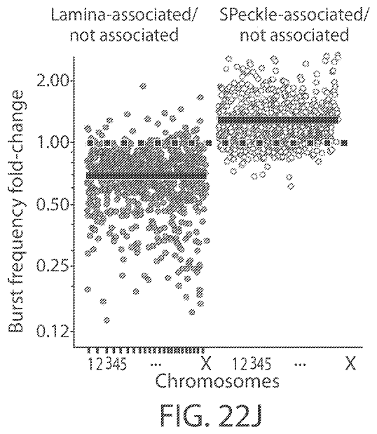

Multi-modal imaging of chromatin, nascent RNA and nuclear structures. To place the 3D organization of chromatin in the context of its functional activity and other nuclear structures, the combinatorial imaging method was expanded to allow simultaneous measurements of chromatin organization together with transcriptional activity of the imaged genomic loci, as well as nuclear landmarks in single cells. Specifically, the aforementioned 1,041 genomic loci together with the nascent RNA transcribed from each of the 1,137 genes located at these loci were imaged and simultaneously with important nuclear structures, including nuclear speckles and nucleoli (FIG. 22A).

To allow DNA, RNA and nuclear-structure imaging within the same cells, multiplexed imaging of the intronic RNAs of the 1,137 genes was performed, by adopting a similar combinatorial imaging strategy to the one described above for chromatin (FIG. 22A). Considering that not all genes would be transcribed in each individual cell, and hence the density of transcription foci should not be as high as that of the chromatin loci, the RNAs were encoded with a 54-bit, Hamming weight 2 code, and 1,137 of the possible barcodes to encode the genes were selected, in a way similar to how the barcodes for chromatin imaging were selected to minimize the chance of imaging spatially proximal genes in the same bit. After RNA imaging was completed, the RNA transcripts were enzymatically digested (a step also carried out in the single-modal chromatin imaging experiments) and multiplexed DNA FISH was performed as described above to image the 1,041 genomic loci (FIG. 22A). Decoding of genomic loci and nascent RNA transcripts was performed largely independently, with the additional constraint for the transcripts to colocalize with their harboring genomic loci (See Example 19). This procedure further improved detection accuracy for RNA transcripts and allowed the estimation of the detection efficiency (˜90%) for the transcription bursts at each genomic locus (see Example 19). Finally, nuclear speckles and nucleoli were imaged, using immunofluorescence against known molecular components of these structures (FIG. 22A). The fluorescent signals for nuclear speckles and nucleoli displayed a high signal-to-noise ratio (>25) even with immunofluorescence staining performed after DNA FISH. The positions of nuclear lamina were estimated by computing a convex hull surface encompassing all imaged genomic loci. Together, these multi-modal measurements allowed an integrated single-cell view of 3D genome structure, transcriptional activity and nuclear organization (FIG. 22B). These multi-modal imaging experiments were performed on ˜3700 individual cells, in two biological replicates. Chromatin imaging data from these multi-modal experiments were also included in the 5 replicates and −5,400 cells described above for 3D genome organization analyses.

From the nascent RNA transcript measurements of these multi-modal experiments, both the transcriptional burst frequency as the fraction of cells actively transcribing the gene (FIG. 22C) and the median burst size (FIG. 22D) from the brightness of the RNA intron signals were quantified for each gene. These measures showed high correlation across replicate experiments (FIGS. 27I-27J). The burst frequency displayed a bimodal behavior, with high burst frequency genes primarily harbored in the A compartment and low burst frequency genes present in both compartments (FIG. 22C). Furthermore, it was estimated whether specific chromatin loci were associated with nuclear bodies using a spatial distance cut-off of 250 nm, and higher association frequency of B loci with nuclear lamina (FIG. 22E) and higher association frequency of A loci with nuclear speckles were observed (FIG. 22F). These results were consistent with previous observations of preferential association of inactive and active chromatin with lamina and nuclear speckles, respectively. For individual loci, their median local trans A/B density ratio exhibited a negative correlation with the lamina association frequency (FIG. 22G) and a positive correlation with nuclear speckle association frequency (FIG. 22H). Nucleoli additionally showed preferential association with centromeres, telomeres of certain chromosomes, and chromosomes containing ribosome-encoding genes (FIG. 22I), as has been shown previously. These biological results provided further validation to the multi-modal measurements.

Notably, for most imaged loci, lamina association correlated with lower transcriptional activity, while nuclear speckle association correlated with higher transcription activity (FIG. 22J). These results were consistent with a recent single-cell sequencing study showing that lamina association is negatively correlated with gene expression in single cells. Moreover, it was observed that treatment with the transcription inhibitor alpha-amanitin perturbed the nuclear speckles and reduced the nuclear speckle association rates and increased the lamina association rates of the imaged loci (FIGS. 29D-29F). Together, these results expanded upon previous imaging studies on the nuclear repositioning of single or a few genomic loci upon transcriptional activation or inhibition and provided a genome-scale view of the relationship between transcriptional activity and interactions with nuclear structures.

FIGS. 22A-22J show multi-modal genome-scale imaging of chromatin and transcription activity in the context of nuclear structures. FIG. 22A, top: Illustration of the multi-modal imaging scheme that combines chromatin (left panel), nascent RNA transcripts (middle panel) and nuclear bodies (right panel) imaging to generate an integrated view of chromatin organization in the context of nuclear structures and functional activity. ˜1000 genomic loci, nascent RNA transcripts of ˜1100 genes in the targeted loci, and two types of nuclear bodies (nuclear speckles and nucleoli) are imaged. Bottom: Representative raw images for each imaging modality—chromatin loci across multiple imaging rounds (left), nascent RNA transcripts across multiple imaging rounds (middle) and nuclear bodies (right: nuclear speckles, imaged using an anti-SC35 antibody; and nucleoli, imaged using an anti-fibrillarin antibody). Scale bar: 5 micrometer.

FIG. 22B is a 3D rendering of chromatin loci, transcriptional bursts and nuclear bodies in a single cell. Left: All detected chromatin loci, coded by chromosome (based on the chromosome index shown below). Middle: All detected intronic RNAs shown as colored spheres, with shadings indicating the identities of the imaged genes and sphere size representing transcription burst size. Right: Volume-filling representations of detected nuclear bodies. The nuclear lamina is identified as the surface of the convex hull surrounding all detected chromatin loci (shaded gray area).

FIGS. 22C and 22D show distributions of transcription burst frequencies (FIG. 22C) and burst sizes (FIG. 22D) for genes residing in the imaged compartment-A loci (n=558 genes) and compartment-B loci (n=569 genes).

FIGS. 22E and 22F show distributions of association rates for A loci (n=382 loci) and B loci (n=623 loci) with nuclear lamina (FIG. 22E) and nuclear speckle (FIG. 22F). A chromatin locus is considered associated with nuclear lamina or a nuclear speckle if the distance of the locus to the nuclear periphery or the nearest speckle is <250 nm.

FIGS. 22G and 22H show scatter plots of the local trans A/B density ratio for each imaged genomic locus as a function of the frequency with which the locus is found associated with the nuclear lamina (FIG. 22G, Pearson correlation coefficient=−0.87) and nuclear speckles (FIG. 22H, Pearson correlation coefficient=0.66). The values of trans A/B density ratio shown are the median values across all imaged cells.

FIG. 22I shows the association frequency with nucleoli for all imaged genomic loci, ordered by genomic position. Black vertical lines indicate the locations of centromeres and brackets highlight chromosomes containing ribosome-encoding genes (rDNAs).

FIG. 22J shows the correlation of transcription with nuclear structure association. Circles are the fold-change in the transcriptional burst frequency for individual genomic loci when comparing the populations of cells in which the locus is lamina associated versus non-lamina-associated (left) and speckle-associated versus non-speckle associated (right). The dotted line highlights no change and the solid lines represent the median fold-change in each case.

FIGS. 271 and 27J show a correlation between replicates of RNA imaging for each gene's burst frequency (FIG. 27I) and burst size (FIG. 27J). Pearson correlation coefficients are 0.94 and 0.81, respectively.

FIGS. 29D and 29E show representative images of individual nuclei with imaged chromatin loci, nucleoli, and nuclear speckles shown for untreated cells (FIG. 29D) and cells treated with alpha-amanitin (FIG. 29E).

FIG. 29F shows a fold change in the rate of associate of each locus with lamina (left) and nuclear speckles (right) upon alpha-amanitin treatment. The data point for each genomic locus is shown in circles, the solid lines are the median fold changes of all loci in each case, and the dotted line represents no change. It is noted that the nuclear volume and the size and number of nuclear speckles also changed upon treatment with alpha-amanitin, and these might partially contribute to the changes in nuclear body association.

### Example 15

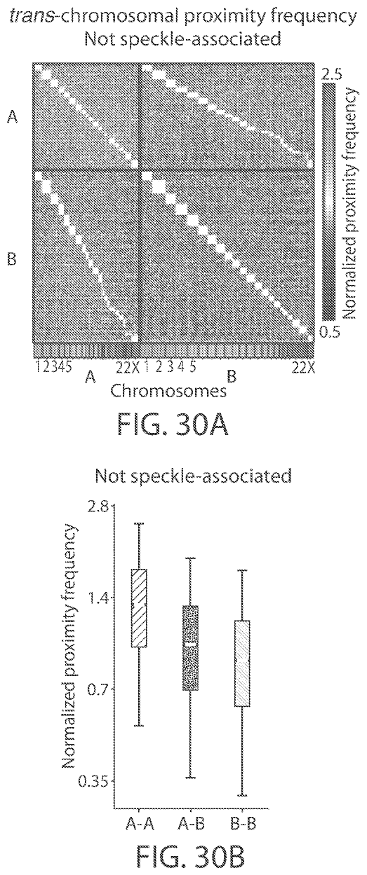

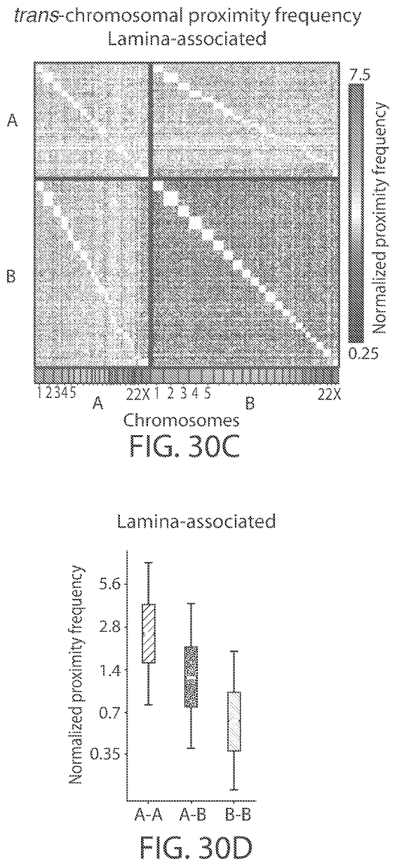

Examination of trans-chromosomal interactions in various nuclear contexts. Simultaneous imaging of chromatin organization and landmark nuclear structures in the same cells further allowed for examination of how the observed enrichment for trans-chromosomal A-A interactions depended on nuclear context. Because nuclear speckles are one of the most prominent nuclear bodies that concentrate actively transcribed loci, it was speculated whether the observed enrichment for trans-chromosomal A-A interactions was simply a result of this local concentration effect at the nuclear speckles. To address this question, analysis was restricted to loci that were not associated with nuclear speckles, i.e. for each locus pair, only those cells in which neither locus was associated with a nuclear speckle were considered. Interestingly, the same trend was still observed for enrichment of trans-chromosomal A-A interactions over A-B and B-B interactions under this constraint (FIGS. 30A-30B), indicating that association with nuclear speckles was not sufficient to account for the observed enrichment for trans-chromosomal A-A interactions.

Next, a relatively trivial scenario due to local concentration effects was considered: since compartment-A loci were depleted from lamina and more concentrated in the interior regions of the nucleus (FIGS. 28A-28B), it was speculated whether the enrichment of trans-chromosomal A-A interactions was simply caused by the local enrichment of compartment-A chromatin in the nuclear interior. To test this, only those cells in which both loci were associated with nuclear lamina were considered for each locus pair. Notably, the enrichment for trans-chromosomal A-A interactions over A-B and B-B interactions was observed even for these lamina-associated loci (FIGS. 30C-30D), despite the fact that the lamina is an environment enriched for inactive, compartment-B chromatin.

Overall, these results suggested a non-trivial molecular mechanism for the observed trans-chromosomal interactions between active chromatin. As noted earlier, the enrichment for A-A over A-B and B-B interactions was also observed for cis-chromosomal interactions at large genomic distances (FIGS. 19F-19G and FIG. 21C). It is possible that the long-range cis A-A interactions have a common underlying mechanism to that of the trans-chromosomal A-A interactions. What molecular factors give rise to these active chromatin interactions remains an open question.

FIGS. 28A-28B show that compartment-A and compartment-B loci display distinct spatial distributions in the nucleus. In FIG. 28A, the left panels show example images displaying A loci and B loci in a single z-plane of single cells. The right panel shows the distribution of distances to the nuclear periphery for A loci and B loci in these single cells. The nuclear periphery is identified as a convex hull surrounding all detected chromatin loci.

FIG. 28B shows p-averaged distributions of the distance to nuclear periphery for A loci (n=382) and B loci (n=623).

FIGS. 30A-30D show enrichment of trans-chromosomal active chromatin interactions in different nuclear environments. FIG. 30A shows normalized trans-chromosomal proximity frequency matrix, as in FIG. 21A, but considering only loci that are not associated with nuclear speckle. For each locus pair, only cells in which neither locus is associated with nuclear speckles are considered.

FIG. 30B shows trans-chromosomal proximity frequency for pairs of A loci (A-A), pairs of B loci (B-B), and pairs comprised of one A and one B locus (A-B), as in FIG. 21 5B, but considering only the cells in which neither locus is associated with nuclear speckles.

FIG. 30C is the same as FIG. 30A, but for pairs of lamina-associated loci. For each locus pair, only cells in which both loci are associated with nuclear lamina are considered.

FIG. 30D is the same as FIG. 30B, but for pairs of lamina-associated loci. For each locus pair, only cells in which both loci are associated with nuclear lamina are considered.

### Example 16

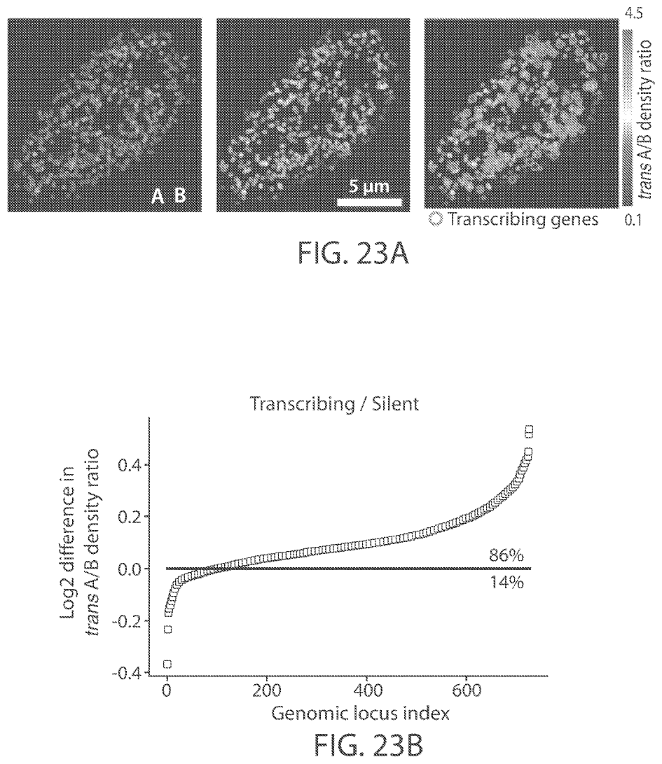

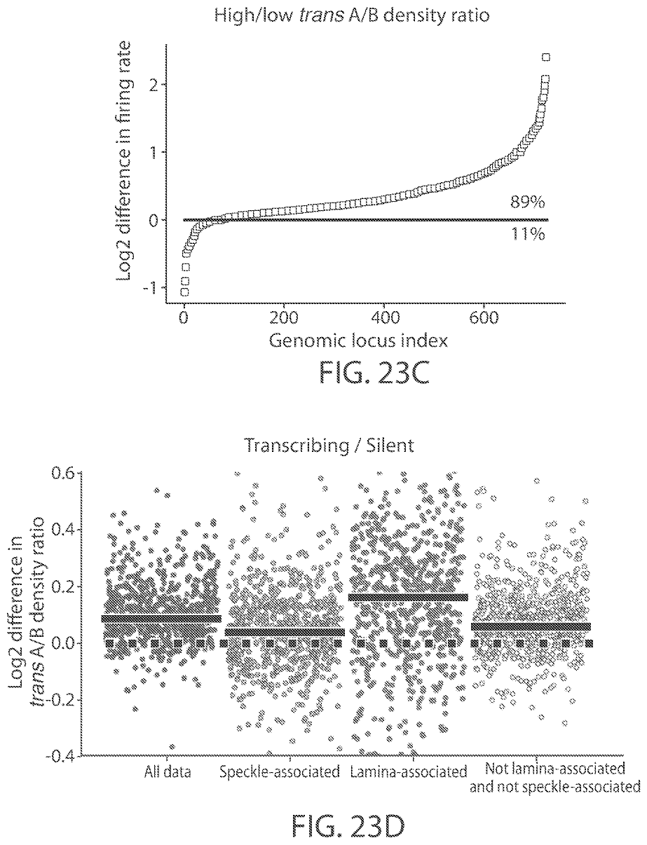

Correlation between trans-chromosome interactions and transcriptional activity. Next, these multi-modal single-cell measurements were used to characterize the relationship between transcriptional activity of individual chromatin loci and their local chromatin environment defined by trans-chromosomal contributions. To this end, the trans A/B density ratios were calculated and the median values of this quantity were determined for two populations of cells (determined independently for each genomic locus): (i) the cells where the locus under consideration exhibited transcriptional activity, and (ii) the cells where the locus appeared transcriptionally silent (FIG. 23A). Notably, a consistent trend for a higher trans A/B density ratio was observed when the locus was actively transcribed: 86% of the imaged loci exhibited a greater trans A/B density ratio when in the actively transcribing state as compared to the silent state (FIG. 23B); likewise, 89% of the loci exhibited a greater transcription firing rate when having a higher trans A/B density ratio (FIG. 23C). This positive correlation between transcription activity and local compartment-A chromatin enrichment was observed across multiple distinct nuclear environments, including for loci associated with nuclear speckles, loci associated with nuclear lamina, and loci not associated with either nuclear speckles or lamina (FIG. 23D), albeit that the correlation was weaker for speckle-associated loci.

These observations expanded upon the aforementioned results on the relationship between transcriptional activity and cis A/B density ratio within chromosomes (FIGS. 18G-181), and together, revealed a widespread positive correlation between the transcriptional activity of genes and the enrichment for active chromatin in their local environment.

FIGS. 23A-23D show the correlation between transcriptional activity and local enrichment of trans-chromosomal active chromatin. FIG. 23A shows single-cell images of chromatin loci and transcriptional activities. Left: Locations of all imaged A and B loci in a single z-plane from a single nucleus. Middle: Local trans A/B density ratios for the same loci, coded based on the scale bar. Right: Same as the middle panel, with detected transcriptional bursts overlaid and displayed as circles. Scale bar: 3 micrometer

FIG. 23B shows the change (measured in log difference) in the trans A/B density ratios for each imaged locus between actively firing and non-firing states. For each genomic locus containing at least one imaged gene, the trans A/B density ratio was calculated for the cells in which the genomic locus was actively transcribed (designated as transcribed) and for the cells in which it was not transcribed (designated as silent). The log-difference of the medians of these values for each imaged locus was rank-ordered according to the magnitude of change. 86% of the imaged loci exhibited an increase in the A/B density ratio when they are actively firing, compared to not firing.

FIG. 23C shows the change (measured in log difference) in the firing rates of the imaged genes between cells in which the trans-A/B density ratio at the locus harboring the gene changes from low (bottom quartile) and high (top quartile). The log-difference in firing rate for all genes imaged was rank-ordered according to the magnitude of firing rate. 89% of the imaged genes showed a higher firing rate when their harboring locus was in the top quartile, compared to the bottom quartile, of trans-A/B density ratios.

FIG. 23D shows swarm plots showing the fold change of local trans A/B density ratios between transcribed and silent states for the imaged gene-containing loci, conditioned on their nuclear body association status. For each genomic locus, the fold change was computed in the trans A/B density ratio between transcribed and silent states of the locus, considering, from left to right, respectively: all cells, only the cells in which the locus was associated with a nuclear speckle, only cells in which the locus was associated with the lamina, and only cells in which the locus was not associated with a nuclear speckle nor with the lamina (empty circles). The median trans A/B density ratio in each state (transcribed or silent) was determined for each locus and each association condition, and the log 2 of the fold change between the two states is shown. The dotted line represents no change and the solid lines represent the median fold change across all loci in each case. Some outliers were omitted to allow a clearer visualization of the median fold change (5 loci above and 9 loci below the presented scale for the speckle-associated data, 37 loci above and 17 loci bellow for the lamina-associated data and 1 locus above and 2 loci bellow for the not lamina-associated and not-speckle-associated data).

### Example 17

Chromosome-wide and genome-scale chromatin imaging. Reported herein is massively multiplexed chromatin imaging for determining the 3D conformation of chromatin across multiple scales of genome organization in single cells. The ability to image >1000 genomic loci in thousands of individual cells was demonstrated. The approach further allowed the placement of the 3D chromatin organization in its native functional and structural context by combining chromatin tracing with nascent-transcript and nuclear-structure imaging, and the ability to simultaneously image >1000 genomic loci, the transcription activity of >1000 genes residing in these loci, as well as landmark nuclear structures, including nuclear speckles and nucleoli was demonstrated.

Specifically, two complementary strategies for high-throughput chromatin tracing were demonstrated. First, the capability of the previously reported multiplexed FISH technology based on sequential hybridization was expanded, and imaging of hundreds of genomic loci with hundreds of rounds of hybridization and multi-color imaging was shown. The capability of this approach was demonstrated by providing a high-resolution view of the conformation of whole chromosomes, and systematic characterizations of chromatin domains, compartments and the relationship of transcription to chromatin organization in single cells. Second, for structures that span a space that is substantially larger than the diffraction-limited resolution and hence allow many loci to be resolved in each imaging round, a combinatorial labelling strategy for chromatin imaging that allows for a much more rapid, non-linear increase of the number of imaged loci with the number of imaging rounds was developed. The power of this latter approach was demonstrated by performing genome-scale imaging of both chromatin organization and transcription, simultaneously imaging >1000 genomic loci and nascent transcripts of >1000 genes in individual cells with only tens of hybridization rounds. These data revealed genome-wide trans-chromosome interactions, and their relationships with nuclear structures and transcription. Putting together this combinatorial imaging approach with the demonstrated ability to perform hundreds of rounds of hybridization and imaging, it should be possible to simultaneously image >10,000 genomic loci to provide a high-resolution whole-genome view of chromatin structures in single cells.

### Example 18

The high-throughput imaging technology shown in this example has several advantages for studying chromatin organization. First, compared with high-throughput sequencing-based approaches, which rely on proximity information to infer chromatin structure, the method provides direct visualization of chromatin organization, and direct measurements of the spatial locations of individual imaged loci in the nuclear context and the physical distances between pairs of imaged loci. Second, the method is intrinsically a single-cell approach and can reveal detailed chromatin structures in individual cells. The high (nearly 100%) detection efficiency of individual chromatin loci by the imaging methods allows a high capture rate of pair-wise chromatin interactions, which can provide a high-definition view of chromatin structures in single cells. The large number of cells measured by the method allows robust statistical analysis of common structural organizations across cells as well as cell-to-cell variations. Third, the chromatin tracing technology can be readily combined with other imaging modalities. This includes multiplexed transcriptional imaging and nuclear structure imaging as demonstrated in this study, but could also be further expanded to include other modalities such as imaging of epigenetic modifications or the degree of chromatin accessibility. Such multi-modal imaging can provide key insights into the relationships between chromatin structure, nuclear organization, and transcriptional activity.

There are many possible applications of the high-throughput chromatin imaging method reported here. While in the current work, loci were targeted uniformly across chromosomes to provide an unbiased view of the overall 3D chromosome and genome organization, this method could also be used to target genomic loci with specific structural and functional properties. An interesting direction would be to target loci that either contain specific genes or regulatory sequences, or are bound by specific nuclear architecture proteins, such as CTCF or cohesin, to study the interactions between these loci and their relationship with transcription. As a more specific example, a large set of potential promoters and enhancers can be targeted, and their interactions can be studied while simultaneously imaging the transcription activity of the genes governed by the promoters in the same cells. This would allow for inferring which enhancers control which promoters and reveal the rules governing how networks of promoters and enhancers differentially interact to regulate transcription. In another direction, many transcription factors and associated proteins, together with non-coding RNAs, have been reported to take part in physical condensates that organize chromatin within the nucleus, which may in turn be important for gene expression regulation. Imaging chromatin organization simultaneously with the structures formed by these factors and together with transcriptional output will provide a promising avenue to decipher the relationship between chromatin structures, multiple-component assembly, condensate formation and transcription regulation. Moreover, different cell types exhibit different gene expression profiles that are likely regulated, in part, by 3D genome organization. Thus, imaging chromatin organization together with gene expression profiles of individual cells in tissues promise to provide critical insights into chromatin organizations that are important for cell-type-specific gene expression patterns.

### Example 19

This example illustrates certain experimental models and subject details used in some of the above examples.

Cell culture and lines used in the analysis. Cells were prepared similarly to previous studies. IMR-90 cells were purchased from American Type Culture Collection (ATCC, CCL-186) and grown according to the recommended protocol.

Oligonucleotide probe design: Choice of target genomic regions. For high-resolution whole-chromosome imaging by sequential hybridization, the target chromosome were first partitioned into 50-kb segments. After screening out repetitive elements and regions where <100 unique probes can be designed per 50-kb segments, a total of 651 target genomic loci were kept for Chr21 and 4,500 target genomic loci for Chr2. Primary probes were then designed for each 50-kb segment (˜500 oligonucleotide probes) and the 350 most centrally positioned probes per segment were kept for sequential imaging. For Chr21 imaging, all 651 genomic loci were imaged. For Chr2 imaging, 250-kb genomic resolution was aimed for, and hence only designed primary probes for one in every five 50-kb segments.

For imaging nascent RNA transcripts on Chr21, genes were selected for which >50 primary probes (see “Primary/encoding probe design” section, below) could be designed on their first introns from all the protein-coding genes on Chr21. A total of 86 genes that are interspersed across Chr21 were selected. In order to facilitate the accurate detection of the spatial positions of transcription initiation events, probes to target a 5-kb segment of DNA around the transcription start site (TSS) of each gene were designed.

For genome-scale chromatin imaging by the combinatorial imaging strategy, genomic loci were chosen for imaging in the following way. For each human chromosome (except the Y chromosome), a 30-kb segment every ˜3 Mb of spacing was selected. If this spacing resulted in less than 30 selected loci on a given chromosome, the spacing was reduced for that chromosome, until all chromosomes had at least 30 loci selected. This resulted in a total of 1,041 target genomic loci for imaging, and the number of loci in individual chromosomes ranged from 30-80. Encoding probes were then designed for each 30-kb segment (˜400 oligonucleotide probes) for the combinatorial FISH imaging.

For imaging of nascent RNA transcripts in the genome-scale imaging, all intron-containing genes that completely or partially overlapped with the 1,041 targeted genomic loci were chosen. Encoding probes for the introns of all of these RNAs were chosen, such that each RNA had ˜20 encoding probes and that the targeting sequences of the encoding probes were kept as close as possible to the transcription start site. A total of 1,137 genes were targeted.

Barcode design for genome-scale imaging by combinatorial FISH. Binary barcodes for imaging the 1,041 genomic loci were chosen in the following fashion. First, all possible 100-bit binary barcodes with a Hamming weight of 2 (i.e. each barcode containing two “1” bits and 98 “0” bits) were generated and 1,041 barcodes from this list were randomly selected. The selected barcodes were then arbitrarily assigned to the 1041 genomic loci first. Next, barcodes were exchanged randomly between the used and unused code pool, as well as between loci from different chromosomes, in order to minimize, for each chromosome, the variance in the number of loci appearing (i.e. reading “1”) across different bits. This resulted in an approximately equal number of loci imaged per bit for each chromosome. To optimize association of barcodes to loci within each chromosome, loci within the same chromosome were allowed to exchange barcodes and the largest minimal genomic distance between loci with barcodes reading “1” at the same code position were optimized. When comparing code assignments with identical minimal genomic distances, the one that minimized the coefficient of variation of genomic distances was selected (so that genomic distances have both larger means and smaller standard deviations).

Barcodes for imaging the nascent RNA transcripts of the 1,137 genes were chosen similarly, but using a 54-bit, Hamming distance 2 code instead of a 100-bit, Hamming distance 2 code.

Primary/encoding probe design. Primary/encoding probes for chromatin imaging were synthesized from a pool of oligonucleotides purchased from Twist Biosciences. Each oligo in this pool used the following sub-sequences (from 5′ to 3′): a 20-nucleotide (nt) or 19-nt forward priming region for PCR amplification and reverse transcription (RT), a 20-nt readout sequence corresponding to the genomic locus targeted by the probe in the case of sequential imaging or one of the bits in which the genomic locus targeted by the probe will be imaged in the case of combinatorial imaging, a 42-nt or 40-nt target sequence (for sequential or combinatorial imaging, respectively), designed to bind uniquely to a single targeted genomic locus, an additional 1-2 copies of the 20-nt readout sequence described above, and a 20-nt or 19-nt reverse priming sequence for PCR amplification.

Similar designs with minor modifications were used for nascent RNA imaging. The forward and reverse priming sequences were chosen from a previously generated list of random 20-nt sequences optimized for PCR, as described previously.

The readout sequences were chosen via the following process. First, a list of 30-nt sequences with minimal homology to the human genome was created, as previously described. Then, a subset of these sequences was ranked by observed signal to noise ratio (SNR) and the top 100 were chosen as DNA readout probes. For sequential imaging, substantially more readout sequences were needed due to the larger number of hybridization rounds. Hence, the same procedure outlined previously was followed to select −1,200 candidate readout sequences. Then these candidates were filtered to ensure a GC content of 40-60% and a melting temperature of 57-67 degrees Celsius. These sequences were further filtered using BLAST such that no readout sequence had hits with HSP score larger or equal to 17. Lastly, the readout sequences were chosen by reverse-complementing the last 20-nt of each of these sequences.

The 42-nt or 40-nt target sequence was chosen similarly to a procedure described previously. Briefly, the following procedure was repeated for each genomic region of interest (see the “Target genomic regions” section above). First, a list of all 42-nt or 40-nt sequences complementary to the genomic region of interest was created (starting at each possible base in the targeted region). Then, sequences were filtered by requiring them to be within a defined range of melting temperatures and GC content. The remaining sequences were then further filtered by limiting the allowed degree of homology to the human genome, the human transcriptome and a database containing repetitive sequences using the same procedure as previously. The sequences used for whole-chromosome imaging by sequential hybridization had an additional filtering step using BLAST in which each target sequence was ensured to match uniquely the intended genomic locus. Finally, target sequences were selected from the remaining sequences after the final filtering step such that no genomic overlap exists between any pair of target sequences.

For whole-chromosome imaging by sequential hybridization, all of the 42-nt target sequences for each target genomic locus was matched with a unique readout sequence associated with that locus. To generate primary probe sequences, each target sequence was concatenated to two identical copies of the readout sequence assigned and then concatenated to the forward and reverse PCR primers. For generating the full-length encoding probes for genome-scale imaging by combinatorial FISH, each of the chosen 40-nt target sequences for each target genomic locus was alternatingly assigned to 2 groups spanning the entire target locus. Each of these groups was associated with a single readout sequence, corresponding to one of the two bits in which the locus would be imaged. Then, each target sequence was concatenated to two identical copies of the readout sequence assigned to its group, and then concatenated to the forward and reverse PCR primers.

Probes for RNA imaging were designed similarly, with the exception that they contained 3 copies of an identical readout sequence on every probe, one at the 5′ end and two at the 3′ end of the target region. Readout sequences for RNA imaging were orthogonal to those used for DNA imaging and were selected from the same ranked list of tested readout sequences.

Overview of experimental system. The physical setup used for performing these experiments used several components. A custom-built fluorescence microscope was used to acquire images, while a custom-built fluidics system was used to automatically perform buffer exchanges on the microscope stage. Custom software was used to synchronize and control the various components, and to automate many experimental steps. Below is a detailed description of each of these elements.

Microscope setup for image acquisition. Image acquisition was performed using a custom-built microscope system. The system was built around a Nikon Ti-U microscope body with a Nikon CFI Plan Apo Lambda 60× oil immersion objective with 1.4 NA. Illumination was based on one of two alternatives: solid-state, single-mode lasers with the following wavelengths: 405 nm (Coherent, Obis 405 nm LX 200 mW), 560 nm (MPB Communications, 2RU-VFL-P-2000-560-B1R), 647 nm (MPB Communication, 2RU-VFL-P-1500-647-B1R) and 750 nm (MPB Communication, 2RU-VFL-P-500-750-B1R). In this case, the output of the 560-nm, 647-nm and 750-nm lasers were controlled by an acousto-optic tunable filter (AOTF) while the 405-nm laser was controlled directly via its laser control box. A custom dichroic (Chroma, zy405/488/561/647/752RP-UF1) and emission filter (Chroma, ZET405/488/461/647-656/752m) were used to separate excitation and emission illuminations. Or, a Lumencor CELESTA light engine (a fiber-coupled solid-state laser based illumination system) with the following wavelengths: 405 nm, 446 nm, 477 nm, 520 nm, 546 nm, 638 nm and 749 nm. This system was used with a penta-bandpass dichroic (IDEX, FF421/491/567/659/776-Di01-25×36) and a penta-bandpass filter (IDEX, FF01-441/511/593/684/817-25). In most experiments, the illumination was flattened using a Refractive Beam Shaper (Newport Optics, GBS-AR14) or a vibrating optical fiber (Errol, custom Albedo unit).

A scientific CMOS camera (Hamamatsu FLASH4.0 or Hamamatsu C13440 with factory calibration for single-molecule imaging) was used for image acquisition. Sample position in three dimensions was controlled using a XYZ stage (Ludl). A custom-built auto-focus system was used to maintain a constant focal plane over prolonged periods of time. This was achieved by comparing the relative position of two IR laser (Thorlabs, LP980-SF15) beams reflected from the glass-fluid interface and imaged on a separate CMOS camera (Thorlabs, uc480).

The different components were synchronized and controlled using a National Instruments Data Acquisition card (NI PCIe-6353) and custom software (see “Software for controlling experimental components” below).

Fluidics system configuration. The fluidics system used several main components: a pump, a set of valves connected in series, a flow chamber in which the sample was mounted, and tubing and connectors. A peristaltic pump (Gilson, MINIPLUS 3) was used to generate flow in the system. The pump was connected to an array of 8-way valves (Hamilton, MVP and HVXM 8-5), connected in series. In this study, 3-5 valves connected in such a manner were used. Each valve's last connection was used as the input of the next valve in the series (except for the last one), while the rest were connected to a tube containing the buffer for a single round of hybridization. A fixed subset of the valves was used for imaging, bleaching and wash buffers (see “Experimental procedures and protocols” section). This valve system was used to flow the various buffers into the flow chamber (Bioptechs, 060319-2), in which the sample was placed. The chamber output was connected to a waste collection vessel, forming an open flow system. Components were connected using elastic plastic tubing, and connections were sealed using a pressure adhesive (Blu-tack). The system was controlled using a custom software (see “Software for controlling experimental components” below). Overall, this system allowed for 20-36 rounds of hybridization (depending on the number of valves and the number of spots reserved for special buffers). In experiments where the number of hybridization rounds exceeded the capacity of the flow system, the buffers were replaced with new ones via the following procedure: the output from the valve system was directly connected to the waste collection vessel, bypassing the sample-containing chamber. Then all valves were washed using 30% formamide and double-distilled water. Next, the new set of buffers was introduced, and the chamber was reconnected to the flow system. Lastly, the experiment resumed for the next round of hybridization.

Software for controlling experimental components. All system components were controlled using custom-built software. This software package was composed of the several following main modules, which work in concert: “Hal,” which was the software package used to control and synchronize all illumination and microscope components. It was noted that in some cases it is necessary to write drivers for components, which are not included in this package. Hal is also used to define imaging parameters, such as illumination strength, sequence of stage and illuminations operations during imaging (e.g. during a z-scan), exposure time etc. “Steve,” which was a module used to take mosaic images (i.e. a composite image made up of many individual fields of view) and select regions for imaging in experiments. “Kilroy,” which was the software used to control the fluidics components, and to define pre-programmed sequences of operations to be performed as sets (e.g. the set of operations that happens when a new round of hybridization is performed). “Dave,” which can issue commands to both Hal and Kilroy, and is used to automate the performance of data collection by defining in advance a complete set of fluidics system and microscope operations, the order and time-lag in which they are to be performed.

The general flow of an experiment is that, before the experiment starts, Hal and Kilroy are loaded with the parameters and specifications to be used. After the sample is loaded and the chamber is filled with imaging buffer, a mosaic image of the DAPI channel is taken using Steve, and regions of interest are selected. A file is then generated to specify the sequence of operations throughout the entire experiment and is loaded to Dave, together with the coordinates of the selected regions of interest. The rest of the experiment is run automatically, without manual intervention. If the number of rounds in the experiment exceeds the capacity of the flow system, the automatic sequence specifies actions up to the capacity of the system. The buffers are then replaced (see “Fluidics system configuration” section above), a new Dave file is created, and this is repeated until all rounds of imaging are completed.

Primary/encoding probe synthesis. Primary/encoding probes were amplified from the template library described above (see “Primary/encoding probe design” above). This was done using a previously described amplification protocol involving the following steps: first, the initial oligo pool was expanded using limited-cycle PCR for approximately 20 cycles. The reverse primer used in this step also introduced a T7 promoter sequence via primer extension. Then, the resulting product was purified via column purification and underwent further amplification and conversion to RNA by a high-yield in-vitro transcription reaction. Next, the RNA product was converted back to single-stranded DNA by a reverse transcription reaction. Then, the product of the previous step was subjected to alkaline hydrolysis (to remove residual RNA) and column purified (DNA Clean & Concentrator Kit, Zymo Research D4003 and D4033). Lastly, if necessary, the product of the previous step was dried in vacuum and resuspended in water to achieve the desired concentration of primary probe. All primers were purchased from Integrated DNA Technologies (IDT).

Cell culture preparation and primary/encoding probe hybridization. Cells were prepared similarly to the previous studies. IMR-90 cells were purchased from American Type Culture Collection (ATCC, CCL-186) and grown according to the recommended protocol. To avoid potential alterations to chromatin structure, all cells in this study were plated within 6 weeks of culture initiation at the density specified below.

To prepare for DNA imaging, cells were plated on 40-mm, round #1.5 coverslips (Bioptechs, 0420-0323-2), at a density of ˜500,000 cells per coverslip. Cells were allowed to grow for ˜2 days until confluency at 37° C. and 5% CO₂. In the transcription-inhibition experiments, cell media was replaced with fresh media containing 100 microgram/mL alpha-amanitin (Sigma-Aldrich, A2263) 6 hours prior to cell fixation. For experiments with 1,6-hexanediol (Sigma-Aldrich, 240117), we coated coverslips with 10 microgram/mL fibronectin (Sigma-Aldrich, F1141) prior to cell plating and replaced media with fresh media containing 2% w/v 1,6-hexanediol for 45 minutes. The culture was then fixed using 4% paraformaldehyde (PFA) in PBS for 10 minutes at room temperature and washed in PBS 2-3 times. Cells were then permeabilized in two steps: first, they were treated with 0.5% v/v Triton-X (Sigma-Aldrich, T8787) in PBS for 10 minutes at room temperature. Then, cells were treated with 0.1 M hydrochloric acid (HCl) for 5 minutes at room temperature and washed in PBS 2-3 times. Following HCl treatment, cells were treated with a solution of 0.1 mg/mL RNase A (ThermoFisher, EN0531) dissolved in PBS for 30-45 minutes at 37° C., to remove potential sources of off-target binding to RNA. Following this treatment, cells were incubated in pre-hybridization buffer, used 2× saline-sodium citrate buffer (SSC; Ambion, AM9763) and 50% formamide (Ambion, AM9342) for approximately 10 minutes. Next, the cell coverslip was inverted and placed on a drop of 50 microliters of hybridization buffer (2× SSC, 50% formamide, 10% dextran sulfate (Sigma-Aldrich, D8906) containing a mixture of primary/encoding probes at −25 micromolar total concentration with or without 10 microgram Human Cot-1 DNA (ThermoFisher, 15279011)) in a 60-mm petri dish. The dish was partially submerged in a water bath at −90° C. for 3 minutes and incubated at 47° C. in a humidified chamber for 16-36 hours. After incubation with primary/encoding probes, the sample was washed in 2×SSC and 40% formamide for 30 minutes and post-fixed with 4% PFA in 2×SSC for 10 minutes at room temperature. The sample was then incubated for 2-3 minutes with fiducial beads (either ThermoFisher F8805 or ThermoFisher F8792) in 2×SSC and stained with 1 micromolar 4′,6-diamidino-2-phenylindole (DAPI; ThermoFisher D1306) in 2×SSC for 5-10 minutes, and then stored in 2×SSC until imaging.

For experiments including RNA imaging, all buffers used from the point at which cells were fixed contained a 1:10-1:1,000 dilution of RNAse inhibitor (either NEB M0314 or Fisher Scientific N2615). Treatment for RNA staining was identical to the above-described protocol up to treatment with HCl. After this step, cells were incubated in pre-hybridization buffer for 10 minutes, and the cell coverslip was then inverted and placed on a drop of hybridization buffer containing primary/encoding probes targeting the RNA introns at −1 micromolar total concentration, as described for DNA staining. In this case, however, no 90° C. heat denaturation was performed, and cells were immediately incubated at 47° C. in a humidified chamber for 16-36 hours. After incubation with primary/encoding probes, the sample was washed in a formamide solution and post-fixed with PFA as described for DNA above. It was then incubated with fiducial beads and stained with 1 micromolar DAPI, before being stored in 2×SSC until imaging. After RNA imaging, the sample was removed from the microscope, the cells were treated with RNase A and then the DNA hybridization proceeded in the same manner as described above for DNA imaging without RNA imaging.

Sequential hybridization of readout probes for sequential or combinatorial FISH imaging. All fluid exchanges in this part of the protocol were achieved via the use of a custom-built fluidics system, with the coverslip mounted in a FCS2 flow chamber (Bioptechs, 060319-2). The setup of this system is described in detail in the “Fluidics system configuration” section. Briefly, the fluidics system used 3-5 computer-controlled eight-way valves (Hamilton, MVP and HVXM 8-5) and a computer-controlled peristaltic pump (Gilson, MINIPLUS 3). Put together, these components allow control of both the rate of fluid flow and of the type of fluid flowing at any given time. Each round of hybridization used the following general steps: first, the hybridization buffer was flowed in with a set of oligonucleotide probes specific to each round, as described below. Then, it was incubated for 10 minutes at room temperature. Next, the wash buffer was flowed through, and it was incubated for ˜200 seconds, and lastly, the imaging buffer was flowed through.

Imaging buffer was prepared as described previously, and used 60 mM Tris pH 8.0, 10% w/v glucose, 1% Glucose Oxidase Oxygen Scavenger Solution (containing −100 mg/mL Glucose Oxidase (Sigma-Aldrich, G2133) and a 1:3 dilution of catalase (Sigma-Aldrich, C3155)), 0.5 mg/mL 6-hydroxy-2,5,7,8-tetramethylchroman-2-carboxylic acid (Trolox; Sigma-Aldrich, 238813) and 50 μM Trolox Quinone (generated by UV irradiation of a Trolox solution). Trolox was dissolved in methanol before being added to the solution. After preparation, the imaging buffer was covered by a ˜0.5 cm thick layer of mineral oil to prevent exposure to oxygen.

The hybridization buffer and wash buffer were made up of 35% and 30% formamide in 2×SSC, respectively, with the hybridization buffer also containing 0.01% v/v Triton-X. The hybridization buffer was kept separately for each hybridization round and contained two (for genome-scale chromatin imaging by combinatorial FISH) or three (for whole-chromosome imaging by sequential hybridization and for genome-scale chromatin and RNA imaging by combinatorial FISH) sets of readout probes. Fluorescent signal was introduced in the following ways: for whole-chromosome imaging by sequential hybridization, the hybridization buffer contained three fluorescent readout probes (Alexa750, Alexa647 or Cy5, and Cy3) added at 30 nM concentration. All experiments involved the sequential hybridization of oligonucleotide adaptors and fluorescent readout probe as described in FIG. 24A. During each round of hybridization, a set of adaptor probes (each at 100 nM concentration) were first flowed in for the detection of three targeted genomic loci in three distinct color channels (Alexa750, Alexa647 or Cy5, and Cy3). Each adaptor probe comprised of a segment complementary to the readout sequence unique to one of the targeted loci and a segment containing a color-channel-specific common readout sequence. Next, three distinct dye-conjugated, readout probes, each complementary to one of the three color-channel-specific common readout sequences were flown in at 30 nM concentration for each probe. This procedure allows three genomic loci to be imaged, respectively in three color channels, during each round of hybridization. Fluorescent readout probes contained a disulfide bond linking the fluorophore to the oligonucleotide, as described previously, to allow efficient removal of signal between rounds. For genome-scale chromatin imaging by combinatorial FISH, each round's hybridization buffer included two fluorescent readout probes, one labeled with Cy5 or Alexa647 and the other labeled with Alexa750. Fluorescent readout probes used either: 1) a fluorescently labeled oligo complementary to a readout sequence common to all encoding probes imaged in a given bit, added at 100 nM concentration, or 2) a combination of an adaptor oligo having the sequence complementary to a readout sequence, concatenated to an additional common readout sequence (common to all adaptors in each color channel), as described above, and a fluorescently labeled readout probe complementary to this common readout sequence. For some experiments, the adaptor and common readout probes were pre-mixed in a 1:1.5 ratio and added to a final concentration of ˜100 nM. For other experiments the adaptor and readout probes were hybridized sequentially to the sample. For RNA imaging, each round's hybridization buffer contained three adaptor probes, one for each color channel, as described above. Each round included two discrete hybridization steps—first the adaptors were flowed in, hybridized, and excess material was washed. Then three fluorescent readout probes, complementary to the common readout sequences on the adaptors, respectively labeled with Cy3, Cy5 (or Alexa647) and Alexa750, were flown in sequentially. After the fluorescent readouts were hybridized, imaging buffer was flowed in and signal was collected.

Before the next round of readout probe or adaptor probe hybridization, fluorescent signals from the readout probes in the current round were removed as described in the “Signal removal between rounds of hybridization” section below.

Before the first round of hybridization, a round of imaging was performed to acquire the DAPI signal and identify nuclear boundaries. For whole-chromosome imaging by sequential hybridization, 651 genomic loci on Chr21 over −220 rounds or 935 genomic loci on Chr2 over −320 rounds were imaged, all in 3 color channels. For genome-scale chromatin imaging by combinatorial FISH, the entire set of 1,041 genomic loci were imaged in 50 rounds of hybridization and 2 color channels per round. In each round, the genomic loci were imaged in 3D by stepping the stage in the z-dimension. Nascent RNA transcripts for the 86 genes on Chr21 were imaged sequentially over 31 rounds in 3 colors, and the genome-scale imaging of the RNA transcripts of 1,137 genes were imaged in 3D in 18 rounds in 3 colors. For sequential imaging of Chr21 specifically, the TSS of the 86 genes over 29 rounds was imaged in 3 colors. Additional rounds were used to relabel sets of genomic loci and assess chromatic aberration and bleedthrough between color channels, as well as stability of the sample and imaging instrument. Imaging of ˜60 fields of view containing a total of ˜1,000-3,000 cells took ˜12-18 days for sequential imaging of whole-chromosome imaging by sequential hybridization and 3 days for genome-scale chromatin imaging by combinatorial FISH.

The 3-5 valve system allowed loading up to 20-36 different hybridization solutions. As a result, after exhausting all the fluidic system's channels the sample chamber was bypassed and all the channels used for hybridization were washed with 30% formamide in water. Next the chamber was reconnected and the next set of hybridization and imaging rounds proceeded.

For whole-chromosome imaging by sequential hybridization, a gentle post-fixation step was performed with 2% PFA in 2×SSC for 5 minutes periodically (every ˜4 days) to maintain the structural integrity of the sample.

Antibody labeling and imaging. Antibody imaging was performed immediately after RNA or DNA imaging. Following completion of imaging via the protocols described above, samples underwent the following steps: Samples were incubated with blocking solution (PBS with 0.1% v/v Tween-20 (Sigma-Aldrich P9416) and 1% w/v bovine serum albumin (BSA; Jackson Immunoresearch 001-000-162)) for 30 minutes. Samples were incubated with primary antibody diluted in blocking solution for 1 hour. Samples were washed 3 times in PBS with 0.1% Tween-20 for 5 minutes each. Steps 2 and 3 were repeated for a fluorescently tagged secondary antibody.

All buffer exchanges were done on the microscope, using the microfluidic system described in the “Fluidics system setup” section below. The Cy5 color channel was used for imaging and the signal between sequential antibody labelling was extinguished using photobleaching.

The following sets of primary and secondary antibodies were used: for imaging nuclear speckles, a primary antibody against SC35 (Abcam, ab11826) was used—a splicing factor commonly used as a marker of nuclear speckles—at 1:200 dilution from stock and a donkey anti-mouse secondary antibody labeled by a Cy5 dye (Jackson Immunoresearch, 715-175-150) diluted 1:1,000 from stock concentration. For imaging nucleoli, anti-fibrillarin antibody (Abcam, ab5821), at 1:200 dilution from stock, and a donkey anti-rabbit secondary antibody labeled by an Alexa 657 dye (Jackson Immunoresearch, 711-605-152), diluted 1:1,000 from stock concentration was used. For cell-cycle state determination, the immunofluorescence staining was performed immediately after RNA imaging using the anti-geminin antibody (Abcam, ab195047), at 1:100 dilution from stock, and a donkey anti-rabbit secondary antibody labeled by an Alexa 657 dye (Jackson Immunoresearch, 711-605-152), diluted 1:1,000 from stock concentration.

Image acquisition. For each experiment, approximately ˜60 fields of view (FOVs) were selected for imaging, avoiding regions where cells are sparse (we typically identified 10-50 cells per FOV). Each camera FOV used either 1,000×1,000 pixels, with a camera pixel corresponding to 153 nm in each dimension in the imaging plane, or 2048×2048 pixels, with a camera pixel corresponding to 108 nm in each dimension in the imaging plane.

After each round of hybridization (see “Sequential hybridization of readout probes for FISH imaging” above), z-stack images of each FOV were acquired in 3 or 4 colors: 647 nm and 750 nm illumination (or 560 nm, 647 nm, and 750) were used to acquire FISH images, 560 nm illumination (or 405 nm illumination) was used to image fiducial beads. For the first round of imaging, 405 nm illumination was used to image the DAPI signal, while for antibody imaging, the 647 nm excitation channel was used after RNA or DNA imaging. Consecutive z-sections were separated by 85, 100, 150 or 200 nm, covering the entirety of the nuclear volume for all imaged cells. At each z position, images were acquired in all channels before the stage was moved and images were acquired at a rate of ˜10 Hz.

Signal removal between rounds of hybridization. Before each round of imaging, the signal from the previous round (or endogenous background, in the case of the first round) was extinguished. This was achieved via cleaving the disulfide bond connecting fluorophores to readout probes as described previously with an optional photobleaching step. The buffer used for cleaving contained 50 mM tris(2-carboxyethyl)phosphine (TCEP; Sigma-Aldrich, C4706) to reduce the disulfide bond connecting fluorophores to readout probes, as well as 1 mM dye-free common readout probes in 35% formamide to block any unoccupied readout sequences from interfering with the next round of hybridization. In experiments where photobleaching was performed, it was accompanied by changing the buffer to 2×SSC with or without 35% formamide and illuminating each field of view with the maximum available power of the 560, 647 and 750 lasers for 3-10 seconds. The photobleaching step in the sequential tracing experiments was done during the hybridization step with oligonucleotide adaptors, to minimize total experimental time. The DAPI signal was extinguished as a result of the high formamide concentration in the hybridization and wash buffers.

Relabeling of genomic regions in sequential DNA-FISH imaging. After finishing the sequential DNA-FISH imaging experiment for the entire chromosome, a subset of regions was re-labelled and re-imaged. The sample was treated with 57% formamide in 2×SSC for 4 minutes and this treatment was repeated 3 times to strip off the readout oligonucleotide probes (whose fluorescence signal was removed after the first round of imaging by first cleaving the dye from the oligonucleotide probe using TCEP and then by photobleaching). After stripping off the readout probes, 1 mM dye-free common readout probes were added in 35% formamide 2×SSC to block any unoccupied readout sequences on adaptor probes that were not stripped off. Next, relabeling of selected regions was achieved by following the standard readout probe hybridization protocol (described in section “Sequential hybridization of readout probes for sequential or combinatorial FISH imaging”).

Image analysis: Overview of analysis pipeline. The image analysis pipeline used in this study was implemented in Python. The overall pipeline used the following steps: identify and segment all imaged nuclei, fit 3D-Gaussians to all detected fluorescent spots in imaging channels used for DNA or RNA imaging, as well as for fiducial beads. DNA and RNA spots which did not overlap with identified nuclei were rejected, correct sample drift using the fiducial beads, correct chromatic effects between different color channels, and assign identities to DNA loci and RNA molecules using custom algorithms and software (these are described separately below for DNA and RNA imaging, both for chromosome-wide and for genome-wide imaging).

Nuclei segmentation. DAPI images from the first round of imaging were used to identify the volume of individual nuclei and allowed for cell segmentation. This was achieved via a convolutional neural network, built and trained similarly to a previous published work, which took the maximum projection of the DAPI image onto the xy plane as input.

Spot fitting for DNA and RNA imaging. The following analysis pipeline was applied to each imaged FOV in order to obtain the three-dimensional (3D) positions of all loci of interest: fiducials were fitted in all rounds of imaging and used for image alignment (see “Drift correction” section below). In the first imaging round (preceding the first round of hybridization), DAPI signal was used to identify the borders of individual nuclei, as well as for image registration between RNA and DNA imaging. See “Nuclei segmentation” and “Image registration between DNA and RNA imaging” sections for details. Diffraction-limited spots within each identified nucleus were fitted to a 3D Gaussian function to identify their center of mass and brightness above local background. To make analysis more manageable, the number of fitted spots per image that will be retained for decoding was fixed to 125 or fewer in genome-scale imaging by combinatorial FISH (˜3-fold greater than the number of distinct loci expected without noise). For whole-chromosome imaging by sequential hybridization, the number of fitted spots per chromosome per image was fixed to 6 or fewer. The fitted spots from step 3 were then used for identifying DNA loci and transcription foci and determining their positions, as described in the corresponding sections below.

Drift correction. Fiducial bead spot fitting was performed in the same way as described above. The set of fiducial bead positions was then compared between rounds of hybridization and a rigid transformation was applied to minimize the sum of square difference of the relative position of beads.

Correction of chromatic effects. Bleedthrough and chromatic aberration for multi-color imaging were performed by labeling the same set of genomic loci in each imaging channel independently and comparing the signals of the same loci in the different color channels, respectively.

Image registration between DNA and RNA imaging. DAPI signal was first used for rough image-registration across the two sets of images (i.e. chromatin and RNA) via 2D image correlation (all images within each set were aligned to the DAPI image using fiducial beads). After an initial round of RNA decoding was performed (see “Identification of transcription foci from fitted RNA spots in sequential imaging” and “Decoding algorithm for fitted RNA spots in combinatorial, genome-scale imaging” below), a finer alignment was calculated by assuming that the displacement between nascent RNA localization and their harboring DNA loci should average to zero when considered across all imaged genes and cells in a field of view. In accordance, an additional rigid transformation was calculated to minimize the mean displacement between imaged nascent RNA and their corresponding DNA loci and used this as the final alignment.

Identification of chromatin loci from fitted DNA spots in sequential imaging. Identification and 3D localization of each locus were achieved through the following steps: a list was generated for the drift- and aberration-corrected locations of all fitted spots in each image. Because the spot finding algorithm was allowed to find up to 6 candidates for each chromosome in each sequential image corresponding to a specific color channel of a specific hybridization round (see “Spot fitting for DNA and RNA imaging”), the following additional steps were performed to identify the candidate spots most likely to originate from the imaged chromatin locus. An initial tentative chromatin trace was generated by selecting the brightest spot corresponding to each chromosome copy in each cell in each color channel of each hybridization round. For each fitted spot, regardless of whether it was selected for the initial tentative chromatin trace, three quality metrics were calculated: the spot's brightness above local background, the spot's distance to the local center of mass, which was calculated from five loci upstream and five downstream along the tentative chromosome trace, and the distance to the center of mass of the entire tentative chromosome trace. For each spot the three quality metrics described above were combined into a single measure by calculating the combined Fisher p-value for every candidate spot against the distribution of quality metric values for spots included in the tentative chromatin trace (which we will term the “valid distribution”). This can be thought of as the overall quality score of each spot, and was calculated per spot in the following way: for each of the metrics the fraction of other spots in the “valid distribution” that had lower quality metric was calculated and these three fractions were multiplied. An expectation-maximization procedure was then used to sequentially select the spot with the highest quality score corresponding to each targeted chromatin locus and the “valid distribution” was re-updated based on the resulting chromatin trace. This optimization procedure was repeated until convergence. After convergence, the final sets of spots, each corresponding to a chromatin locus, were used to determine the 3D spatial positions of the targeted loci. Finally, the spots from step 4 were filtered to remove those whose quality scores were below a set cutoff (and thus were of low confidence). To set a cutoff value for the combined quality score, the quality scores of loci that had been included in the re-imaging experiment were first computed to determine displacement error (see “Identification of re-imaged loci in sequential imaging” section, below). Then, the distribution of quality scores for spots with low displacement error (<500 nm) between the original was calculated and loci were re-imaged for spots with high displacement error (>500 nm). Finally, the quality score threshold was set such that the fraction of loci in the final chromatin trace (after applying the threshold) expected to be in the high displacement error category is <5%. The remaining spots after step 5 were used to determine the final positions of the chromatin loci and trace the chromatin structure.

Identification of transcription foci from fitted RNA spots in sequential imaging. Signal from RNA imaging rounds was analyzed using the following procedure: first, the positions of fitted RNA spots for each cell was corrected for chromatic aberration and drift using first a coarse DAPI-based alignment and the brightest RNA spot within a distance of 1000 nm to the corresponding DNA locus was kept. Then, the registration between DNA and RNA imaging was refined based on the displacement between the initial selected RNA localizations (from step 1 and the location of the DNA locus harboring them as described in the “Image registration between DNA and RNA imaging” section above. Lastly, the locations of all the candidate RNA spots, after the fine registration from step 2, were compared to the location of the 50 kb DNA locus harboring the gene and the corresponding kb DNA transcription start site. At this stage, a more stringent distance cutoff of 500 nm from either the 50-kb locus or the 5-kb transcription start site, as well as a signal-to-noise ratio threshold of 1, was applied. If the nascent RNA localization passed both thresholds, it was considered as a detected transcriptional burst.

Identification of re-imaged loci in sequential imaging and estimate of displacement error. Identification of re-imaged loci was performed similarly to the description in the “Identification of chromatin loci from fitted DNA spots in sequential imaging” section, except that the re-imaged loci were used to replace the corresponding subset of loci in the original imaging rounds.

For computing the displacement error between the original and re-imaging rounds (FIGS. 24B-24D), only loci that passed a brightness threshold, based on the following observation, were considered. It was noticed that the fluorescent signals in the re-imaging rounds were substantially dimmer than in the original imaging rounds. This was potentially due to the incomplete removal of the original readout probes and/or due to the partial removal of the primary probes bound to the genomic DNA during the formamide treatment to strip off the fluorescent readout probes bound in the original imaging rounds. The lower brightness reduced the localization accuracy of the re-imaged loci and caused an artificial overestimation of the localization error of the initial imaging rounds. To mitigate this effect, in estimating the localization error, only those re-imaged loci that were >20% in brightness compared to the original signal were selected.

As an additional note, when examining loci with >1000 nm from both neighboring loci, a fraction (˜20%) of these exhibit a large re-imaging displacement error and have lower brightness. Thus, loci that are far away from both of their genomic neighbors may have a relatively low confidence.

Decoding algorithm for fitted DNA spots in combinatorial, genome-scale imaging. Identification and 3D localization of each locus were achieved through the following steps: first, a list was generated for the drift- and aberration-corrected locations of all identified spots in each bit-image (corresponding a specific color channel in a specific round of imaging). For each detected spot in every bit-image, all spots from other bit-images that were within a set cutoff distance (˜150 nm in x, y and z) from its location were found. All such pairs of spots were retained for further analysis, whether or not the barcode produced by a spot pair (based on which round and color channel they appeared in) corresponded to a valid barcode (i.e. a barcode that was assigned to a genomic locus). Then, for each pair of spots, three quality metrics were calculated: the displacement between the 3D localizations of the two spots, the difference in brightness between the two spots, and the mean brightness of the two spots. The brightness of each spot was normalized by the median brightness of all spots in the corresponding bit-image. Spot pairs were then separated into two groups, based upon whether they correspond to a valid barcode (and hence potentially to a genomic locus) or not. Within each group, the distributions of the quality metrics were calculated. For convenience, the distribution of spot-pair quality metrics from the invalid barcodes is referred to as the “invalid distribution” and from all valid barcodes as the “valid distribution.” For each spot pair the three quality metrics in step 3 were combined into a single measure by calculating the combined Fisher p-value for every candidate spot pair against the “valid distributions.” This can be thought of as the overall quality score of each spot-pair, and was calculated per pair in the following way: for each of the three metrics the fraction of other spot-pairs in the “valid distribution” that had lower quality metric were calculated and these three fractions were multiplied. An expectation-maximization procedure was used to sequentially select the two spot-pairs with the highest quality score corresponding to each targeted chromatin locus and reupdated the “valid distribution,” and repeated this optimization procedure until convergence. After convergence, the final sets of spot pairs, each corresponding to a chromatin locus, were used to determine the 3D spatial positions of the loci. After step 5, a modified K-means algorithm was used to separate the chromatin loci belonging to the same chromosome into two homologs. As opposed to the standard K-means clustering algorithm which splits points into two groups and minimizes the radius of gyration within each group, the points between the groups were progressively switched to first maximize the fraction of assigned points in each homolog and then minimize the radius of gyration of each homolog. After separating the two homologs their center of mass and the distance of each spot-pair from step 2 to their parent chromosome's center of mass were calculated. The distance to the chromosome center was added as another quality metric in addition to the 3 metrics considered in step 3 and repeated steps 3-6. Finally, the spot pairs from step 7 were filtered to remove the pairs whose quality scores remained similar to the “invalid distribution.” The remaining spot pairs after step 8 were used to determine the final positions of the chromatin loci and trace the chromatin structure.

Decoding algorithm for fitted RNA spots in combinatorial, genome-scale imaging. Signal from RNA imaging rounds was decoded using the following procedure: first, a list was generated for the drift- and aberration-corrected locations of all identified spots in each round of imaging. For each detected spot in every imaging round, all spots from other rounds that were within a set cutoff distance from its location were found and these spot pairs were retained as candidate RNA bursts if they formed a valid barcode. Then, the location of each of these candidate RNA bursts was then compared to the location of the DNA locus harboring the relevant gene, after initial image registration (based on DAPI images) and drift and aberration correction, and kept if they were within a set threshold distance. Next, the registration between DNA and RNA imaging was refined based on the displacement between the initial decoded RNA localizations (from step 3) and the location of the DNA locus harboring them as described in the “Image registration between DNA and RNA imaging” section above. Lastly, locations of all candidate RNA bursts were compared again to the location of the DNA locus harboring the gene to which they decode, this time with the refined image registration. If the nascent RNA localization was within a cutoff distance from its harboring DNA locus at this stage, it was considered as a detected transcriptional burst.

Identification of re-imaged loci in combinatorial, genome-scale imaging. In the combinatorial, genome-scale imaging approach, a subset of the targeted genomic regions on chromosome 6 were assigned probes such that they could be re-imaged individually using sequential multi-color FISH, after the combinatorial imaging. For each decoded instance of one of these loci in the combinatorial imaging, the displacement error was estimated as the distance between the localization determined in combinatorial imaging and the nearest spot in the sequential re-imaging rounds.

Identification of nuclear bodies from immunofluorescence imaging. The location of nuclear bodies (nuclear speckles and nucleoli) was extracted from immunofluorescence signals by applying a threshold to the intensity of the immunofluorescence signals, resulting in a pixelated mask identifying high immunofluorescence signals. This was then treated as a pixelated set of locations “containing” nuclear bodies.

Cell cycle stage determination from anti-geminin and DAPI images. First, cells undergoing mitosis were eliminated by visual inspection and not considered for the analysis. Next, using a combination of geminin immunofluorescence signal and nuclear signal measured using DAPI, cells were classified as G1 (low geminin signal, low DAPI signal) or G2/S (high geminin signal) similarly to the previous study.

Estimation of the nuclear lamina location. The position of the nuclear lamina was estimated by generating the minimal 3D convex hull surface (using Python's SciPy package) surrounding the locations of all decoded chromatin loci in a given cell.

Spatial distance. The spatial distance between any pair of loci was simply calculated as the Euclidean distance between their fitted 3D Gaussian centers, multiplied by the appropriate ratios relating camera pixels and z steps to physical distance. In the case of distance to nuclear bodies, the minimal Euclidean distance to all identified nuclear body “locations” or the minimal distance to the surface of the convex hull defining the nuclear lamina was calculated.

Proximity frequency matrices from imaging. To calculate the proximity frequency between any given pair of loci, the number of measured distances between that locus-pair that was smaller than a set cut-off distance was first counted (500 nm in this study, unless otherwise mentioned). This number was then divided by the total number of distances measured for that pair of loci. The cut-off distance was determined by assessing the Pearson correlation between the proximity frequency matrices resulting from a range of cut-off thresholds with the Hi-C contact matrix, as well as the alignment of ensemble structural features, such as TADs and compartments, derived from imaging and Hi-C data for Chr21.

It was noticed that the Pearson correlation coefficient between the proximity frequency map and Hi-C map remained largely constant in the range of 0.82-0.88 for cut-off distances between 200 nm to 800 nm and reached a maximum at ˜400-500 nm. Furthermore, for cut-off thresholds <500 nm, the TAD boundaries derived from the imaging data aligned with the TAD boundaries derived from Hi-C data with a similarly high precision, while for cut-off distances larger than 600 nm, the alignment degraded. At the larger scale, the A/B compartment calling displayed the highest level of agreement for cut-off distances between 400 to 600. Hence, a cut-off distance in the range of 400-500 nm was considered to be optimal for an accurate calling of both TADs and A/B compartments and selected a cut-off distance of 500 nm for all analysis.

Local density analysis. To calculate the local density of compartment-A and compartment-B loci at each decoded location, the spatial distances between each pair of chromatin loci for each cell were calculated. For each locus, the local A/B density ratio was computed in the following way: first, a Gaussian probability density function centered around each A or B locus with a standard deviation of 100 nm (for Chr21 imaging), 125 nm (for Chr2 imaging), or 500 nm (for genomic-scale imaging) was placed. Then, the total A density at the locus was then computed as the sum of this Gaussian probability density function values from all A loci excluding itself in whole-chromosome imaging. For genome-scale imaging, the total trans A density at the locus were summed from all trans-chromosomal A loci (i.e. all A loci from other chromosomes). The total B density was computed in an analogous way. Lastly, the total density of compartment-A loci was divided by the density of compartment-B loci to find the A/B density ratio at the locus. The trans A/B density ratio was computed analogously.

Insulation score from imaging data. Insulation score has been previously defined for ensemble Hi-C. An analogous definition was used for the imaging results and applied to compute insulation scores of neighboring or non-neighboring domains in individual chromosomes in single cells.

To calculate the insulation score between two domains, an intra-domain distance distribution was calculated by considering all distances between each pair of loci within the first domain and all distances between each pair of loci within the second domain. An inter-domain distance distribution was then calculated by considering all distances between pairs of loci that reside in different domains. The insulation score was then defined as the median of all inter-domain distances divided by the median of intra-domain distances. Two highly intermixed domains would have insulation score close to 1, while domains that are just contacting will have an insulation score of ˜2.

Normalized insulation score for TAD calling. To allow comparison of TAD calling in imaging and Hi-C data, the insulation score definition above was slightly modified such that insulation scores from these data would fit into the same dynamic range. For TAD-calling in median pairwise distance matrices from imaging data, for each genomic locus upstream and downstream loci with a fixed window were selected (i.e. a fixed number of loci on each side of selected locus). These two chromatin regions, up- and down-stream of the selected locus, were treated as two domains and computed insulation score, as described above. The normalized insulation score was then defined as the difference between median of inter-region distances and median of intra-region distances normalized by the sum of these two median values. Therefore, a normalized insulation score will always be between 0 and 1. With this definition of normalized insulation score, a sliding window was applied along the chromosome to calculate a vector of insulation scores corresponding to genomic loci, and local maxima were found by standard peak-calling algorithm from Scipy and these positions were considered as the TAD boundaries. TAD boundary calling using the proximity frequency matrix derived from imaging or Hi-C contact matrix were performed similarly.

A/B segregation score within chromosomes. A/B segregation score quantifies the level of spatial separation between A and B loci within a chromosome. To calculate this quantity, the A-dense volume was first operationally defined within each chromosome as the 3D space that contains all A loci with A density scores being in the top ⅔ range. The B-dense volume was operationally defined in an analogous fashion. A purity metric of the A- and B-dense volumes was defined as the fraction of all loci within these volumes being A and B loci, respectively. Finally, the A/B segregation score was defined as the mean of purity values of the A-dense and the B-dense volumes. This segregation score would be 1 if A and B loci are entirely segregated, and a chromosome with A and B loci completely intermixed would have segregation score around 0.5.

Estimation of detection efficiency in RNA imaging by combinatorial FISH. Estimation of the detection efficiency of transcriptional burst events was performed in the following way: first, all targeted genomic loci that harbor a gene whose RNA introns were imaged were considered. For any of these genomic loci, their corresponding RNA signal should appear in two pre-defined bits if the gene is transcribed. Knowing the rate with which each of these two bits is not detected (p) allows the detection efficiency of the RNA to be derived. The set of genomic loci that colocalized (within ˜150 nm) with RNA signal in at least one of the two expected bits of their corresponding genes was identified. Then, from the total set of chromatin loci identified in step 1, the fraction (f) of loci that colocalized with RNA signal was determined from exactly one of its gene's corresponding bits (and not with both bits). From the measured f (8.4%), which should be equal to

$$\frac{2 p ( 1 - p )}{1 - {p}^{2}} ,$$

p (4.4%) was estimated. Lastly, the overall detection efficiency for detecting a colocalized signal in both bits was calculated using the equation: η=(1−p)², and was found to be ˜92%.

Hi-C data analysis. Hi-C data for IMR-90 cells was procured from and loaded using straw. For identification of A/B compartments in individual chromosomes, established published protocols were followed. For identification of TADs, the method described in the “Normalized insulation score for TAD calling” section was used. For comparison of proximity frequencies derived from the imaging data to Hi-C number of contacts, bins centered around the regions targeted were created and Hi-C data for these bins was procured by summing the number of reads in higher resolution Hi-C data.

CTCF and Rad21 ChIP-seq data analysis. CTCF and Rad21 ChIP-seq data were downloaded from ENCODE dataset and converted to wig format by UCSC Genome Browser Utilities. Read counts for the targeted genomic segment were collected and normalized by input correspondingly. Local maxima of CTCF or Rad21 ChIP-seq signal enrichment over input along the chromosome were called by standard peak calling algorithm from Scipy.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
associating a plurality of nucleic acid targets of a genome with a plurality of codewords, wherein the codewords comprise a number of positions and values for each position;
exposing a sample containing the genome to a plurality of nucleic acid probes;
for each nucleic acid probe of the plurality of nucleic acid probes, determining binding of the nucleic acid probe within the sample;
creating codewords corresponding to the binding of the plurality of nucleic acid probes within the sample; and
determining the identities of the nucleic acid targets based on the codeword assigned.

2. The method of claim 1, further determining the spatial positions of the identified nucleic acid targets.

3. The method of any one of claim 1 or 2, further determining the three-dimensional organization of the chromatin or the genome based on the spatial positions of the identified nucleic acid targets.

4. The method of any one of claims 1-3, where in the codewords form an error-checking and/or error-correcting code space.

5. The method of claim 4, wherein the error-checking and/or error-correcting detection technique comprises MERFISH.

6. The method of any one of claims 1-5, wherein at least some of the plurality of nucleic acid probes comprises a first portion comprising a target sequence and a second portion comprising one or more readout sequences, wherein each readout sequence represents a value of a position within the codewords.

7. The method of claim 6, further comprising exposing the sample to readout probes that can bind to the readout sequences.

8. The method of claim 7, wherein the readout probes contain signaling entities.

9. The method of claim 8, wherein the signaling entities are fluorescent molecules.

10. The method of any one of claims 7-9, further comprising exposing the sample to a plurality of readout probes sequentially.

11. The method of any one of claims 7-10, further comprising creating codewords corresponding to the binding of the plurality of nucleic acid probes within the nucleus, wherein the values of the digits of the codewords are based on the readout sequences present on the nucleic acid probes.

12. The method of any one of claims 7-11, wherein for at least some of the codewords, matching the codeword to a valid codeword wherein, if no match is found, either discarding the codeword or applying error correction to the codeword to form a valid codeword, the valid codewords being a plurality of codewords assigned to the plurality of the nucleic acid targets.

13. The method of any one of claims 1-12, wherein the plurality of nucleic acid targets are separated by at least 100,000 nucleotides within the genome

14. The method of any one of claims 1-13, wherein the plurality of nucleic acid targets are separated by at least 300,000 nucleotides within the genome.

15. The method of any one of claims 1-14, wherein the plurality of nucleic acid targets are separated by at least 1,000,000 nucleotides within the genome.

16. The method of any one of claims 1-15, wherein the plurality of nucleic acid targets are separated by at least 10,000,000 nucleotides within the genome.

17. The method of any one of claims 1-16, wherein the plurality of nucleic acid targets of the genome are distributed such that each chromosome of the genome contains no more than 10,000 nucleic acid targets.

18. The method of any one of claims 1-17, wherein the genome has between 10 and 100,000 nucleic acid targets.

19. The method of any one of claims 1-18, further comprising determining nascent RNA within the nucleus of the cell.

20. The method of any one of claims 1-19, further comprising determining nuclear speckles within the nucleus of the cell.

21. The method of any one of claims 1-20, further comprising determining nucleoli within the nucleus of the cell.

22. The method of any one of claims 1-21, further comprising determining nuclear lamina within the nucleus of the cell.

23. The method of any one of claims 1-22, further comprising determining other protein and nucleic acids species within the nucleus of the cell.

24. The method of any one of claims 1-23, further comprising determining other protein and nucleic acids species within the sample.

25. The method of any one of claims 1-24, comprising exposing the sample to at least 10 nucleic acid probes.

26. The method of any one of claims 1-25, comprising exposing the nucleus to at least 30 nucleic acid probes.

27. The method of any one of claims 1-26, comprising exposing the nucleus to at least 100 nucleic acid probes.

28. The method of any one of claims 1-27, comprising exposing the nucleus to at least 1,000 nucleic acid probes.

29. The method of any one of claims 1-28, comprising exposing the nucleus to at least 10,000 nucleic acid probes.

30. The method of any one of claims 1-29, comprising exposing the nucleus to at least 100,000 nucleic acid probes.

31. The method of any one of claims 1-30, comprising exposing the nucleus to at least 1,000,000 nucleic acid probes.

32. The method of any one of claims 1-31, comprising exposing the sample to the nucleic acid probes sequentially.

33. The method of any one of claims 1-32, wherein the plurality of nucleic acid probes comprises a combinatorial combination of nucleic acid probes with different sequences.

34. The method of claim 33, wherein the plurality of nucleic acid probes comprises at least 8 readout sequences.

35. The method of any one of claim 33 or 34, wherein the plurality of nucleic acid probes comprises at least 32 readout sequences.

36. The method of any one of claim 33-35, wherein the plurality of nucleic acid probes comprises no more than 32 possible readout sequences.

37. The method of claim 33, wherein the plurality of nucleic acid probes comprises no more than 8 possible readout sequences.

38. The method of any one of claims 33-37, wherein the plurality of readout sequences are distributed on the plurality of nucleic acid probes so as to define an error-checking code.

39. The method of any one of claims 33-38, wherein the plurality of readout sequences are distributed on the plurality of nucleic acid probes so as to define an error-correcting code.

40. The method of any one of claims 33-39, wherein the plurality of readout sequences have an average length of between 5 nucleotides and 50 nucleotides.

41. The method of any one of claims 33-40, wherein at least some of the plurality of nucleic acid probes comprise no more than 50 readout sequences.

42. The method of any one of claims 33-41, wherein at least some of the plurality of nucleic acid probes comprise no more than 10 readout sequences.

43. The method of any one of claims 33-42, wherein determining binding of the nucleic acid probe within the nucleus comprises:
exposing the nucleus to a first readout probe comprising a first signaling entity, the first readout probe able to bind to one or more of the readout sequences of the nucleic acid probes; and
determining binding of the nucleic acid probes by determining the first signaling entity within the nucleus.

44. The method of claim 43, wherein the first signaling entity is fluorescent.

45. The method of any one of claim 43 or 44, wherein the first signaling entity is a protein.

46. The method of any one of claims 43-45, wherein the first signaling entity is a dye.

47. The method of any one of claims 43-46, wherein the first signaling entity is a nanoparticle.

48. The method of any one of claims 43-47, further comprising:
exposing the nucleus to a second readout probe comprising a second signaling entity, the second readout probe able to bind to some of readout sequences of the nucleic acid probes; and
determining binding of the nucleic acid probes by determining the second signaling entity within the nucleus.

49. The method of claim 48, wherein the first signaling entity and the second signaling entity are identical.

50. The method of any one of claim 48 or 49, wherein the first signaling entity and the second signaling entity are not identical.

51. The method of any one of claims 48-50, further comprising inactivating the first signaling entity prior to exposing the nucleus to the second secondary probe.

52. The method of claim 51, comprising inactivating the first signaling entity by photobleaching at least some of the first signaling entity.

53. The method of any one of claim 51 or 52, comprising inactivating the first signaling entity by chemically bleaching at least some of the first signaling entity.

54. The method of any one of claims 51-53, comprising inactivating the first signaling entity by exposing the first signaling entity to a reactant able to alter the structure of the signaling entity.

55. The method of any one of claims 51-54, comprising inactivating the first signaling entity by removing at least some of the first signaling entity.

56. The method of any one of claim 51-55, comprising inactivating the first signaling entity by dissociating the first signaling entity from the first readout probe.

57. The method of any one of claims 51-56, comprising inactivating the first signaling entity by dissociating the first readout probe that contains the first signaling entity from the sample.

58. The method of any one of claims 51-57, comprising inactivating the first signaling entity by chemically cleaving it from the first readout probe.

59. The method of any one of claims 51-58, comprising inactivating the first signaling entity by enzymatically cleaving it from the first readout probe.

60. The method of any one of claims 51-59, comprising inactivating the first signaling entity by exposing the signaling entity or the first readout probe to an enzyme.

61. The method of any one of claims 43-60, comprising determining a centroid of the first signaling entity using an algorithm for determining non-overlapping single emitters.

62. The method of any one of claims 43-61, comprising determining a centroid of the first signaling entity using an algorithm for determining partially overlapping single emitters.

63. The method of any one of claims 43-62, comprising determining a centroid of the first signaling entity using a maximum likelihood algorithm.

64. The method of any one of claims 43-63, comprising determining a centroid of the first signaling entity using a least squares algorithm.

65. The method of any one of claims 43-64, comprising determining a centroid of the first signaling entity using a Bayesian algorithm.

66. The method of any one of claims 43-65, comprising determining a centroid of the first signaling entity using a compressed sensing algorithm.

67. The method of any one of claims 1-66, wherein at least some of the plurality of nucleic acid probes comprise DNA.

68. The method of any one of claims 1-67, wherein at least some of the plurality of nucleic acid probes comprise RNA.

69. The method of any one of claims 1-68, wherein at least some of the plurality of nucleic acid probes comprise PNA.

70. The method of any one of claims 1-69, wherein at least some of the plurality of nucleic acid probes comprise LNA.

71. The method of any one of claims 1-70, wherein the plurality of nucleic acid probes have an average length of between 10 and 300 nucleotides.

72. The method of any one of claims 1-71, wherein at least some of the binding of the nucleic acid probes within the nucleus is specific binding.

73. The method of any one of claims 1-72, wherein at least some of the binding of the nucleic acid probes within the nucleus is via Watson-Crick base pairing.

74. The method of any one of claims 1-73, comprising determining binding of the nucleic acid probes within the sample at a resolution better than 300 nm.

75. The method of any one of claims 1-74, comprising determining binding of the nucleic acid probes within the sample at a resolution better than 100 nm.

76. The method of any one of claims 1-75, comprising determining binding of the nucleic acid probes within the sample at a resolution better than 80 nm.

77. The method of any one of claims 1-76, comprising determining binding of the nucleic acid probes within the sample at a resolution better than 50 nm.

78. The method of any one of claims 1-77, wherein the sample is a cell.

79. The method of claim 78, wherein the cell is fixed.

80. The method of any one of claims 1-79, comprising determining binding of the nucleic acid probes by imaging at least a portion of the sample.

81. The method of any one of claims 1-80, comprising determining binding of the nucleic acid probes using an optical imaging technique.

82. The method of any one of claims 1-81, comprising determining binding of the nucleic acid probes using a fluorescence imaging technique.

83. The method of any one of claims 1-82, comprising determining binding of the nucleic acid probes using a multi-color fluorescence imaging technique.

84. The method of any one of claims 1-83, comprising determining binding of the nucleic acid probes using a super-resolution fluorescence imaging technique.

85. The method of any one of claims 1-84, comprising determining binding of the nucleic acid probes using stochastic optical reconstruction microscopy (STORM).

86. A method, comprising:
determining positions of nascent RNA within a nucleus;
applying RNAse to the nucleus; and
determining positions of DNA within the nucleus.

87. A method comprising:
determining positions of nascent RNA within a nucleus;
determining positions of DNA within the nucleus; and
determining positions of a protein within the nucleus.

88. A method comprising:
determining positions of nascent RNA within a nucleus;
determining positions of DNA within the nucleus; and
determining positions of a nucleic acid within the nucleus, wherein the nucleic acid is not the nascent RNA or the DNA.

89. A method, comprising:
using MERFISH to image chromatin in a cell.

90. The method of claim 89, comprising imaging the chromatin in 3 dimensions.

91. The method of any one of claim 89 or 90, further comprising determining a nuclear structure of the cell.

92. The method of any one of claims 89-91, further comprising determining transcriptional activity in the cell.

93. The method of claim 92, further comprising determining at least 100 distinct transcription sites within the cell.

94. The method of any one of claims 89-93, further comprising determining at least 100 distinct genomic loci within the cell.

95. A method, comprising:
imaging at least 100 distinct genomic loci in a single cell.

96. A method, comprising:
associating a plurality of nucleic acid targets of a genome with a plurality of codewords;
exposing a sample containing a cell suspected of containing the genome to a plurality of nucleic acid probes, wherein at least some of the plurality of nucleic acid probes comprises a first portion comprising a target sequence and a second portion comprising one or more readout sequences, wherein each readout sequence represents a value of a position within the plurality of codewords;
exposing the sample to a round of one or more adaptors, wherein each adaptor comprises a first portion substantially complementary to one of the readout sequences, and a second portion comprising one identification sequence;
exposing the sample to a round of one or more readout probes to determine one or more identification sequences, wherein each readout probe comprises a first portion comprising a sequence substantially complementary to one of the identification sequences, and a second portion comprising a signaling entity;
determining the signaling entity in at least some locations in the sample; and
inactivating the signaling entity in at least some locations in the sample;
repeating the steps of exposing the sample to a round of one or more adaptors and one or more readout probes, determining the signaling entity, and inactivating the signaling entity, wherein one or more distinct signaling entities are used in each of the rounds;
determining codewords at the locations based on determining the signaling entity in the sample; and
determining nucleic acid targets in the sample based on the codewords.

97. The method of claim 96, wherein determining nucleic acid targets comprises determine spatial positions of the nucleic acid targets.

98. The method of claim 97, further comprising determining three-dimensional organization of the chromatin or genome from the spatial positions of the nucleic acid targets.

99. The method of any one of claims 96-98, wherein no more than 10 signaling entities are used in all of the rounds.

100. The method of any one of claims 96-99, wherein no more than 5 signaling entities are used in all of the rounds.

101. The method of any one of claims 96-100, wherein no more than 3 signaling entities are used in all of the rounds.

102. The method of any one of claims 96-101, wherein exposing the sample to a round of one or more readout probes further comprises exposing the sample to blocking probes comprising a sequence substantially complementary to one of the identification sequences determined in a previous round.

103. The method of any one of claims 96-102, comprising determining at least 20 identification sequences using no more than the 10 signaling entities.

104. The method of any one of claims 96-103, comprising determining at least 100 identification sequences using no more than the 10 signaling entities.

105. The method of any one of claims 96-104, comprising determining at least 1,000 identification sequences using no more than the 10 signaling entities.

106. The method of any one of claims 96-105, comprising determining at least 100 identification sequences using no more than 3 signaling entities.

107. The method of any one of claims 96-106, comprising determining at least 1,000 identification sequences using no more than 3 signaling entities.

108. The method of any one of claims 96-107, comprising fixing the sample after exposing the nucleus of the cell to the plurality of nucleic acid probes.

109. The method of any one of claims 96-108, comprising fixing the sample between the rounds of exposing the sample to one or more readout probes.

110. The method of any one of claims 96-109, comprising fixing the sample at least 5 times.

111. The method of any one of claims 96-110, comprising fixing the sample at least once every 4 days.

112. The method of any one of claims 96-111, comprising fixing the sample at least once every 2 days.

113. The method of any one of claims 96-112, comprising fixing the sample at least once every 24 hours.

114. The method of any one of claims 96-113, comprising fixing the sample at least once every 12 hours.

115. The method of any one of claims 96-114, comprising fixing the sample at least once every 6 hours.

116. The method of any one of claims 96-115, comprising fixing the sample using formaldehyde.

117. The method of any one of claims 96-116, comprising repeating the repeating step at least 10 times.

118. The method of any one of claims 96-117, comprising repeating the repeating step at least 50 times.

119. The method of any one of claims 96-118, comprising repeating the repeating step at least 100 times.

120. The method of any one of claims 96-119, comprising repeating the repeating step at least 200 times.

121. The method of any one of claims 96-120, comprising exposing the sample to a round of one or more readout probes that is identical to a previous round of one or more readout probes.

122. The method of claim 121, further comprising determining degradation of the sample based on the two identical rounds of one or more readout probes.

123. The method of any one of claims 96-122, wherein the readout sequences do not exhibit specific binding towards the genome.

124. The method of any one of claims 96-123, wherein the readout sequences do not exhibit specific binding towards each other.

125. The method of any one of claims 96-124, wherein the identification sequences do not exhibit specific binding towards each other.

126. The method of any one of claims 96-125, wherein the identification sequences do not exhibit specific binding towards the genome.

127. The method of any one of claims 96-126, comprising exposing the sample to at least 50 distinguishable nucleic acid probes.

128. The method of any one of claims 96-127, comprising exposing the sample to at least 100 distinguishable nucleic acid probes.

129. The method of any one of claims 96-128, comprising exposing the sample to at least 1,000 distinguishable nucleic acid probes.

130. The method of any one of claims 96-129, comprising exposing the sample to at least 10,000 distinguishable nucleic acid probes.

131. The method of any one of claims 96-130, comprising exposing the sample to at least 100,000 distinguishable nucleic acid probes.

132. The method of any one of claims 96-131, comprising exposing the sample to at least 1,000,000 distinguishable nucleic acid probes.

133. The method of any one of claims 96-132, further comprising determining nascent RNA within the nucleus of the cell.

134. The method of any one of claims 96-133, further comprising determining nuclear speckles within the nucleus of the cell.

135. The method of any one of claims 96-134, further comprising determining nucleoli within the nucleus of the cell.

136. The method of any one of claims 96-135, further comprising determining nuclear lamina within the nucleus of the cell.

137. The method of any one of claims 96-136, further comprising determining other protein and nucleic acids species within the nucleus of the cell.

138. The method of any one of claims 96-137, further comprising determining other protein and nucleic acids species within the sample.

139. The method of any one of claims 96-138, wherein at least some of the signaling entities are fluorescent.

140. The method of any one of claims 96-139, comprising imaging the sample to determine the signaling entity in at least some locations in the sample.

141. A method, comprising:
associating a plurality of nucleic acid targets of a genome with a plurality of codewords;
exposing a sample containing a cell suspected of containing the genome to a plurality of nucleic acid probes, wherein at least some of the plurality of nucleic acid probes comprises a first portion comprising a target sequence and a second portion comprising one or more readout sequences, wherein each readout sequence represents a value of a position within the plurality of codewords;
exposing the sample to a round of one or more adaptors, wherein each adaptor comprises a first portion substantially complementary to one of the readout sequences, and a second portion comprising one identification sequence;
exposing the sample to a round of one or more readout probes to determine one or more identification sequences, wherein each readout probe comprises a first portion comprising a sequence substantially complementary to one of the identification sequences, and a second portion comprising a signaling entity;
determining the signaling entity in at least some locations in the sample; and
inactivating the signaling entity in at least some locations in the sample;
repeating the steps of exposing the sample to a round of one or more adaptors and one or more readout probes, determining the signaling entity, and inactivating the signaling entity, wherein at least one of the signaling entities is used in more than one of the rounds;
determining codewords at the locations based on determining the signaling entity in the sample; and
determining nucleic acid targets in the sample based on the codewords.

142. A method, comprising:
exposing a sample containing a cell suspected of containing the genome to a plurality of nucleic acid probes, wherein at least some of the plurality of nucleic acid probes comprises a first portion comprising a target sequence and a second portion comprising one or more readout sequences;
exposing the sample to a round of one or more adaptors, wherein each adaptor comprises a first portion substantially complementary to one of the readout sequences, and a second portion comprising one identification sequence;
exposing the sample to a round of one or more readout probes to determine one or more identification sequences, wherein each readout probe comprises a first portion comprising a sequence substantially complementary to one of the identification sequences, and a second portion comprising a signaling entity;
determining the signaling entity in at least some locations in the sample; and
inactivating the signaling entity in at least some locations in the sample;
repeating the steps of exposing the sample to a round of one or more adaptors and one or more readout probes, determining the signaling entity, and inactivating the signaling entity, wherein one or more distinct signaling entities are used in each of the rounds;
determining nucleic acid targets in the sample based on the signaling entities determined in each round.

143. The method of claim 142, wherein determining nucleic acid targets comprises determine the spatial positions of the nucleic acid targets.

144. The method of claim 143, further comprising determining the three-dimensional organization of the chromatin or genome from the spatial positions of the nucleic acid targets.

145. The method of any one of claims 142-144, wherein no more than 10 signaling entities are used in all of the rounds.

146. The method of any one of claims 142-145, wherein no more than 5 signaling entities are used in all of the rounds.

147. The method of any one of claims 142-146, wherein no more than 3 signaling entities are used in all of the rounds.

148. The method of any one of claims 142-147, wherein no more than 2 signaling entities are used in all of the rounds.

149. The method of any one of claims 142-148, wherein no more than 1 signaling entity is used in all of the rounds.

150. The method of any one of claims 142-149, wherein exposing the sample to a round of one or more readout probes further comprises exposing the sample to blocking probes comprising a sequence substantially complementary to one of the identification sequences determined in a previous round.

151. The method of any one of claims 142-150, comprising determining at least 20 identification sequences using no more than the 10 signaling entities.

152. The method of any one of claims 142-151, comprising determining at least 100 identification sequences using no more than the 10 signaling entities.

153. The method of any one of claims 142-152, comprising determining at least 1,000 identification sequences using no more than the 10 signaling entities.

154. The method of any one of claims 142-153, comprising determining at least 100 identification sequences using no more than 3 signaling entities.

155. The method of any one of claims 142-154, comprising determining at least 1,000 identification sequences using no more than 3 signaling entities.

156. The method of any one of claims 142-155, comprising fixing the sample after exposing the nucleus of the cell to the plurality of nucleic acid probes.

157. The method of any one of claims 142-156, comprising fixing the sample between the rounds of exposing the sample to one or more readout probes.

158. The method of any one of claims 142-157, comprising fixing the sample at least 5 times.

159. The method of any one of claims 142-158, comprising fixing the sample at least once every 4 days.

160. The method of any one of claims 142-159, comprising fixing the sample at least once every 2 days.

161. The method of any one of claims 142-160, comprising fixing the sample at least once every 24 hours.

162. The method of any one of claims 142-161, comprising fixing the sample at least once every 12 hours.

163. The method of any one of claims 142-162, comprising fixing the sample at least once every 6 hours.

164. The method of any one of claims 142-163, comprising fixing the sample using formaldehyde.

165. The method of any one of claims 142-164, comprising repeating the repeating step at least 10 times.

166. The method of any one of claims 142-165, comprising repeating the repeating step at least 50 times.

167. The method of any one of claims 142-166, comprising repeating the repeating step at least 100 times.

168. The method of any one of claims 142-167, comprising repeating the repeating step at least 200 times.

169. The method of any one of claims 142-168, comprising exposing the sample to a round of one or more readout probes that is identical to a previous round of one or more readout probes.

170. The method of claim 169, further comprising determining degradation of the sample based on the two identical rounds of one or more readout probes.

171. The method of any one of claims 142-170, wherein the readout sequences do not exhibit specific binding towards the genome.

172. The method of any one of claims 142-171, wherein the readout sequences do not exhibit specific binding towards each other.

173. The method of any one of claims 142-172, wherein the identification sequences do not exhibit specific binding towards each other.

174. The method of any one of claims 142-173, wherein the identification sequences do not exhibit specific binding towards the genome.

175. The method of any one of claims 142-174, comprising exposing the sample to at least 50 distinguishable nucleic acid probes.

176. The method of any one of claims 142-175, comprising exposing the sample to at least 100 distinguishable nucleic acid probes.

177. The method of any one of claims 142-176, comprising exposing the sample to at least 1,000 distinguishable nucleic acid probes.

178. The method of any one of claims 142-177, comprising exposing the sample to at least 10,000 distinguishable nucleic acid probes.

179. The method of any one of claims 142-178, comprising exposing the sample to at least 100,000 distinguishable nucleic acid probes.

180. The method of any one of claims 142-179, comprising exposing the sample to at least 1,000,000 distinguishable nucleic acid probes.

181. The method of any one of claims 142-180, wherein at least some of the signaling entities are fluorescent.

182. The method of any one of claims 142-181, comprising imaging the sample to determine the signaling entity in at least some locations in the sample.

183. The method of any one of claims 142-182, further comprising determining nascent RNA within the nucleus of the cell.

184. The method of any one of claims 142-183, further comprising determining nuclear speckles within the nucleus of the cell.

185. The method of any one of claims 142-184, further comprising determining nucleoli within the nucleus of the cell.

186. The method of any one of claims 142-185, further comprising determining nuclear lamina within the nucleus of the cell.

187. The method of any one of claims 142-186, further comprising determining other protein and nucleic acids species within the nucleus of the cell.

188. The method of any one of claims 142-187, further comprising determining other protein and nucleic acids species within the sample.

189. A method, comprising:
exposing a sample containing a cell suspected of containing the genome to a plurality of nucleic acid probes, wherein at least some of the plurality of nucleic acid probes comprises a first portion comprising a target sequence and a second portion comprising one or more readout sequences;
exposing the sample to a round of one or more readout probes to determine one or more readout sequences, wherein each readout probe comprises a first portion comprising a sequence substantially complementary to one of the readout sequences, and a second portion comprising a signaling entity;
determining the signaling entity in at least some locations in the sample; and
inactivating the signaling entity in at least some locations in the sample;
repeating the steps of exposing the sample to a round of one or more readout probes, determining the signaling entity, and inactivating the signaling entity, wherein one or more distinct signaling entities are used in each of the rounds;
determining nucleic acid targets in the sample based on the signaling entities determined in each round.

190. The method of claim 189, wherein determining nucleic acid targets comprises determine the spatial positions of the nucleic acid targets.

191. The method of claim 190, further comprising determining the three-dimensional organization of the chromatin or genome from the spatial positions of the nucleic acid targets.

192. The method of any one of claims 189-191, wherein no more than 10 signaling entities are used in all of the rounds.

193. The method of any one of claims 189-192, wherein no more than 5 signaling entities are used in all of the rounds.

194. The method of any one of claims 189-193, wherein no more than 3 signaling entities are used in all of the rounds.

195. The method of any one of claims 189-194, wherein no more than 2 signaling entities are used in all of the rounds.

196. The method of any one of claims 189-195, wherein no more than 1 signaling entity are used in all of the rounds.

197. The method of any one of claims 189-196, wherein exposing the sample to a round of one or more readout probes further comprises exposing the sample to blocking probes comprising a sequence substantially complementary to one of the identification sequences determined in a previous round.

198. The method of any one of claims 189-197, comprising fixing the sample after exposing the nucleus of the cell to the plurality of nucleic acid probes.

199. The method of any one of claims 189-198, comprising fixing the sample between the rounds of exposing the sample to one or more readout probes.

200. The method of any one of claims 189-199, comprising fixing the sample at least 5 times.

201. The method of any one of claims 189-200, comprising fixing the sample at least once every 4 days.

202. The method of any one of claims 189-201, comprising fixing the sample at least once every 2 days.

203. The method of any one of claims 189-202, comprising fixing the sample at least once every 24 hours.

204. The method of any one of claims 189-203, comprising fixing the sample at least once every 12 hours.

205. The method of any one of claims 189-204, comprising fixing the sample at least once every 6 hours.

206. The method of any one of claims 189-205, comprising fixing the sample using formaldehyde.

207. The method of any one of claims 189-206, comprising repeating the repeating step at least 10 times.

208. The method of any one of claims 189-207, comprising repeating the repeating step at least 50 times.

209. The method of any one of claims 189-208, comprising repeating the repeating step at least 100 times.

210. The method of any one of claims 189-209, comprising repeating the repeating step at least 200 times.

211. The method of any one of claims 189-210, comprising exposing the sample to a round of one or more readout probes that is identical to a previous round of one or more readout probes.

212. The method of claim 211, further comprising determining degradation of the sample based on the two identical rounds of one or more readout probes.

213. The method of any one of claims 189-212, wherein the readout sequences do not exhibit specific binding towards the genome.

214. The method of any one of claims 189-213, wherein the readout sequences do not exhibit specific binding towards each other.

215. The method of any one of claims 189-214, comprising exposing the sample to at least 50 distinguishable nucleic acid probes.

216. The method of any one of claims 189-215, comprising exposing the sample to at least 100 distinguishable nucleic acid probes.

217. The method of any one of claims 189-216, comprising exposing the sample to at least 1,000 distinguishable nucleic acid probes.

218. The method of any one of claims 189-217, comprising exposing the sample to at least 10,000 distinguishable nucleic acid probes.

219. The method of any one of claims 189-218, comprising exposing the sample to at least 100,000 distinguishable nucleic acid probes.

220. The method of any one of claims 189-219, comprising exposing the sample to at least 1,000,000 distinguishable nucleic acid probes.

221. The method of any one of claims 189-220, wherein at least some of the signaling entities are fluorescent.

222. The method of any one of claims 189-221, comprising imaging the sample to determine the signaling entity in at least some locations in the sample.

223. The method of any one of claims 189-222, further comprising determining nascent RNA within the nucleus of the cell.

224. The method of any one of claims 189-223, further comprising determining nuclear speckles within the nucleus of the cell.

225. The method of any one of claims 189-224, further comprising determining nucleoli within the nucleus of the cell.

226. The method of any one of claims 189-225, further comprising determining nuclear lamina within the nucleus of the cell.

227. The method of any one of claims 189-226, further comprising determining other protein and nucleic acids species within the nucleus of the cell.

228. The method of any one of claims 189-227, further comprising determining other protein and nucleic acids species within the sample.

229. A method, comprising:
exposing a sample containing a cell suspected of containing the genome to a round of a plurality of nucleic acid probes, wherein at least some of the plurality of nucleic acid probes comprises a first portion comprising a target sequence and a second portion comprising a signaling entity;
determining the signaling entity in at least some locations in the sample; and
inactivating the signaling entity in at least some locations in the sample;
repeating the steps of exposing the sample to a round of a plurality of nucleic acid probes, determining the signaling entity, and inactivating the signaling entity, wherein one or more distinct signaling entities are used in each of the rounds;
determining nucleic acid targets in the sample based on the signaling entities determined in each round.

230. The method of claim 229, wherein determining nucleic acid targets comprises determine the spatial positions of the nucleic acid targets.

231. The method of claim 230, further comprising determining the three-dimensional organization of the chromatin or genome from the spatial positions of the nucleic acid targets.

232. The method of any one of claims 229-231, wherein no more than 10 signaling entities are used in all of the rounds.

233. The method of any one of claims 229-232, wherein no more than 5 signaling entities are used in all of the rounds.

234. The method of any one of claims 229-233, wherein no more than 3 signaling entities are used in all of the rounds.

235. The method of any one of claims 229-234, wherein no more than 2 signaling entities are used in all of the rounds.

236. The method of any one of claims 229-235, wherein no more than 1 signaling entity are used in all of the rounds.

237. The method of any one of claims 229-236, comprising fixing the sample after exposing the nucleus of the cell to the plurality of nucleic acid probes.

238. The method of any one of claims 229-237, comprising fixing the sample between the rounds of exposing the sample to one or more readout probes.

239. The method of any one of claims 229-238, comprising fixing the sample at least 5 times.

240. The method of any one of claims 229-239, comprising fixing the sample at least once every 4 days.

241. The method of any one of claims 229-240, comprising fixing the sample at least once every 2 days.

242. The method of any one of claims 229-241, comprising fixing the sample at least once every 24 hours.

243. The method of any one of claims 229-242, comprising fixing the sample at least once every 12 hours.

244. The method of any one of claims 229-243, comprising fixing the sample at least once every 6 hours.

245. The method of any one of claims 229-244, comprising fixing the sample using formaldehyde.

246. The method of any one of claims 229-245, comprising repeating the repeating step at least 10 times.

247. The method of any one of claims 229-246, comprising repeating the repeating step at least 50 times.

248. The method of any one of claims 229-247, comprising repeating the repeating step at least 100 times.

249. The method of any one of claims 229-248, comprising repeating the repeating step at least 200 times.

250. The method of any one of claims 229-249, comprising exposing the sample to at least 50 distinguishable nucleic acid probes.

251. The method of any one of claims 229-250, comprising exposing the sample to at least 100 distinguishable nucleic acid probes.

252. The method of any one of claims 229-251, comprising exposing the sample to at least 1,000 distinguishable nucleic acid probes.

253. The method of any one of claims 229-252, comprising exposing the sample to at least 10,000 distinguishable nucleic acid probes.

254. The method of any one of claims 229-253, comprising exposing the sample to at least 100,000 distinguishable nucleic acid probes.

255. The method of any one of claims 229-254, comprising exposing the sample to at least 1,000,000 distinguishable nucleic acid probes.

256. The method of any one of claims 229-255, wherein at least some of the signaling entities are fluorescent.

257. The method of any one of claims 229-256, comprising imaging the sample to determine the signaling entity in at least some locations in the sample.

258. The method of any one of claims 229-257, further comprising determining nascent RNA within the nucleus of the cell.

259. The method of any one of claims 229-258, further comprising determining nuclear speckles within the nucleus of the cell.

260. The method of any one of claims 229-259, further comprising determining nucleoli within the nucleus of the cell.

261. The method of any one of claims 229-260, further comprising determining nuclear lamina within the nucleus of the cell.

262. The method of any one of claims 229-261, further comprising determining other protein and nucleic acids species within the nucleus of the cell.

263. The method of any one of claims 229-262, further comprising determining other protein and nucleic acids species within the sample.
